SEQUENCE LISTING (11 sequences)

SEQ ID NO: 1 - protein, 594 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 2 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 3 - protein, 593 aa, synthetic construct
GSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 4 - protein, 593 aa, synthetic construct
GSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 5 - RNA, 5124 nt, synthetic construct
  source 1..5124: mol_type = other RNA
acatttgcttctgacacaactgtgttcactagcaacctcaaacagacaccaagcttgccaccatggctcccaagaagaagcggaaggtcgagggcatcaagagcaacatcagcctgctgaaggacgagctgagaggccagatcagccacatctcccacgagtacctgagcctgatcgacctggccttcgaccccaagcagaaccggctgttcgagatgaaggtgctggaactgctggtcaacgagtacggcttcaagggcagacacctcggcggcagcagaaagcctgatggcatcgtgtacagcaccacactcgaggacaacttcggcatcatcgtggacaccaaggcctacagcgagggctacagcctgcctatctctcaggccgacgagatggaaagatacgtgcgcgagaacagcaaccgcgacgaggaagtgaaccccaacaagtggtgggagaacttcagcgaggaagtcaaaaagtactacttcgtgttcatcagcggcagctttaagggcaagttcgaggaacagctgcggcggctgtctatgaccacaggcgttaacggcagcgccgtgaacgtggtcaatctgctgctgggcgccgagaagattagaagcggcgagatgaccatcgaggaactggaacgggccatgttcaacaacagcgagttcatcctgaagtacggcggaggcggcagcgacaagaagtactctatcggactggccatcggcaccaactctgttggatgggccgtgatcaccgacgagtacaaggtgcccagcaagaaattcaaagtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggaaatatcgtggacgaggtggcctaccacgagaagtaccccaccatctaccacctgagaaagaaactggtggacagcaccgacaaggccgacctgcgactgatctatctggccctggctcacatgatcaagttccggggccacttcctgatcgagggcgacctgaatcctgacaactccgacgtggacaagctgttcatccagctggtgcagacctacaatcagctgttcgaagagaatcccatcaacgcctctggcgtggacgccaaagccatcctgtctgccagactgagcaagagcagacggctggaaaacctgatcgctcagctgcccggcgagaagaagaatggcctgttcggcaacctgattgccctgtctctgggcctgacacctaacttcaagtccaacttcgatctggccgaggatgccaaactgcagctgtccaaggacacctacgacgacgacctggataacctgctggcccagatcggcgatcagtacgccgacttgtttctggccgccaagaacctgtctgacgccatcctgctgagcgacatcctgagagtgaacaccgagatcacaaaggcccctctgagcgcctctatgatcaagagatacgacgagcaccaccaggatctgaccctgctgaaagctctcgtcaggcagcagctgccagagaagtacaaagagattttcttcgaccagagcaagaacggctacgccggctacattgatggcggagccagccaagaggaattctacaagttcatcaagcccatcctcgagaagatggacggcacagaggaactgctcgtgaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaagaggtggtggacaagggcgccagcgctcagtccttcatcgagcggatgaccaatttcgacaagaatctgcccaacgagaaagtgctgcccaagcactccctgctgtacgagtacttcaccgtgtacaacgagctgaccaaagtgaaatacgtgaccgagggaatgagaaagcccgcctttctgtccggcgagcagaaaaaggccatcgtggatctgctgttcaagaccaaccggaaagtgaccgtgaagcagctgaaagaggactacttcaagaaaatcgagtgcttcgactccgtggaaatcagcggcgtggaagatcggttcaatgccagcctgggcacataccacgatctgctgaaaattatcaaggacaaggacttcctggacaacgaggaaaacgaggacatccttgaggacatcgtgctgaccctgacactgttcgaggacagagagatgatcgaggaaaggctgaaaacatacgcccacctgttcgacgacaaagtcatgaagcaactgaagcggcggcgctacacaggctggggcagactgtctagaaagctgatcaacggcatccgggacaagcagtccggcaagaccatcctggactttctgaagtccgacggcttcgccaacagaaacttcatgcagctgattcacgacgacagcctcaccttcaaagaggacattcagaaggcccaggtttccggccagggcgattctctgcacgagcacattgccaatctggccggctctcccgccattaagaagggcattctgcagacagtgaaagtggtggatgagctggtcaaagtgatggggagacacaagcccgagaacatcgtgatcgaaatggccagagagaaccagaccacacagaagggccagaagaactcccgcgagagaatgaagcggatcgaagagggaatcaaagagctggggagccagatcctgaaagaacaccccgtggaaaacacccagctgcagaacgagaagctgtacctgtactacctccagaacggccgggatatgtacgtggaccaagagctggacatcaaccgcctgagcgactacgatgtggacgctatcgtgccccagtcttttctgaaagatgactccatcgacaacaaggtgctgaccagaagcgataagaaccggggcaagagcgacaacgtgccctctgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacaccaaatacgatgagaacgacaaactgatccgggaagtgaaagtcatcaccctgaagtccaagctggtgtccgatttccggaaggatttccagttctacaaagtgcgggaaatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggaaaggctaccgccaaatacttcttctactccaacatcatgaactttttcaagacagagatcaccctcgccaacggcgagatcagaaagcggcctctgatcgagacaaacggcgaaaccggcgagattgtgtgggataagggcagagactttgccacagtgcggaaggtgctcagcatgccccaagtgaatatcgtgaaaaagaccgaggtgcagacaggcggcttcagcaaagagtccattctgcctaagcggaactccgacaagctgatcgcccggaagaaggactgggaccccaagaaatacggcggcttcgatagccctaccgtggcctattctgtgctggtggtggccaaagtggaaaagggaaagtccaagaagctcaagagcgtcaaagaactcctgggcatcaccatcatggaacggtccagcttcgagaagaaccctatcgactttctggaagccaagggctacaaagaagtcaagaaggacctgatcatcaagctccccaagtacagcctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcagaagcagcttttcgtcgagcagcacaagcactacctggacgagatcatcgagcagatctccgagttctccaagagagtgatcctggccgacgccaacctggacaaggttctgtccgcctacaacaagcaccgggataagcccatcagagagcaggccgagaatatcatccacctgtttaccctgaccaacctgggagcccctgccgccttcaagtacttcgacaccaccatcgaccggaagcgctacaccagcaccaaagaagtgctggacgccacactgatccaccagagcatcaccggcctgtacgagacacggatcgatctgtctcagcttggaggcgacggcagccctaagaagaagagaaaggtttccagctaataaggcggccgccctcgccccggacctgccctcccgccaggtgcacccacctgcaataaatgcagcgaagccgggagaattccctcgccccggacctgccctcccgccaggtgcacccacctgcaataaatgcagcgaagccgggagcggccgcggatccccgggtaccgaattcgatatctctatagtgtcacctaaatttaattaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 20 nt, synthetic construct
  source 1..20: mol_type = other RNA
cctgggcaccagcgccggtg

SEQ ID NO: 7 - RNA, 20 nt, synthetic construct
  source 1..20: mol_type = other RNA
gatgctcgccctcccgtccc

SEQ ID NO: 8 - DNA, 4609 bp, synthetic construct
  source 1..4609: mol_type = other DNA
tgtacatagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgaagatctggtaccgatcacatatgcctttaattaaacactggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctggtctcatgacgcgtagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggta

SEQ ID NO: 9 - DNA, 4609 bp, synthetic construct; identical to SEQ ID NO: 8
  source 1..4609: mol_type = other DNA

SEQ ID NO: 10 - DNA, 5179 bp, synthetic construct
  source 1..5179: mol_type = other DNA
tgtacatacatccggagtgcactctagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccatggccggcttttgggttggaacagctcctctggttgccgctgggagaagaggaagatggcctcctcagcagctgatgctgtctgccgctctgagaaccctgaaacacgtgctgtactacagccggcagtgcctgatggtgtccagaaatctgggcagcgtgggctacgaccccaacgagaaaaccttcgacaagatcctggtggccaaccggggagagatcgcctgcagagtgatccggacctgcaagaagatgggcatcaagaccgtggccatccactccgatgtggatgcctctagcgtgcacgtgaaaatggccgatgaggccgtgtgtgtgggccctgctcctacaagcaagagctacctgaacatggacgccatcatggaagctatcaagaaaacaagagcccaggccgtgcatcccggctacggatttctgagcgagaacaaagaattcgcccggtgcctggccgccgaggatgtggtttttatcggccctgacacacacgccatccaggccatgggagacaagatcgagtctaagctgctggccaagaaagccgaagtgaacacaatccccggcttcgacggcgtggtcaaggatgctgaagaagccgtgcggatcgccagagaaatcggctaccccgtgatgatcaaagcctctgctggcggaggcggcaagggaatgagaatcgcctgggacgacgaagagacacgcgacggctttagactgagcagccaagaagccgccagctccttcggagatgacagactgctgatcgagaagttcatcgacaaccccagacacatcgagatccaggtgctgggcgacaagcacggaaatgccctgtggctgaacgagagagagtgcagcatccagcggcggaaccagaaagtggtggaagaggcccctagcatcttcctggacgccgaaactcggagagccatgggcgaacaagctgtggctcttgccagagccgtgaagtacagctctgccggcaccgtggaatttctggtggacagcaagaagaacttctacttcctcgagatgaacacccggctgcaggtcgagcaccctgtgaccgagtgtatcacaggcctggacctggtgcaagagatgatcagagtggccaagggctaccctctgagacacaagcaggccgacatccggatcaatggctgggccgttgagtgcagagtgtacgctgaggacccctacaagagcttcggcctgccttctatcggccggctgagccagtatcaagaaccccttcatctgcccggcgtcagagtggattctggcatccagcctggcagcgacatcagcatctactacgaccctatgatctccaagctgatcacctacggcagcgaccggacagaggccctgaagagaatggctgacgccctggacaactacgtgatcagaggcgtgacccacaatatcgccctgctgcgggaagtgatcatcaacagcagattcgtgaaaggcgatatcagcaccaagtttctgtccgacgtgtaccccgacggcttcaagggacacatgctgaccaagagcgagaagaaccagctgctcgccattgccagcagcctgttcgtggcctttcagctgagagcccagcacttccaagagaacagcagaatgcccgtgatcaagcccgatatcgccaactgggagctgagcgtgaagctgcacgataaggtgcacacagtggtggccagcaacaacggctccgtgttcagcgtggaagtggacggcagcaagctgaacgtgacctccacctggaatctggcctctccactgctgtccgtgtctgtggatggcacccagagaaccgtgcagtgtctgagcagagaagcaggcggcaatatgagcatccagtttctgggcaccgtgtacaaagtgaacatcctgaccagactggccgctgagctgaacaagttcatgctggaaaaagtgaccgaggacaccagcagcgtgctgagatctcctatgcctggtgtcgtggtggccgtgtcagtgaaacctggggatgctgtggccgagggccaagagatctgtgtgatcgaggccatgaagatgcagaacagcatgaccgccggcaagaccggcacagtgaagtctgtgcattgtcaggccggcgatacagtcggagaaggcgatctgctggtggaactggaatacccatacgatgttccagattacgcttgaggtaccgatcacatatgcctttaattaaacactggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctggtctcatgacgcgtagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggta

SEQ ID NO: 11 - protein, 1591 aa, synthetic construct
MAPKKKRKVEGIKSNISLLKDELRGQISHISHEYLSLIDLAFDPKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

(19) United States
(12) Patent Application Publication
(10) Pub. No.: US 2026/0069539 A1
(43) Pub. Date: Mar. 12, 2026

(54) LIPIDOID COMPOUNDS AND RELATED COMPOSITIONS AND USES

(71) Applicant: Poseida Therapeutics, Inc., San Diego, CA (US)

(72) Inventors: Joshua Rychak, San Diego, CA (US); Khalid A. Hajj, San Diego, CA (US); Minghao Xu, San Diego, CA (US); Ling Li, San Diego, CA (US); Lijun Huang, San Diego, CA (US); Gopi Nath Vemuri, San Diego, CA (US); Alicia M. Davis, San Diego, CA (US); Hua Wang, San Diego, CA (US); Michael J. Bennett, San Diego, CA (US)

(21) Appl. No.: 19/272,808

(22) Filed: Oct. 7, 2025

Related U.S. Application Data
(63) Continuation of application No. PCT/US2024/012245, filed on Jan. 19, 2024.
(60) Provisional application No. 63/480,821, filed on Jan. 20, 2023.
(60) Provisional application No. 63/515,987, filed on Jul. 27, 2023.
(60) Provisional application No. 63/600,961, filed on Nov. 20, 2023.

(51) Int. Cl. A61K 9/127; A61K 9/51; A61K 31/7088; C07C 229/16; C12N 15/88

(52) U.S. Cl. CPC A61K 9/127; A61K 9/5123; A61K 31/7088; C07C 229/16; C12N 15/88; C07C 2601/14

(57) ABSTRACT

Compositions comprising lipidoid compounds, methods of preparing such compositions, and the use of these compositions in gene delivery applications are disclosed.

### RELATED APPLICATIONS

This application is a continuation of International Application No. PCT/US2024/012245, filed Jan. 19, 2024, which claims the benefit of U.S. Provisional Application No. 63/480,821, filed on Jan. 20, 2023, U.S. Provisional Application No. 63/515,987, filed on Jul. 27, 2023, and U.S. Provisional Application No. 63/600,961, filed on Nov. 20, 2023. The contents and disclosure of each of the aforementioned patent applications are incorporated herein by reference in their entireties.

### SEQUENCE LISTING

The Sequence Listing XML associated with this application is provided electronically in XML format and is hereby incorporated by reference into the specification. The name of the XML file containing the Sequence Listing XML is “000218-0137-101-SL.xml”. The XML file is 34,015 bytes in size, was created on Jul. 17, 2025, and is being submitted electronically via USPTO Patent Center.

### FIELD

The present invention relates generally to lipidoid compounds, compositions containing such compounds, methods of preparing these compounds, and the use of these compositions in gene delivery.

### BACKGROUND

There has been a long-felt but unmet need in the art for compositions and methods for delivering nucleic acids to cells and for genetically modifying cells in vivo, ex vivo and in vitro. Widely accepted gene delivery and genetic modification techniques, such as the use of viral vectors, including AAVs, can cause acute toxicity and harmful side-effects in patients. The present disclosure provides improved compositions, methods and kits for the delivery of nucleic acids to various types of cells, including hepatocytes, in vivo, ex vivo and in vitro. More specifically, the present disclosure provides improved lipid nanoparticle compositions and methods of using the same. These lipid nanoparticle compositions and methods allow for the delivery of nucleic acids to cells with high efficiency and low toxicity. Thus, the compositions and methods of the present disclosure have wide applicability to a diverse number of fields, including gene therapy.

### SUMMARY

In some aspects, provided are novel compounds. In one aspect, the novel compound is a compound of Formula (I):

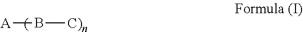

or a salt thereof, wherein:
A is:

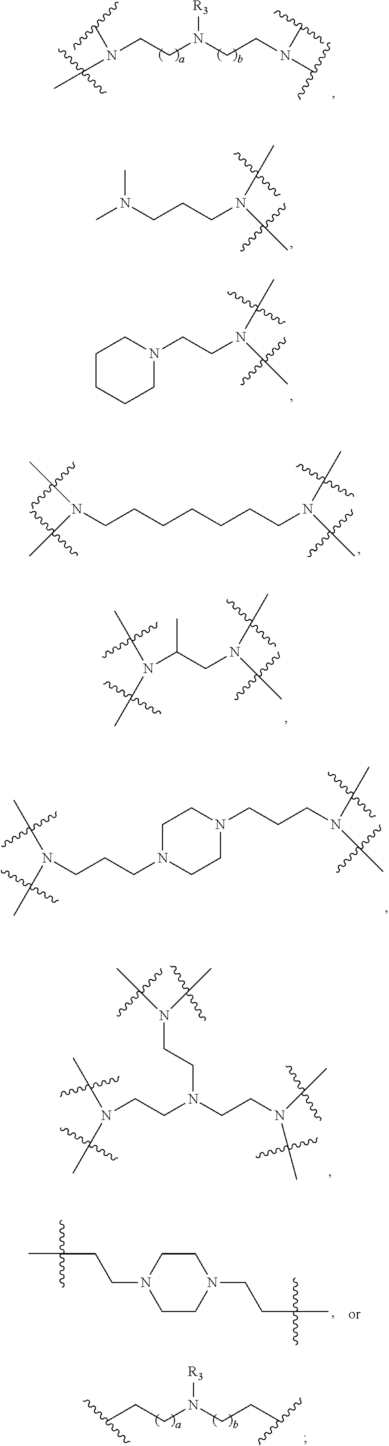

each B is independently

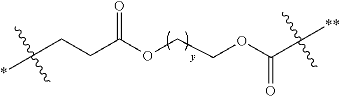

in which * indicates attachment to A and ** indicates attachment to C,
each C is independently

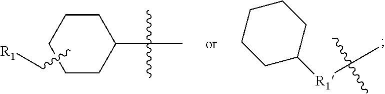

n is an integer ranging from 2 to 6;
a is an integer ranging from 1 to 5;
b is an integer ranging from 1 to 5;
each y is independently an integer ranging from 1 to 10;
each R₁ is independently unbranched C₁-C₁₈ alkyl optionally substituted with one or more C₃-C₁₂ cycloalkyl;
each R₁′ is independently unbranched C₁-C₁₈ alkylene; and
R₃ is C₁-C₁₀ alkyl optionally substituted with one or more hydroxyl or —NH—(C—O)—(C₁-C₆ alkyl).

In another aspect, the novel compound is a compound of Formula (II):

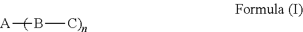

or a salt thereof, wherein:

A is:

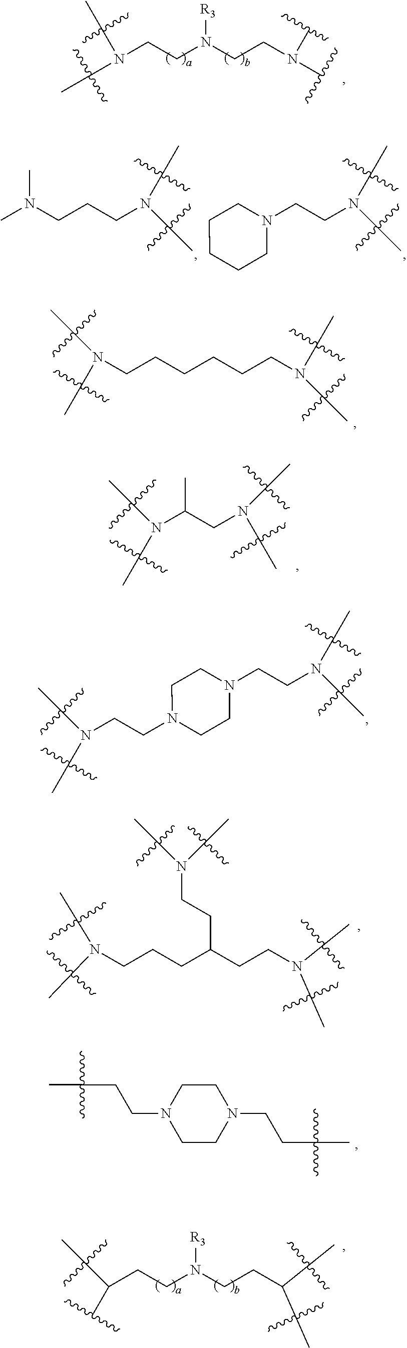

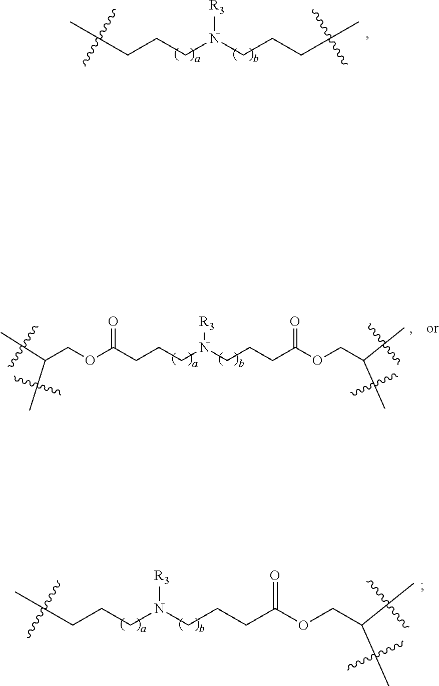

each B is independently

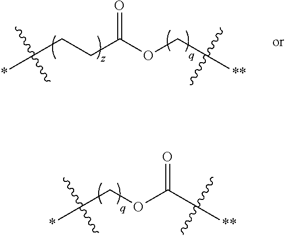

in which * indicates attachment to A and ** indicates attachment to C,
each C is independently

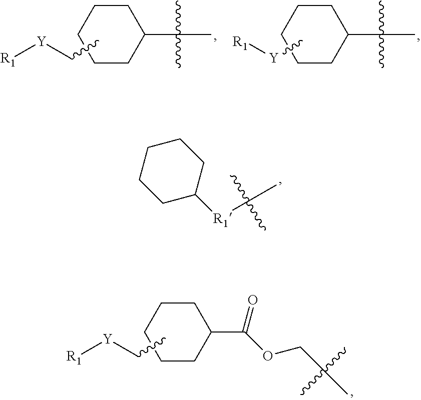

or C₁-C₁₈ alkyl;
n is an integer ranging from 2 to 6;
a is an integer ranging from 1 to 5;
b is an integer ranging from 1 to 5;
each R₁ is independently C₁-C₁₈ alkyl or C₂-C₁₈ alkenyl, wherein the C₁-C₁₈ alkyl or C₂-C₁₈ alkenyl is optionally substituted with one or more C₃-C₁₂ cycloalkyl;
each R₁′ is independently unbranched C₁-C₁₈ alkylene;
R₃ is (i) C₁-C₁₀ alkyl optionally substituted with one or more hydroxyl, —NH—(C═O)—(C₁-C₆ alkyl), or phenyl, or (ii) cyclohexyl optionally substituted with one or more hydroxyl or —(C₁-C₆ alkylene)-hydroxyl;
each Y is independently

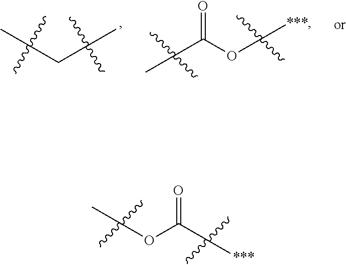

in which *** indicates attachment to R₁;
each p is independently an integer ranging from 0 to 3;
each q is independently 0 or 1; and
each z is independently 0 or 1.

In some aspects, provided are novel lipid nanoparticles (“LNPs”) comprising a novel compound. In one aspect, the novel compound is a compound of Formula (I). In another aspect, the novel compound is a compound of Formula (II).

In some aspects, provided are pharmaceutical compositions, comprising a composition of the present disclosure and at least one pharmaceutically-acceptable excipient or diluent.

In some aspects, provided are methods of delivering at least one nucleic acid to at least one cell comprising contacting the at least one cell with at least one composition of the present disclosure.

In some aspects, provided are methods of genetically modifying at least one cell comprising contacting the at least one cell with at least one composition of the present disclosure.

In some aspects, provided are methods of treating at least one disease or disorder in a subject in need thereof comprising administering to the subject at least one therapeutically effective amount of at least one composition of the present disclosure.

In some aspects, provided are methods of delivering at least one nucleic acid to at least one cell comprising contacting the at least one cell with at least one composition of the present disclosure.

In some aspects, provided are cells modified according to methods of the present disclosure.

Any of the aspects and/or embodiments described herein can be combined with any other aspect and/or embodiment described herein.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF THE DRAWINGS

FIG. 1 shows LNP compositions of the present disclosure, in the presence or absence of recombinant ApoE4, demonstrated increasing percentages of GFP positive HepG2 cells with increasing tannic acid concentrations.

FIG. 2A and FIG. 2B show whole body luminescence imaging (BLI) measurements at 48 hours post-administration (FIG. 2A) and body weight loss (BWL) measurements at 24 hours post-administration (FIG. 2B) of mice treated with LNP compositions of the present disclosure either comprising, or lacking, tannic acid.

FIG. 3A, FIG. 3B and FIG. 3C show whole body luminescence imaging (BLI) measurements at 48 hours post-administration (FIG. 3A); body weight loss (BWL) measurements at 24 hours post-administration (FIG. 3B); and the cytokine levels of mice treated with LNP compositions of the present disclosure either comprising, or lacking, tannic acid (FIG. 3C).

FIGS. 4A and 4B show whole body luminescence imaging (BLI) measurements at 48 hours (FIG. 4A) or one week (FIG. 4B) post-administration of mice treated with LNP compositions of the present disclosure either comprising, or lacking, tannic acid.

FIG. 5A and FIG. 5B show LNP compositions of the present disclosure, in the presence (FIG. 5B) or absence (FIG. 5A) of recombinant ApoE4, demonstrated higher luciferase expression in HepG2 cells with the addition of proanthocyanidin.

FIG. 6A and FIG. 6B show LNP compositions of the present disclosure, in the presence (FIG. 6B) or absence (FIG. 6A) of recombinant ApoE4, demonstrated higher luciferase expression in HepG2 cells with the addition of proanthocyanidin.

FIG. 7A and FIG. 7B shows LNP compositions of the present disclosure, in the presence (FIG. 7B) or absence (FIG. 7A) of recombinant ApoE4, demonstrated higher luciferase expression in HepG2 cells with the addition of ellagic acid or punicalagin.

FIG. 8 shows whole body luminescence imaging (BLI) measurements at 48 hours post-administration of mice treated with LNP compositions of the present disclosure either comprising, or lacking, proanthocyanidin, ellagic acid or punicalagin.

FIG. 9 shows LNP compositions of the present disclosure demonstrated higher PCCA-HA expression in mice with the addition of tannic acid.

### DETAILED DESCRIPTION

The present disclosure provides novel lipidoid compounds, novel lipid nanoparticle compositions (LNPs) comprising the novel lipidoid compounds, methods for preparing the LNPs, and methods for using same. In a non-limiting example, the compositions and methods of the present limiting disclosure can be used for gene delivery. In a non-limiting example, the compositions and methods of the present disclosure can be broadly used to deliver a nucleic acid to liver cells, in vivo, ex vivo or in vitro, for the treatment of certain diseases and disorders, including, but not limited to liver disorders. In a non-limiting example, the compositions and methods of the present disclosure can be broadly used to deliver a nucleic acid to induce the expression of a secreted therapeutic protein.

### Compounds of the Present Disclosure

In one aspect, the present disclosure provides compounds of Formula (I):

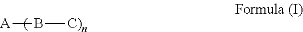

or a salt thereof, wherein:

A is:

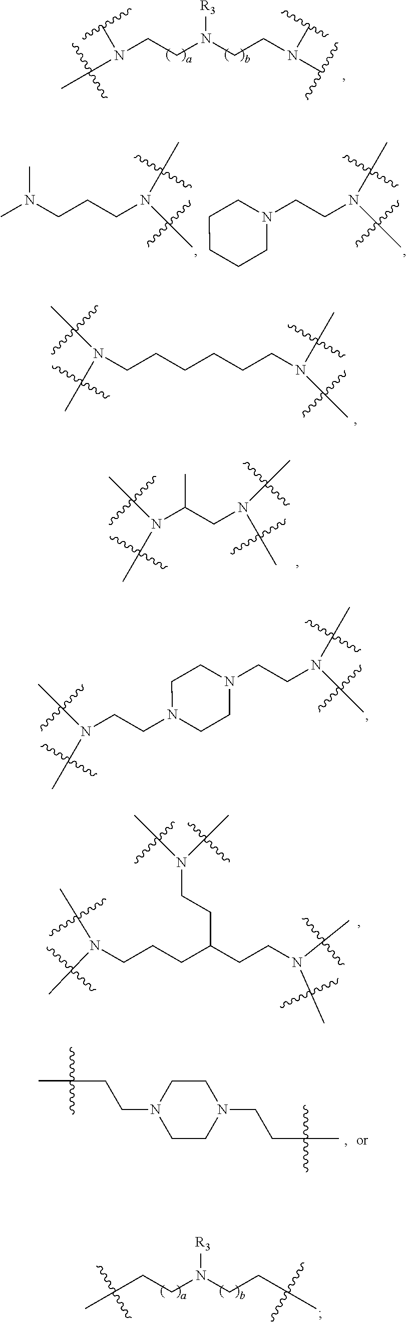

each B is independently

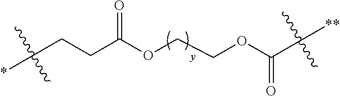

in which * indicates attachment to A and ** indicates attachment to C,
each C is independently

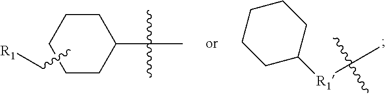

n is an integer ranging from 2 to 6;
a is an integer ranging from 1 to 5;
b is an integer ranging from 1 to 5;
each y is independently an integer ranging from 1 to 10;
each R₁ is independently unbranched C₁-C₁₈ alkyl optionally substituted with one or more C₃-C₁₂ cycloalkyl;
each R₁′ is independently unbranched C₁-C₁₈ alkylene; and
R₃ is C₁-C₁₀ alkyl optionally substituted with one or more hydroxyl or —NH—(C—O)—(C₁-C₆ alkyl).

In some aspects, A is

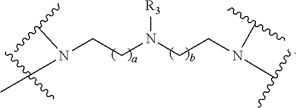

In some aspects, each C is

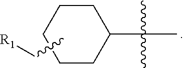

In some embodiments, each C is

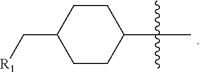

In some embodiments, each R₁ is C₄ alkyl. In some embodiments, each R₁ is

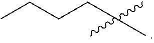 [SMILES: *CCCC]

In some embodiments, y is 1. In some embodiments, a is 1 and b is 1. In embodiments, a is 2 and b is 2.

In some aspects, each C is

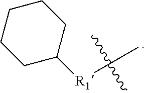

In some embodiments, each R₁′ is C₁ alkylene. In some embodiments, each R₁′ is C₂ alkylene. In some embodiments, each R₁′ is C₄ alkylene. In some embodiments, y is 7.

In some aspects, A is

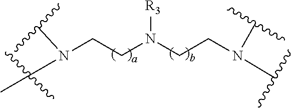

and each C is

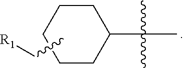

In some embodiments, each R₁ is C₄ alkyl. In some embodiments, each R₁ is

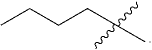 [SMILES: *CCCC]

In some embodiments, y is 1. In some embodiments, a is 1 and b is 1. In some embodiments, a is 2 and b is 2.

In some aspects, A is

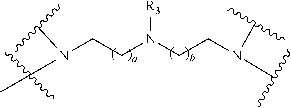

and each C is

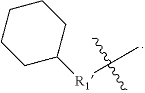

In some embodiments, each R₁′ is C₁ alkylene. In some embodiments, each R₁′ is C₂ alkylene. In some embodiments, each R₁′ is C₄ alkylene. In some embodiments, each R₁′ is C₁ alkylene and y is 7. In some embodiments, each R₁′ is C₂ alkylene and y is 7. In some embodiments, each R₁′ is C₄ alkylene and y is 1.

In some aspects, each R₁ is C₄ alkyl.

In some aspects, each R₁′ is C₁ alkylene.

In some aspects, each R₁′ is C₂ alkylene.

In some aspects, each R₁′ is C₄ alkylene.

In some aspects, R₃ is CH₃.

In some aspects, R₃ is C₁-C₁₀ alkyl substituted with one or more hydroxyl. In some embodiments, R₃ is

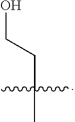 [SMILES: *CCO]

In some embodiments, R₃ is

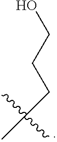 [SMILES: *CCCO]

In some embodiments, R₃ is

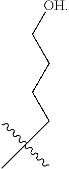 [SMILES: *CCCCO]

In some aspects, R₃ is

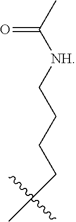 [SMILES: *CCCCNC(C)=O]

In some aspects when each R₁ is C₄ alkyl, y is 1. In some embodiments, a is 1 and b is 1. In some embodiments, a is 2 and b is 2.

In some aspects when each R₁′ is C₁ alkylene or C₂ alkylene, y is 7. In some embodiments, a is 2 and b is 2.

In some aspects when each R₁′ is C₄ alkylene, y is 1. In some embodiments, a is 1 and b is 1. In some embodiments, a is 2 and b is 2.

In some aspects, a is 1.

In some aspects, b is 1.

In some aspects, a is 1 and b is 1.

In some aspects, a is 2.

In some aspects, b is 2.

In some aspects, a is 2 and b is 2.

In some aspects, n is 4.

In some aspects, y is 1.

In some aspects, y is 7.

In some aspects, the compound of Formula (I) is a compound selected from:

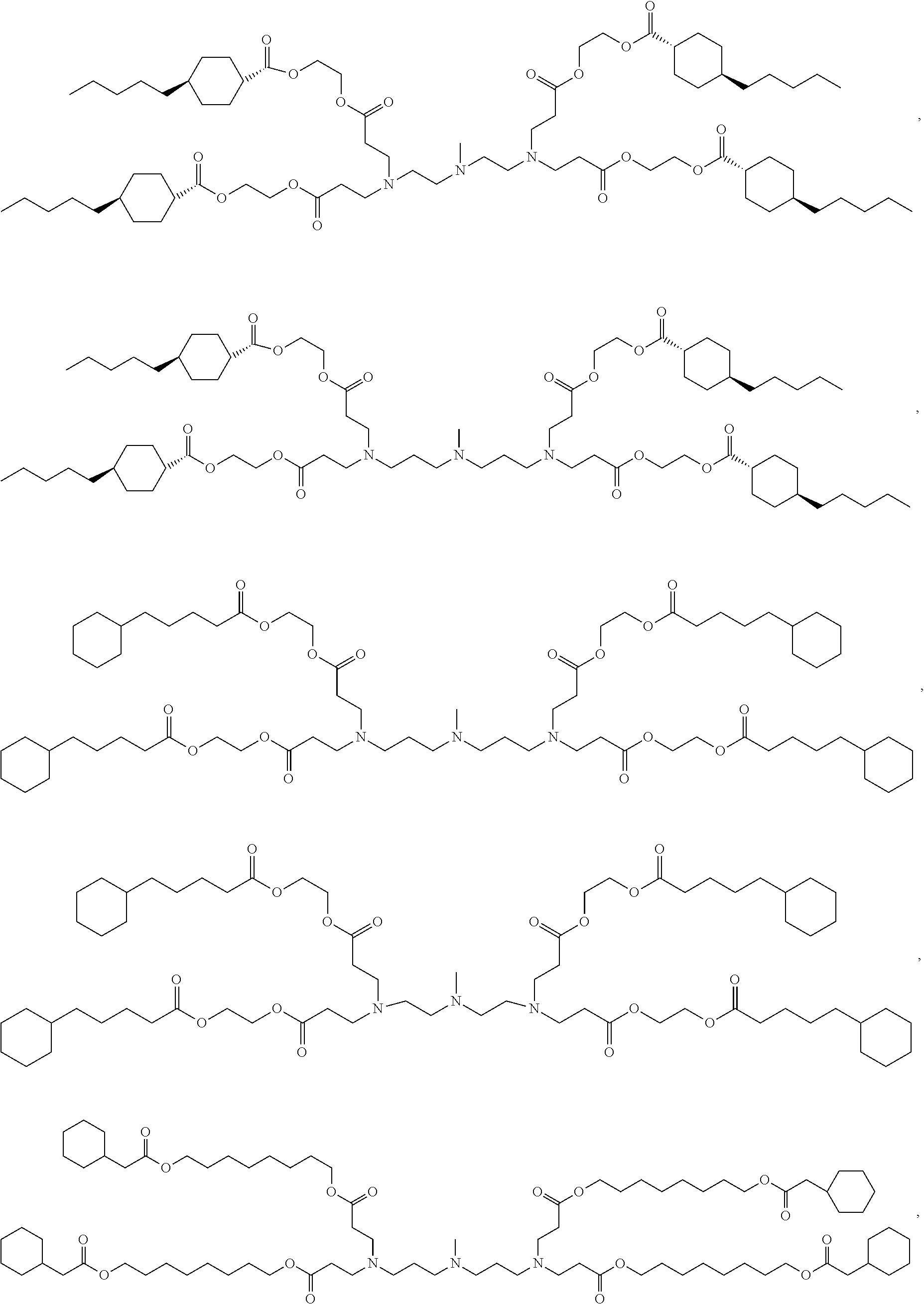

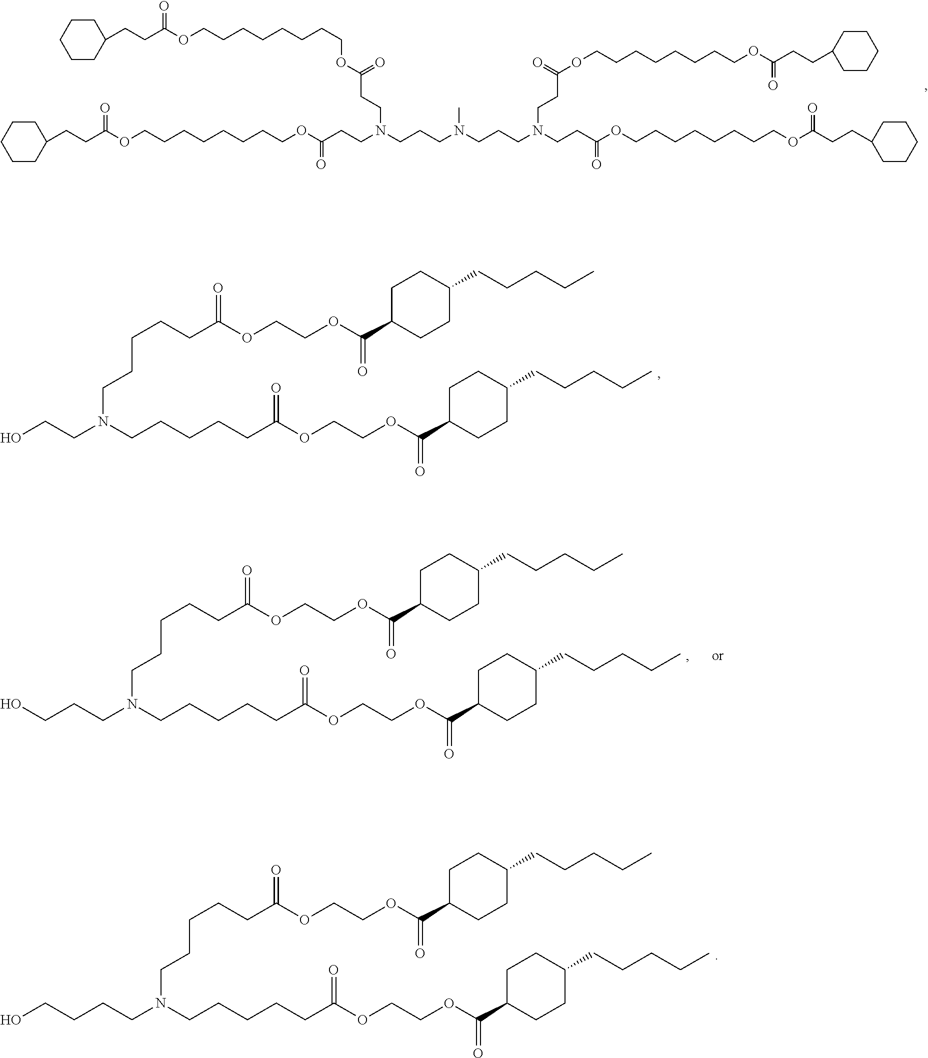 [SMILES: CN(CCCN(CCC(=O)OCCCCCCCCOC(=O)CCC1CCCCC1)CCC(=O)OCCCCCCCCOC(=O)CCC1CCCCC1)CCCN(CCC(=O)OCCCCCCCCOC(=O)CCC1CCCCC1)CCC(=O)OCCCCCCCCOC(=O)CCC1CCCCC1 ; CCCCC[C@H]1CC[C@H](C(=O)OCCOC(=O)CCCCCN(CCO)CCCCCC(=O)OCCOC(=O)[C@H]2CC[C@H](CCCCC)CC2)CC1 ; CCCCC[C@H]1CC[C@H](C(=O)OCCOC(=O)CCCCCN(CCCO)CCCCCC(=O)OCCOC(=O)[C@H]2CC[C@H](CCCCC)CC2)CC1 ; CCCCC[C@H]1CC[C@H](C(=O)OCCOC(=O)CCCCCN(CCCCO)CCCCCC(=O)OCCOC(=O)[C@H]2CC[C@H](CCCCC)CC2)CC1]

In one aspect, the present disclosure provides compounds of Formula (II):

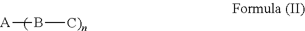

or a salt thereof, wherein:
A is:

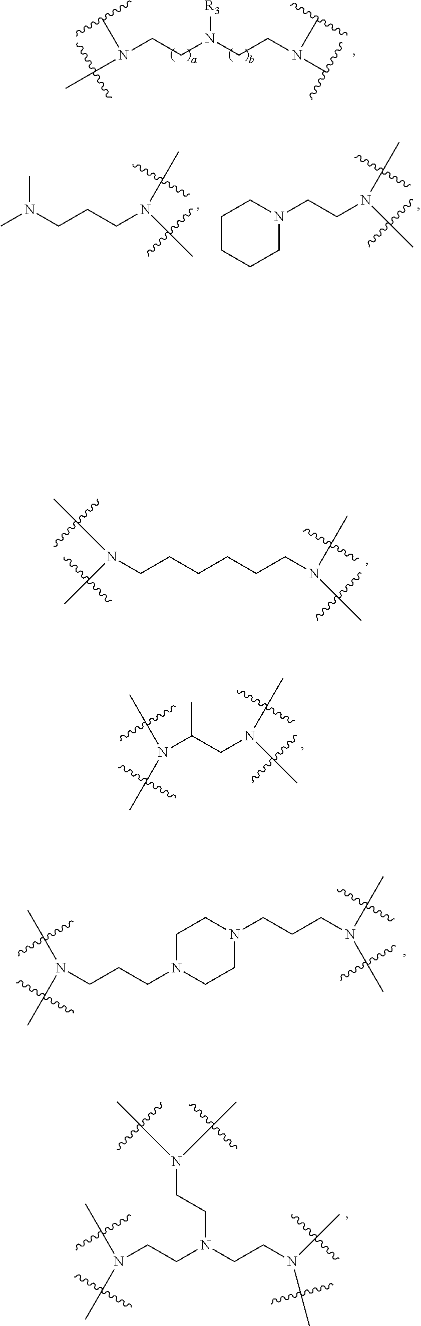

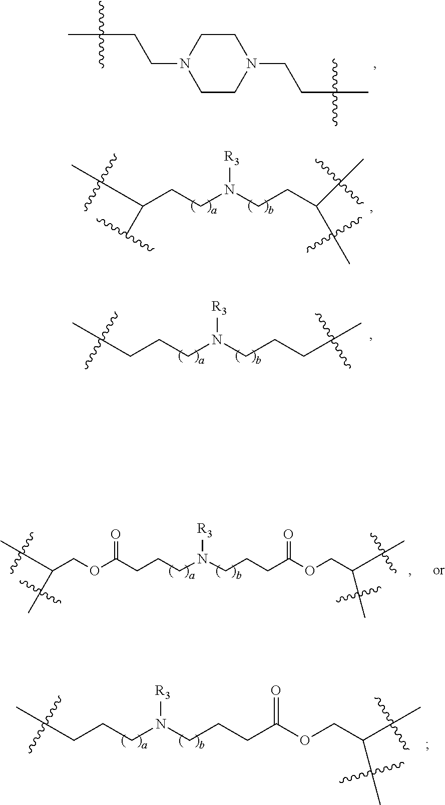

each B is independently

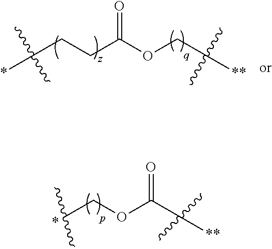

in which * indicates attachment to A and ** indicates attachment to C,
each C is independently

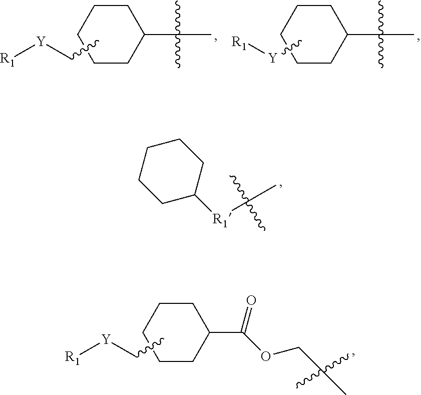

or C₁-C₁₈ alkyl;
n is an integer ranging from 2 to 6;
a is an integer ranging from 1 to 5;
b is an integer ranging from 1 to 5;
each R₁ is independently C₁-C₁₈ alkyl or C₂-C₁₈ alkenyl, wherein the C₁-C₁₈ alkyl or C₂-C₁₈ alkenyl is optionally substituted with one or more C₃-C₁₂ cycloalkyl;
each R₁′ is independently unbranched C₁-C₁₈ alkylene;
R₃ is (i) C₁-C₁₀ alkyl optionally substituted with one or more hydroxyl, —NH—(C═O)—(C₁-C₆ alkyl), or phenyl, or (ii)-cyclohexyl optionally substituted with one or more hydroxyl or —(C₁-C₆ alkylene)-hydroxyl;
each Y is independently

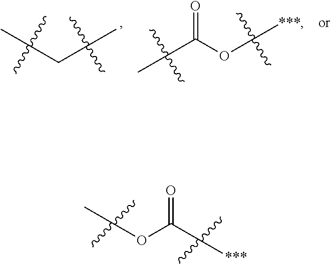

in which *** indicates attachment to R₁;
each p is independently an integer ranging from 0 to 3;
each q is independently 0 or 1; and
each z is independently 0 or 1.

In some aspects, A is

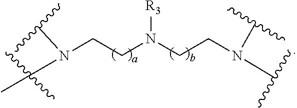

In some aspects, A is

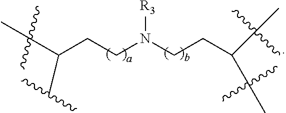

In some aspects, A is

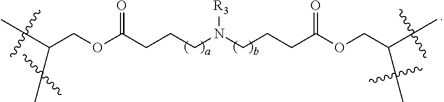

In some aspects, each B is

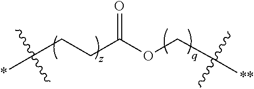

in which * indicates attachment to A and ** indicates attachment to C.

In some aspects, each B is

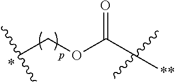

** indicates attachment to C.

In some aspects, each C is

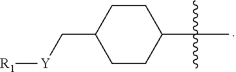

In some embodiments, each C is

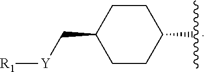

In some embodiments, each C is

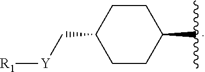

In some aspects, each C is

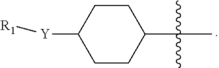

In some embodiments, each C is

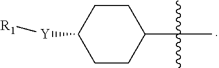

In some embodiments, each C is

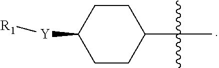

In some aspects, each Cis

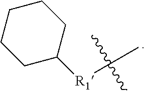

In some aspects, each C is

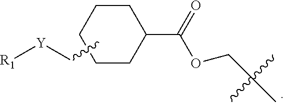

In some aspects, each C is

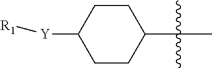

or C₁-C₁₈ alkyl.

In some aspects, each Y is

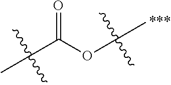 [SMILES: *OC(*)=O]

in which *** indicates attachment to R₁.

In some aspects, each Y is

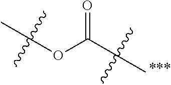 [SMILES: *OC(*)=O]

in which *** indicates attachment to R₁.

In some aspects, each Y is

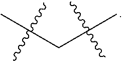 [SMILES: *C*]

In some aspects, a is 2.

In some aspects, b is 2.

In some aspects, a is 2 and b is 2.

In some aspects, each R₁ is C₁-C₁₈ alkyl. In some embodiments, each R₁ is

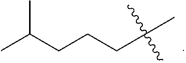 [SMILES: *CCCC(C)C]

In some embodiments, each R₁ is

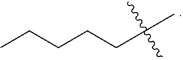 [SMILES: *CCCCC]

In some embodiments, each R₁ is

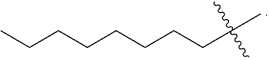 [SMILES: *CCCCCCCC]

In some aspects, each C is

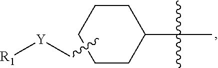

each Y is

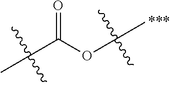 [SMILES: *OC(*)=O]

in which *** indicates attachment to R₁ and each R₁ is C₁-C₁₈ alkyl. In some embodiments, each R₁ is

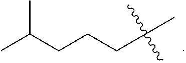 [SMILES: *CCCC(C)C]

In some embodiments, each R₁ is

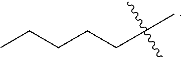 [SMILES: *CCCCC]

In some embodiments, each R₁ is

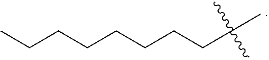 [SMILES: *CCCCCCCC]

In some aspects, each R₁ is C₂-C₁₈ alkenyl. In some embodiments, each R₁ is

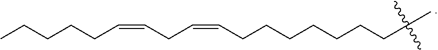 [SMILES: *CCCCCCC/C=C\C/C=C\CCCCC]

In some aspects, each C is

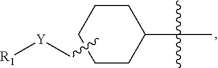

each Y is

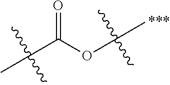 [SMILES: *OC(*)=O]

in which *** indicates attachment to R₁ and each R₁ is C₂-C₁₈ alkenyl. In some embodiments, each R₁ is

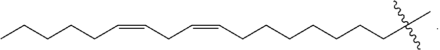 [SMILES: *CCCCCCC/C=C\C/C=C\CCCCC]

In some aspects, each R₁ is C₁-C₁₈ alkyl. In some embodiments, each R₁ is

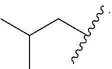 [SMILES: *CC(C)C]

In some embodiments, each R₁ is

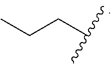 [SMILES: *CCC]

In some embodiments, each R₁ is C₁-C₁₈ alkyl substituted with one or more C₃-C₁₂ cycloalkyl. In some embodiments, each R₁ is

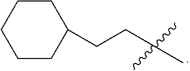 [SMILES: *CCC1CCCCC1]

In some aspects, each C is

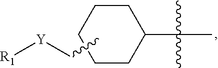

each Y is

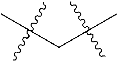 [SMILES: *C*]

and each R₁ is C₁-C₁₈ alkyl. In some embodiments, each R₁ is

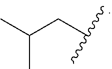 [SMILES: *CC(C)C]

In some embodiments, each R₁ is

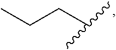 [SMILES: *CCC]

In some embodiments, each R₁ is C₁-C₁₈ alkyl substituted with one or more C₃-C₁₂ cycloalkyl. In some embodiments, each R₁ is

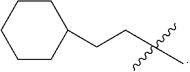 [SMILES: *CCC1CCCCC1]

In some aspects, each C is

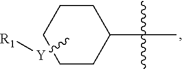

each Y is

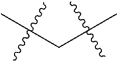 [SMILES: *C*]

and each R₁ is C₁-C₁₈ alkyl. In some embodiments, each R₁ is

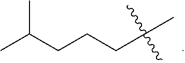 [SMILES: *CCCC(C)C]

In some embodiments, each R₁ is

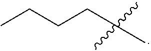 [SMILES: *CCCC]

In some aspects, each C is

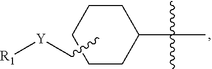

each Y is

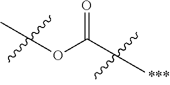 [SMILES: *OC(*)=O]

in which *** indicates attachment to R₁ and each R₁ is C₁-C₁₈ alkyl. In some embodiments, each R₁ is

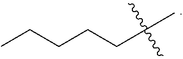 [SMILES: *CCCCC]

In some aspects, z is 1.

In some aspects, A is

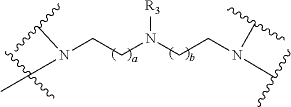

and z is 1.
In some aspects, z is 0.

In some aspects, A is

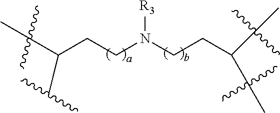

and z is 0.

In some aspects, q is 0.

In some aspects, q is 1.

In some aspects, p is 0.

In some aspects, A is

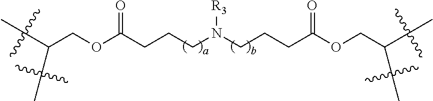

and p is 1.

In some aspects, p is 1.

In some aspects, p is 2.

In some aspects, p is 3.

In some aspects, n is 4.

In some aspects, R₃ is CH₃.

In some aspects, R₃ is CH₂CH₂CH₃.

In some aspects, R₃ is C₁-C₁₀ alkyl substituted with one or more hydroxyl. In some embodiments, R₃ is

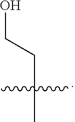 [SMILES: *CCO]

In some embodiments, R₃ is

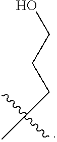 [SMILES: *CCCO]

In some embodiments, R₃ is

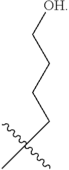 [SMILES: *CCCCO]

In some embodiments, R₃ is

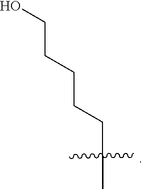 [SMILES: *CCCCCO]

In some embodiments, R₃ is

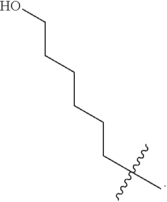 [SMILES: *CCCCCCO]

In some embodiments, R₃ is

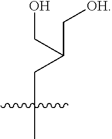 [SMILES: *CC(CO)CO]

In some embodiments, R₃ is

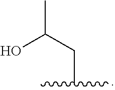 [SMILES: *CC(C)O]

In some embodiments, R₃ is

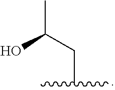 [SMILES: *C[C@H](C)O]

In some embodiments, R₃ is

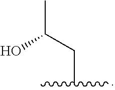 [SMILES: *C[C@@H](C)O]

In some embodiments, R₃ is

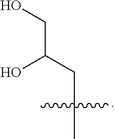 [SMILES: *CC(O)CO]

In some embodiments, R₃ is

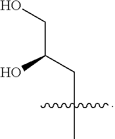 [SMILES: *C[C@@H](O)CO]

In some embodiments, R₃ is

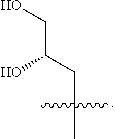 [SMILES: *C[C@H](O)CO]

In some embodiments, R₃ is

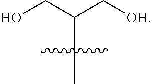 [SMILES: *C(CO)CO]

In some embodiments, R₃ is C₁-C₁₀ alkyl substituted with one or more phenyl.

In some embodiments, R₃ is-CH₂-phenyl.

In some aspects, R₃ is cyclohexyl substituted with one or more hydroxyl or —(C₁-C₆ alkylene)-hydroxyl. In some embodiments, R₃ is

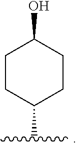 [SMILES: *[C@H]1CC[C@H](O)CC1]

In some embodiments, R₃ is

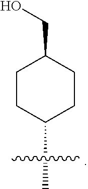

In some aspects, R₃ is

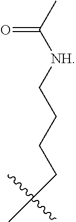 [SMILES: *CCCCNC(C)=O]

In some aspects, the compound of Formula (II) is a compound selected from:

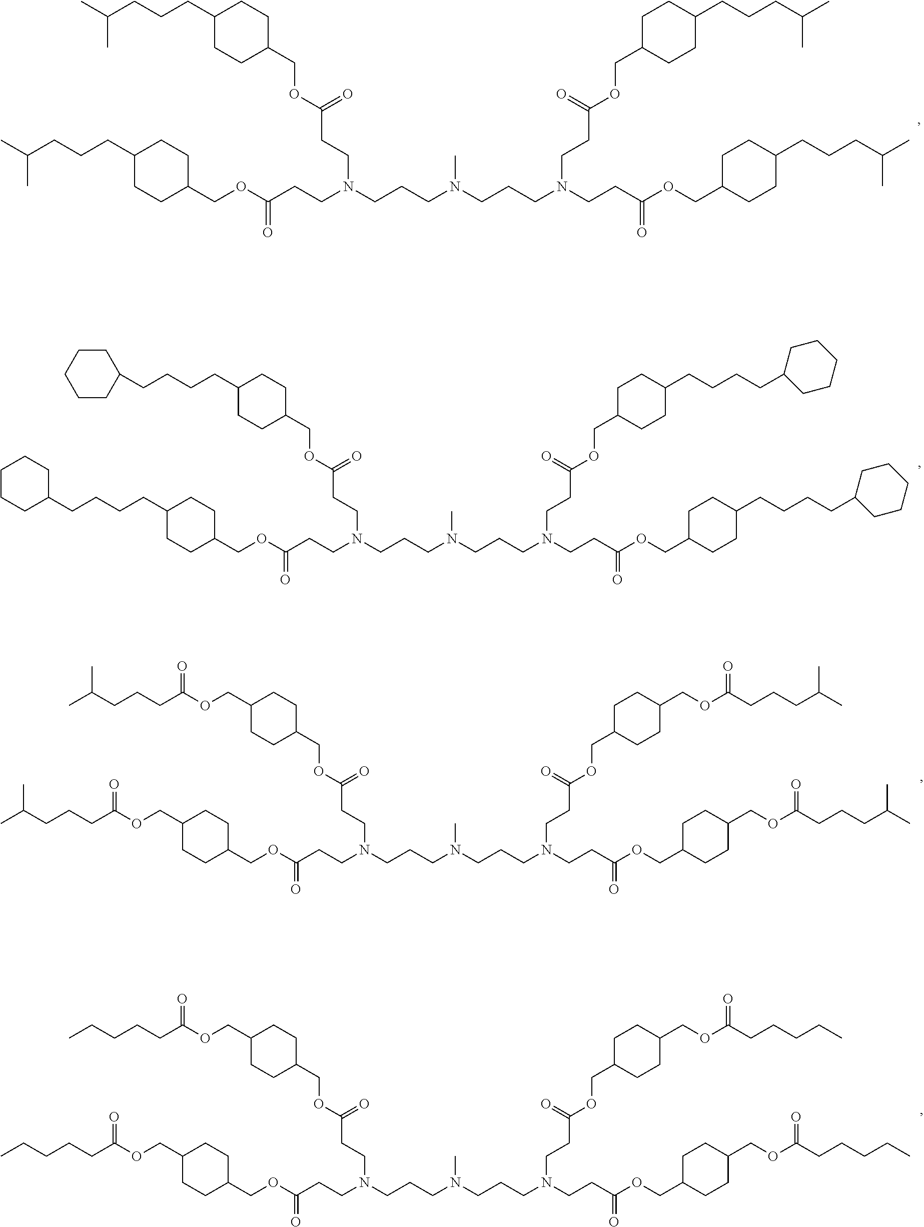

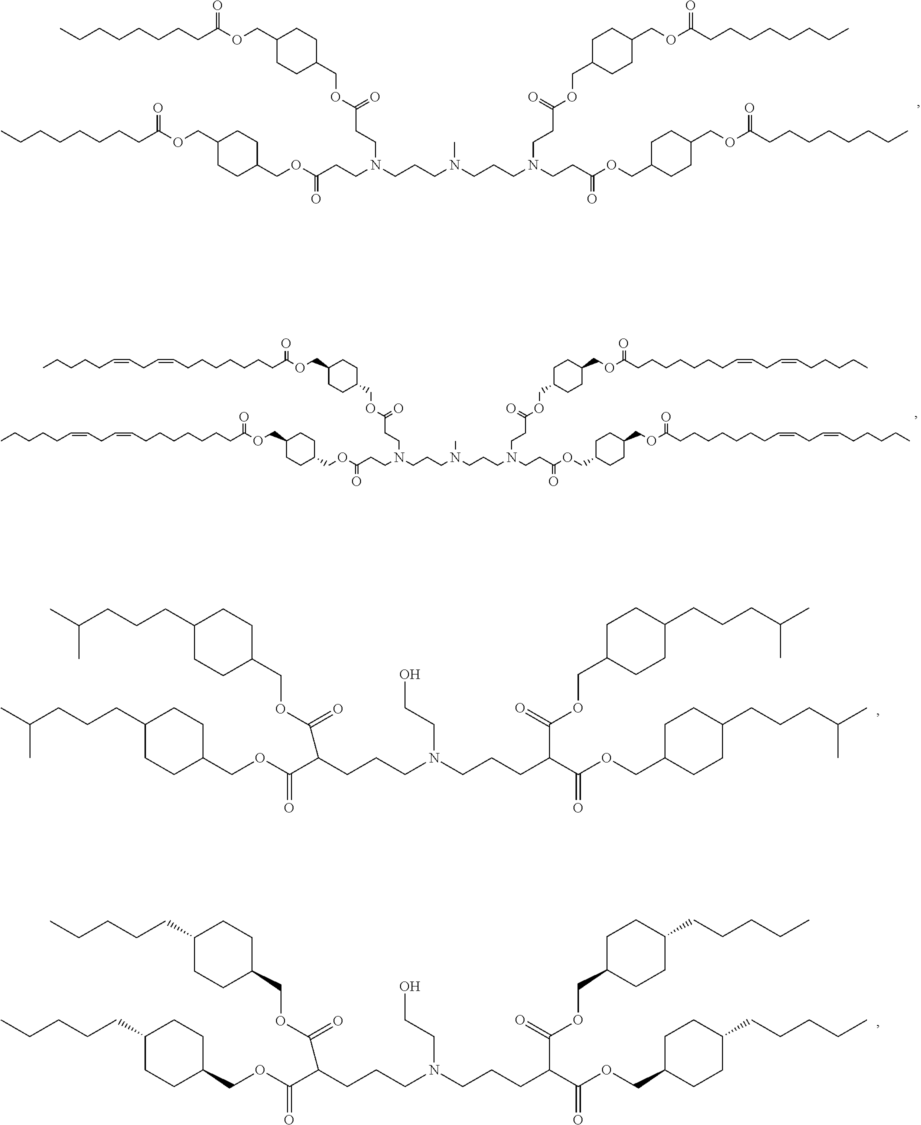

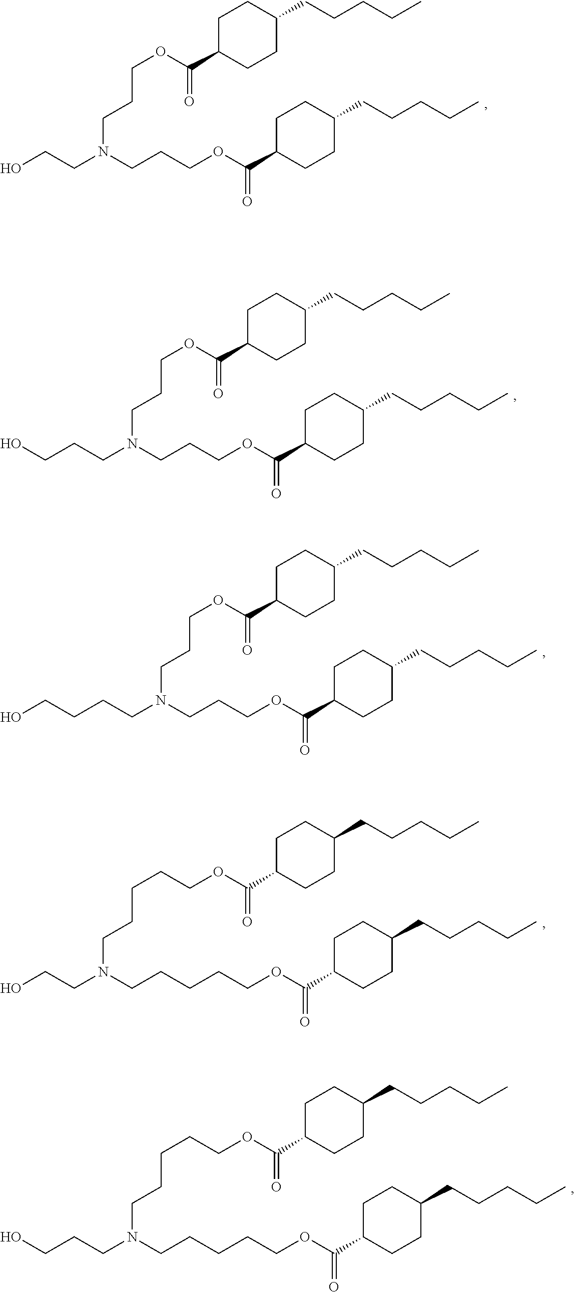 [SMILES: CCCCC[C@H]1CC[C@H](C(=O)OCCCN(CCO)CCCOC(=O)[C@H]2CC[C@H](CCCCC)CC2)CC1 ; CCCCC[C@H]1CC[C@H](C(=O)OCCCN(CCCO)CCCOC(=O)[C@H]2CC[C@H](CCCCC)CC2)CC1 ; CCCCC[C@H]1CC[C@H](C(=O)OCCCN(CCCCO)CCCOC(=O)[C@H]2CC[C@H](CCCCC)CC2)CC1 ; CCCCC[C@H]1CC[C@H](C(=O)OCCCCCN(CCO)CCCCCOC(=O)[C@H]2CC[C@H](CCCCC)CC2)CC1 ; CCCCC[C@H]1CC[C@H](C(=O)OCCCCCN(CCCO)CCCCCOC(=O)[C@H]2CC[C@H](CCCCC)CC2)CC1]

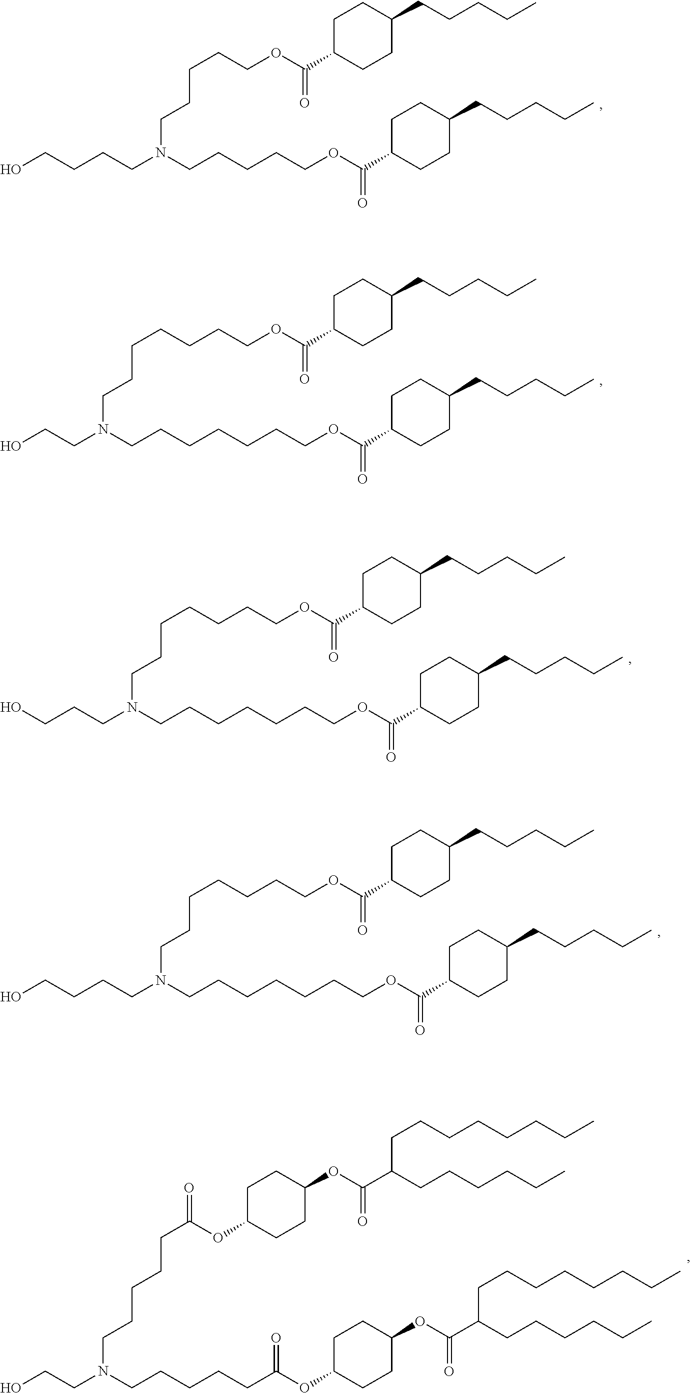 [SMILES: CCCCC[C@H]1CC[C@H](C(=O)OCCCCCN(CCCCO)CCCCCOC(=O)[C@H]2CC[C@H](CCCCC)CC2)CC1 ; CCCCC[C@H]1CC[C@H](C(=O)OCCCCCCCN(CCO)CCCCCCCOC(=O)[C@H]2CC[C@H](CCCCC)CC2)CC1 ; CCCCC[C@H]1CC[C@H](C(=O)OCCCCCCCN(CCCO)CCCCCCCOC(=O)[C@H]2CC[C@H](CCCCC)CC2)CC1 ; CCCCC[C@H]1CC[C@H](C(=O)OCCCCCCCN(CCCCO)CCCCCCCOC(=O)[C@H]2CC[C@H](CCCCC)CC2)CC1 ; CCCCCCCCC(CCCCCC)C(=O)O[C@H]1CC[C@H](OC(=O)CCCCCN(CCO)CCCCCC(=O)O[C@H]2CC[C@H](OC(=O)C(CCCCCC)CCCCCCCC)CC2)CC1]

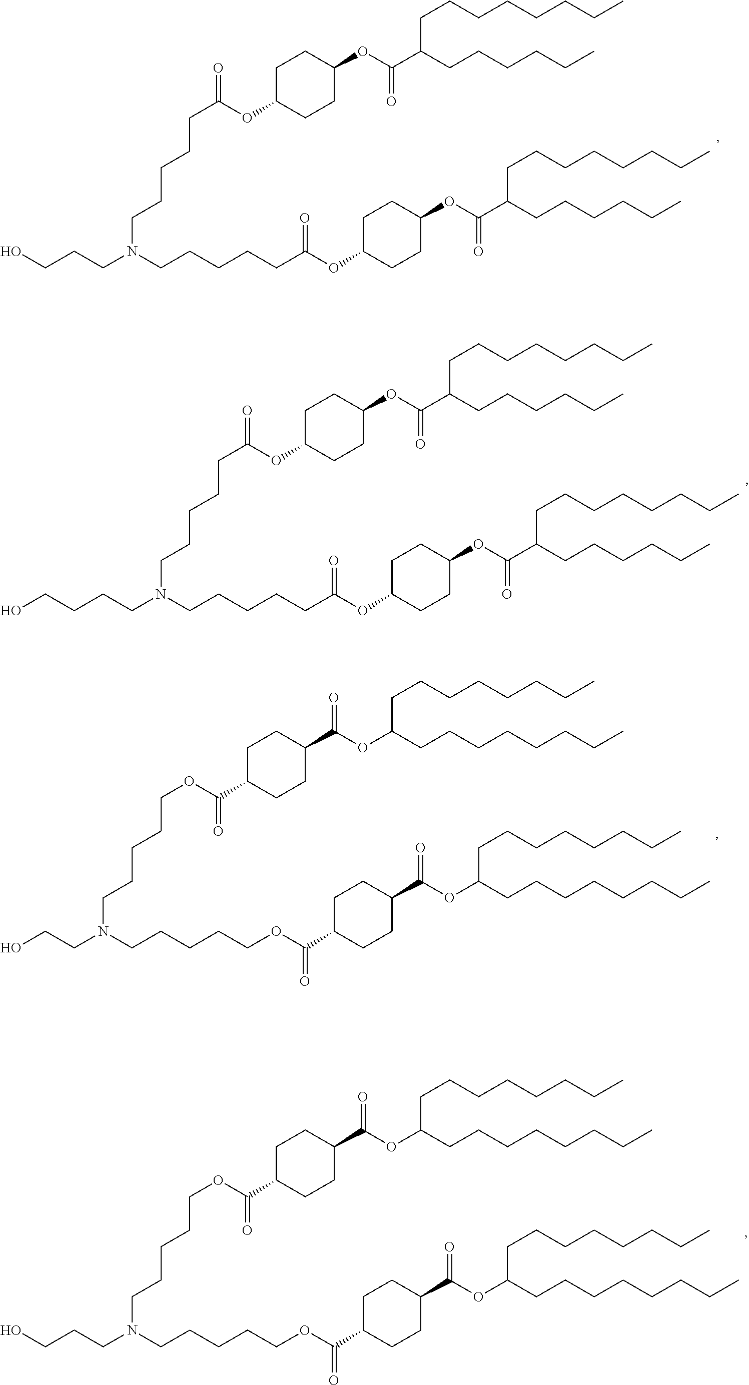 [SMILES: CCCCCCCCC(CCCCCC)C(=O)O[C@H]1CC[C@H](OC(=O)CCCCCN(CCCO)CCCCCC(=O)O[C@H]2CC[C@H](OC(=O)C(CCCCCC)CCCCCCCC)CC2)CC1 ; CCCCCCCCC(CCCCCC)C(=O)O[C@H]1CC[C@H](OC(=O)CCCCCN(CCCCO)CCCCCC(=O)O[C@H]2CC[C@H](OC(=O)C(CCCCCC)CCCCCCCC)CC2)CC1 ; CCCCCCCCC(CCCCCCCC)OC(=O)[C@H]1CC[C@H](C(=O)OCCCCCN(CCO)CCCCCOC(=O)[C@H]2CC[C@H](C(=O)OC(CCCCCCCC)CCCCCCCC)CC2)CC1 ; CCCCCCCCC(CCCCCCCC)OC(=O)[C@H]1CC[C@H](C(=O)OCCCCCN(CCCO)CCCCCOC(=O)[C@H]2CC[C@H](C(=O)OC(CCCCCCCC)CCCCCCCC)CC2)CC1]

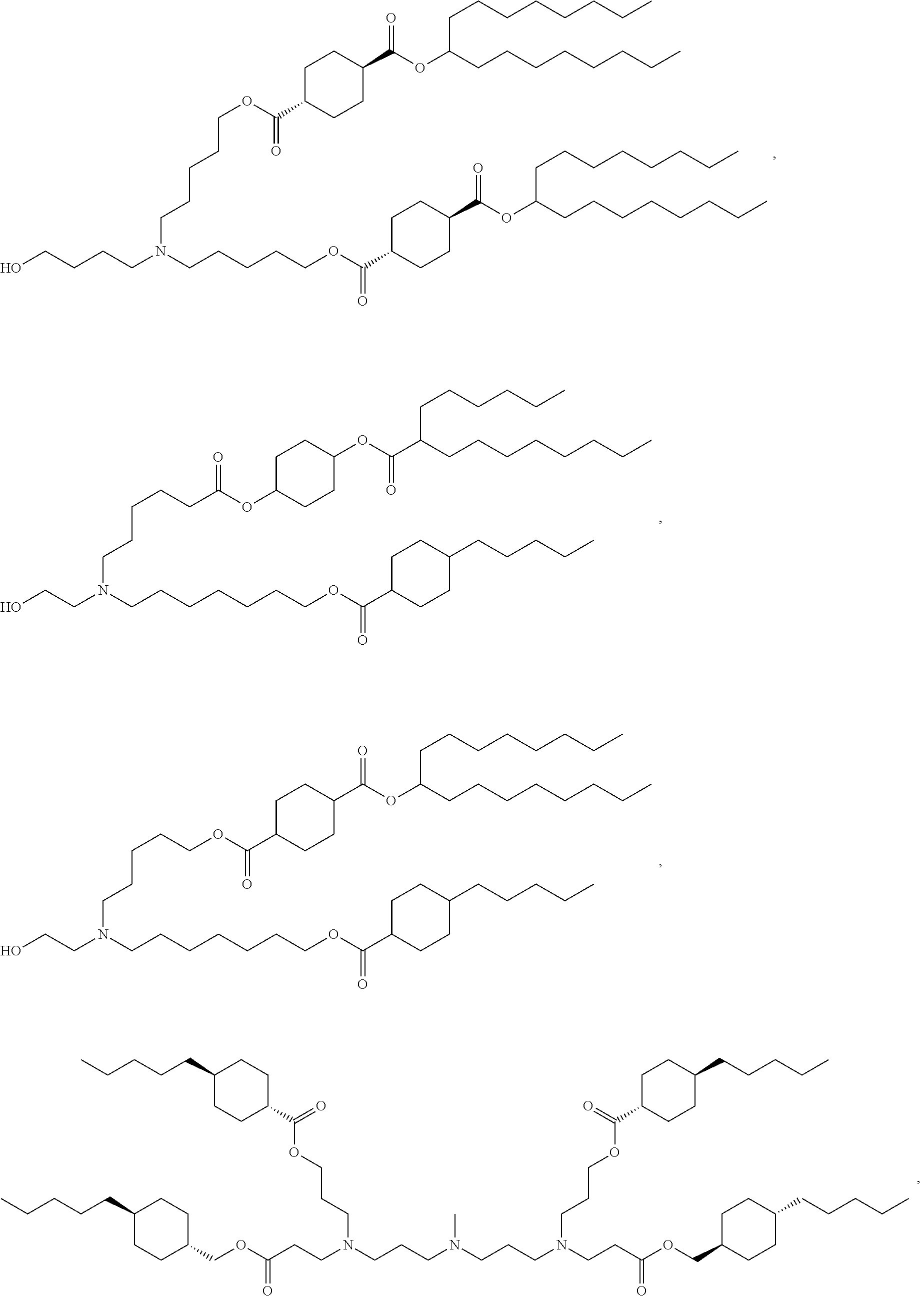

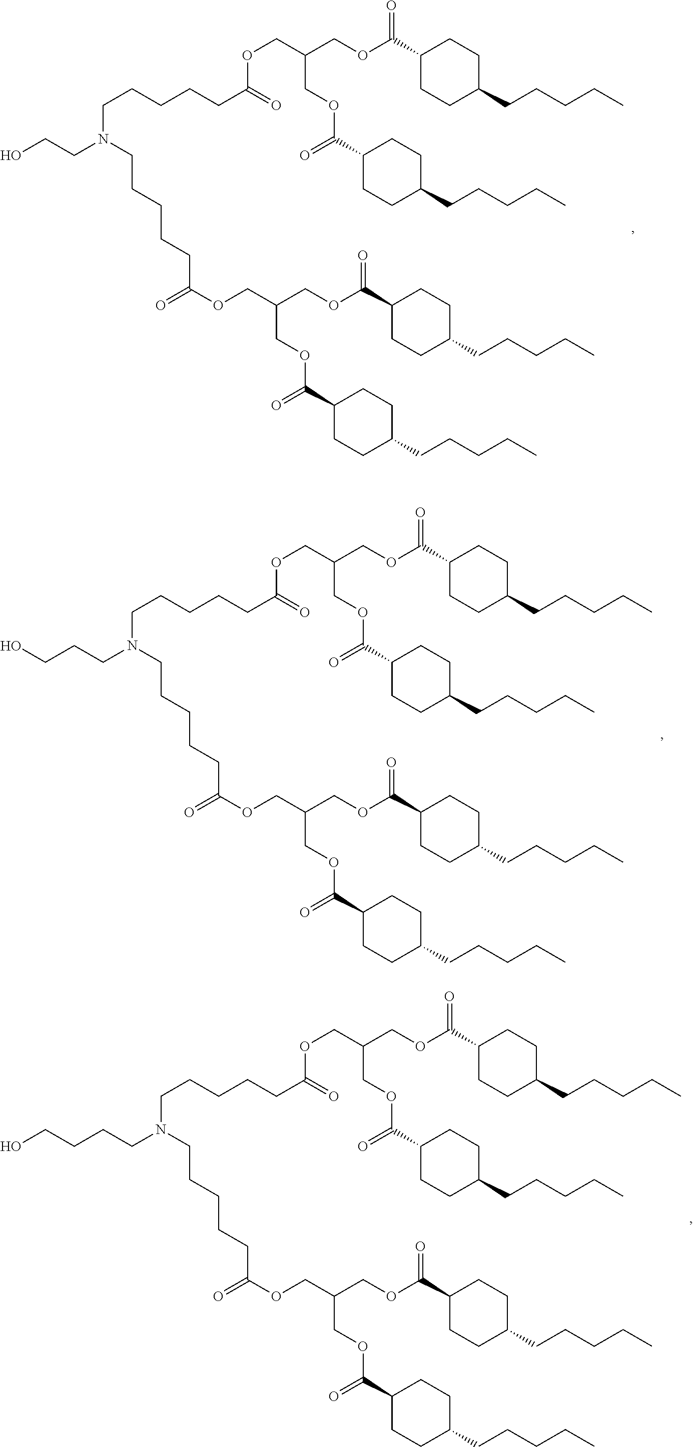

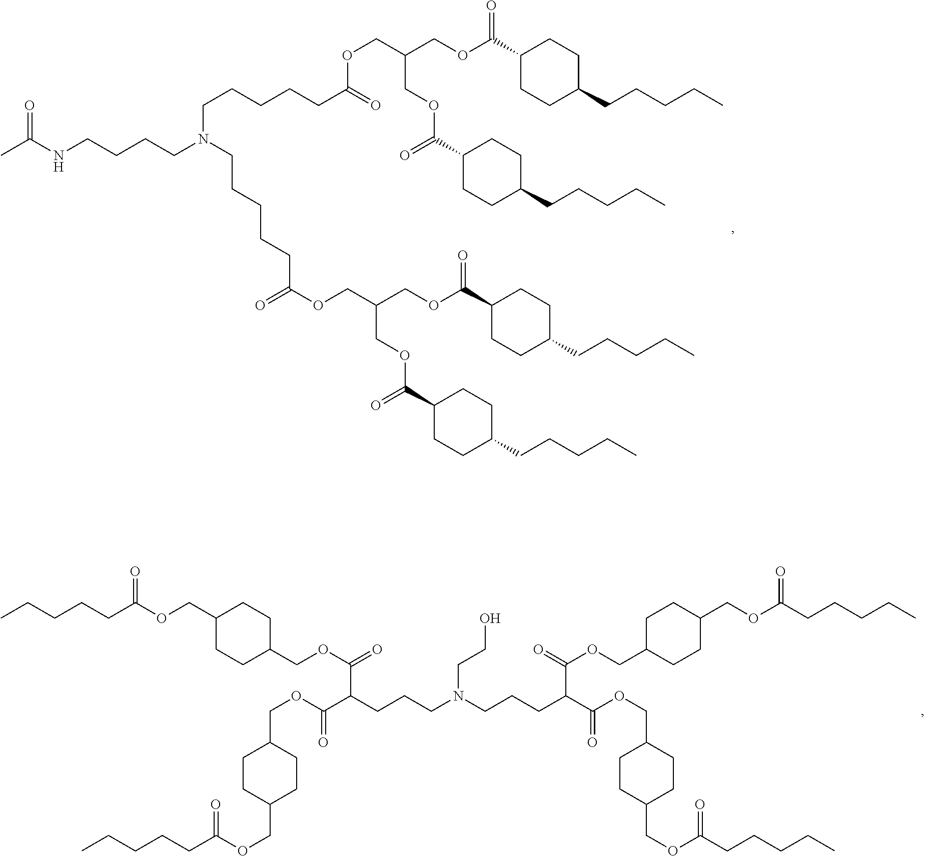

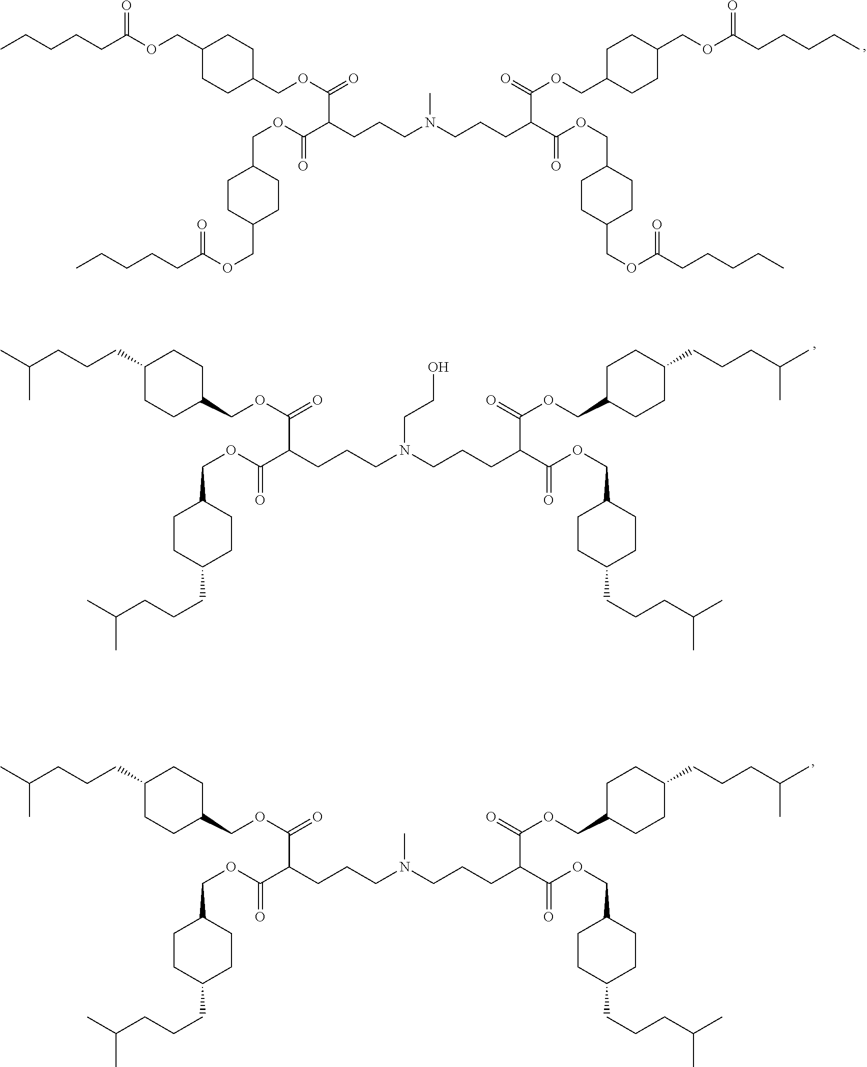

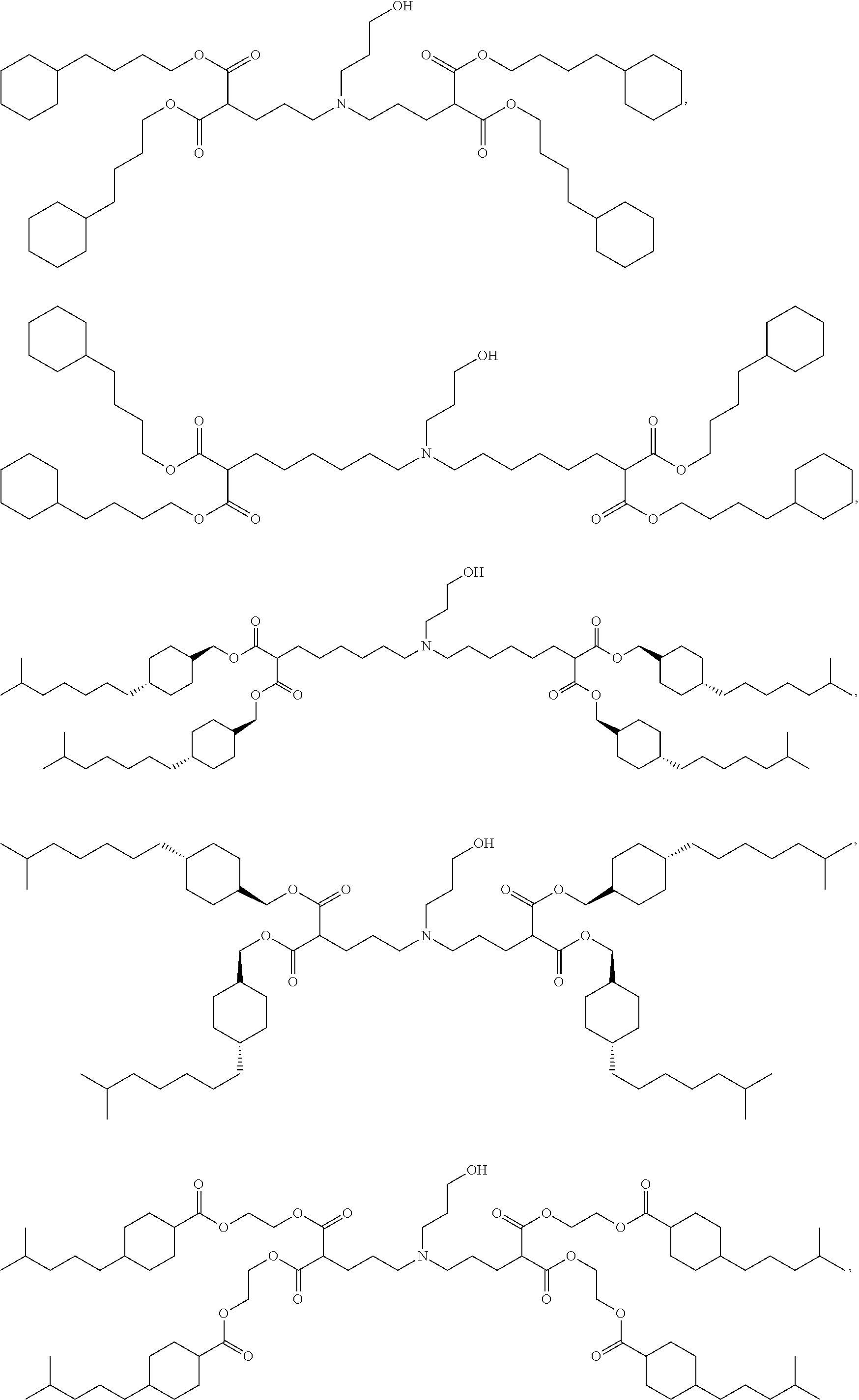

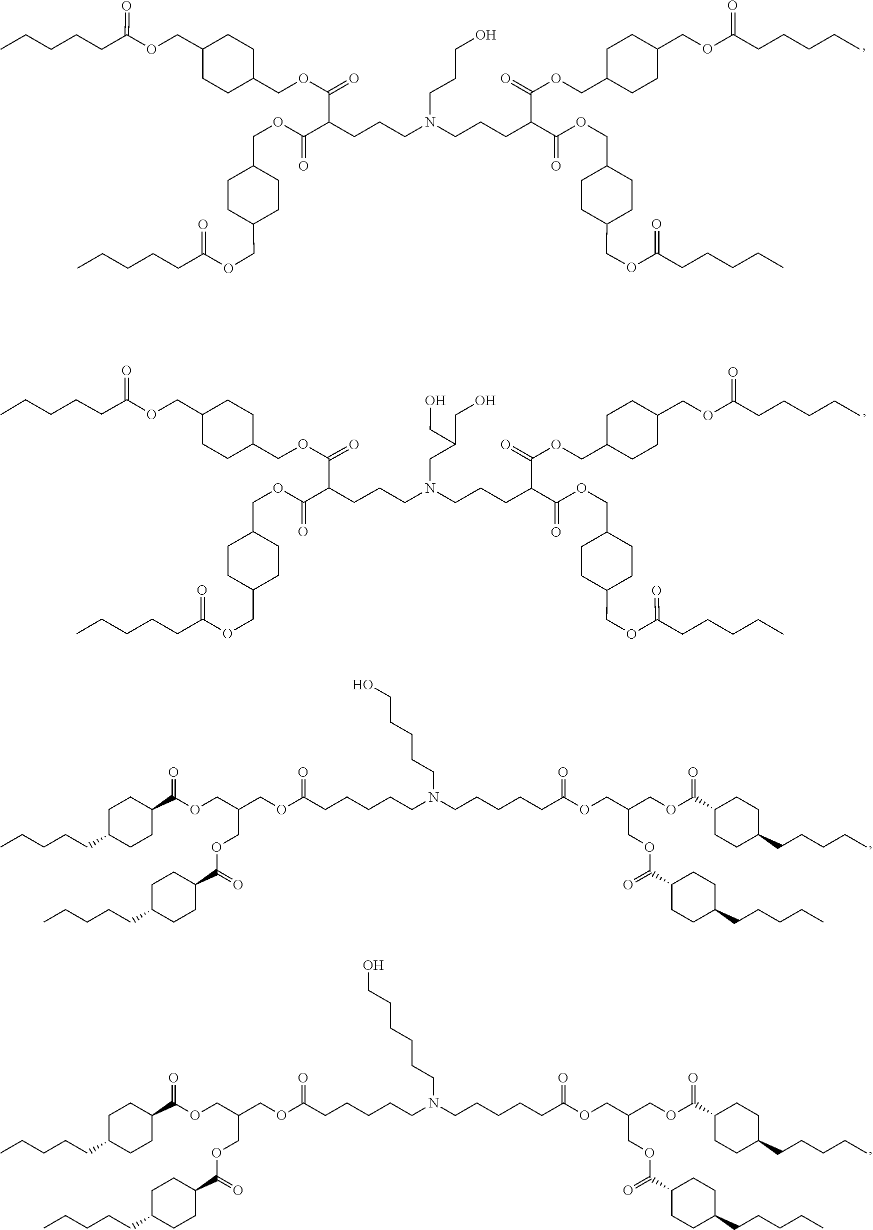

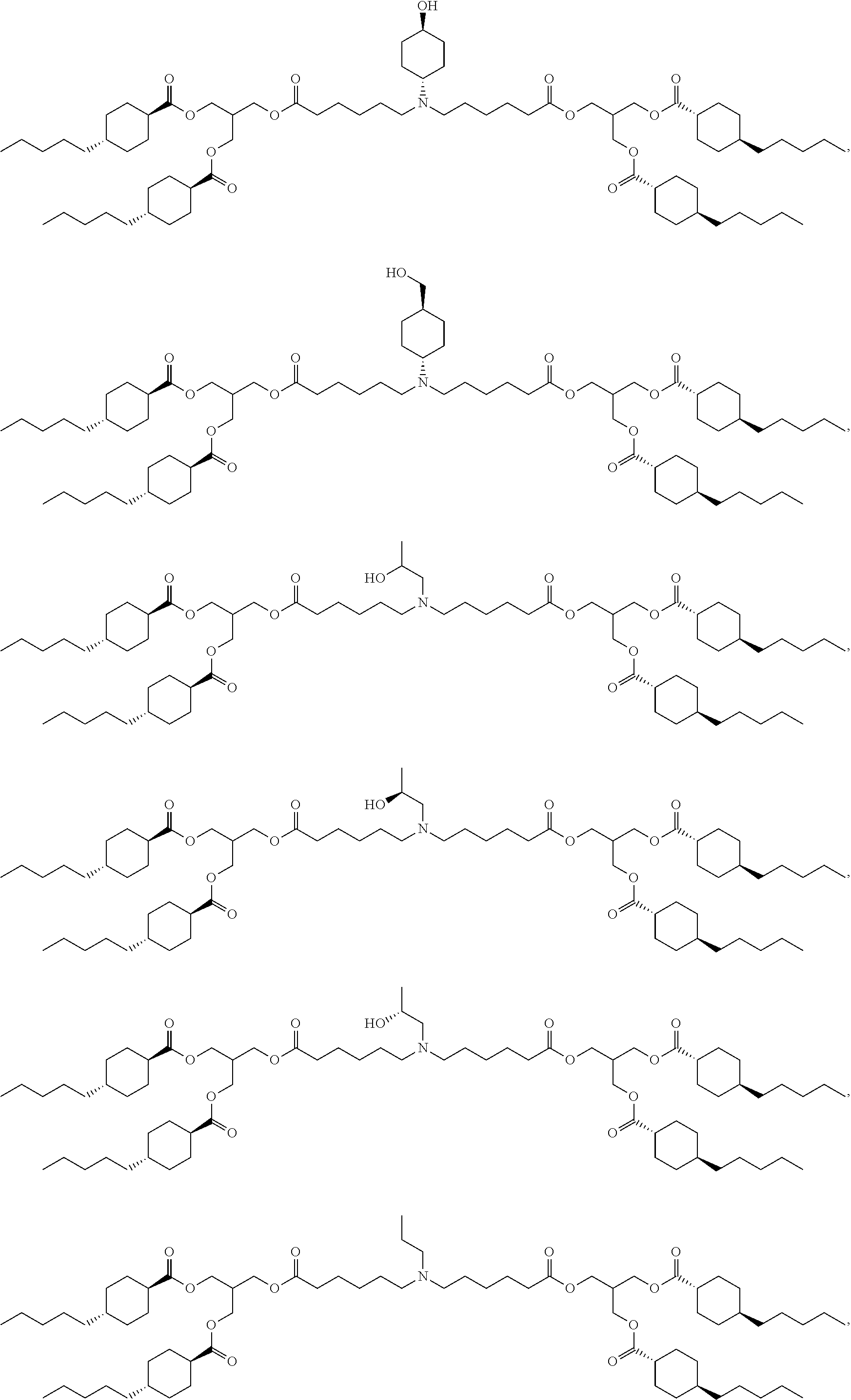

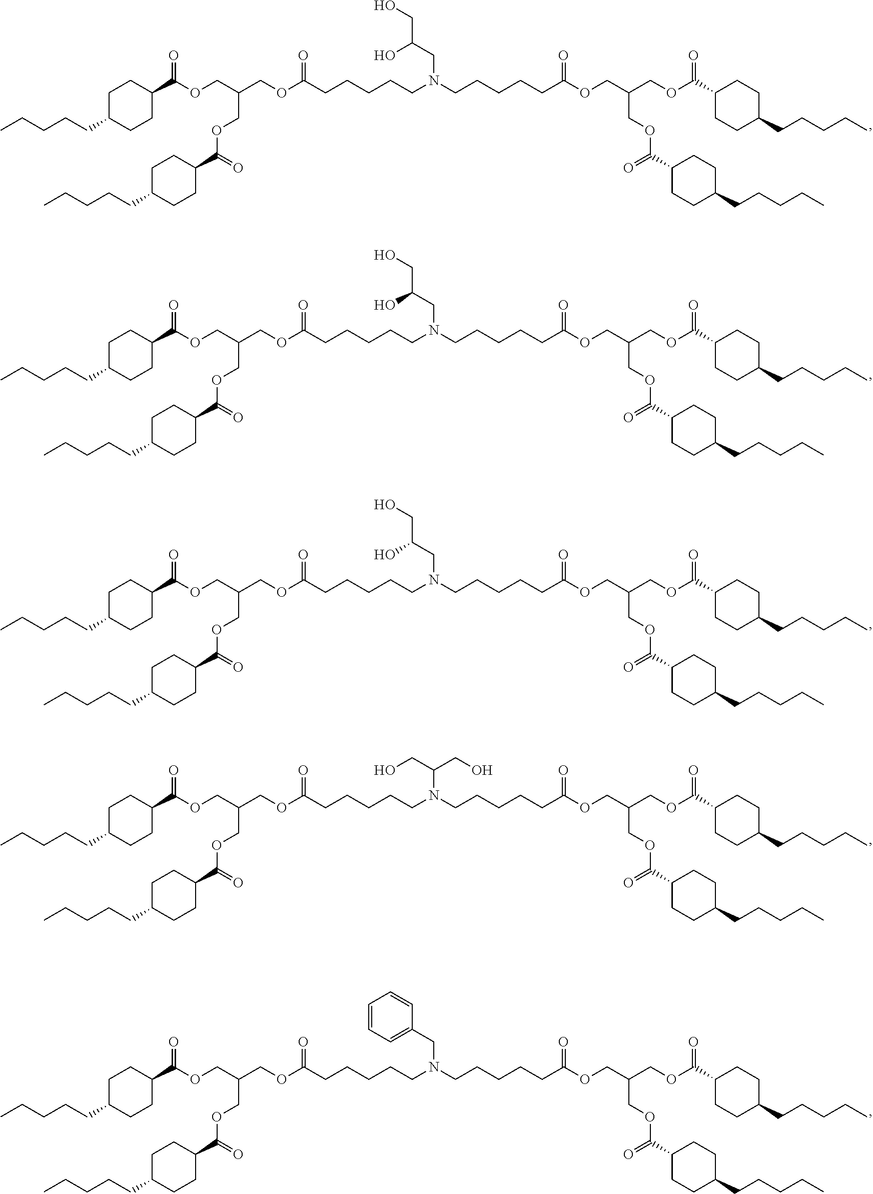

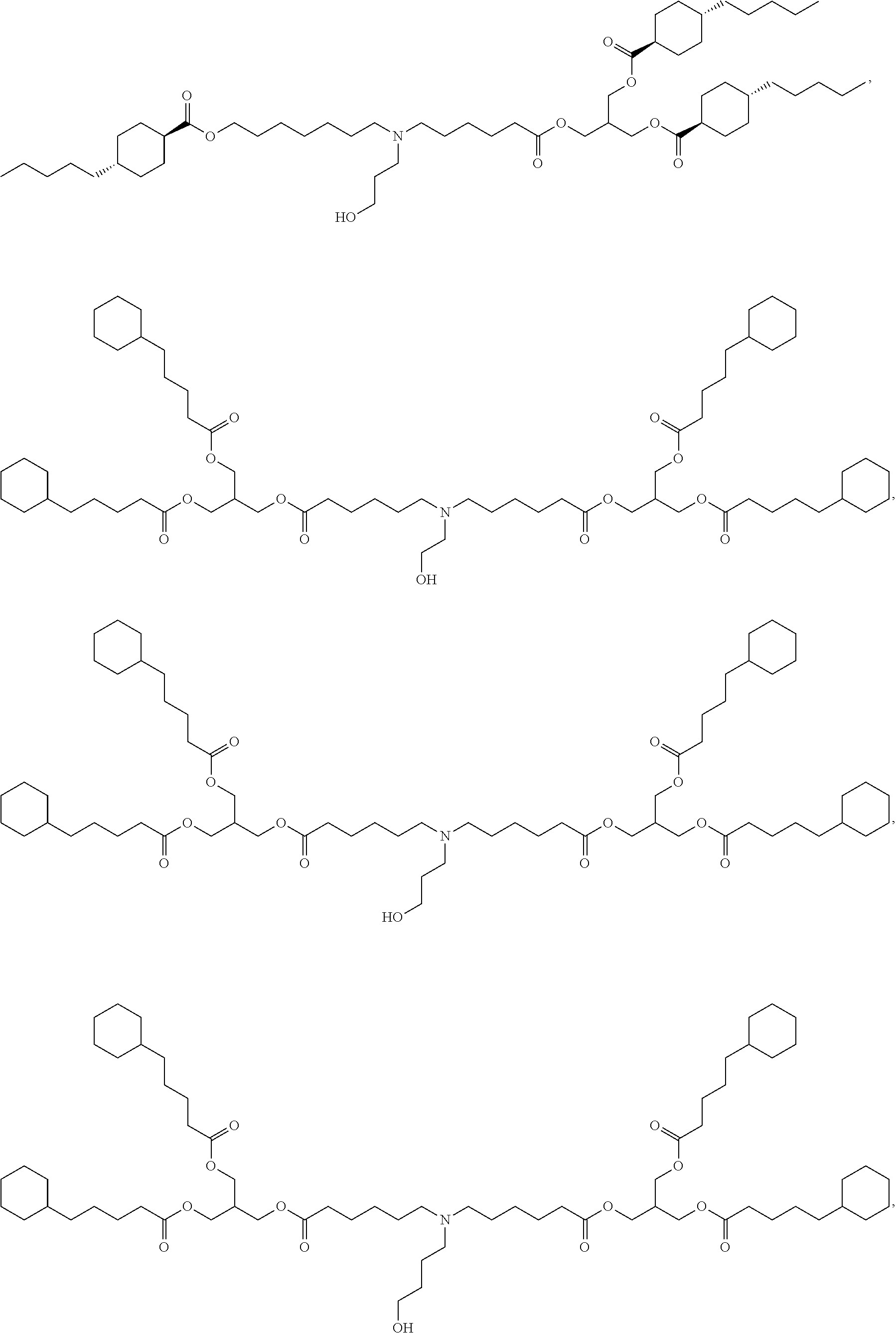 [SMILES: CCCCC[C@H]1CC[C@H](C(=O)OCCCCCCCN(CCCO)CCCCCC(=O)OCC(COC(=O)[C@H]2CC[C@H](CCCCC)CC2)COC(=O)[C@H]2CC[C@H](CCCCC)CC2)CC1 ; O=C(CCCCCN(CCO)CCCCCC(=O)OCC(COC(=O)CCCCC1CCCCC1)COC(=O)CCCCC1CCCCC1)OCC(COC(=O)CCCCC1CCCCC1)COC(=O)CCCCC1CCCCC1 ; O=C(CCCCCN(CCCO)CCCCCC(=O)OCC(COC(=O)CCCCC1CCCCC1)COC(=O)CCCCC1CCCCC1)OCC(COC(=O)CCCCC1CCCCC1)COC(=O)CCCCC1CCCCC1 ; O=C(CCCCCN(CCCCO)CCCCCC(=O)OCC(COC(=O)CCCCC1CCCCC1)COC(=O)CCCCC1CCCCC1)OCC(COC(=O)CCCCC1CCCCC1)COC(=O)CCCCC1CCCCC1]

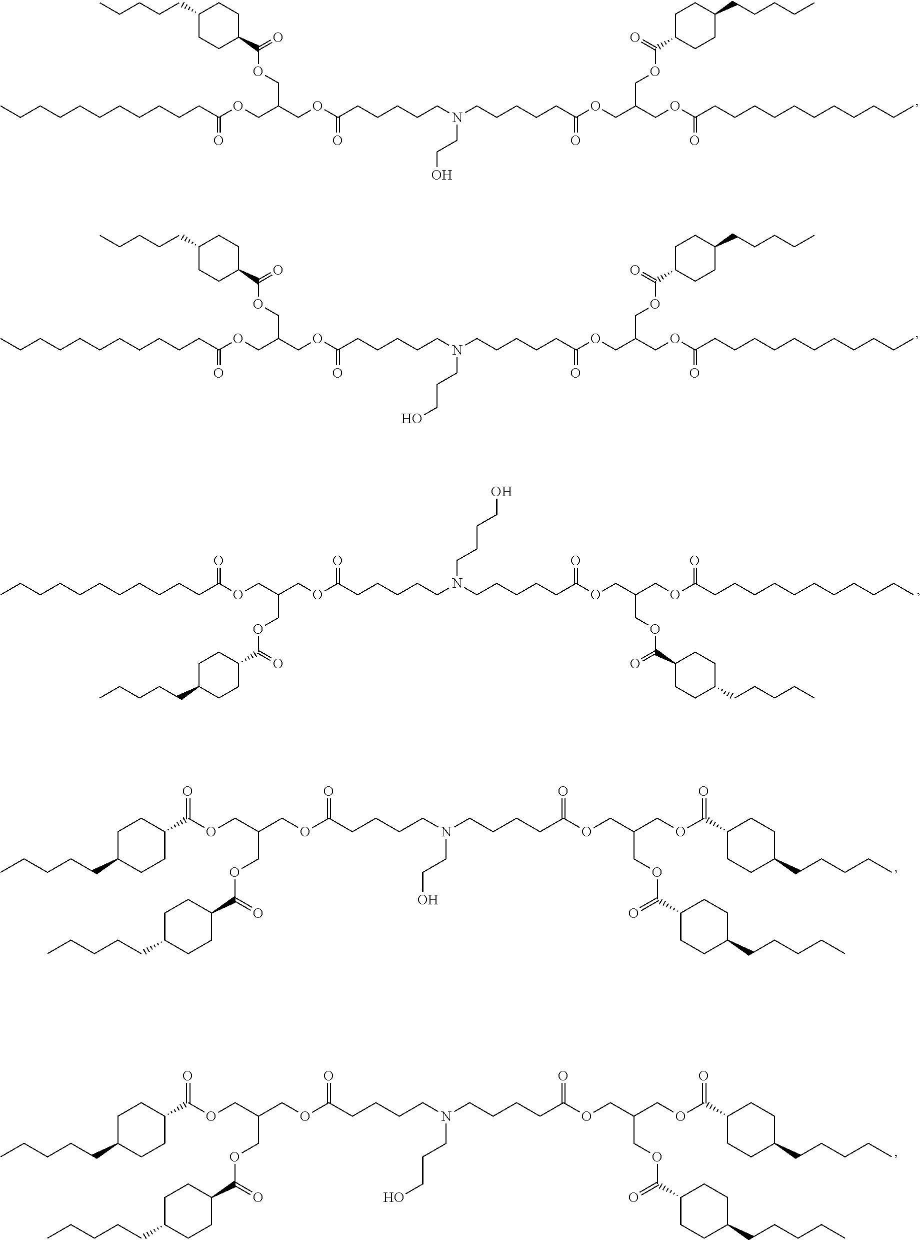

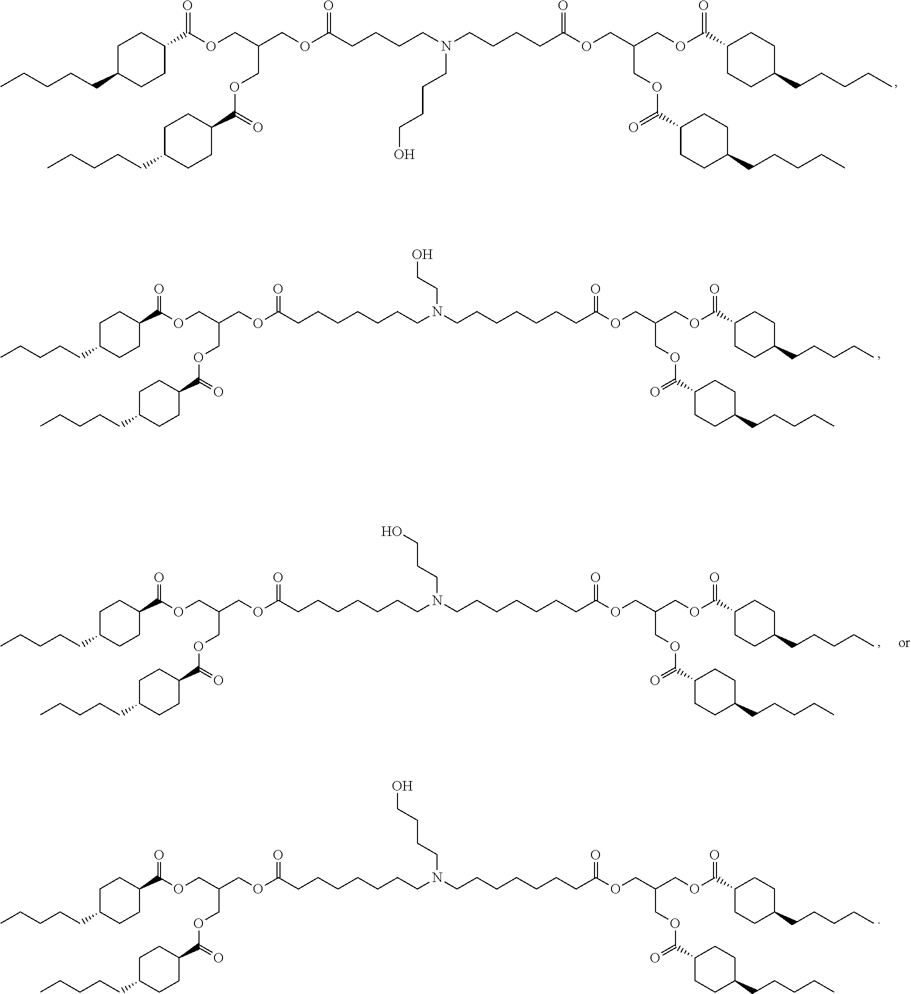

It will be understood that the compounds of any one of the Formulas disclosed herein and any pharmaceutically acceptable salts thereof, comprise stereoisomers, mixtures of stereoisomers, polymorphs of all isomeric forms of said compounds.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

It will be understood that in any of the formulae described herein, when a “-” is used to indicate linkage between two variables (e.g., A-B), the linkage could be one or more covalent bonds.

### General Methods for the Preparation of Compounds of Formula (I) or Formula (II) of the Present Disclosure

Compounds of Formula (I) or Formula (II) can be prepared using the reagents, intermediates, precursors, methods and schemes disclosed herein or using other commercially available reagents and methods known to those skilled in the art.

### General Procedure for Synthesis of Lipidoids (A)

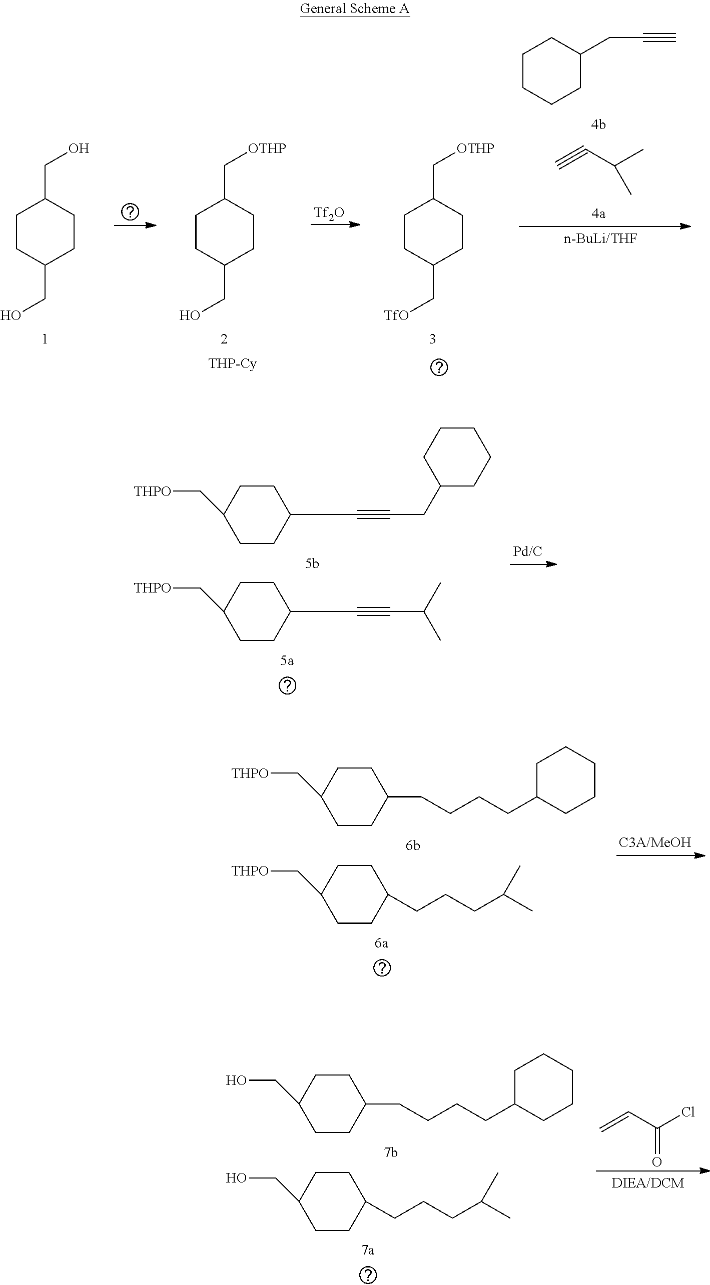

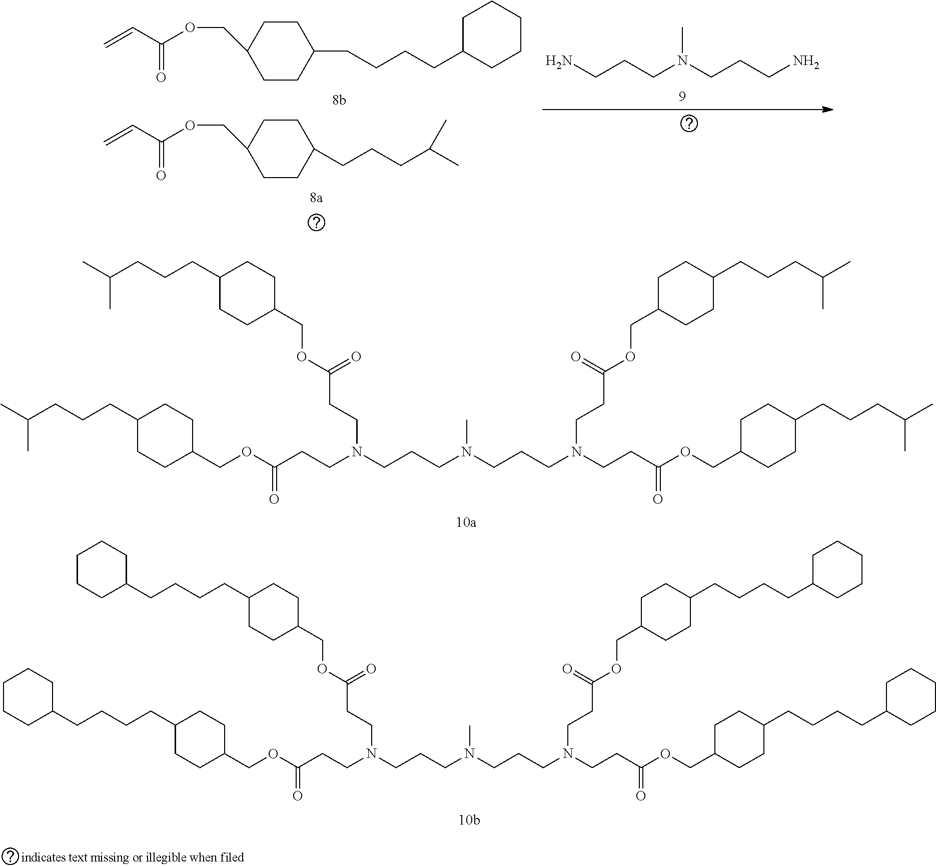

### Intermediate 2

Trans-cyclohexyl 1,4-di-methanol 1 (23 g, 0.16 mol) was dissolved in 200 ml dry THF and dihydropyran (13.4 g, 1.0 eq) was then added. The reaction mixture was cooled with an ice-bath followed by addition of pyridinium p-Toluene sulfonate (PPTS) (2 g, 0.05 eq). The resulting reaction mixture was stirred at room temperature for 24 hrs. The reaction mixture was quenched with sat. NaHCO₃ (200 ml) followed by sequential extraction with EtOAc (150 ml×3), washing with brine and drying over anhydrous sodium sulfate to give crude product after solvent was stripped off under reduced vacuum. The crude product was purified by silica gel column (150 g, gradient elution from 10%-30% EtOAC in hexane) to provide desired intermediate 2 (Cy-THP) (23.7 g, yield 65%) ¹H NMR (500 MHz, CDCl₃): δ 4.56-4.54 (m, 1H), 3.87-3.82 (m, 1H), 3.56-3.52 dd, J=9.8 Hz, 9.8 Hz), 3.51-3.46 (m, 1H), 3.46 (d, 1H, J=6 Hz), 3.21-3.218 (dd, 1H, J=3 hz, 6 Hz), 1.92-1.76 (m, 5H), 1.72-1.67 (m, 1H) 1.60-1.38 (m, 7H), 0.98-0.93 (m, 4H). MS: m/z 251.2 (M+Na).

### Intermediate 3

THP-Cy 2 (4 g, 17.54 mmol) was dissolved in dry DCM (100 mL) and dry pyridine (9 ml) at room temperature, and the mixture was cooled to −78° C. with an acetone-dry ice bath followed by addition of triflic anhydride (4.7 ml, 1.6 eq) dropwise at −78° C. in 30 mins. The reaction mixture was stirred from −78 to −30° C. in 3 hrs until TLC showed most of the starting material was used up. The reaction mixture was diluted with 100 ml DCM followed by quenching with 1N HCl (150 ml), sequential extraction with DCM (100 ml×2) and washing with NaHCO₃, brine and drying over NaSO₄ to provide crude triflate 3.

### Intermediate 5

Alkyne 4 (17.54 mmol) was placed in 50 ml dry THF and cooled down to −78° C. followed by addition of 2.5 M n-BuLi in hexane (8.4 ml, 1.2 eq) dropwise in 30 mins. The resulting mixture was stirred at −78° C. for 2 hrs followed by addition of HMPA (hexamethylphosphoramide) (6.3 g, 2 eq) at the same temperature, which was kept stirring for another 1.0 hr at −78° C. Crude triflate 3 in THF (10 ml) was added in a dropping funnel in 30 mins at −78° C. The reaction mixture was stirred for another 2-3 hrs until the temperature reached −30° C. and monitored with TLC until the triflate disappeared. The reaction mixture was quenched with sat. NH₄Cl (150 ml) and extracted with EtOAc (100 ml×3), washed with NaHCO₃, brine and dried over Na₂SO₄. The crude product was purified by silica gel column to get pure intermediate 5 in 90% yield.

¹H NMR (500 MHz, CDCl₃) of 5a: δ 4.57-4.55 (m, 1H), 3.86-3.83 (m, 1H), 3.56-3.52 (dd, 1H, J=9.8 Hz, 9.8.0 Hz), 3.49-3.48 (m, 1H), 3.26 (dd, 1H, J=4.8 Hz, 9.8 Hz), 2.53-2.51 (m, 1H), 2.06-2.04 (m, 2H), 1.88-1.79 (m, 5H), 1.75-1.67 (m, 1H), 1.62-1.49 (m, 5H), 1.42-1.33 (m, 1H), 1.14-1.12 ((dd, 6H, 6H, J=9.8 Hz), 1.04-0.96 (m, 4H). MS: m/z 301.1 (M+Na).

¹H NMR (500 MHz, CDCl₃) of 5b: δ 4.56 (T, 1H, J=4.8 Hz), 3.86-3.83 (m, 1H), 3.56-3.52 (dd, 1H, J=9.8 Hz, 8.0 Hz), 3.49-3.48 (m, 1H), 3.21-3.18 (dd, 1H, J=4.8 Hz, 9.6 Hz), 2.07-2.03 (m, 4H), 1.88-1.75 (m, 7H), 1.73-1.59 (m, 4H), 1.58-1.45 (m, 5H), 1.44-1.33 (m, 2H), 1.31-1.15 (m, 2H), 1.12-1.05 (m, 1H), 1.04-0.93 (m, 6H). MS: m/z 355.1 (M+Na).

### Intermediate 6

Intermediate 5 was dissolved in a mixture of solvents EtOAc/MeOH (2/1) and 10% weight of Pd/C was added at room temperature. The reaction mixture was treated with H₂ in a balloon overnight to give quantitative intermediate 6 without purification after filtration via a celite.

### Intermediate 7

Intermediate 6 was dissolved in MeOH (1 g/10 MeOH) followed by addition of CSA (0.05 eq). The resulting mixture was stirred at room temperature to give desired deprotected intermediate 7 after neutralization with TEA. After concentration and purification by silica gel column, intermediate 7 was obtained in 85% yield.

¹H NMR (500 MHz, CDCl₃) of 7a: δ 3.45 (d, 2H, J=4.8 Hz), 1.79-1.77 (m, 4H), 1.54-1.30 (m, 4H), 1.28-1.23 (m, 3H), 1.17-1.12 (m, 4H), 0.95-0.88 (m, 2H), 087-0.86 (d, 6H, J=3 Hz). MS: m/z 221.1 (M+Na).

¹H NMR (500 MHz, CDCl₃) of 7b: δ 3.45 (d, 2H, J=4.8 Hz), 1.78-1.76 (m, 4H), 1.69-1.64 (m, 5H), 1.56-1.42 (m, 2H), 1.26-1.09 (m, 12H), 0.97-0.81 (m, 6H). MS (APCI): m/z 275.2 (M+Na).

### Intermediate 8

Intermediate 7 was mixed with dry DCM (15 ml/g) and 1.5 eq DIEA followed by addition of acryloyl chloride (1.6 eq) in an ice-bath. The resulting reaction mixture was stirred at room temperature for 2-3 hrs until starting material was used up. The reaction mixture was quenched with cold 1N HCl (2 eq) and extracted with DCM twice followed by washing with sat. NaHCO₃, brine and drying over Na₂SO₄. After purification by silica gel column, desired intermediate 8 was obtained in 85% yield.

### Products 10a and 10b

Acrylate 7 and amine 404 (0.225 eq per acrylate) were mixed in neat condition and heated to 90° C. for 48 hrs until the major product was the desired product as monitored by LCMS. The reaction mixture was cooled to room temperature and diluted with DCM, then purified by silica gel column with DCM/EtOAC to get desired product 10 in 40-50% yield.

¹H NMR (500 MHz, CDCl₃) of 10a: δ 3.87 (d, 8H, J=9.8 Hz), 2.77 (t, 8H, J=9.8 Hz), 2.43 (t, 12H, J=4.8 Hz), 2.28 (m, 4H), 2.18 (s, 3H), 1.78-1.74 (m, 14H), 1.58-1.49 (m, 20H), 1.31-1.22 (m, 16H), 1.16-1.11 (m, 14H), 0.97-0.88 (m, 8H), 0.86 (d, 24H, J=9.8 Hz). MS: m/z 1155.2 (M+H).

¹H NMR (500 MHz, CDCl₃) of 10b: δ 3.87 (d, 8H, J=6 Hz), 2.77 (t, 8H, J=6 Hz), 2.43 (t, 12H, J=4.8 Hz), 2.19 (m, 4H), 2.18 (s, 3H), 1.77-1.61 (m, 30H), 1.25-1.19 (m, 20H), 1.18-1.12 (m, 32H), 0.98-0.82 (m, 24H). MS: m/z 1371.2 (M+H).

### General Procedure for Synthesis of Lipidoids (B)

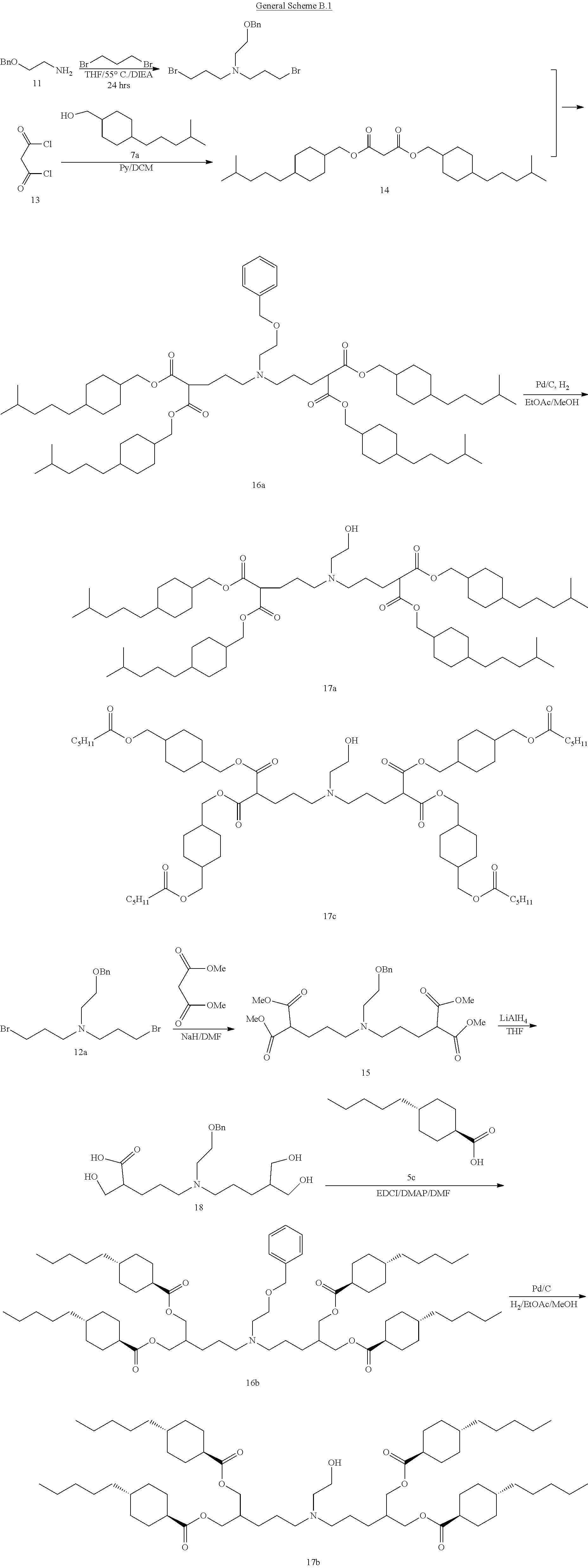

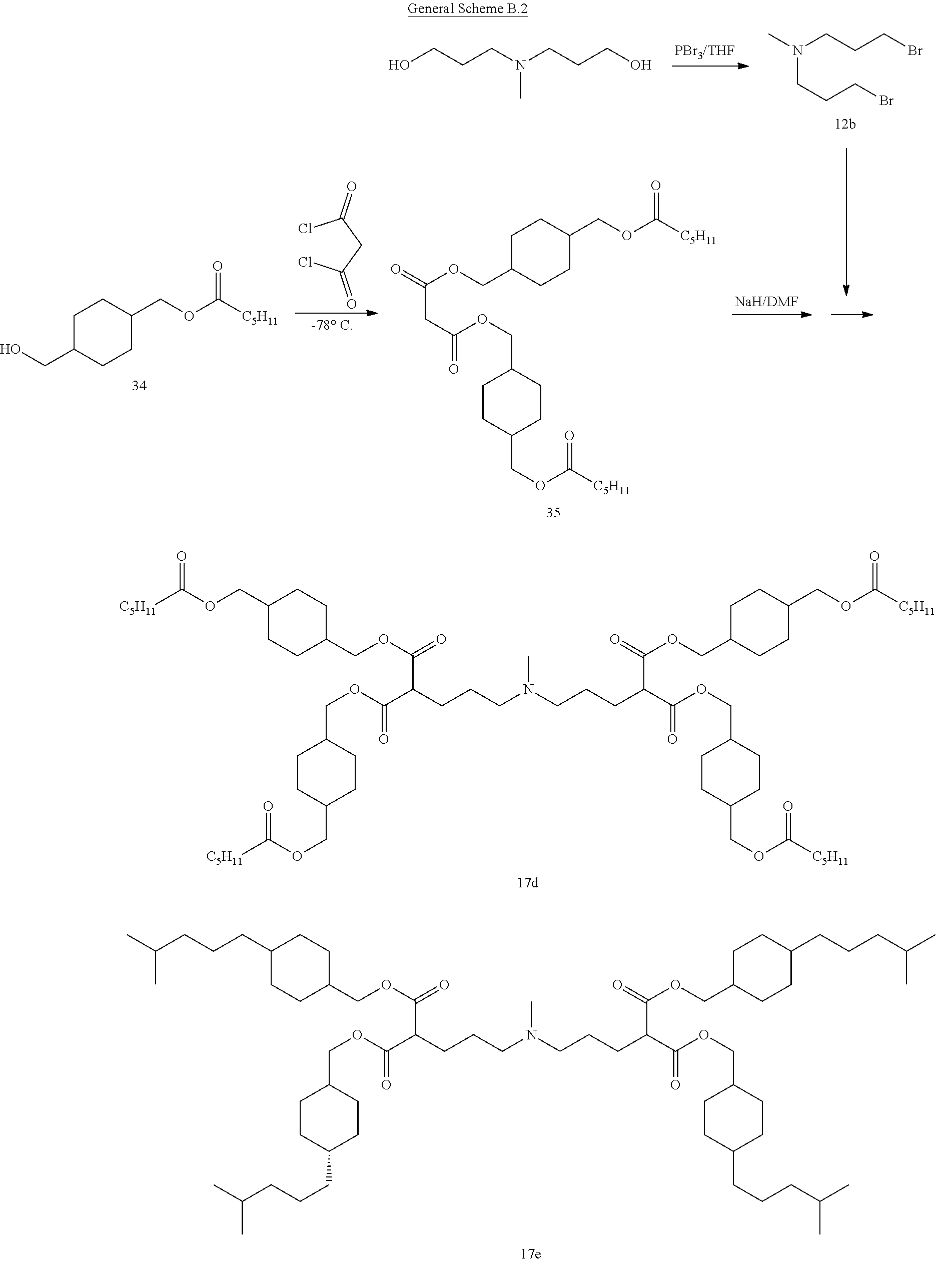

### Intermediate 12a

Starting material 11 (1.0 g, 6.62 mmol) was dissolved in dry THF (30 ml) and 1,3 dibromo propane (2.94 g, 2.2 eq) and DIEA (3.37 ml, 3.0 eq) were added. The mixture was heated to 55° C. for 24 hrs and cooled down to room temperature. The mixture was diluted with EOTAc (100 ml) and then washed with sat. NaHCO₃ and brine to give crude product. After purification by silica gel column, 910 mg of intermediate 12a was obtained as pale-yellow oil (35% yield). ¹H NMR (500 MHz, CDCl₃) of 12a: 87.37-7.26 (m, 5H), 4.52 (s, 2H), 2.68-2.60 (m, 6H), 2.05-1.95 (m, 4H). MS: m/z 392.0 (M+H).

### Intermediate 14

Pyridine (0.29 ml, 5.0 eq) and alcohol 7a (300 mg, 2.1 eq) were dissolved in dry DCM (10 ml) followed by addition of malonic chloride 13 (100 mg, 0,714 mmol) dropwise in an ice bath. The resulting reaction mixture was stirred at room temperature for 2-3 hrs until starting material was used up. The reaction mixture was quenched with sat. NaHCO₃ and extracted with DCM. The crude product was purified by silica gel after drying and concentrated to afford desired intermediate 14 (225 mg, 68% yield).

### Intermediate 16a

Intermediate 14 (100 mg, 0.215 mmol) was dissolved in dry DMF (5 ml) followed by addition of NaH (60%, 9.46 mg, 1.1 eq in ice bath). The resulting mixture was stirred for 30 mins at room temperature followed by addition of intermediate 12a (38 mg, 0.45 eq) at room temperature. The mixture was warmed to 40° C. for 2-3 hrs until the reaction went to completion. The reaction mixture was quenched with sat. NH₄Cl (20 ml) and extracted with EtOAc (20 mL×3) followed by washing with NaHCO₃ and brine and dried over anhydrous sodium sulfate to get crude product, which was purified to get desired intermediate 16a (57 mg, 51% yield).

### Product 17a

A solution of intermediate 14 (360 mg, 0.78 mmol) in dry DMF (1 mL) and NaH (1 mmol, 40 mg) was stirred at room temperature for 30 min before N-(2-(benzyloxy)ethyl)-3-bromo-N-(3-bromopropyl) propan-1-amine (0.44 mmol, 134 mg) was added. The reaction was kept at 40° C. for 12 h before being quenched by diluted ice old HCl solution (1 N). The mixture was washed by EtOAc (3×100 mL) and brine (3×100 mL). The combined organic layer was dried over Na₂SO₄ and concentrated under vacuum. The crude was purified by column chromatography (DCM:MeOH=10:1 to 5:1, v/v) followed by reverse phase Biotage to afford the desired intermediate compound in a yield of 10%.

The intermediate compound was then treated with 10% Pd/C (80 mg) under H₂ gas and the reaction was filtered after 12 h. The crude was purified by reverse phase Biotage to afford the desired compound 17a in a yield of 75%, ¹H NMR (500 MHz, CDCl₃) δ 4.05-3.83 (m, 9H), 3.38 (t, J=7.1 Hz, 2H), 3.22-2.99 (m, 5H), 1.91 (dd, J=10.4, 5.3 Hz, 4H), 1.89-1.66 (m, 19H), 1.54 (ddq, J=26.6, 13.3, 6.7 Hz, 9H), 1.39-1.22 (m, 10H), 1.24-1.08 (m, 19H), 1.01-0.88 (m, 12H), 0.86 (d, J=6.6 Hz, 26H). MS (ESI): calcd. for C₆₆H₁₁₉NO₉ [M+H]⁺ 1070.9, found 1071.1.

### Intermediate 15

Intermediate 15 was prepared in accordance with the procedure for preparation of intermediate 16a.

### Intermediate 18

Intermediate 15 was dissolved in dry THF and cooled down with ice-water followed by addition of 1M LiAlH₄ (1.5 eq) drop wise. The resulting reaction mixture was stirred at room temperature overnight and then quenched sequentially with water, 15% aq NaOH and water under argon. After filtration via celite, the filtrate was extracted with EtOAc and sat. NaHCO₃. The organic phases were combined and dried over Na₂SO₄. The crude product was applied to the next step without separation.

### Product 17b

Intermediate 18 and cyclohexyl acid (4.4 eq) were mixed in dry DMF followed by addition of EDCl (6.0 eq) and DMAP (8.0 eq) at room temperature. The resulting reaction mixture was stirred at room temperature for 24 hrs to give desired product 17b after purification by silica gel column (Hex/EtOAc).

### Product 17c

The Bn-protected precursor (100 mg) was prepared in accordance with the procedure for preparation of intermediate 16a. The precursor was then dissolved in MeOH (1 mL) and treated with Pd/C (10% by weight) under H₂ balloon for 12 hrs to get product 17c after filtration and concentration. The crude was purified by reverse phase Biotage to afford the desired compound in a yield of 75%, ¹H NMR (500 MHz, CDCl₃) δ 4.12-3.91 (m, 13H), 3.89 (d, J=6.5 Hz, 6H), 3.43-3.33 (m, 2H), 3.13 (d, J=16.1 Hz, 6H), 2.29 (t, J=7.6 Hz, 8H), 1.96-1.71 (m, 22H), 1.68-1.47 (m, 19H), 1.46-1.24 (m, 22H), 1.06-0.94 (m, 11H), 0.89 (t, J=6.8 Hz, 12H). MS (ESI): calcd. for C₇₀H₁₁₉NO₁₇ [M+H]⁺ 1246.8, found 1247.0.

### Intermediate 35

To a solution of alcohol 34 (4.5 g, 18.5 mmol) and malonyl dichloride (1.06 g, 7.6 mmol) in dry DCM (70 mL) was added TEA (66 mmol, 7 g) at −70° C. The reaction was kept at −70° C. to −30° C. for 6 h before being quenched by 1 N HCl (200 mL). The mixture was washed with EtOAc (3×200 mL), saturated NaHCO₃ (2×200 mL) and brine (3×200 mL). The combined organic layer was dried over Na₂SO₄ and concentrated under vacuum. The crude was purified by column chromatography (Hex:EA=10:1, v/v) to afford the desired intermediate 35 as colorless oil (2.9 g) in a yield of 70%. ¹H NMR (500 MHz, CDCl₃) δ 4.06 (d, J=7.2 Hz, 1H), 3.97 (t, J=7.1 Hz, 3H), 3.88 (d, J=6.5 Hz, 2H), 3.37 (s, 2H), 2.29 (t, J=7.6 Hz, 4H), 1.95-1.75 (m, 6H), 1.71-1.47 (m, 9H), 1.45-1.23 (m, 11H), 1.05-0.96 (m, 5H), 0.88 (t, J=6.9 Hz, 6H).

### 3-bromo-N-(3-bromopropyl)-N-methylpropan-1-amine (12b)

To a solution of 3,3′-(Methylazanediyl)bis(propan-1-ol) (10 mmol) in dry DCM at 0° C. was added PBr₃ (50 mmol); the mixture was heated at 37° C. for 12 h before being quenched by saturated NaHCO₃ under an bath. The mixture was washed by DCM (3×200 mL) and brine (3×200 mL). The combined organic layer was dried over Na₂SO₄ and concentrated under vacuum. The crude was purified by column chromatography (DCM: MeOH=5:1, v/v) to afford desired crude. The crude was then purified by reverse phase Biotage to afford 12b in a yield of 35%, ¹H NMR (500 MHz, CDCl₃) δ 3.52 (ddd, J=7.6, 5.0, 2.4 Hz, 2H), 3.28 (tt, J=11.3, 4.5 Hz, 1H), 3.20-3.10 (m, 1H), 2.82 (d, J=4.6 Hz, 2H), 2.66-2.52 (m, 1H), 2.44 (tdd, J=14.2, 7.3, 5.2 Hz, 1H). MS (ESI): calcd. for C₇H₁₅Br₂N [M+H]⁺ 272.0, found 271.9.

### Product 17d

Product 17d was prepared in accordance with the procedure for preparation of intermediate 16a. ¹H NMR (500 MHz, CDCl₃) δ 4.13-3.92 (m, 11H), 3.89 (d, J=6.5 Hz, 6H), 3.43-3.33 (m, 3H), 3.14 (s, 3H), 2.94 (s, 2H), 2.76 (s, 3H), 2.29 (t, J=7.6 Hz, 8H), 1.97-1.73 (m, 23H), 1.67-1.57 (m, 14H), 1.53 (tq, J=8.3, 4.4 Hz, 6H), 1.41 (q, J=7.3 Hz, 7H), 1.37-1.25 (m, 17H), 1.06-0.94 (m, 12H), 0.93-0.85 (m, 12H). MS (ESI): calcd. for C₆₉H₁₁₇NO₁₆ [M+H]⁺ 1216.8, found 1216.6.

### Product 17e

Product 17e was prepared in accordance with the procedure for preparation of intermediate 16a. ¹H NMR (500 MHz, CDCl₃) δ 3.99-3.86 (m, 6H), 3.35 (t, J=7.6 Hz, 2H), 2.38-2.29 (m, 4H), 2.15 (s, 3H), 1.88 (q, J=7.6 Hz, 4H), 1.81-1.69 (m, 11H), 1.64-1.43 (m, 29H), 1.26 (t, J=5.2 Hz, 8H), 1.21-1.08 (m, 14H), 1.02-0.88 (m, 8H), 0.86 (d, J=6.6 Hz, 18H). MS (ESI): calcd. for C₆₅H₁₁₇NO₈ [M+H]⁺ 1040.9, found 1040.9.

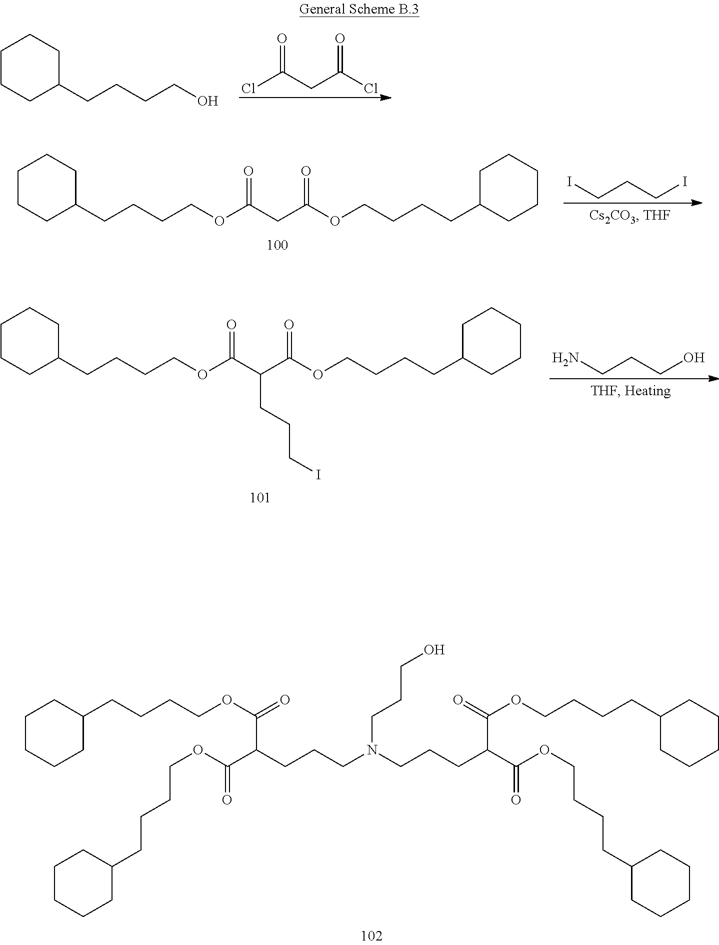

To a solution of 4-cyclohexylbutan-1-ol (2.26 g, 14 mmol) and malonyl dichloride (0.85 g, 6 mmol) in dry DCM (50 mL) was added TEA (40 mmol, 5 g) at −70° C. The reaction was kept at −70° C. to −30° C. for 6 h before being quenched by 1 N HCl (200 mL). The mixture was washed by EtOAc (3×200 mL), sat. NaHCO₃ (2×200 mL) and brine (3×200 mL). The combined organic layer was dried over Na₂SO₄ and concentrated under vacuum. The crude was purified by column chromatography (Hex:EA=10:1, v/v) to afford desired product 100 as a colorless oil (1.7 g) in a yield of 75%, ¹H NMR (500 MHz, CDCl₃) δ 4.13 (t, J=6.8 Hz, 4H), 3.36 (s, 2H), 1.65 (dddd, J=30.4, 14.9, 8.2, 4.7 Hz, 14H), 1.41-1.28 (m, 4H), 1.26-1.10 (m, 12H), 0.94-0.76 (m, 5H).

To a solution of bis(4-cyclohexylbutyl) malonate (570 mg, 1.4 mmol) and Cs₂CO₃ (550 mg, 1.68 mmol) in dry THF (6 mL) was added 1,3-diiodopropane (498 mg, 1.68 mmol); the mixture was kept at room temperature for 6 h. The mixture was concentrated under vacuum and directly purified by column chromatography (Hex: (EA:DCM=1:6, v/v)=50:1, v/v) to afford desired product 101 as a colorless oil (260 mg) in a yield of 35%.

To a solution of 101 (150 mg, 0.27 mmol) and 3-Amino-1-propanol (8.3 mg, 0.11 mmol) in THF/MeCN (1:1, v/v) was added DIEA (116 mg, 0.9 mmol). The mixture was heated at 60° C. for 5 days. The mixture was purified by reverse phase column chromatography (mobile A: 0.1% TFA in H2O and mobile B: MeCN:IPA=1:1, v/v) to afford product 102. ¹H NMR (500 MHz, CDCl₃) δ 4.12 (qt, J=10.8, 6.8 Hz, 4H), 3.08 (d, J=9.5 Hz, 2H), 1.91 (q, J=7.3 Hz, 11H), 1.79 (d, J=7.1 Hz, 2H), 1.73-1.55 (m, 14H), 1.33 (tdd, J=9.6, 7.1, 5.3 Hz, 4H), 1.28-1.07 (m, 12H), 0.86 (q, J=11.3 Hz, 4H). MS (ESI): calcd. for C₅₅H₉₇NO₉ [M+H]⁺ 916.7, found 917.0.

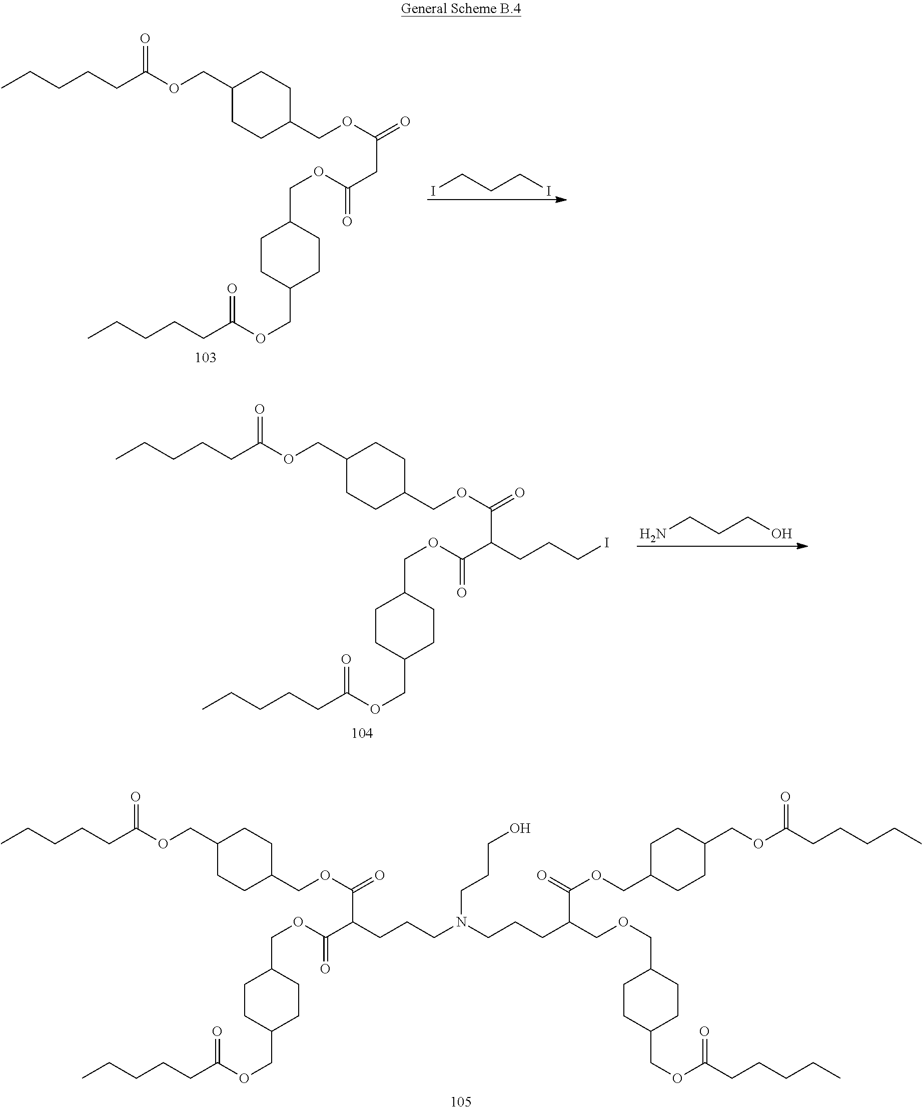 [SMILES: CCCCCC(=O)OCC1CCC(COC(=O)CC(=O)OCC2CCC(COC(=O)CCCCC)CC2)CC1 ; ICCCI ; CCCCCC(=O)OCC1CCC(COC(=O)C(CCCI)C(=O)OCC2CCC(COC(=O)CCCCC)CC2)CC1 ; NCCCO ; CCCCCC(=O)OCC1CCC(COCC(CCCN(CCCO)CCCC(C(=O)OCC2CCC(COC(=O)CCCCC)CC2)C(=O)OCC2CCC(COC(=O)CCCCC)CC2)C(=O)OCC2CCC(COC(=O)CCCCC)CC2)CC1]

To a solution of 103 (1.656 g, 3 mmol) and Cs₂CO₃ (1174 mg, 3.6 mmol) in dry THF (10 mL) was added 1,3-diiodopropane (1066 mg, 3.6 mmol) and the mixture was kept at room temperature for 6 h. The mixture was concentrated under vacuum and directly purified by column chromatography (Hex: (EA:DCM=1:6, v/v)-50:1, v/v) to afford desired product 104 as a colorless oil (496 mg) in a yield of 30%. ¹H NMR (500 MHz, CDCl₃) δ 4.06 (dd, J=11.7, 7.2 Hz, 1H), 4.00-3.93 (m, 3H), 3.89 (d, J=6.5 Hz, 3H), 3.36 (t, J=7.4 Hz, 1H), 3.18 (t, J=6.8 Hz, 2H), 2.30 (t, J=7.5 Hz, 4H), 2.05-1.98 (m, 2H), 1.91-1.75 (m, 9H), 1.62 (p, J=7.5 Hz, 7H), 1.52 (d, J=5.3 Hz, 1H), 1.41 (d, J=10.1 Hz, 3H), 1.37-1.24 (m, 12H), 1.00 (td, J=8.9, 2.8 Hz, 6H), 0.90 (t, J=7.0 Hz, 8H). MS (ESI): calcd. for C₃₄H₅₇IO₈ [M+Na]⁺743.3, found 743.2.

To a solution of 104 (160 mg, 0.22 mmol) and 3-Amino-1-propanol (7.5 mg, 0.1 mmol) in THF/MeCN (1:1, v/v) was added DIEA (116 mg, 0.9 mmol) and the mixture was heated at 60° C. for 5 days. The mixture was purified by reverse phase column chromatography (mobile A: 0.1% TFA in H2O and mobile B: MeCN:IPA=1:1, v/v) to yield product 105.

### General Procedure for Synthesis of Lipidoids (C)

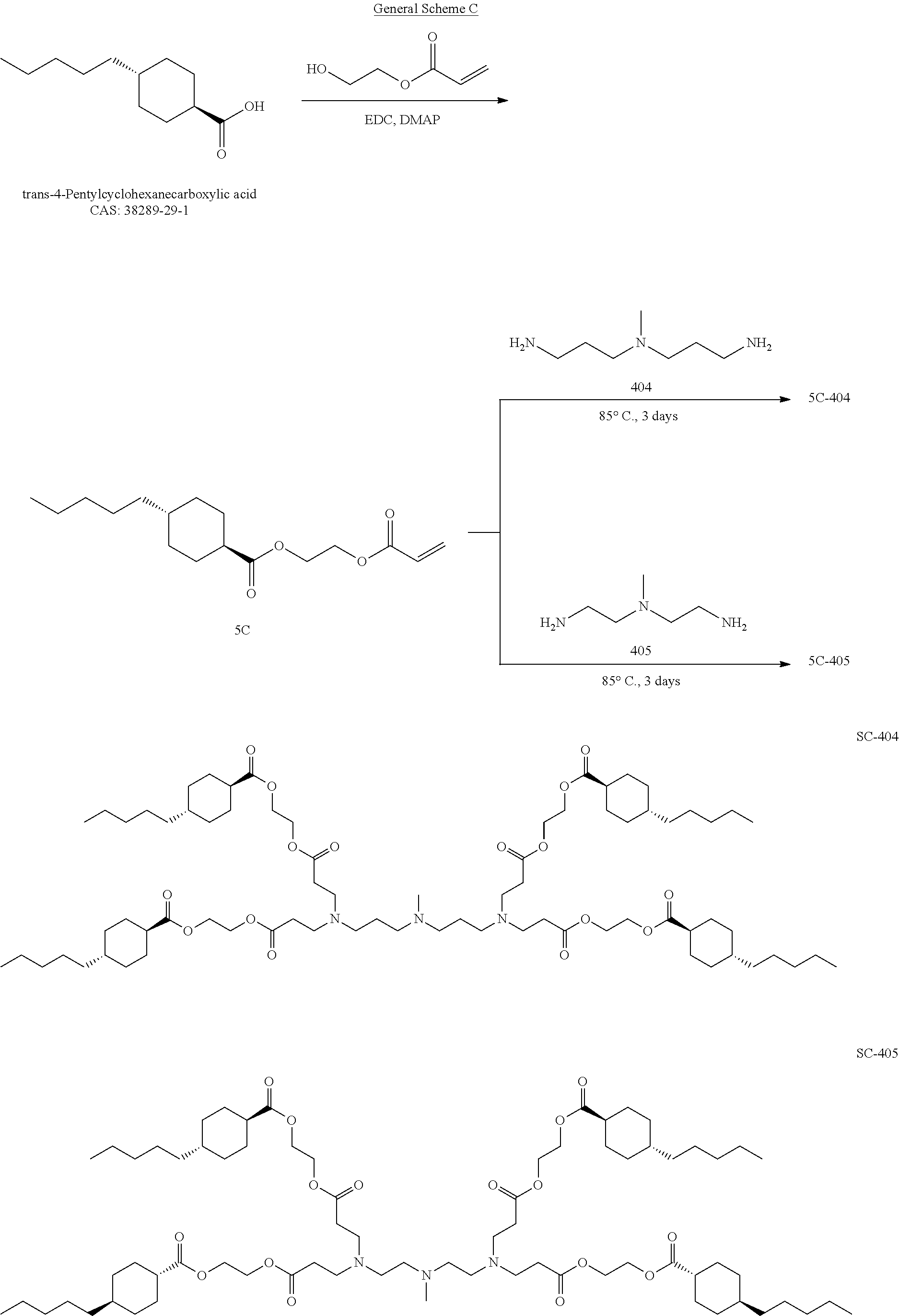

### Synthesis of Acrylates

### General Protocol

In a round bottomed flask, cyclohexyl-based carboxylic acid (1.0 eq), 4-dimethylaminopyridine (DMAP) (0.4 eq), and N-(3-Dimethylaminopropyl)-N′-ethylcarbodiimide hydrochloride (EDC) (1.5 eq) were dissolved in dichloromethane. The resulting solution was stirred for 20 mins at room temperature before adding 2-hydroxyethyl acrylate (1.5 eq) drop wise. The resulting solution was stirred for 20 h at room temperature. Water was added and extracted with dichloromethane (3×). Combined organic extracts were washed with brine; dried over Na₂SO₄; filtered and evaporated. The crude was then purified by silica gel flash column chromatography using 3-5% EtOAc/hexane eluants to give acrylates as colorless oils.

### Intermediate 5C

In a 100 mL round bottomed flask, trans-4-pentylcyclohexane carboxylic acid (2.5 g, 1.0 eq), 4-dimethylaminopyridine (DMAP) (0.62 g, 0.4 eq), and N-(3-Dimethylaminopropyl)-N′-ethylcarbodiimide hydrochloride (EDC) (3.62 g, 1.5 eq) were dissolved in dichloromethane (30 mL). The resulting solution was stirred for 20 mins at room temperature before adding 2-hydroxyethyl acrylate (2.2 mL, 1.5 eq) drop wise. The resulting solution was stirred for 20 h at room temperature. Water (50 mL) was added and extracted with dichloromethane (3×50 mL). Combined organic extracts were washed with brine (20 mL); dried over Na₂SO₄; filtered and evaporated. The crude was then purified by silica gel flash column chromatography using 5% EtOAc/hexane eluants to give 5C as a colorless oil.

Colorless oil, 3.08 g; Yield: 82%; ¹H NMR (499 MHz, CDCl₃) δ 6.42 (dd, J=17.4, 1.4 Hz, 1H), 6.13 (dd, J=17.3, 10.5 Hz, 1H), 5.86 (dd, J=10.5, 1.4 Hz, 1H), 4.38-4.28 (m, 4H), 2.24 (tt, J=12.3, 3.6 Hz, 1H), 2.00-1.91 (m, 2H), 1.84-1.76 (m, 2H), 1.46-1.34 (m, 2H), 1.34-1.12 (m, 9H), 0.95-0.83 (m, 5H).

### Intermediate C4

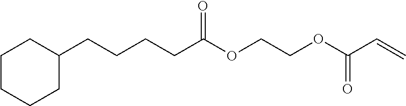 [SMILES: C=CC(=O)OCCOC(=O)CCCCC1CCCCC1]

In a 100 mL round bottomed flask, cyclohexanepentanoic acid (1.0 g, 1.0 eq), 4-dimethylaminopyridine (DMAP) (0.26 g, 0.4 eq), and N-(3-Dimethylaminopropyl)-N′-ethylcarbodiimide hydrochloride (EDC) (1.25 g, 1.5 eq) were dissolved in dichloromethane (10 mL). The resulting solution was stirred for 20 mins at room temperature before adding 2-hydroxyethyl acrylate (0.76 mL, 1.5 eq) drop wise. The resulting solution was stirred for 20 h at room temperature. Water (50 mL) was added and extracted with dichloromethane (3×50 mL). Combined organic extracts were washed with brine (20 mL); dried over Na₂SO₄; filtered and evaporated. The crude was then purified by silica gel flash column chromatography using 5% EtOAc/hexane eluants to give C₄ as a colorless oil.

Colorless oil, 0.86 g; Yield: 59%; ¹H NMR (499 MHz, CDCl₃) δ 6.43 (dd, J=17.3, 1.4 Hz, 1H), 6.14 (dd, J=17.3, 10.5 Hz, 1H), 5.86 (dd, J=10.5, 1.4 Hz, 1H), 4.38-4.29 (m, 4H), 2.32 (t, J=7.5 Hz, 2H), 1.70-1.57 (m, 7H), 1.35-1.27 (m, 2H), 1.25-1.09 (m, 6H), 0.90-0.79 (m, 2H).

### Synthesis of Lipidoids

### General Protocol

Polyamine (404 or 405, shown in general Scheme C) (1.0 eq) and acrylate 5C or 3C or C₄ (5.0 eq) were combined in a scintillation glass vial. The vial was capped, and the reaction mixture was stirred for 3 days at 85° C. The cooled reaction crude was purified by silica gel flash column chromatography using 5-10% MeOH/CH₂Cl₂ eluants to give pale brown oils as target compounds.

### Product 5C-404

Amine 404 (116 mg, 1.0 eq) and 5C (1.21 g, 5.0 eq) were combined in a 20 mL scintillation glass vial. The capped vial was stirred for 3 days at 85° C. Cooled reaction was purified by silica gel flash column chromatography with 4% MeOH/CH₂Cl₂.

Pale brown oil, 0.91 g; Yield: 85%; ¹H NMR (499 MHz, CDCl₃) δ 4.25 (s, 16H), 2.76 (t, J=7.2 Hz, 8H), 2.46 (t, J=7.3 Hz, 11H), 2.35-2.12 (m, 10H), 1.98-1.92 (m, 8H), 1.83-1.77 (m, 8H), 1.66-1.53 (m, 6H), 1.40 (qd, J=13.3, 3.4 Hz, 8H), 1.32-1.14 (m, 36H), 0.96-0.84 (m, 20H); Mass:

### Product 5C-405

Amine 405 (107 mg, 1.0 eq) and 5C (1.38 g, 5.0 eq) were combined in a 20 mL scintillation glass vial. The capped vial was stirred for 3 days at 85° C. Cooled reaction was purified by silica gel flash column chromatography with 4% MeOH/CH₂Cl₂.

Pale brown oil, 0.80 g; Yield: 67%; ¹H NMR (499 MHz, CDCl₃) δ 4.25 (s, 16H), 2.79 (t, J=7.2 Hz, 8H), 2.59-2.38 (m, 15H), 2.30-2.15 (m, 7H), 1.99-1.91 (m, 8H), 1.84-1.77 (m, 8H), 1.40 (qd, J=13.1, 3.5 Hz, 8H), 1.34-1.12 (m, 37H), 0.97-0.82 (m, 20H).

### Product C₄-404

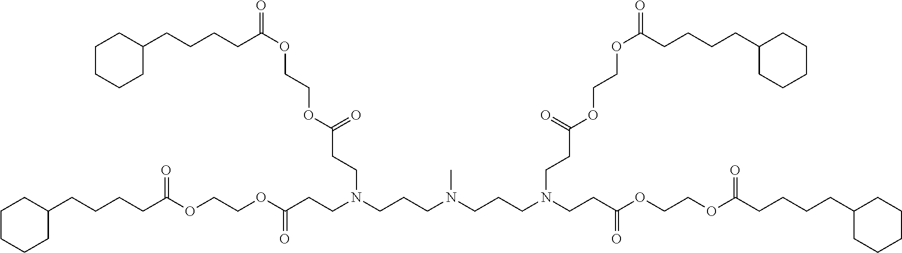 [SMILES: CN(CCCN(CCC(=O)OCCOC(=O)CCCCC1CCCCC1)CCC(=O)OCCOC(=O)CCCCC1CCCCC1)CCCN(CCC(=O)OCCOC(=O)CCCCC1CCCCC1)CCC(=O)OCCOC(=O)CCCCC1CCCCC1]

Amine 404 (29 mg, 1.0 eq) and C₄ (0.3 g, 5.0 eq) were combined in a 4 mL scintillation glass vial. The capped vial was stirred for 3 days at 85° C. Cooled reaction was purified by silica gel flash column chromatography with 4% MeOH/CH₂Cl₂.

Pale brown oil, 0.136 mg; Yield: 60%; ¹H NMR (499 MHz, CDCl₃) δ 4.26 (s, 16H), 2.77 (t, J=7.2 Hz, 8H), 2.50-2.40 (m, 12H), 2.36-2.25 (m, 12H), 2.18 (s, 3H), 1.71-1.55 (m, 33H), 1.35-1.28 (m, 8H), 1.23-1.12 (m, 23H), 0.89-0.80 (m, 8H).

### Product C₄-405

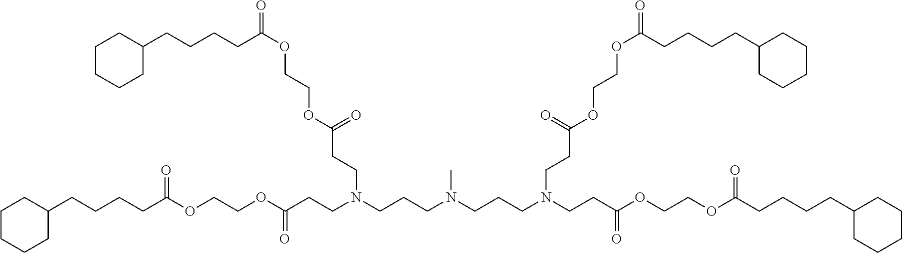 [SMILES: CN(CCCN(CCC(=O)OCCOC(=O)CCCCC1CCCCC1)CCC(=O)OCCOC(=O)CCCCC1CCCCC1)CCCN(CCC(=O)OCCOC(=O)CCCCC1CCCCC1)CCC(=O)OCCOC(=O)CCCCC1CCCCC1]

Amine 405 (25 mg, 1.0 eq) and C₄ (0.3 g, 5.0 eq) were combined in a 4 mL scintillation glass vial. The capped vial was stirred for 3 days at 85° C. Cooled reaction was purified by silica gel flash column chromatography with 4% MeOH/CH₂Cl₂.

Pale brown oil, 0.101 mg; Yield: 45%; ¹H NMR (499 MHz, CDCl₃) δ 4.26 (s, 16H), 2.79 (t, J=7.2 Hz, 8H), 2.58-2.40 (m, 15H), 2.32 (t, J=7.5 Hz, 8H), 2.22 (s, 3H), 1.71-1.56 (m, 29H), 1.34-1.10 (m, 32H), 0.89-0.80 (m, 8H).

### General Procedure for Synthesis of Lipidoids (D)

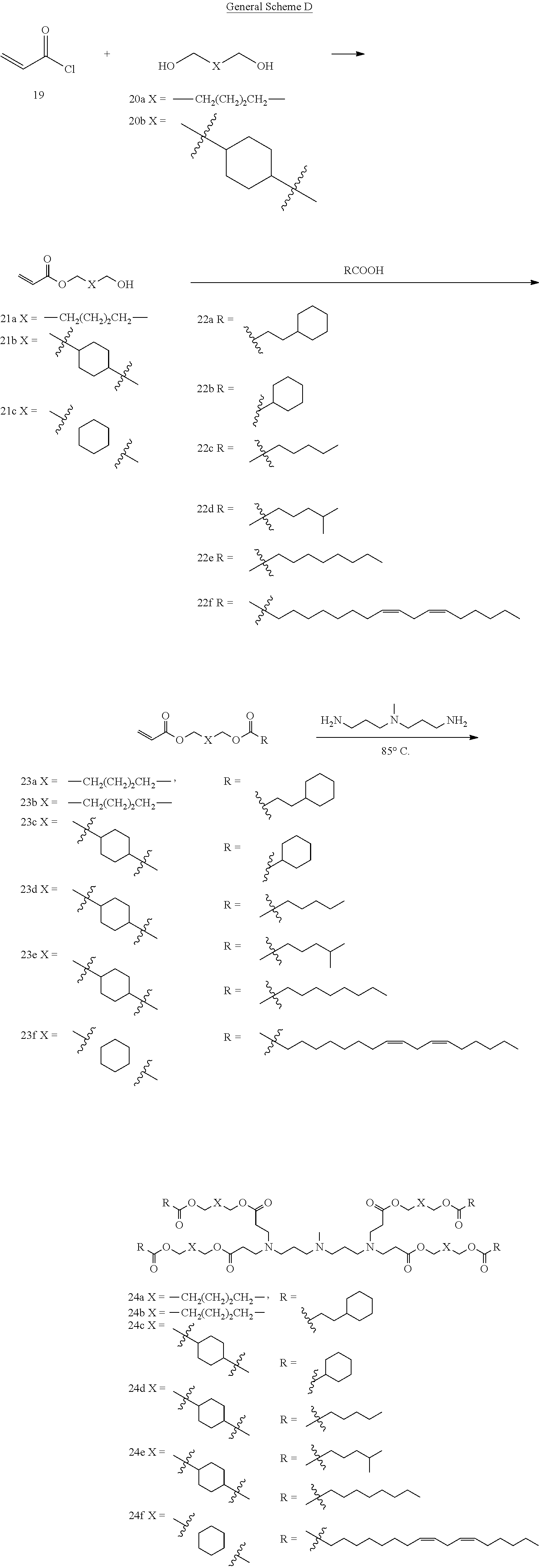

### Intermediate 21a

To diol 20a (2 g, 13.7 mmol) in DCM (200 mL) at 0° C. was added TEA (1.38 g, 13.7 mmol) and 19 (1.08 g, 11.9 mmol) dropwise. The resulting mixture was stirred from 0° C. to room temperature overnight, washed with 1N HCl, saturated NaHCO₃ and brine. The combined organic phase was dried over Na₂SO₄ and concentrated. Purification by column chromatography (ethyl acetate/hexane) afforded intermediate 21a (861 mg, 36%) as clear oil. ¹H NMR (500 MHz, CDCl₃) δ 6.39 (dd, J=17.4, 1.5 Hz, 1H), 6.11 (dd, J=17.4, 10.4 Hz, 1H), 5.81 (dd, J=10.4, 1.5 Hz, 1H), 4.14 (t, J=6.7 Hz, 2H), 3.63 (t, J=6.6 Hz, 2H), 1.71-1.62 (m, 2H), 1.61-1.52 (m, 2H), 1.42-1.29 (m, 8H).

### Intermediate 21b

Intermediate 21b was synthesized following the procedure for 21a. ¹H NMR (500 MHz, CDCl₃) δ 6.40 (dd, J=17.4, 1.4 Hz, 1H), 6.12 (ddd, J=17.4, 10.4, 0.8 Hz, 1H), 5.82 (dd, J=10.5, 1.4 Hz, 1H), 4.09 (d, J=7.2 Hz, 0.5H), 3.99 (d, J=6.5 Hz, 1.5H), 3.55 (dd, J=6.9, 5.2 Hz, 0.5H), 3.46 (t, J=5.7 Hz, 1.5H), 1.88-1.80 (m, 3H), 1.67 (dddt, J=17.9, 11.3, 7.8, 3.9 Hz, 0.5H), 1.60-1.49 (m, 1H), 1.46 (ddt, J=10.2, 7.1, 3.8 Hz, 1H), 1.45-1.37 (m, 0.5H), 1.30 (dt, J=11.7, 5.5 Hz, 1H), 1.10-0.92 (m, 3H).

### Intermediate 21c

Intermediate 21c was synthesized following the procedure for 21a.

### Intermediate 23a

To 21a (292 mg, 1.46 mmol) in DCM (20 mL) was added 22a (251 mg, 1.6 mmol, 1.1 eq), EDCl (307 mg, 1.6 mmol, 1.1 eq) and DMAP (196 mg, 1.6 mmol, 1.1 eq). The resulting mixture was stirred at room temperature overnight, washed with 1N HCl, saturated NaHCO₃ and brine. The combined organic phase was dried over Na₂SO₄ and concentrated. Purification by column chromatography (ethyl acetate/hexane) afforded intermediate 23a (332 mg, 67%) as clear oil. ¹H NMR (500 MHz, CDCl₃) δ 6.39 (dd, J=17.4, 1.5 Hz, 1H), 6.12 (dd, J=17.3, 10.4 Hz, 1H), 5.81 (dd, J=10.4, 1.5 Hz, 1H), 4.15 (t, J=6.7 Hz, 2H), 4.05 (t, J=6.7 Hz, 2H), 2.33-2.27 (m, 2H), 1.74-1.52 (m, 10H), 1.55-1.47 (m, 2H), 1.41-1.29 (m, 7H), 1.28-1.07 (m, 4H), 0.94-0.83 (m, 2H).

### Intermediate 23b

Intermediate 23b was synthesized following the procedure for 23a. ¹H NMR (500 MHz, CDCl₃) δ 6.40 (dd, J=17.3, 1.5 Hz, 1H), 6.12 (dd, J=17.3, 10.4 Hz, 1H), 5.81 (dd, J=10.5, 1.5 Hz, 1H), 4.15 (t, J=6.7 Hz, 2H), 4.05 (t, J=6.7 Hz, 2H), 2.17 (d, J=7.0 Hz, 2H), 1.83-1.57 (m, 10H), 1.39-1.30 (m, 8H), 1.26 (qt, J=12.2, 3.5 Hz, 2H), 1.21-1.08 (m, 1H), 0.96 (qd, J=12.7, 3.8 Hz, 2H).

### Intermediate 23c

Intermediate 23c was synthesized following the procedure for 23a. H NMR (499 MHz, CDCl₃) δ 6.40 (dd, J=17.4, 1.4 Hz, 1H), 6.12 (dd, J=17.3, 10.4 Hz, 1H), 5.82 (dd, J=10.4, 1.4 Hz, 1H), 4.08 (d, J=7.1 Hz, 0.5H), 3.99 (dd, J=6.8, 4.0 Hz, 2H), 3.90 (d, J=6.5 Hz, 1.5H), 2.33-2.26 (m, 2H), 1.83-1.78 (m, 3H), 1.69-1.58 (m, 4H), 1.57-1.51 (m, 1H), 1.50-1.39 (m, 1H), 1.38-1.23 (m, 4H), 1.09-0.95 (m, 3H), 0.89 (t, J=6.9 Hz, 3H).

### Intermediate 23d

Intermediate 23d was synthesized following the procedure for 23a. H NMR (500 MHz, CDCl₃) δ 6.40 (dd, J=17.3, 1.5 Hz, 1H), 6.12 (ddd, J=17.4, 10.4, 0.8 Hz, 1H), 5.82 (dd, J=10.4, 1.5 Hz, 1H), 4.08 (d, J=7.2 Hz, 0.5H), 3.99 (dd, J=6.9, 4.8 Hz, 2H), 3.90 (d, J=6.5 Hz, 1.5H), 2.28 (td, J=7.5, 1.1 Hz, 2H), 1.89-1.78 (m, 3H), 1.68-1.58 (m, 3H), 1.58-1.51 (m, 3H), 1.48-1.41 (m, 1H), 1.23-1.15 (m, 2H), 1.10-0.95 (m, 3H), 0.88 (d, J=6.6 Hz, 6H).

### Intermediate 23e

Intermediate 23e was synthesized following the procedure for 23a. ¹H NMR (500 MHz, CDCl₃) δ 6.40 (dd, J=17.3, 1.5 Hz, 1H), 6.12 (ddd, J=17.4, 10.4, 0.9 Hz, 1H), 5.82 (dd, J=10.5, 1.4 Hz, 1H), 4.08 (d, J=7.2 Hz, 0.5H), 3.99 (dd, J=6.9, 3.3 Hz, 2H), 3.90 (d, J=6.5 Hz, 1.5H), 2.33-2.26 (m, 2H), 1.89-1.78 (m, 3H), 1.69-1.50 (m, 4H), 1.50-1.38 (m, 1H), 1.34-1.22 (m, 11H), 1.10-0.95 (m, 3H), 0.88 (t, J=6.9 Hz, 3H).

### Intermediate 23f

Intermediate 23f was synthesized following the procedure for 23a. ¹H NMR (500 MHz, CDCl₃) δ 6.41 (dd, J=17.3, 1.5 Hz, 1H), 6.13 (ddd, J=17.4, 10.4, 0.9 Hz, 1H), 5.83 (dd, J=10.5, 1.4 Hz, 1H), 5.59-5.30 (m, 2H), 3.99 (dd, J=6.9, 3.3 Hz, 2H), 3.90 (d, J=6.5 Hz, 3H), 2.33-2.28 (m, 2H), 2.20-2.17 (m, 1H), 2.08-2.03 (m, 2H), 1.85-1.80 (m, 5H), 1.67-1.62 (m, 7H), 1.38-1.25 (m, 14H), 1.05-0.97 (m, 4H), 0.89-0.87 (m, 3H)

### Product 24a

The mixture of N1-(3-aminopropyl)-N1-methylpropane-1,3-diamine (11.8 mg, 0.081 mmol) and 23a (116 mg, 0.34 mmol) was heated at 90° C. for 72 hours. The reaction mixture was cooled to room temperature and loaded onto a column (MeOH/DCM). Product 24a (37 mg, 30%) was purified as a clear gel.

¹H NMR (499 MHz, CDCl₃) δ 4.05 (td, J=6.8, 1.6 Hz, 16H), 2.77 (t, J=7.3 Hz, 8H), 2.43 (td, J=7.0, 2.2 Hz, 12H), 2.29 (q, J=7.0 Hz, 12H), 2.18 (s, 3H), 1.74-1.63 (m, 16H), 1.61-1.54 (m, 32H), 1.52 (dt, J=8.7, 7.0 Hz, 8H), 1.32 (m, 24H), 0.94-0.83 (m, 8H). MS found 1498.6 [M+H]⁺. calcd for [C87H155N3O16=1498.1]

### Product 24b

Product 24b was synthesized following the procedure for 24a. ¹H NMR (500 MHz, CDCl₃) δ 4.05 (td, J=6.8, 2.1 Hz, 16H), 2.76 (t, J=7.3 Hz, 8H), 2.43 (td, J=7.3, 3.0 Hz, 12H), 2.28 (dd, J=8.9, 5.8 Hz, 4H), 2.17 (d, J=7.0 Hz, 12H), 1.80-1.73 (m, 3H), 1.73-1.65 (m, 16H), 1.64-1.55 (m, 24H), 1.32 (s, 32H), 1.37-1.20 (m, 8H), 1.20-1.08 (m, 4H), 1.02-0.90 (m, 8H). MS found 1442.5 [M+H]⁺. calcd for [C83H147N3O16=1442.08]

### Product 24c

Product 24c was synthesized following the procedure for 24a. ¹H NMR (500 MHz, CDCl₃) δ 3.98 (d, J=7.2 Hz, 5H), 3.89 (dd, J=6.6, 1.8 Hz, 11H), 2.77 (t, J=7.3 Hz, 8H), 2.43 (t, J=7.3 Hz, 12H), 2.29 (q, J=7.1 Hz, 12H), 2.17 (s, 3H), 1.85-1.75 (m, 14H), 1.61 (h, J=7.6 Hz, 18H), 1.58-1.49 (m, 4H), 1.45-1.37 (m, 4H), 1.31 (ddt, J=11.2, 8.0, 5.3 Hz, 16H), 1.06-0.93 (m, 12H), 0.89 (t, J=6.9 Hz, 12H). MS found 1330.5 [M+H]⁺. calcd for [C75H131N3O16=1329.95]

### Product 24d

Product 24d was synthesized following the procedure for 24a. ¹H NMR (499 MHz, CDCl₃) δ 3.98 (d, J=7.2 Hz, 5H), 3.89 (dd, J=6.6, 2.2 Hz, 11H), 2.77 (t, J=7.4 Hz, 8H), 2.43 (t, J=7.3 Hz, 12H), 2.28 (t, J=7.5 Hz, 12H), 2.17 (s, 3H), 1.85-1.78 (m, 2H), 1.67-1.49 (m, 34H), 1.46-1.36 (m, 6H), 1.23-1.15 (m, 10H), 1.06-0.94 (m, 12H), 0.88 (d, J=6.6 Hz, 24H). MS found 1386.8 [M+H]⁺. calcd for [C79H139N3O16=1386.02]

### Product 24e

Product 24e was synthesized following the procedure for 24a. ¹H NMR (500 MHz, CDCl₃) δ 3.98 (d, J=7.2 Hz, 5H), 3.89 (dd, J=6.5, 1.8 Hz, 11H), 2.76 (t, J=7.3 Hz, 8H), 2.43 (t, J=7.2 Hz, 12H), 2.30 (q, J=9.8 Hz, 12H), 1.94 (s, 3H), 1.83-1.76 (m, 12H), 1.64-1.56 (m, 18H), 1.59-1.50 (m, 5H, 1.45-1.37 (m, 5H), 1.34-1.22 (m, 40H), 1.06-0.93 (m, 12H), 0.88 (t, J=6.9 Hz, 12H). MS found 1498.6 [M+H]⁺. calcd for [C87H155N3O16=1498.14]

### Product 24f

Product 24f was synthesized following the procedure for 24a. ¹H NMR (500 MHz, CDCl₃): δ 5.27-5.32 (m, 8H), 3.88 (d, 16H, J=9.8 Hz), 2.77 (t, 8H, J=12 Hz), 2.43 (t, 8H, J=9.8 Hz), 2.23 (t, 8H, J=9.8 Hz), 2.18 (s, 3H), 2.06-2.02 (dd, 8H, J=3 Hz, 9.8 Hz), 1.80-1.82 (m, 16H), 1.62-1.60 (m, 34H), 1.36-1.26 (m, 56H), 1.01-0.97 (m, 14H), 0.89 (d, 12H, J=3 Hz). MS: m/z 1988.1 (M+H).

### General Procedure for Synthesis of Lipidoids (E)

### General Formula E.1

The general structure of E.1 compounds is shown below.

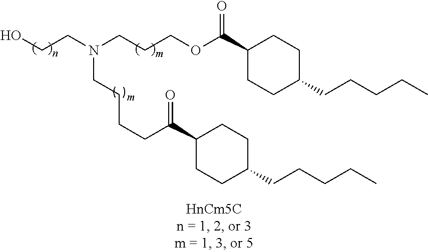

The synthetic route of E.1 compounds is given in the following General Scheme E.1. This two-step sequence begins with an esterification reaction between trans-4-pentylcyclohexane carboxylic acid and hydroxy substituted alkyl bromides of different lengths (C3, C5, and C7) catalyzed by N-Ethyl-N′-(3-dimethylaminopropyl) carbodiimide hydrochloride (EDC-HCl) and N,N-Dimethylpyridin-4-amine (DMAP). The corresponding ester which bears a bromide as a functional handle reacts with hydroxy substituted amines (Hₙ, where n=2, 3, or 4) to give the target compounds.

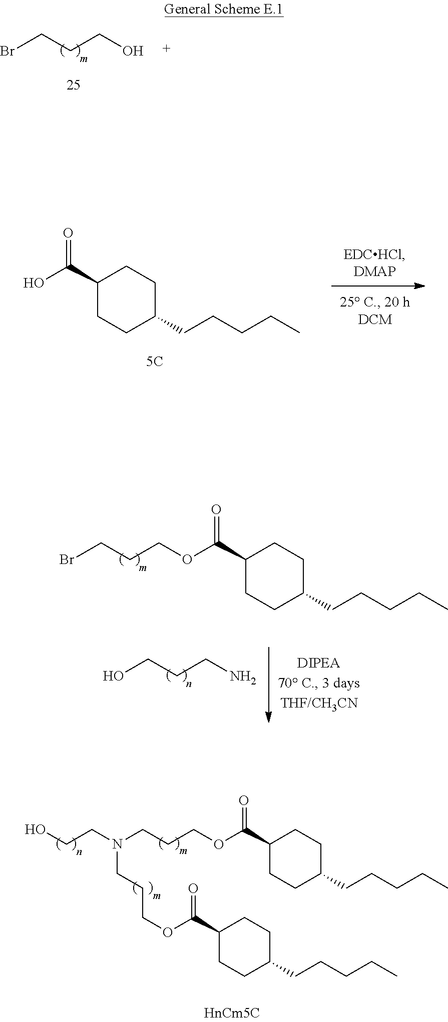

### Experimental Protocols

### Esterification General Protocol

In a 100 mL round-bottom flask, trans-4-pentylcyclohexane carboxylic acid 5C (1.0 eq), EDC-HCl (1.5 eq), and DMAP (0.4 eq) were dissolved in dichloromethane (DCM). The solution was then stirred for 20 mins at room temperature before adding hydroxy substituted alkyl bromide 25 (1.5 eq) and the resulting reaction was stirred for 20 h at room temperature. Brine solution was added and the reaction was extracted with DCM (4×). Combined organic extracts were dried over Na₂SO₄, filtered, and evaporated. The crude was purified by silica gel flash automated chromatography with 5-10% EtOAc/hexane eluants to give bromo substituted esters.

### Amine Alkylation General Protocol

In a 1-dram scintillation vial, amine (2-aminoethanol H2, 3-aminopropanol H3 or 4-aminobutanol H4), and bromo substrate were weighed. Solvents tetrahydrofuran (THF) and acetonitrile (CH₃CN) (1:1) were poured into the vial followed by N,N-Diisopropylethylamine (DIPEA, 3.0 eq). The reaction vial was capped and stirred for 3 days at 70° C. The reaction was then cooled, solvents were evaporated, and water (20 mL) was added. The resulting mixture was extracted with DCM (4×20 mL) and combined DCM extracts were washed with brine (20 mL). The resulting extracts were dried over Na₂SO₄, filtered, and evaporated. The crude was purified by flash silica gel chromatography with 2-10% MeOH/DCM.

### 5CC3

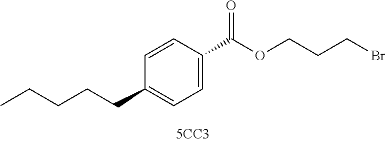

Following the general protocol for esterification, trans-4-pentylcyclohexane carboxylic acid (1.07 g) was combined with EDC (1.21 g), and DMAP (0.26 g) in 10 mL DCM. 3-bromo propanol (0.6 mL) was added after 15 mins of stirring. The crude after workup was purified with 6% EtOAc/hexanes. Colorless oil, 1.26 g (73%); ¹H NMR (499 MHz, CDCl₃) δ 4.20 (q, J=6.2 Hz, 2H), 3.53 (dt, J=74.6, 6.5 Hz, 2H), 2.26-2.05 (m, 3H), 2.00-1.91 (m, 2H), 1.85-1.76 (m, 2H), 1.46-1.35 (m, 2H), 1.32-1.15 (m, 9H), 0.94-0.85 (m, 5H).

### 5CC5

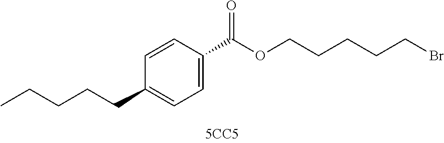

Following the general protocol for esterification, trans-4-pentylcyclohexane carboxylic acid (5.0 g) was combined with EDC (5.8 g), and DMAP (1.23 g) in 50 mL DCM. 7-bromo heptanol (3.8 mL) was added after 15 mins of stirring. The crude after workup was purified with 4% EtOAc/hexanes. Colorless oil, 5.3 g (62%); ¹H NMR (499 MHz, CDCl₃) δ 4.06 (t, J=6.5 Hz, 2H), 3.47 (dt, J=64.6, 6.7 Hz, 2H), 2.21 (tt, J=12.2, 3.6 Hz, 1H), 1.98-1.92 (m, 2H), 1.91-1.85 (m, 1H), 1.84-1.75 (m, 3H), 1.68-1.61 (m, 2H), 1.55-1.46 (m, 2H), 1.40 (qd, J=13.1, 3.5 Hz, 2H), 1.32-1.13 (m, 9H), 0.94-0.86 (m, 5H).

### 5CC7

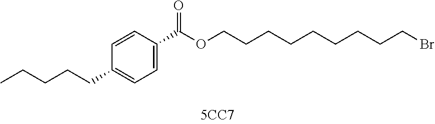

Following the general protocol for esterification, in a 250 mL round-bottom flask, trans-4-pentylcyclohexane carboxylic acid (5.0 g, 1.0 eq), EDC-HCl (7.25 g, 1.5 eq), and DMAP (1.23 g, 0.4 eq) were dissolved in DCM (50 mL). The solution was then stirred for 20 mins at room temperature before adding 1-bromo heptanol (5.8 mL, 1.5 eq). After reaction and workup was completed, the crude was purified by silica gel flash automated chromatography with 3% EtOAc/hexane eluants to give 5CC7 as a colorless oil. Colorless oil, 6.8 g; Yield: 71%; ¹H NMR (499 MHz, CDCl₃) δ 4.04 (t, J=6.6 Hz, 2H), 3.47 (dt, J=62.7, 6.8 Hz, 2H), 2.20 (tt, J=12.3, 3.6 Hz, 1H), 1.99-1.91 (m, 2H), 1.89-1.74 (m, 4H), 1.62 (p, J=6.7 Hz, 2H), 1.48-1.13 (m, 17H), 0.95-0.85 (m, 5H).

### General Formula E.2

The general structure of E.2 compounds is shown below.

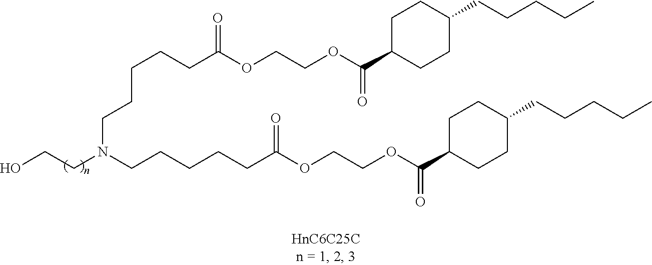

The synthetic route of E.2 compounds is given in the following General Scheme E.2.

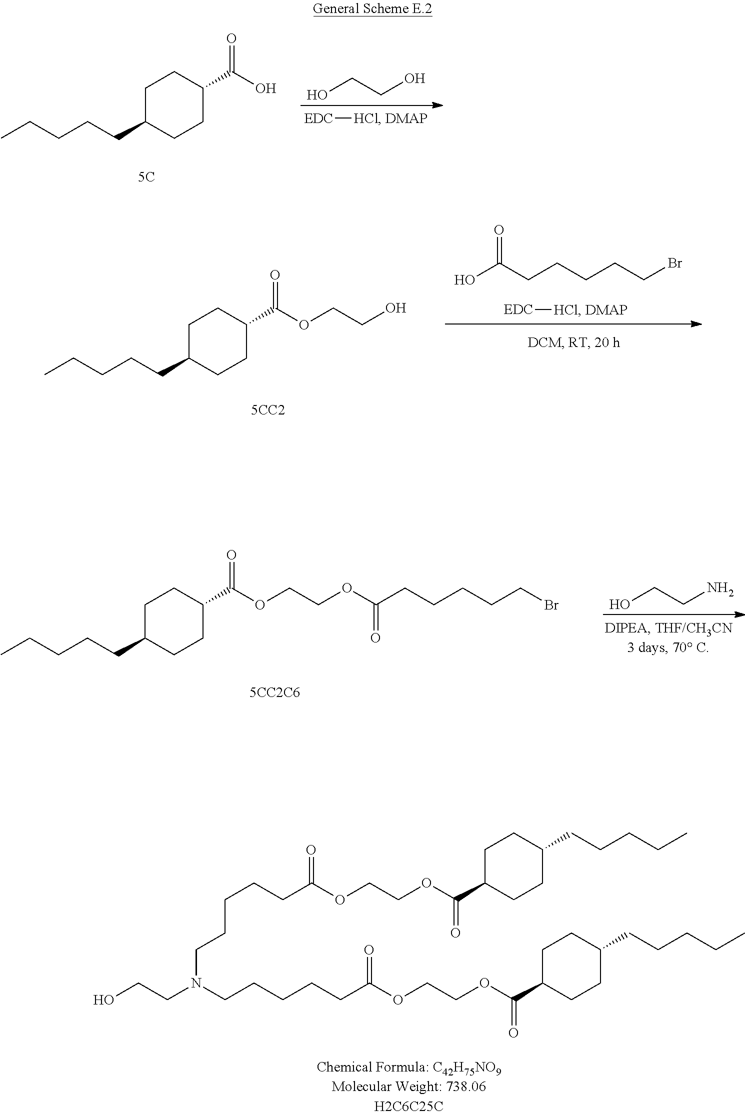

### 5CC2

In a 200 mL round bottom flask, trans-4-pentylcyclohexanecarboxylic acid (5.0 g, 1.0 eq) was combined with EDC (5.8 g, 1.2 eq) and DMAP (1.23 g, 0.4 eq) in DCM (50 mL). The suspension was then stirred for 15 mins at room temperature before ethylene glycol (4.3 mL, 3.0 eq) was added and the resulting mixture was stirred for 20 h at room temperature. Water (20 mL) was added and the reaction extracted with DCM (4×50 mL). Combined DCM extracts were washed with brine (20 mL); dried over Na₂SO₄; filtered and evaporated to dryness. The crude residue was then purified by flash chromatography with 10-15% EtOAc/hexane eluants. Colorless oil, 2.8 g (65%); ¹H NMR (499 MHz, CDCl₃) δ 4.22-4.16 (m, 2H), 3.84-3.78 (m, 2H), 2.26 (tt, J=12.2, 3.6 Hz, 1H), 2.05-1.93 (m, 3H), 1.86-1.76 (m, 2H), 1.48-1.35 (m, 2H), 1.32-1.15 (m, 9H), 0.95-0.84 (m, 5H).

### 5CC2C6

In a 200 mL round bottom flask, 6-bromohexanoic acid (2.0 g, 1.0 eq) was combined with EDC (2.7 g, 1.2 eq) and DMAP (0.51 g, 0.4 eq) in DCM (30 mL). The suspension was then stirred for 15 mins at room temperature before adding 5CC2 (2.7 g, 1.1 eq) and the resulting mixture was stirred for 20 h at room temperature. Water (20 mL) was added and the reaction extracted with DCM (4×50 mL). Combined DCM extracts were washed with brine (20 mL); dried over Na₂SO₄; filtered and evaporated to dryness. The crude residue was then purified by flash chromatography with 6-8% EtOAc/hexane eluants. Colorless oil, 2.8 g (65%); ¹H NMR (499 MHz, CDCl₃) δ 4.31-4.22 (m, 4H), 3.47 (dt, J=64.5, 6.7 Hz, 2H), 2.35 (t, J=7.4 Hz, 2H), 2.24 (tt, J=12.2, 3.6 Hz, 1H), 1.99-1.75 (m, 6H), 1.70-1.61 (m, 2H), 1.52-1.35 (m, 4H), 1.34-1.14 (m, 9H), 0.95-0.85 (m, 5H).

### H2C6C25C

Following the general protocol for amine alkylation described in General Scheme E.1, amine H2 (17 mg) was combined with C6C25C (300 mg) and DIPEA (200 μL) in THF/CH₃CN (1:1, 1.0 mL). After the reaction, the crude was purified by 4% MeOH/DCM eluants.

### General Formula E.3

The general structure of E.3 compounds is shown below.

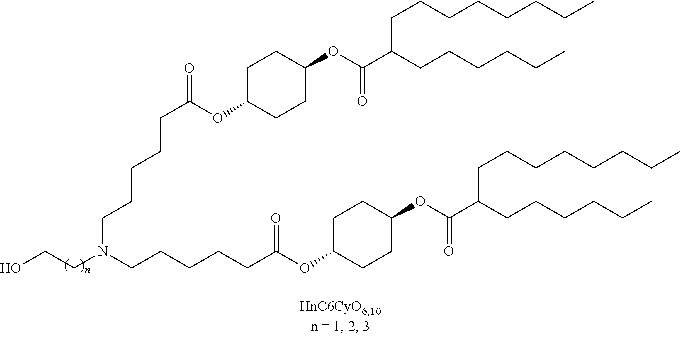

The synthetic route of E.3 compounds is given in the following General Scheme E.3.

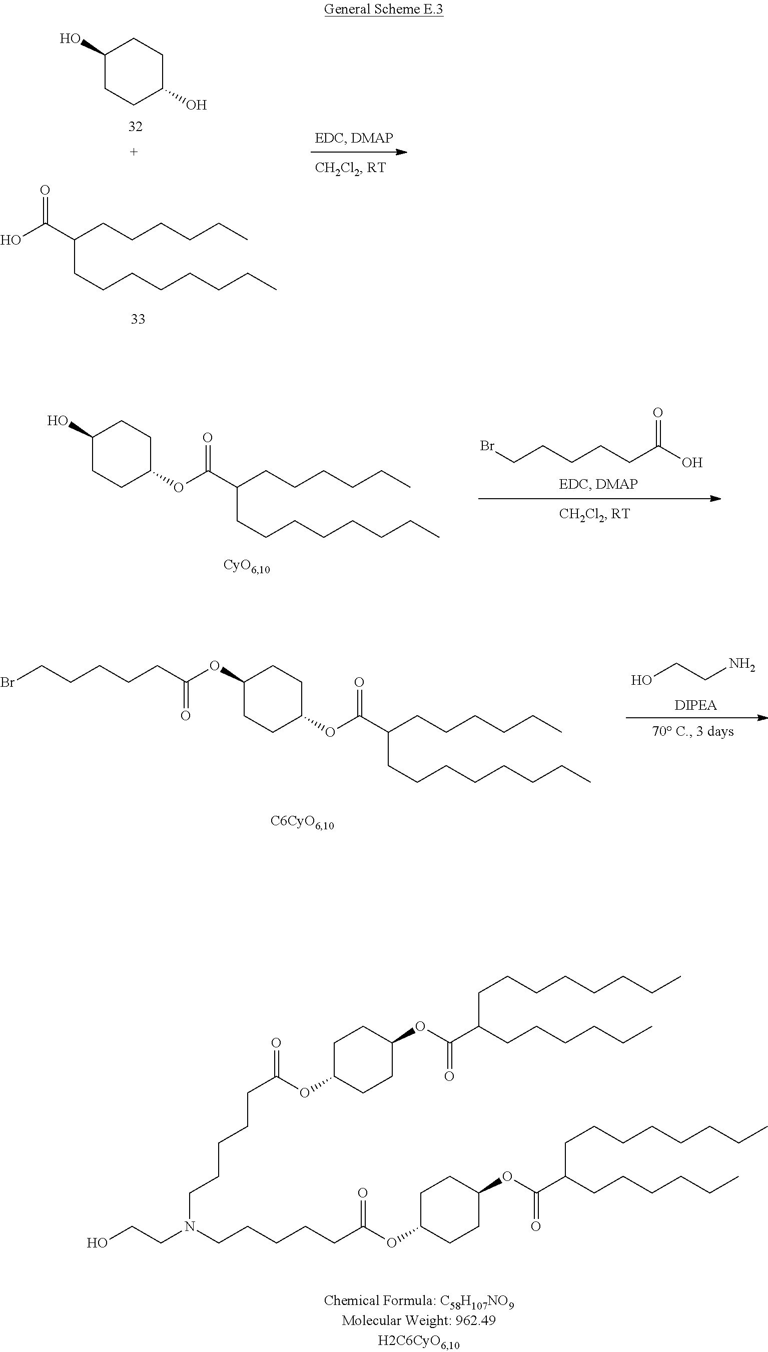

### CyO_{6,10}

In a 200 mL round bottom flask, 2-hexyldecanoic acid 33 (5.0 g, 1.0 eq) was combined with EDC (4.56 g, 1.2 eq) and DMAP (0.95 g, 0.4 eq) in DCM (100 mL). The suspension was then stirred for 15 mins at room temperature before adding trans-1,4-cyclohexanediol 32 (3.4 g, 1.5 eq) and the resulting mixture was stirred for 20 h at room temperature. Water (20 mL) was added and the reaction was extracted with DCM (4×50 mL). Combined DCM extracts were washed with brine (20 mL); dried over Na₂SO₄; filtered and evaporated to dryness. The crude residue was then purified by flash chromatography with 10-15% EtOAc/hexane eluants. Colorless oil, 1.9 g (26%); ¹H NMR (499 MHz, CDCl₃) δ 4.88-4.69 (m, 1H), 3.85-3.63 (m, 1H), 2.36-2.18 (m, 1H), 2.03-1.91 (m, 4H), 1.57-1.38 (m, 8H), 1.32-1.19 (m, 20H), 0.90-0.84 (m, 6H).

### C₆CyO6,10

In a 200 mL round bottom flask, 6-bromohexanoic acid (1.2 g, 1.0 eq) was combined with EDC (1.4 g, 1.2 eq) and DMAP (0.3 g, 0.4 eq) in DCM (20 mL). The suspension was then stirred for 15 mins at room temperature before adding CyO6,10 (2.5 g, 1.1 eq) and the resulting mixture was stirred for 20 h at room temperature. Water (20 mL) was added and the reaction was extracted with DCM (4×50 mL). Combined DCM extracts were washed with brine (20 mL); dried over Na₂SO₄; filtered and evaporated to dryness. The crude residue was then purified by flash chromatography with 4-5% EtOAc/hexane eluants. Colorless oil, 1.4 g (43%); ¹H NMR (499 MHz, CDCl₃) δ 4.92-4.75 (m, 2H), 3.47 (dt, J=64.2, 6.7 Hz, 2H), 2.36-2.24 (m, 3H), 2.01-1.75 (m, 6H), 1.68-1.40 (m, 12H), 1.31-1.20 (m, 20H), 0.87 (td, J=7.0, 1.6 Hz, 6H).

### H2C6CyO_{6,10}

Following the general protocol for amine alkylation described in General Scheme E.1, amine H2 (19 mg) was combined with C₆CyO_{6,10} (470 mg) and DIPEA (200 μL) in THF/CH₃CN (1:1, 1.0 mL). After the reaction, the crude was purified by 4% MeOH/DCM eluants.

### General Formula E.4

The general structure of E.4 compounds is shown below.

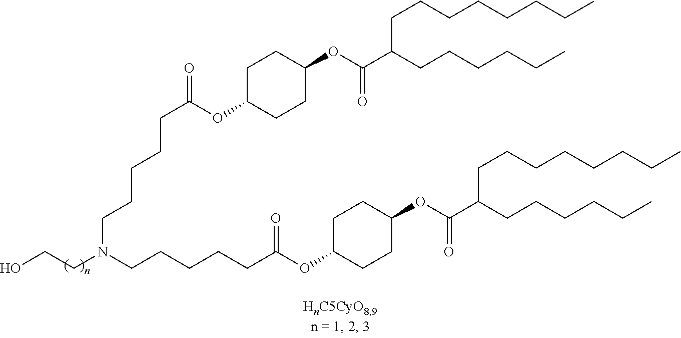

The synthetic route of E.4 compounds is given in the following General Scheme E.4.

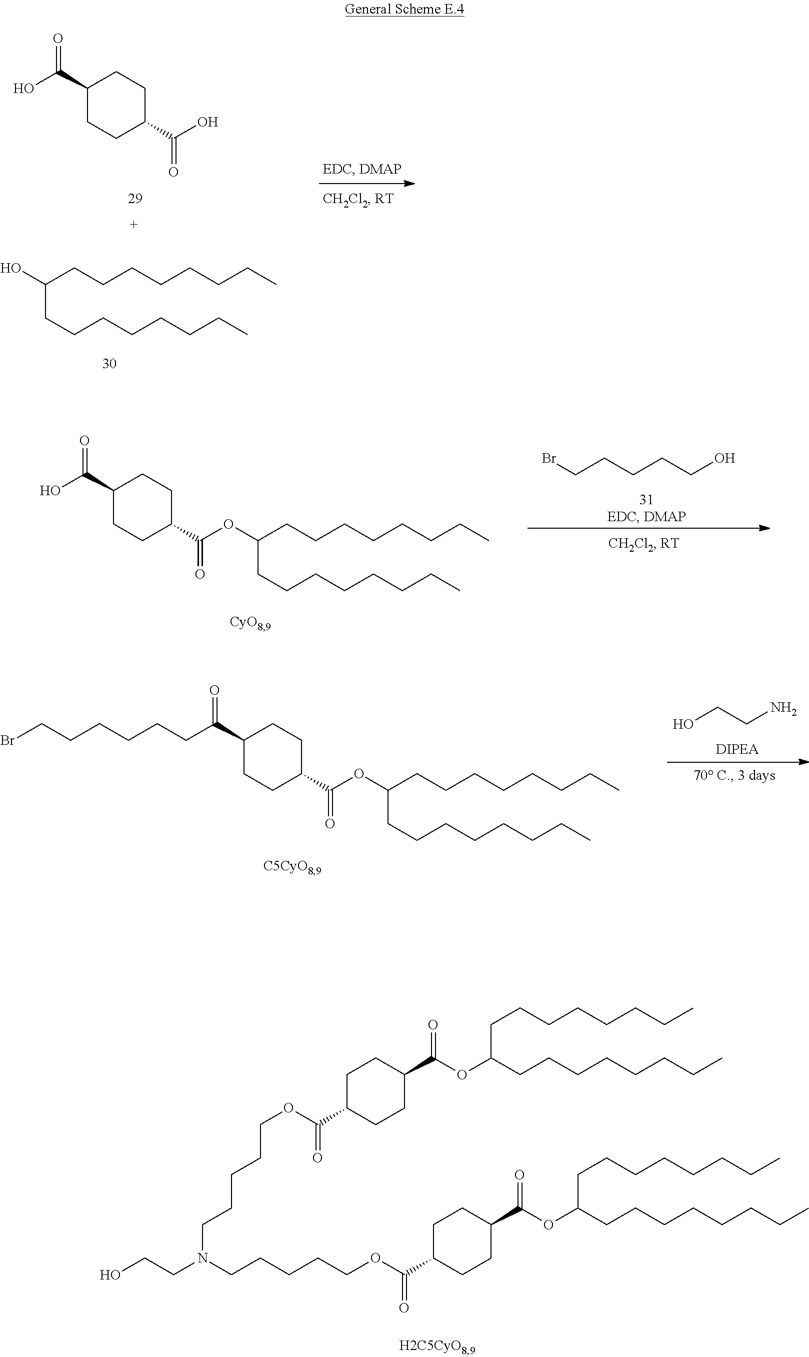

### CyO_{8,9}

In a 200 mL round bottom flask, trans-1,4-cyclohexanedicarboxylic acid 29 (1.0 g, 1.0 eq) was combined with EDC (0.56 g, 0.5 eq) and DMAP (0.15 g, 0.2 eq) in DCM (50 mL). The suspension was then stirred for 15 mins at room temperature before adding 9-heptadecanol 30 (0.74 g, 0.5 eq) and the resulting mixture was stirred for 20 h at room temperature. Water (20 mL) was added and the reaction was extracted with DCM (4×50 mL). Combined DCM extracts were washed with brine (20 mL); dried over Na₂SO₄; filtered and evaporated to dryness. The crude residue was then purified by flash chromatography with 10-15% EtOAc/hexane eluants. Colorless oil, 1.45 g (61%); ¹H NMR (499 MHz, CDCl₃) δ 4.86 (p, J=6.3 Hz, 1H), 2.37-2.21 (m, 2H), 2.14-2.01 (m, 4H), 1.55-1.41 (m, 8H), 1.31-1.21 (m, 24H), 0.88 (t, J=6.9 Hz, 6H).

### C5CyO_{8,9}

In a 200 mL round bottom flask, CyO_{8,9} (1.45 g, 1.0 eq) was combined with EDC (0.81 g, 1.2 eq) and DMAP (0.2 g, 0.4 eq) in DCM (20 mL). The solution was then stirred for 15 mins at room temperature before adding 5-bromo pentanol 31 (0.6 mL, 1.2 eq) and the resulting mixture was stirred for 20 h at room temperature. Water (20 mL) was added and the reaction was extracted with DCM (4×50 mL). Combined DCM extracts were washed with brine (20 mL); dried over Na₂SO₄; filtered and evaporated to dryness. The crude residue was then purified by flash chromatography with 5-10% EtOAc/hexane eluants. Colorless oil, 1.1 g (57%); ¹H NMR (499 MHz, CDCl₃) δ 4.85 (p, J=6.3 Hz, 1H), 4.07 (t, J=6.5 Hz, 2H), 3.41 (t, J=6.7 Hz, 2H), 2.36-2.18 (m, 2H), 2.12-1.96 (m, 4H), 1.94-1.81 (m, 2H), 1.71-1.61 (m, 2H), 1.56-1.39 (m, 10H), 1.30-1.21 (m, 24H), 0.87 (t, J=6.9 Hz, 6H).

### H2C5CyO_{8,9}

Following the general protocol for amine alkylation described in General Scheme E.1, amine H2 (11 mg) was combined with C5Cy_{8,9} (289 mg) and DIPEA (150 μL) in THF/CH₃CN (1:1, 0.8 mL). After the reaction, the crude was purified by 4% MeOH/DCM eluants.

### General Formula E.5

The synthetic route of E.5 compounds is given in the following General Scheme E.5.

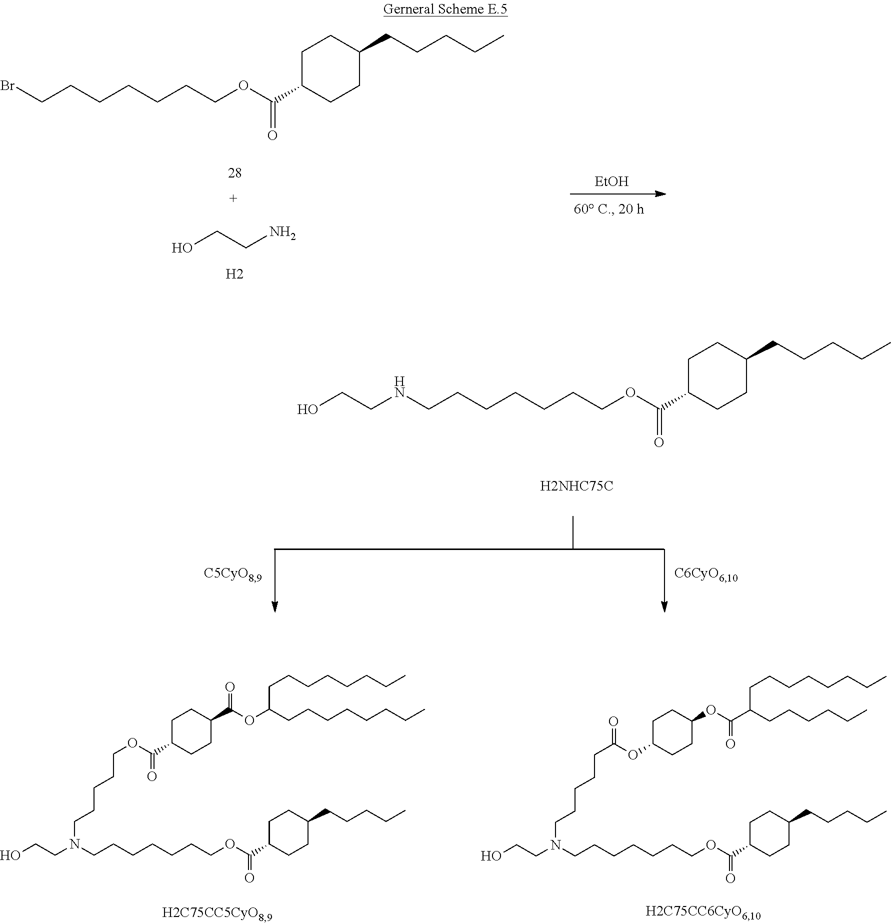

### Intermediate H2NHC75C

In a 20 mL scintillation vial, 5CC7 (1.0 g, 1.0 eq) was combined with H2 (4.83 mL, 30.0 eq) in ethanol (1.0 mL). The vial was then capped and heated to 60° C. for 20 h. The cooled reaction was then evaporated to dryness and the resulting residue was purified by flash silica gel chromatography with 50-60% MeOH/DCM. Colorless oil, 0.34 g (36%); ¹H NMR (499 MHz, CDCl₃) δ 4.04 (t, J=6.6 Hz, 2H), 3.67-3.60 (m, 2H), 2.80-2.74 (m, 2H), 2.64-2.58 (m, 2H), 2.20 (tt, J=12.2, 3.6 Hz, 1H), 2.02-1.86 (m, 4H), 1.84-1.76 (m, 2H), 1.65-1.57 (m, 2H), 1.53-1.12 (m, 20H), 0.95-0.84 (m, 5H).

### General Formula E.6

The general structure of E.6 compounds is shown below.

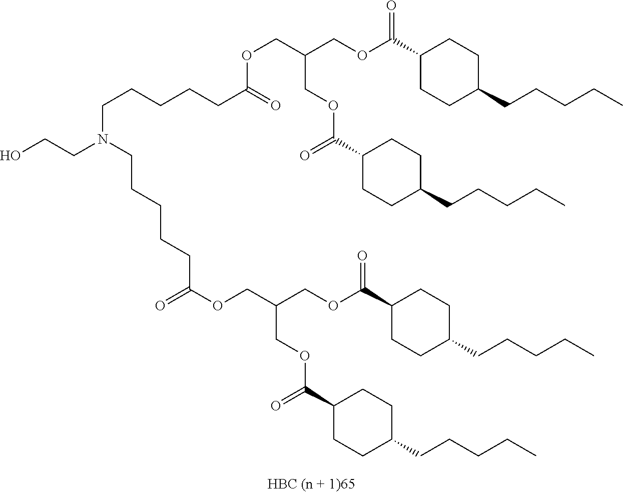 [SMILES: CCCCC[C@H]1CC[C@H](C(=O)OCC(COC(=O)CCCCCN(CCO)CCCCCC(=O)OCC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1]

The synthetic route of E.6 compounds is given in the following General Scheme E.6.

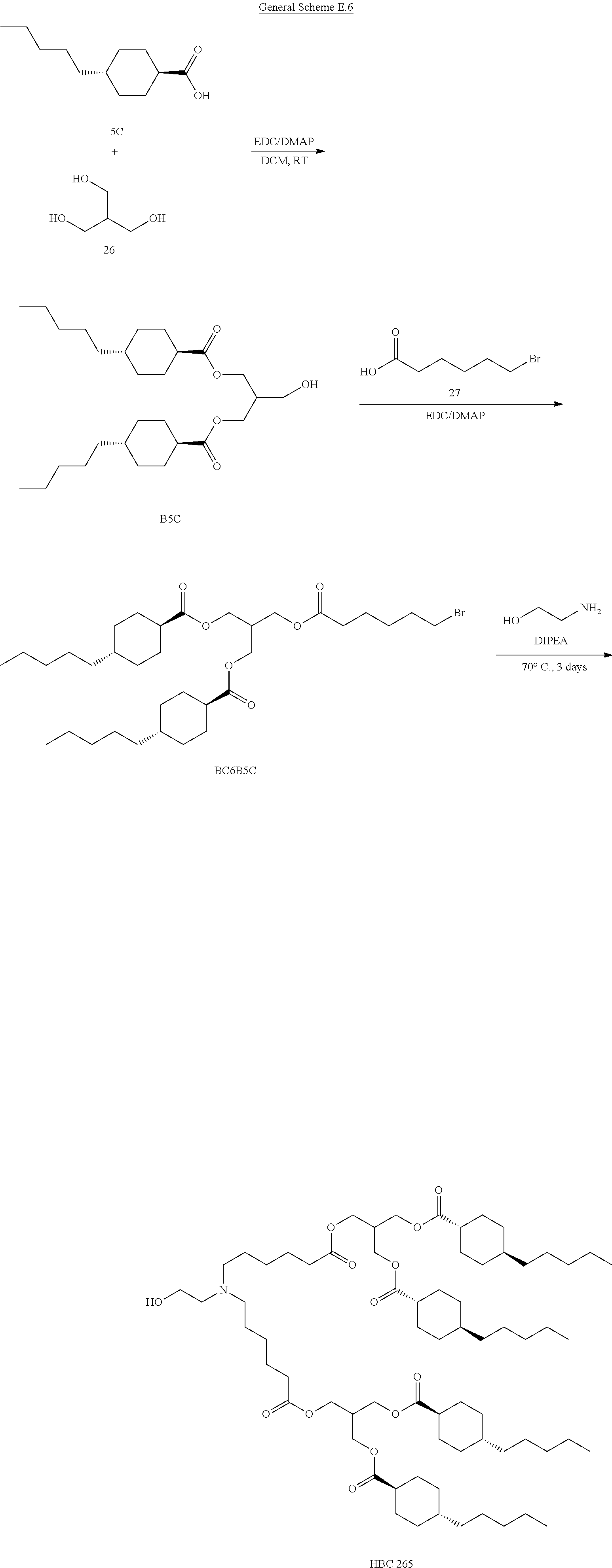

### B5C

In a 200 mL round bottom, trans-4-pentylcyclohexanecarboxylic acid 5C (3.95 g, 2.1 eq) was combined with EDC (3.91 g, 2.1 eq) and DMAP (1.2 g, 1.0 eq) in DCM (100 mL). The solution was then stirred for 20 minutes at room temperature before adding 2-hydroxymethyl-1,3-propanediol 26 (1.02 g, 1.0 eq). The resulting suspension was stirred for 20 h at room temperature. The hydroxy starting material slowly dissolves as the reaction progresses. 100 mL brine solution was added and the reaction was stirred for 20 mins at ambient temperature. Both layers were separated, and the aqueous layer was extracted with DCM (4×50 mL). Combined extracts were dried over Na₂SO₄, filtered, and evaporated to dryness. The crude was purified by silica gel flash chromatography with 15-20% EtOAc/hexane eluants. Colorless oil, 1.51 g (34%). ¹H NMR (499 MHz, CDCl₃) δ 4.24-4.09 (m, 4H), 3.59 (t, J=6.0 Hz, 2H), 2.29-2.14 (m, 4H), 1.95 (dt, J=12.2, 3.6 Hz, 4H), 1.80 (dt, J=15.3, 3.2 Hz, 4H), 1.40 (qd, J=13.1, 3.5 Hz, 4H), 1.34-1.13 (m, 18H), 0.96-0.83 (m, 10H).

### BC65C

In a 200 mL round bottom, 6-bromohexanoic acid 27 (0.4 g, 1.0 eq) was combined with EDC (0.5 g, 1.2 eq) and DMAP (0.12 g, 0.5 eq) in DCM (20 mL). The solution was stirred for 20 mins at room temperature before adding B5C (1.05 g, 1.1 eq) dissolved in 10 mL of DCM. The resulting solution was stirred for 20 h at room temperature. Brine (50 mL) was added and the reaction was stirred for 20 mins at room temperature. Both layers were separated, and the aqueous layer was extracted with DCM (3×50 mL). Combined extracts were dried over Na₂SO₄, filtered, and evaporated to dryness. The crude was purified by silica gel flash column chromatography with 6% EtOAc/hexane eluants. Colorless oil, 0.95 g (73%); ¹H NMR (499 MHz, CDCl₃) δ 4.12 (t, J=6.4 Hz, 6H), 3.47 (dt, J=64.3, 6.7 Hz, 2H), 2.40 (h, J=6.0 Hz, 1H), 2.34 (t, J=7.5 Hz, 2H), 2.22 (tt, J=12.2, 3.6 Hz, 2H), 1.99-1.76 (m, 10H), 1.65 (p, J=7.5 Hz, 2H), 1.52-1.44 (m, 2H), 1.39 (qd, J=13.1, 3.5 Hz, 4H), 1.32-1.14 (m, 18H), 0.94-0.84 (m, 10H).

### HBC 265

Following the general protocol for amine alkylation described in General Scheme E.1, amine H2 (12 mg) was combined with BC6B5C (292 mg) and DIPEA (200 μL) in THF/CH₃CN (1:1, 0.8 mL). After the reaction, the crude was purified by 6% MeOH/DCM eluants.

### H5-65C

In a 20 mL scintillation glass vial, 5-amino-1-pentanol (19 mg, 1.0 eq), BC6B5C (272 mg, 2.2 eq), K₂CO₃ (165 mg, 4.4 eq) and KI (62 mg, 1.0 eq) were combined and poured in CH₃CN/THF (1:1, 3 mL). To the suspension, 4 Å molecular sieves were added and the capped vial was stirred at 75° C. for 3 days. The cooled reaction was filtered, deionized water (20 mL) was added, and the filtrate was extracted with DCM (4×40 mL). Combined extracts were washed with brine (20 mL), dried over Na₂SO₄, filtered, and evaporated. The crude was purified by silica gel flash chromatography and the desired product was eluted with 4-5% MeOH/DCM eluants.

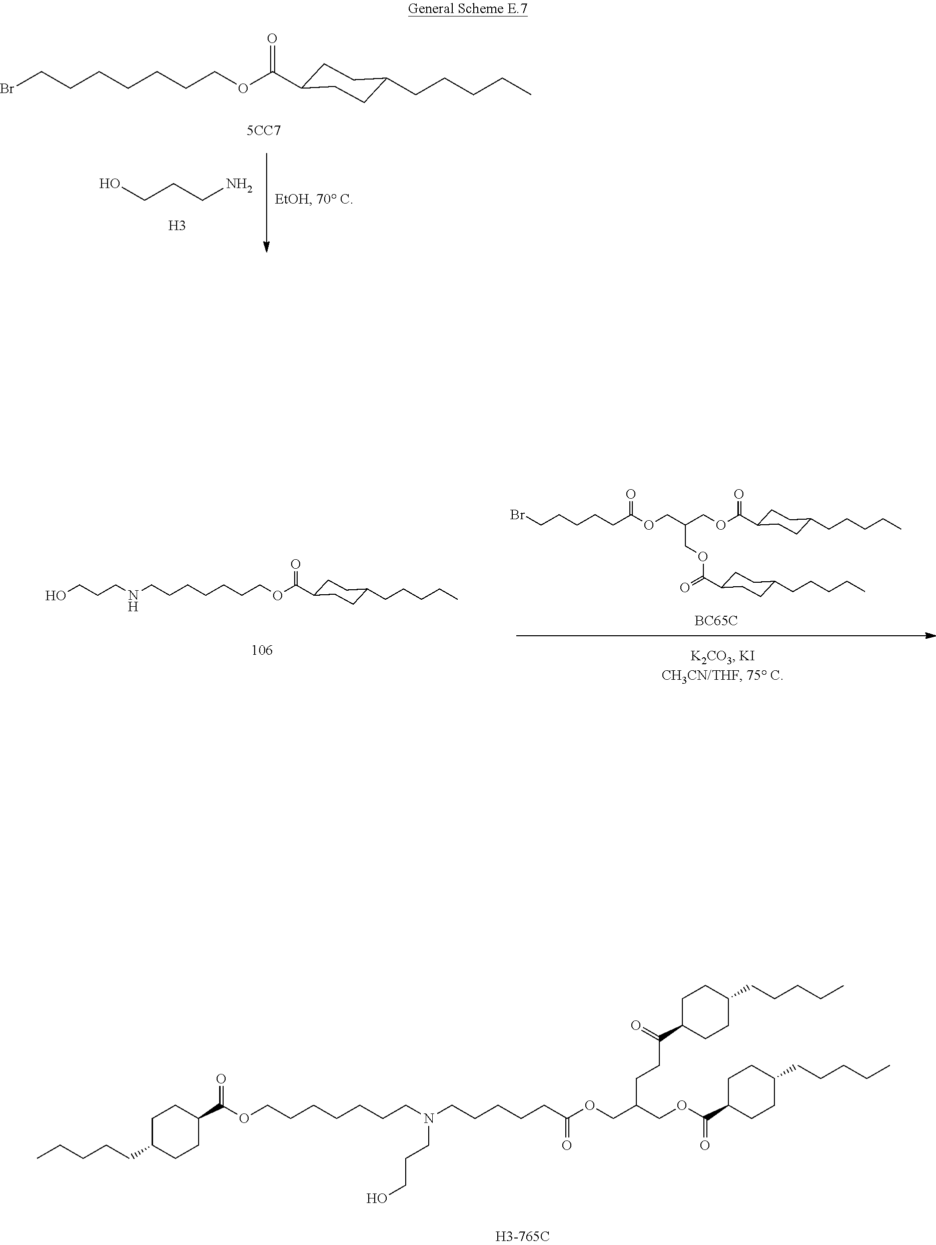

### Intermediate 106

In a 20 mL scintillation glass vial, 3-aminopropan-1-ol (4.0 g, 20.0 eq), and 5CC7 (1.0 g, 1.0 eq) were combined in EtOH (1.0 mL). The vial was capped and stirred for 20 h at 70° C. The cooled reaction was transferred to a 100 mL round bottom flask and the solvent was evaporated to dryness. The residue was then dissolved in 100 mL of DCM and washed with 5% aqueous sodium bicarbonate solution (2×100 mL) followed by brine (50 mL); dried over Na₂SO₄; filtered and evaporated. The crude was purified by silica gel flash chromatography and the desired product 106 was eluted with 40% MeOH/DCM eluants. Colorless oil, 301 mg (%); ¹H NMR (499 MHz, CDCl₃) δ 4.03 (t, J=6.6 Hz, 2H), 3.81 (t, J=5.0 Hz, 2H), 2.87 (t, J=5.0 Hz, 2H), 2.59 (t, J=7.1 Hz, 2H), 2.20 (tt, J=12.3, 3.6 Hz, 1H), 1.99-1.90 (m, 2H), 1.83-1.76 (m, 2H), 1.72-1.65 (m, 2H), 1.64-1.56 (m, 2H), 1.50-1.13 (m, 20H), 0.95-0.83 (m, 5H); LC-MS: Rt 7.6 min, m/z calculated [M+H]: 370.33, found 370.2.

### H3-765C

In a 20 mL scintillation glass vial, 106 (120 mg, 1.0 eq), BC6B5C (229 mg, 1.1 eq), K₂CO₃ (161 mg, 2.2 eq) and KI (84 mg, 1.0 eq) were combined and poured in CH₃CN/THF (1:1, 3 mL). To the suspension, 4 Å molecular sieves were added and the capped vial was stirred at 75° C. for 3 days. The cooled reaction was filtered, deionized water (20 mL) was added and the filtrate was extracted with DCM (4×40 mL). Combined extracts were washed with brine (20 mL), dried over Na₂SO₄, filtered, and evaporated. The crude was purified by silica gel flash chromatography and the desired product was eluted with 4-5% MeOH/DCM eluants.

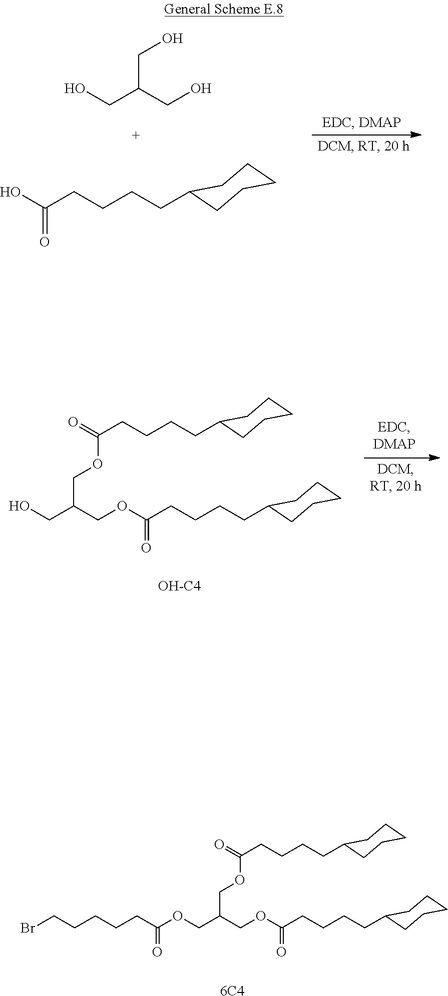

### OH-C4

In a 200 mL round bottom flask, cyclohexanepentanoic acid (2.2 g, 1.0 eq) was combined with EDC (2.8 g, 1.2 eq) and DMAP (0.8 g, 0.5 eq) in DCM (25 mL). The solution was then stirred for 20 minutes at room temperature before adding 2-hydroxymethyl-1,3-propanediol (0.86 g, 0.68 eq) in N,N-dimethyl formamide (5 mL). The resulting solution was stirred for 20 h at room temperature. Brine solution (100 mL) was poured in and stirred for 20 mins at ambient temperature. Both layers were separated, and the aqueous layer was extracted with DCM (4×50 mL). Combined extracts were dried over Na₂SO₄, filtered, and evaporated to dryness. The crude was purified by silica gel flash chromatography with 15-20% EtOAc/hexane eluants. Colorless oil, 1.8 g (35%); ¹H NMR (499 MHz, CDCl₃) δ 4.23-4.11 (m, 4H), 3.61 (t, J=6.0 Hz, 2H), 2.32 (t, J=7.5 Hz, 4H), 2.25-2.14 (m, 2H), 1.70-1.56 (m, 14H), 1.35-1.27 (m, 4H), 1.25-1.08 (m, 12H), 0.92-0.77 (m, 4H).

### 6C4

In a 200 mL round bottom flask, 6-bromohexanoic acid (0.46 g, 1.0 eq) was combined with EDC (0.6 g, 1.2 eq) and DMAP (0.2 g, 0.5 eq) in DCM (15 mL). The solution was then stirred for 20 minutes at room temperature before adding OH-C4 (2.2 g, 1.0 eq) in DCM (5 mL). The resulting solution was stirred for 20 h at room temperature. Brine solution (100 mL) was poured in and stirred for 20 mins at ambient temperature. Both layers were separated, and the aqueous layer was extracted with DCM (4×50 mL). Combined extracts were dried over Na₂SO₄, filtered, and evaporated to dryness. The crude was purified by silica gel flash chromatography with 5-10% EtOAc/hexane eluants. Colorless oil, 1.2 g (83%); ¹H NMR (499 MHz, CDCl₃) δ 6.09 (dd, J=6.0, 3.1 Hz, 6H), 5.43 (dt, J=64.4, 6.7 Hz, 2H), 4.40-4.23 (m, 7H), 3.88-3.71 (m, 2H), 3.66-3.52 (m, 16H), 3.48-3.40 (m, 2H), 3.31-3.23 (m, 4H), 3.23-3.03 (m, 12H), 2.87-2.75 (m, 4H).

### H2-6C4

In a 20 mL scintillation glass vial, H2 (17 mg, 1.0 eq), 6C₄ (351 mg, 2.2 eq), K₂CO₃ (180 mg, 4.4 eq) and KI (49 mg, 1.0 eq) were combined and poured in CH₃CN/THF (1:1, 3 mL). To the suspension, 4 Å molecular sieves were added and the capped vial was stirred for 75° C. for 3 days. The cooled reaction was filtered, deionized water (20 mL) was added and the filtrate was extracted with DCM (4×40 mL). Combined extracts were washed with brine (20 mL), dried over Na₂SO₄, filtered, and evaporated. The crude was purified by silica gel flash chromatography and the desired product was eluted with 4-5% MeOH/DCM eluants.

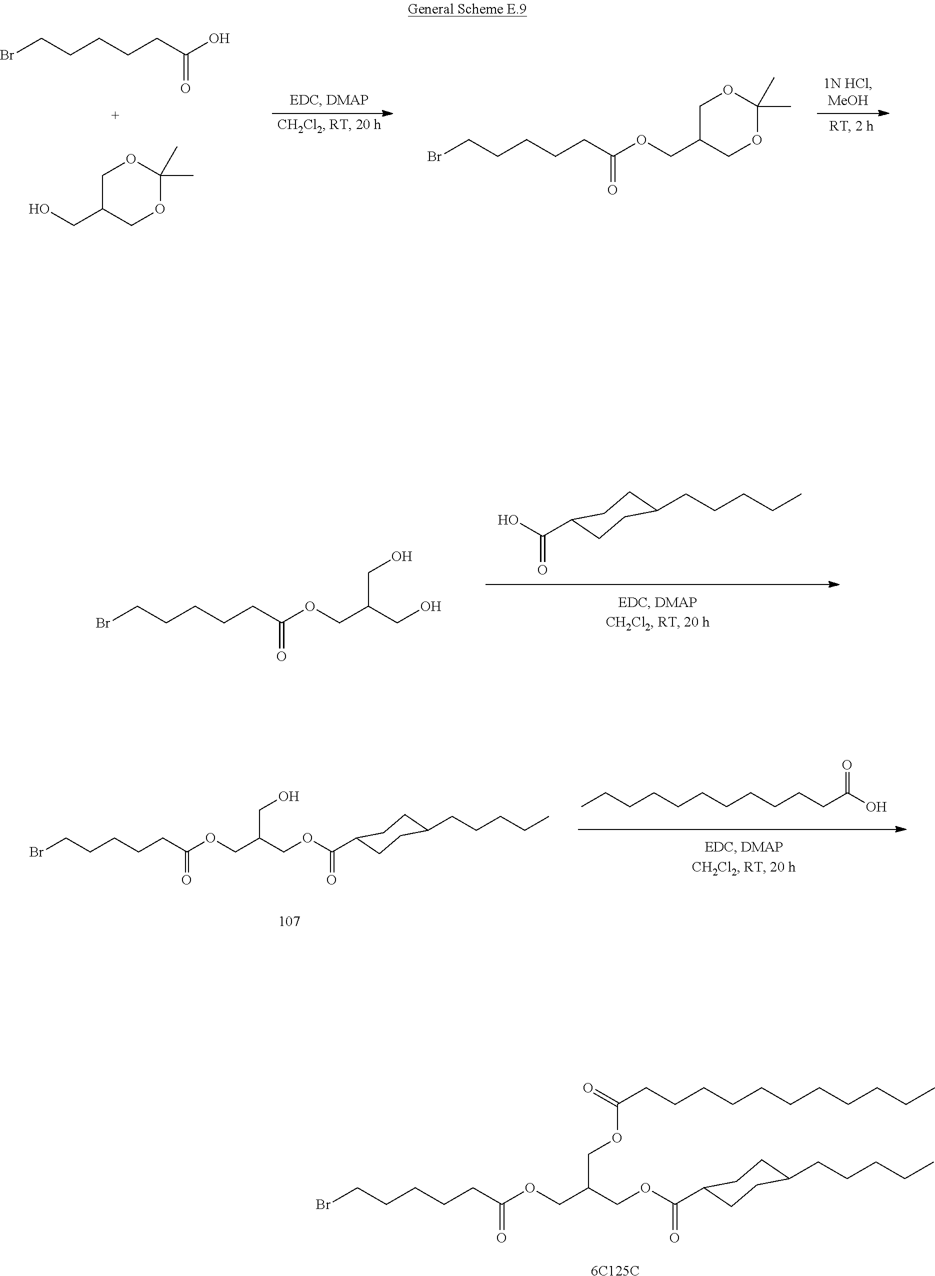

### Intermediate 107

In a 1000 mL round bottom flask, 6-bromohexanoic acid (25 mg, 1.0 eq), EDC (29.5 g, 1.2 eq), DMAP (7.9 g, 0.6 eq) were combined in DCM (250 mL) and stirred for 30 mins at room temperature. The solution of (2,2-dimethyl-1,3-dioxan-5-yl) methanol (20.6 g, 1.1 eq) in DCM (15 mL) was added, and the resulting solution was stirred for 20 h at room temperature. DI water (150 mL) was added and the solution was extracted with DCM (4×150 mL). Combined DCM extracts were washed with brine (50 mL); dried over Na₂SO₄; filtered and evaporated. The crude was dissolved in a mixed solution of methanol (200 mL) and 1N HCl (200 mL). The resulting solution was stirred for 2 h at room temperature. Methanol was evaporated on a rotavapor and extracted with EtOAc (4×200 mL). Combined extracts were washed with brine (100 mL); dried over Na₂SO₄; filtered and evaporated. The crude proceeded to the next step without any purification.

In a 500 mL round bottom flask, trans-4-n-pentylcyclohexanecarboxylic acid (10.0 g, 1.0 eq), EDC (11.7 g, 1.2 eq), DMAP (3.1 g, 0.5 eq) were combined in DCM (100 mL) and stirred for 30 mins at room temperature. The solution of the above crude (17.0 g, 1.2 eq) in DCM (50 mL) was added, and the resulting solution was stirred for 20 h at room temperature. DI water (100 mL) was added and the solution was extracted with DCM (4×100 mL). Combined extracts were washed with brine (50 mL); dried over Na2SO4; filtered and evaporated. The crude was purified by silica gel flash chromatography with 15-20% EtOAc/hexanes to give 107 as a colorless oil (7.8 g, 34%). 6C125C

In a 100 mL round bottom flask, dodecanoic acid (490 mg, 1.0 eq), EDC (0.62 g, 1.2 eq), DMAP (0.2 g, 0.6 eq) were combined in DCM (10 mL) and stirred for 15 mins at room temperature. The solution of 107 in DCM (5 mL) was added, and the resulting solution was stirred for 20 h at room temperature. DI water (50 mL) was added and the solution was extracted with DCM (4×50 mL). Combined DCM extracts were washed with brine (50 mL); dried over Na₂SO₄; filtered and evaporated. The crude was purified by flash chromatography with 5-10% EtOAc/hexane. Colorless oil, 1.13 g (83%); ¹H NMR (499 MHz, CDCl₃) δ 4.14-4.10 (m, 6H), 3.47 (dt, J=64.3, 6.7 Hz, 2H), 2.39 (p, J=6.0 Hz, 1H), 2.36-2.27 (m, 4H), 2.22 (tt, J=12.3, 3.6 Hz, 1H), 1.99-1.91 (m, 2H), 1.89-1.75 (m, 4H), 1.66-1.58 (m, 4H), 1.51-1.35 (m, 4H), 1.32-1.16 (m, 25H), 0.95-0.83 (m, 8H).

### H2-6C125C

In a 20 mL scintillation glass vial, H2 (11 mg, 1.0 eq), 6C125C (250 mg, 2.2 eq), K₂CO₃ (162 mg, 4.4 eq) and KI (85 mg, 1.0 eq) were combined and poured in CH₃CN/THF (1:1, 3 mL). To the suspension, 4 Å molecular sieves were added and the capped vial was stirred for 75° C. for 3 days. The cooled reaction was filtered, deionized water (20 mL) was added and the filtrate was extracted with DCM (4×40 mL). Combined extracts were washed with brine (20 mL), dried over Na₂SO₄, filtered, and evaporated. The crude was purified by silica gel flash chromatography and the desired product was eluted with 4-5% MeOH/DCM eluants.

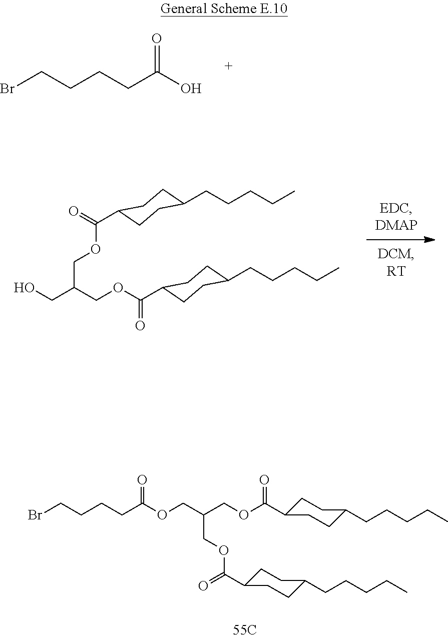 [SMILES: O=C(O)CCCCBr ; CCCCCC1CCC(C(=O)OCC(CO)COC(=O)C2CCC(CCCCC)CC2)CC1 ; "EDC, DMAP" ; CCCCCC1CCC(C(=O)OCC(COC(=O)CCCCBr)COC(=O)C2CCC(CCCCC)CC2)CC1 ; "55C"]

In a 200 mL round bottom flask, 6-bromopentanoic acid (1.17 g, 1.0 eq) was combined with EDC (1.4 g, 1.2 eq) and DMAP (0.4 g, 0.5 eq) in DCM (20 mL). The solution was then stirred for 20 minutes at room temperature before adding OH-5C (3.2 g, 1.0 eq) in DCM (10 mL). The resulting solution was stirred for 20 h at room temperature. Brine solution (100 mL) was added and the solution was stirred for 20 mins at ambient temperature. Both layers were separated, and the aqueous layer was extracted with DCM (4×50 mL). Combined extracts were dried over Na₂SO₄, filtered, and evaporated to dryness. The crude was purified by silica gel flash chromatography with 5-10% EtOAc/hexane eluants. Colorless oil, 1.8 g (45%); ¹H NMR (499 MHz, CDCl₃) δ 4.17-4.06 (m, 6H), 3.47 (dt, J=67.5, 6.4 Hz, 2H), 2.43-2.32 (m, 3H), 2.22 (tt, J=12.2, 3.5 Hz, 2H), 1.98-1.85 (m, 5H), 1.84-1.74 (m, 7H), 1.39 (qd, J=13.1, 3.5 Hz, 4H), 1.33-1.13 (m, 18H), 0.96-0.83 (m, 10H).

### H2-55C

In a 20 mL scintillation glass vial, H2 (14.6 mg, 1.0 eq), 55C (322 mg, 2.2 eq), K₂CO₃ (170 mg, 4.4 eq) and KI (41 mg, 1.0 eq) were combined and poured in CH₃CN/THF (1:1, 3 mL). To the suspension, 4 Å molecular sieves were added and the capped vial was stirred for 75° C. for 3 days. The cooled reaction was filtered, deionized water (20 mL) was added and the filtrate was extracted with DCM (4×40 mL). Combined extracts were washed with brine (20 mL), dried over Na₂SO₄, filtered, and evaporated. The crude was purified by silica gel flash chromatography and the desired product was eluted with 4-5% MeOH/DCM eluants.

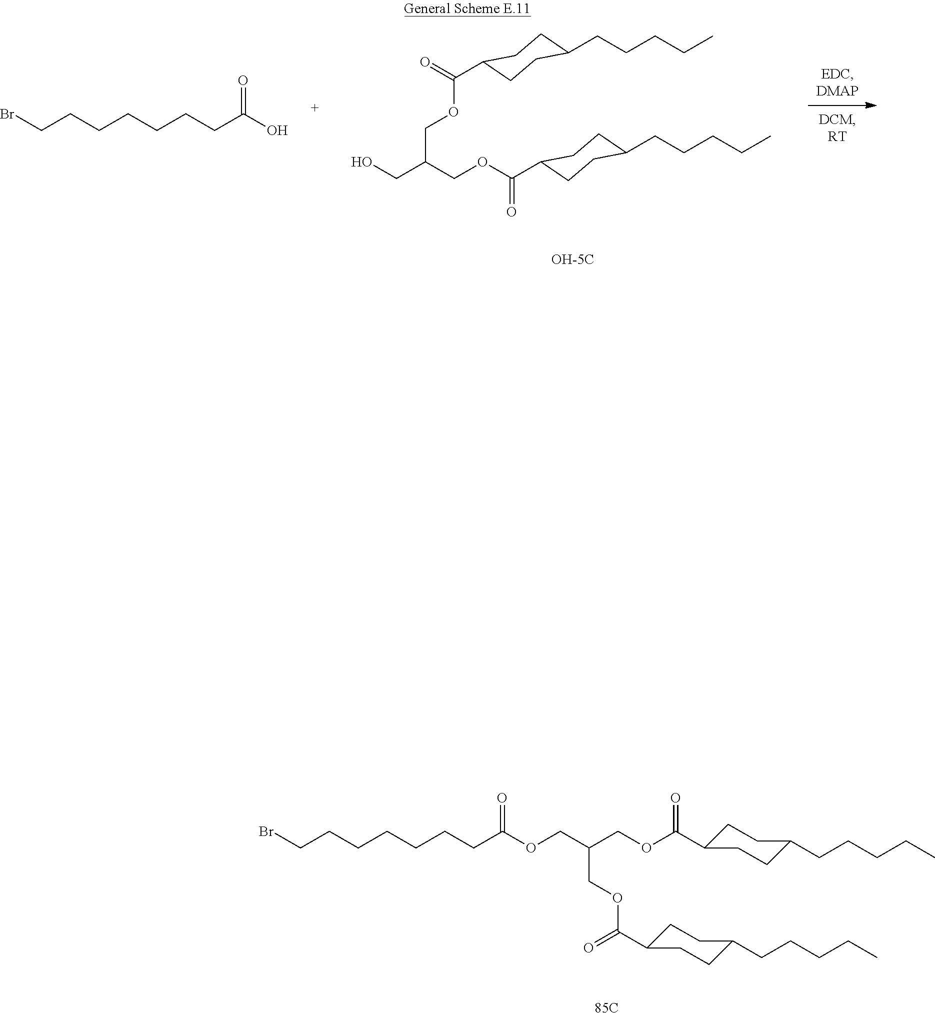 [SMILES: O=C(O)CCCCCCCBr ; CCCCCC1CCC(C(=O)OCC(CO)COC(=O)C2CCC(CCCCC)CC2)CC1 ; "EDC, DMAP" ; "OH-5C" ; CCCCCC1CCC(C(=O)OCC(COC(=O)CCCCCCCBr)COC(=O)C2CCC(CCCCC)CC2)CC1 ; "85C"]

In a 200 mL round bottom flask, 6-bromooctanoic acid (1.4 g, 1.0 eq) was combined with EDC (1.5 g, 1.2 eq) and DMAP (0.4 g, 0.5 eq) in DCM (20 mL). The solution was then stirred for 20 minutes at room temperature before adding OH-5C (3.1 g, 1.0 eq) in DCM (10 mL). The resulting solution was stirred for 20 h at room temperature. Brine solution (100 mL) was added and the solution was stirred for 20 mins at ambient temperature. Both layers were separated, and the aqueous layer was extracted with DCM (4×50 mL). Combined extracts were dried over Na₂SO₄, filtered, and evaporated to dryness. The crude was purified by silica gel flash chromatography with 5-10% EtOAc/hexane eluants. Colorless oil, 2.2 g (55%); ¹H NMR (499 MHz, CDCl₃) δ 4.11 (t, J=5.5 Hz, 6H), 3.46 (dt, J=62.5, 6.7 Hz, 2H), 2.42-2.35 (m, 1H), 2.30 (t, J=7.5 Hz, 2H), 2.22 (tt, J=12.2, 3.6 Hz, 2H), 1.98-1.90 (m, 4H), 1.88-1.72 (m, 6H), 1.65-1.58 (m, 2H), 1.47-1.12 (m, 28H), 0.94-0.84 (m, 10H).

### H2-85C

In a 20 mL scintillation glass vial, H2 (11.4 mg, 1.0 eq), 85C (282 mg, 2.2 eq), K₂CO₃ (136 mg, 4.4 eq) and KI (37 mg, 1.0 eq) were combined and poured in CH₃CN/THF (1:1, 3 mL). To the suspension, 4 Å molecular sieves were added and the capped vial was stirred for 75° C. for 3 days. The cooled reaction was filtered, deionized water (20 mL) was added and the filtrate was extracted with DCM (4×40 mL). Combined extracts were washed with brine (20 mL), dried over Na₂SO₄, filtered, and evaporated. The crude was purified by silica gel flash chromatography and the desired product was eluted with 4-5% MeOH/DCM eluants.

### Lipid Nanoparticles of the Present Disclosure

The present disclosure provides lipid nanoparticles (LNPs) comprising one or more compounds of Formula (I) and/or Formula (II). In addition to the one or more compounds of Formula (I) and/or Formula (II), the LNPs of the present disclosure can comprise one or more additional LNP components, as described below.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one compound of the present disclosure by moles. In some aspects, the at least one compound is at least one compound of Formula (I) or Formula (II), as described herein. In some aspects, the at least one compound of the present disclosure is a mixture of two or more compounds of Formula (I) or Formula (II).

### Structural Lipid

In some aspects, an LNP can further comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one structural lipid by moles.

In some aspects, a structural lipid can be a steroid. In some aspects, a structural lipid can be a sterol. In some aspects, a structural lipid can comprise cholesterol. In some aspects, a structural lipid can comprise ergosterol. In some aspects, a structural lipid can be a phytosterol.

In some aspects, the at least one structural lipid is a mixture of two structural lipids.

### Phospholipid

In some aspects, an LNP can further comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one phospholipid by moles.

As used herein, the term “phospholipid” is used in its broadest sent to refer to any amphiphilic molecule that comprises a polar (hydrophilic) headgroup comprising phosphate and two hydrophobic fatty acid chains. In some aspects, a phospholipid can comprise dioleoylphosphatidylethanolamine (DOPE). In some aspects, a phospholipid can comprise 1,2-Distearoyl-sn-glycero-3-phosphocholine (DSPC). In some aspects, a phospholipid can comprise 1,2-Dioleoyl-sn-glycero-3-phosphocholine (DOPC). In some aspects, a phospholipid can comprise DPPC (1,2-Dipalmitoyl-sn-glycero-3-phosphocholine). In some aspects, a phospholipid can comprise DDPC (1,2-Didecanoyl-sn-glycero-3-phosphocholine), DEPA-NA (1,2-Dierucoyl-sn-glycero-3-phosphate (Sodium Salt)), DEPC (1,2-Dierucoyl-sn-glycero-3-phosphocholine), DEPE (1,2-Dierucoyl-sn-glycero-3-phosphoethanolamine), DEPG-NA (1,2-Dierucoyl-sn-glycero-3[Phospho-rac-(1-glycerol) (Sodium Salt)), DLOPC (1,2-Dilinoleoyl-sn-glycero-3-phosphocholine), DLPA-NA (1,2-Dilauroyl-sn-glycero-3-phosphate (Sodium Salt)), DLPC (1,2-Dilauroyl-sn-glycero-3-phosphocholine), DLPE (1,2-Dilauroyl-sn-glycero-3-phosphoethanolamine), DLPG-NA (1,2-Dilauroyl-sn-glycero-3[Phospho-rac-(1-glycerol) (Sodium Salt)), DLPG-NH4 (1,2-Dilauroyl-sn-glycero-3[Phospho-rac-(1-glycerol) (Ammonium Salt)), DLPS-NA (1,2-Dilauroyl-sn-glycero-3-phosphoserine (Sodium Salt)), DMPA-NA (1,2-Dimyristoyl-sn-glycero-3-phosphate (Sodium Salt)), DMPC (1,2-Dimyristoyl-sn-glycero-3-phosphocholine), DMPE (1,2-Dimyristoyl-sn-glycero-3-phosphoethanolamine), DMPG-NA (1,2-Dimyristoyl-sn-glycero-3[Phospho-rac-(1-glycerol) (Sodium Salt)), DMPG-NH4 (1,2-Dimyristoyl-sn-glycero-3[Phospho-rac-(1-glycerol) (Ammonium Salt)), DMPG-NH4/NA (1,2-Dimyristoyl-sn-glycero-3[Phospho-rac-(1-glycerol) (Sodium/Ammonium Salt)), DMPS-NA (1,2-Dimyristoyl-sn-glycero-3-phosphoserine (Sodium Salt)), DOPA-NA (1,2-Dioleoyl-sn-glycero-3-phosphate (Sodium Salt)), DOPC (1,2-Dioleoyl-sn-glycero-3-phosphocholine), DOPE (1,2-Dioleoyl-sn-glycero-3-phosphoethanolamine), DOPG-NA (1,2-Dioleoyl-sn-glycero-3[Phospho-rac-(1-glycerol) (Sodium Salt)), DOPS-NA (1,2-Dioleoyl-sn-glycero-3-phosphoserine (Sodium Salt)), DPPA-NA (1,2-Dipalmitoyl-sn-glycero-3-phosphate (Sodium Salt)), DPPE (1,2-Dipalmitoyl-sn-glycero-3-phosphoethanolamine), DPPG-NA (1,2-Dipalmitoyl-sn-glycero-3[Phospho-rac-(1-glycerol) (Sodium Salt)), DPPG-NH4 (1,2-Dipalmitoyl-sn-glycero-3[Phospho-rac-(1-glycerol) (Ammonium Salt)), DPPS-NA (1,2-Dipalmitoyl-sn-glycero-3-phosphoserine (Sodium Salt)), DSPA-NA (1,2-Distearoyl-sn-glycero-3-phosphate (Sodium Salt)), DSPC (1,2-Distearoyl-sn-glycero-3-phosphocholine), DSPE (1,2-Distearoyl-sn-glycero-3-phosphoethanolamine), DSPG-NA (1,2-Distearoyl-sn-glycero-3[Phospho-rac-(1-glycerol) (Sodium Salt)), DSPG-NH4 (1,2-Distearoyl-sn-glycero-3[Phospho-rac-(1-glycerol) (Ammonium Salt)), DSPS-NA (1,2-Distearoyl-sn-glycero-3-phosphoserine (Sodium Salt)), EPC (Egg-PC), HEPC (Hydrogenated Egg PC), HSPC (Hydrogenated Soy PC), LYSOPC MYRISTIC (1-Myristoyl-sn-glycero-3-phosphocholine), LYSOPC PALMITIC (1-Palmitoyl-sn-glycero-3-phosphocholine), LYSOPC STEARIC (1-Stearoyl-sn-glycero-3-phosphocholine), Milk Sphingomyelin (MPPC; 1-Myristoyl-2-palmitoyl-sn-glycero 3-phosphocholine), MSPC (1-Myristoyl-2-stearoyl-sn-glycero-3-phosphocholine), PMPC (1-Palmitoyl-2-myristoyl-sn-glycero-3-phosphocholine), POPC (1-Palmitoyl-2-oleoyl-sn-glycero-3-phosphocholine), POPE (1-Palmitoyl-2-oleoyl-sn-glycero-3-phosphoethanolamine), POPG-NA (1-Palmitoyl-2-oleoyl-sn-glycero-3[Phospho-rac-(1-glycerol)] (Sodium Salt)), PSPC (1-Palmitoyl-2-stearoyl-sn-glycero-3-phosphocholine), SMPC (1-Stearoyl-2-myristoyl-sn-glycero-3-phosphocholine), SOPC (1-Stearoyl-2-oleoyl-sn-glycero-3-phosphocholine), SPPC (1-Stearoyl-2-palmitoyl-sn-glycero-3-phosphocholine), or any combination thereof.

### PEGylated Lipid

In some aspects, an LNP can further comprise at least about 0.25%, or at least about 0.5%, or at least about 0.75%, or at least about 1.0%, or at least about 2.5%, or at least about 5%, or at least about 7.5%, or at least about 10% PEGylated lipid by moles.

As used herein, the term “PEGylated lipid” is used to refer to any lipid that is modified (e.g. covalently linked to) at least one polyethylene glycol molecule. In some aspects, a PEGylated lipid can comprise 1,2-dimyristoyl-rac-glycero-3-methoxypolyethylene glycol-2000, hereafter referred to as DMG-PEG2000 or PEG-DMG.

In some aspects, the at least one PEGylated lipid is a mixture of two PEGylated lipids.

### Exemplary LNP Compositions

The following are exemplary LNP compositions of the present disclosure comprising at least one compound of Formula (I) and/or Formula (II), at least one structural lipid, at least one PEGylated lipid and at least one phospholipid.

In some aspects, a lipid nanoparticle comprising at least one nucleic acid can comprise about 40.75% of at least one compound of Formula (I) by moles, about 51.75% of at least one structural lipid by moles, about 5% of at least one phospholipid by moles, and about 2.5% of at least one PEGylated lipid by moles. In some aspects, a lipid nanoparticle comprising at least one nucleic acid can comprise about 30.75% to about 50.75% of at least one compound of Formula (I) by moles, about 41.75% to about 61.75% of at least one structural lipid by moles, about 0.1% to about 15% of at least one phospholipid by moles, and about 0.1% to about 12.5% of at least one PEGylated lipid by moles. In some aspects, a lipid nanoparticle comprising at least one nucleic acid can comprise about 35.75% to about 45.75% of at least one compound of Formula (I) by moles, about 46.75% to about 56.75% of at least one structural lipid by moles, about 1% to about 10% of at least one phospholipid by moles, and about 1% to about 7.5% of at least one PEGylated lipid by moles.

In some aspects, a lipid nanoparticle comprising at least one nucleic acid can comprise about 40.75% of at least one compound of Formula (II) by moles, about 51.75% of at least one structural lipid by moles, about 5% of at least one phospholipid by moles, and about 2.5% of at least one PEGylated lipid by moles. In some aspects, a lipid nanoparticle comprising at least one nucleic acid can comprise about 30.75% to about 50.75% of at least one compound of Formula (II) by moles, about 41.75% to about 61.75% of at least one structural lipid by moles, about 0.1% to about 15% of at least one phospholipid by moles, and about 0.1% to about 12.5% of at least one PEGylated lipid by moles. In some aspects, a lipid nanoparticle comprising at least one nucleic acid can comprise about 35.75% to about 45.75% of at least one compound of Formula (II) by moles, about 46.75% to about 56.75% of at least one structural lipid by moles, about 1% to about 10% of at least one phospholipid by moles, and about 1% to about 7.5% of at least one PEGylated lipid by moles.

In some aspects, a lipid nanoparticle comprising at least one nucleic acid can comprise about 43.17% of at least one compound of Formula (II) by moles, about 43.17% of at least one structural lipid by moles, about 11.96% of at least one phospholipid by moles, and about 1.7% of at least one PEGylated lipid by moles. In some aspects, a lipid nanoparticle comprising at least one nucleic acid can comprise about 33.17% to about 53.17% of at least one compound of Formula (II) by moles, about 33.17% to about 53.17% of at least one structural lipid by moles, about 1.96% to about 21.96% of at least one phospholipid by moles, and about 0.1% to about 11.7% of at least one PEGylated lipid by moles. In some aspects, a lipid nanoparticle comprising at least one nucleic acid can comprise about 38.17% to about 48.17% of at least one compound of Formula (II) by moles, about 38.17% to about 48.17% of at least one structural lipid by moles, about 6.96% to about 16.96% of at least one phospholipid by moles, and about 1% to about 6.7% of at least one PEGylated lipid by moles.

In some aspects, a lipid nanoparticle comprising at least one nucleic acid can comprise about 54% of at least one compound of Formula (II) by moles, about 35% of at least one structural lipid by moles, about 10% of at least one phospholipid by moles, and about 1% of at least one PEGylated lipid by moles. In some aspects, a lipid nanoparticle comprising at least one nucleic acid can comprise about 44% to about 64% of at least one compound of Formula (II) by moles, about 25% to about 45% of at least one structural lipid by moles, about 0.1% to about 20% of at least one phospholipid by moles, and about 0.1% to about 10% of at least one PEGylated lipid by moles. In some aspects, a lipid nanoparticle comprising at least one nucleic acid can comprise about 49% to about 59% of at least one compound of Formula (II) by moles, about 30% to about 40% of at least one structural lipid by moles, about 5% to about 15% of at least one phospholipid by moles, and about 0.5% to about 5% of at least one PEGylated lipid by moles.

Table 1A shows further exemplary LNP compositions of the present disclosure.

TABLE 1A

| | At least one | | | |
|---|---|---|---|---|
| | compound of | | | |
| | Formula (I) or | Structural | | PEGylated |
| LNP | Formula (II) | lipid | Phospholipid | lipid |
| # | (% by moles) | (% by moles) | (% by moles) | (% by moles) |
| 1 | 40.75 | 51.75 | 5 | 2.5 |
| 2 | about 30.75 to | about 41.75 to | about 0.1 to | about 0.1 to |
| | about 50.75 | about 61.75 | about 15 | about 12.5 |
| 3 | about 35.75 to | about 46.75 to | about 0.5 to | about 0.2 to |
| | about 45.75 | about 56.75 | about 10 | about 7.5 |
| 4 | about 38.25 to | about 49.25 to | about 2.5 to | about 1 to |
| | about 43.25 | about 54.25 | about 7.5 | about 5 |
| 5 | 45.89 | 45.89 | 6.21 | 2 |
| 6 | about 35.89 to | about 35.89 to | about 0.1 to | about 0.1 to |
| | about 55.89 | about 55.89 | about 16.21 | about 12 |
| 7 | about 40.89 to | about 40.89 to | about 1.21 to | about 0.2 to |
| | about 50.89 | about 50.89 | about 11.21 | about 7 |
| 8 | about 43.39 to | about 43.39 to | about 3.71 to | about 1 to |
| | about 48.39 | about 48.39 | about 8.71 | about 4.5 |
| 9 | 60 | 38.02 | 0 | 1.98 |
| 10 | about 50 to | about 28.02 to | about 0 to | about 0.1 to |
| | about 70 | about 48.02 | about 10 | about 11.98 |
| 11 | about 55 to | about 33.02 to | about 0 to | about 0.2 to |
| | about 65 | about 43.02 | about 5 | about 6.98 |
| 12 | about 57.5 to | about 35.52 to | about 0 to | about 1 to |
| | about 62.5 | about 40.52 | about 2.5 | about 4.48 |
| 13 | 54.19 | 39.5 | 3.89 | 2.41 |
| 14 | about 44.19 to | about 29.5 to | about 0.1 to | about 0.1 to |
| | about 64.19 | about 49.5 | about 13.89 | about 12.41 |
| 15 | about 49.19 to | about 34.5 to | about 0.5 to | about 0.2 to |
| | about 59.19 | about 44.5 | about 8.89 | about 7.41 |
| 16 | about 51.69 to | about 37 to | about 1.39 to | about 1 to |
| | about 56.69 | about 42 | about 6.39 | about 4.91 |
| 17 | 43.82 | 53.83 | 0 | 2.33 |
| 18 | about 33.82 to | about 43.83 to | about 0 to | about 0.1 to |
| | about 53.82 | about 63.83 | about 10 | about 12.33 |
| 19 | about 38.82 to | about 48.83 to | about 0 to | about 0.2 to |
| | about 48.82 | about 58.83 | about 5 | about 7.33 |
| 20 | about 41.32 to | about 51.33 to | about 0 to | about 1 to |
| | about 46.32 | about 56.33 | about 2.5 | about 4.83 |
| 21 | 54 | 36 | 7 | 3 |
| 22 | about 44 to | about 26 to | about 0.1 to | about 0.1 to |
| | about 64 | about 46 | about 17 | about 13 |
| 23 | about 49 to | about 31 to | about 2 to | about 0.2 to |
| | about 59 | about 41 | about 12 | about 8 |
| 24 | about 51.5 to | about 33.5 to | about 4.5 to | about 1 to |
| | about 56.5 | about 38.5 | about 9.5 | about 5.5 |
| 25 | 60 | 30 | 7 | 3 |
| 26 | about 50 to | about 20 to | about 0.1 to | about 0.1 to |
| | about 70 | about 40 | about 17 | about 13 |
| 27 | about 55 to | about 25 to | about 2 to | about 0.2 to |
| | about 65 | about 35 | about 12 | about 8 |
| 28 | about 57.5 to | about 27.5 to | about 4.5 to | about 1 to |
| | about 62.5 | about 32.5 | about 9.5 | about 5.5 |
| 29 | 46 | 46.1 | 4.9 | 3 |
| 30 | about 36 to | about 36.1 to | about 0.1 to | about 0.1 to |
| | about 56 | about 56.1 | about 14.9 | about 13 |
| 31 | about 41 to | about 41.1 to | about 0.2 to | about 0.2 to |
| | about 51 | about 51.1 | about 9.9 | about 8 |
| 32 | about 43.5 to | about 43.6 to | about 2.4 to | about 1 to |
| | about 48.5 | about 48.6 | about 7.4 | about 5.5 |
| 33 | 60 | 34.2 | 2.8 | 3 |
| 34 | about 50 to | about 24.2 to | about 0.1 to | about 0.1 to |
| | about 70 | about 44.2 | about 12.8 | about 13 |
| 35 | about 55 to | about 29.2 to | about 0.2 to | about 0.2 to |
| | about 65 | about 39.2 | about 7.8 | about 8 |
| 36 | about 57.5 to | about 31.7 to | about 0.3 to | about 1 to |
| | about 62.5 | about 36.7 | about 5.3 | about 5.5 |
| 37 | 40 | 51 | 7 | 2 |
| 38 | about 30 to | about 41 to | about 0.1 to | about 0.1 to |
| | about 50 | about 61 | about 17 | about 12 |
| 39 | about 35 to | about 46 to | about 2 to | about 0.2 to |
| | about 45 | about 56 | about 12 | about 7 |
| 40 | about 37.5 to | about 48.5 to | about 4.5 to | about 1 to |
| | about 42.5 | about 53.5 | about 9.5 | about 4.5 |
| 41 | 45.3 | 47.8 | 4.9 | 2 |
| 42 | about 35.3 to | about 37.8 to | about 0.1 to | about 0.1 to |
| | about 55.3 | about 57.8 | about 14.9 | about 12 |
| 43 | about 40.3 to | about 42.8 to | about 0.2 to | about 0.2 to |
| | about 50.3 | about 52.8 | about 9.9 | about 7 |
| 44 | about 42.8 to | about 45.3 to | about 2.4 to | about 1 to |
| | about 47.8 | about 50.3 | about 7.4 | about 4.5 |
| 45 | 45.89 | 6.21 | 45.89 | 2 |
| 46 | about 35.89 to | about 0.1 to | about 35.89 to | about 0.1 to |
| | about 55.89 | about 16.21 | about 55.89 | about 12 |
| 47 | about 40.89 to | about 1.21 to | about 40.89 to | about 0.2 to |
| | about 50.89 | about 11.21 | about 50.89 | about 7 |
| 48 | about 43.39 to | about 3.71 to | about 43.39 to | about 1 to |
| | about 48.39 | about 8.71 | about 48.39 | about 4.5 |
| 49 | 43.17 | 43.17 | 11.96 | 1.7 |
| 50 | about 33.17 to | about 33.17 to | about 1.96 to | about 0.1 to |
| | about 53.17 | about 53.17 | about 21.96 | about 11.7 |
| 51 | about 38.17 to | about 38.17 to | about 6.96 to | about 0.2 to |
| | about 48.17 | about 48.17 | about 16.96 | about 6.7 |
| 52 | about 40.67 to | about 40.67 to | about 9.46 to | about 1 to |
| | about 45.67 | about 45.67 | about 14.46 | about 4.2 |
| 53 | 54 | 35 | 10 | 1 |
| 54 | about 44 to | about 25 to | about 0.1 to | about 0.1 to |
| | about 64 | about 45 | about 20 | about 11 |
| 55 | about 49 to | about 30 to | about 5 to | about 0.2 to |
| | about 59 | about 40 | about 15 | about 6 |
| 56 | about 51.5 to | about 32.5 to | about 7.5 to | about 0.5 to |
| | about 56.5 | about 37.5 | about 12.5 | about 3.5 |
| 57 | 50 | 38.5 | 10 | 1.5 |
| 58 | about 40 to | about 28.5 to | about 0.1 to | about 0.1 to |
| | about 60 | about 48.5 | about 20 | about 11.5 |
| 59 | about 45 to | about 33.5 to | about 5 to | about 0.2 to |
| | about 55 | about 43.5 | about 15 | about 6.5 |
| 60 | about 47.5 to | about 36 to | about 7.5 to | about 0.5 to |
| | about 52.5 | about 41 | about 12.5 | about 4 |
| 61 | 50 | 42.5 | 5 | 2.5 |
| 62 | about 40 to | about 32.5 to | about 0.1 to | about 0.1 to |
| | about 60 | about 52.5 | about 15 | about 12.5 |
| 63 | about 45 to | about 37.5 to | about 0.5 to | about 0.2 to |
| | about 55 | about 47.5 | about 10 | about 7.5 |
| 64 | about 47.5 to | about 40 to | about 2.5 to | about 1 to |
| | about 52.5 | about 45 | about 7.5 | about 5 |
| 65 | 45 | 48.5 | 5 | 1.5 |
| 66 | about 35 to | about 38.5 to | about 0.1 to | about 0.1 to |
| | about 55 | about 58.5 | about 15 | about 11.5 |
| 67 | about 40 to | about 43.5 to | about 0.5 to | about 0.2 to |
| | about 50 | about 53.5 | about 10 | about 6.5 |
| 68 | about 42.5 to | about 46 to | about 2.5 to | about 0.5 to |
| | about 47.5 | about 51 | about 7.5 | about 4 |
| 69 | 50 | 37.5 | 10 | 2.5 |
| 70 | about 40 to | about 27.5 to | about 0.1 to | about 0.1 to |
| | about 60 | about 47.5 | about 20 | about 12.5 |
| 71 | about 45 to | about 32.5 to | about 5 to | about 0.2 to |
| | about 55 | about 42.5 | about 15 | about 7.5 |
| 72 | about 47.5 to | about 35 to | about 7.5 to | about 1 to |
| | about 52.5 | about 40 | about 12.5 | about 5 |
| 73 | 50 | 41 | 7.5 | 1.5 |
| 74 | about 40 to | about 31 to | about 0.1 to | about 0.1 to |
| | about 60 | about 51 | about 17.5 | about 11.5 |
| 75 | about 45 to | about 36 to | about 2.5 to | about 0.2 to |
| | about 55 | about 46 | about 12.5 | about 6.5 |
| 76 | about 47.5 to | about 38.5 to | about 5 to | about 0.5 to |
| | about 52.5 | about 43.5 | about 10 | about 4 |
| 77 | 40 | 52.5 | 5 | 2.5 |
| 78 | about 30 to | about 42.5 to | about 0.1 to | about 0.1 to |
| | about 50 | about 62.5 | about 15 | about 12.5 |
| 79 | about 35 to | about 47.5 to | about 0.5 to | about 0.2 to |
| | about 45 | about 57.5 | about 10 | about 7.5 |
| 80 | about 37.5 to | about 50 to | about 2.5 to | about 1 to |
| | about 42.5 | about 55 | about 7.5 | about 5 |
| 81 | 40 | 50 | 7.5 | 2.5 |
| 82 | about 30 to | about 40 to | about 0.1 to | about 0.1 to |
| | about 50 | about 60 | about 17.5 | about 12.5 |
| 83 | about 35 to | about 45 to | about 2.5 to | about 0.2 to |
| | about 45 | about 55 | about 12.5 | about 7.5 |
| 84 | about 37.5 to | about 47.5 to | about 5 to | about 1 to |
| | about 42.5 | about 52.5 | about 10 | about 5 |
| 85 | 50 | 43 | 5 | 2 |
| 86 | about 40 to | about 33 to | about 0.1 to | about 0.1 to |
| | about 60 | about 53 | about 15 | about 12 |
| 87 | about 45 to | about 38 to | about 0.5 to | about 0.2 to |
| | about 55 | about 48 | about 10 | about 7 |
| 88 | about 47.5 to | about 40.5 to | about 2.5 to | about 1 to |
| | about 52.5 | about 45.5 | about 7.5 | about 4.5 |
| 89 | 50 | 38.5 | 10 | 1.5 |
| 90 | about 40 to | about 28.5 to | about 0.1 to | about 0.1 to |
| | about 60 | about 48.5 | about 20 | about 11.5 |
| 91 | about 45 to | about 33.5 to | about 5 to | about 0.2 to |
| | about 55 | about 43.5 | about 15 | about 6.5 |
| 92 | about 47.5 to | about 36 to | about 7.5 to | about 0.5 to |
| | about 52.5 | about 41 | about 12.5 | about 4 |
| 93 | 45 | 45.5 | 7.5 | 2 |
| 94 | about 35 to | about 35.5 to | about 0.1 to | about 0.1 to |
| | about 55 | about 55.5 | about 17.5 | about 12 |
| 95 | about 40 to | about 40.5 to | about 2.5 to | about 0.2 to |
| | about 50 | about 50.5 | about 12.5 | about 7 |
| 96 | about 42.5 to | about 43 to | about 5 to | about 1 to |
| | about 47.5 | about 47 | about 10 | about 4.5 |
| 97 | 40 | 48 | 10 | 2 |
| 98 | about 30 to | about 38 to | about 0.1 to | about 0.1 to |
| | about 50 | about 58 | about 20 | about 12 |
| 99 | about 35 to | about 43 to | about 5 to | about 0.2 to |
| | about 45 | about 53 | about 15 | about 7 |
| 100 | about 37.5 to | about 45.5 to | about 7.5 to | about 1 to |
| | about 42.5 | about 50.5 | about 12.5 | about 4.5 |
| 101 | 40 | 53.5 | 5 | 1.5 |
| 102 | about 30 to | about 43.5 to | about 0.1 to | about 0.1 to |
| | about 50 | about 63.5 | about 15 | about 11.5 |
| 103 | about 35 to | about 48.5 to | about 0.5 to | about 0.2 to |
| | about 45 | about 58.5 | about 10 | about 6.5 |
| 104 | about 37.5 to | about 51 to | about 2.5 to | about 0.5 to |
| | about 42.5 | about 56 | about 7.5 | about 4 |
| 105 | 45 | 42.5 | 10 | 2.5 |
| 106 | about 35 to | about 32.5 to | about 0.1 to | about 0.1 to |
| | about 55 | about 52.5 | about 20 | about 12.5 |
| 107 | about 40 to | about 37.5 to | about 5 to | about 0.2 to |
| | about 50 | about 47.5 | about 15 | about 7.5 |
| 108 | about 42.5 to | about 40 to | about 7.5 to | about 1 to |
| | about 47.5 | about 45 | about 12.5 | about 5 |
| 109 | 40 | 48.5 | 10 | 1.5 |
| 110 | about 30 to | about 38.5 to | about 0.1 to | about 0.1 to |
| | about 50 | about 58.5 | about 20 | about 11.5 |
| 111 | about 35 to | about 43.5 to | about 5 to | about 0.2 to |
| | about 45 | about 53.5 | about 15 | about 6.5 |
| 112 | about 37.5 to | about 46 to | about 7.5 to | about 0.5 to |
| | about 42.5 | about 51 | about 12.5 | about 4 |
| 113 | 50 | 38 | 10 | 2 |
| 114 | about 40 to | about 28 to | about 0.1 to | about 0.1 to |
| | about 60 | about 48 | about 20 | about 12 |
| 115 | about 45 to | about 33 to | about 5 to | about 0.2 to |
| | about 55 | about 43 | about 15 | about 7 |
| 116 | about 47.5 to | about 35.5 to | about 7.5 to | about 1 to |
| | about 52.5 | about 40.5 | about 12.5 | about 4.5 |
| 117 | 40 | 50.5 | 7.5 | 2 |
| 118 | about 30 to | about 40.5 to | about 0.1 to | about 0.1 to |
| | about 50 | about 60.5 | about 17.5 | about 12 |
| 119 | about 35 to | about 45.5 to | about 2.5 to | about 0.2 to |
| | about 45 | about 55.5 | about 12.5 | about 7 |
| 120 | about 37.5 to | about 48 to | about 5 to | about 1 to |
| | about 42.5 | about 53 | about 10 | about 4.5 |
| 121 | 40 | 49 | 10 | 1 |
| 122 | about 30 to | about 39 to | about 0.1 to | about 0.1 to |
| | about 50 | about 59 | about 20 | about 11 |
| 123 | about 35 to | about 44 to | about 5 to | about 0.2 to |
| | about 45 | about 54 | about 15 | about 6 |
| 124 | about 37.5 to | about 46.5 to | about 7.5 to | about 0.5 to |
| | about 42.5 | about 51.5 | about 12.5 | about 3.5 |
| 125 | 45 | 49 | 5 | 1 |
| 126 | about 35 to | about 39 to | about 0.1 to | about 0.1 to |
| | about 55 | about 55 | about 15 | about 11 |
| 127 | about 40 to | about 44 to | about 0.5 to | about 0.2 to |
| | about 50 | about 54 | about 10 | about 6 |
| 128 | about 42.5 to | about 46.5 to | about 2.5 to | about 0.5 to |
| | about 47.5 | about 51.5 | about 7.5 | about 3.5 |

In some aspects, including in the LNP compositions put forth in Tables 1A-1C, the compound of Formula (I) or Formula (II) is one of COMPOUND NOS. 1-76.

In some aspects, including in the LNP compositions put forth in Tables 1A-1C, the structural lipid can be cholesterol.

In some aspects, including in the LNP compositions put forth in Tables 1A-1C, the phospholipid is DOPE.

In some aspects, including in the LNP compositions put forth in Tables 1A-1C, the phospholipid is DSPC.

In some aspects, including in the LNP compositions put forth in Tables 1A-1C, the phospholipid is DOPC.

In some aspects, including in the LNP compositions put forth in Tables 1A-1C, the phospholipid is DPPC.

In some aspects, including in the LNP compositions put forth in Tables 1A-1C, the phospholipid can be a mixture of DSPC and DOPC. In some aspects, the mixture of DSPC and DOPC can comprise DSPC and DOPC at a 1:1 ratio (e.g. a LNP that comprises 10% phospholipid can comprise 5% DOPC and 5% DSPC).

In some aspects of the preceding LNPs, including the LNP compositions put forth in Table 1, the PEGylated lipid can be DMG-PEG2000.

In some aspects, including in the LNP compositions put forth in Tables 1A-1C, the structural lipid can be cholesterol, the phospholipid can be DOPE and the PEGylated lipid can be DMG-PEG2000.

In some aspects, including in the LNP compositions put forth in Tables 1A-1C, the structural lipid can be cholesterol, the phospholipid can be DOPC and the PEGylated lipid can be DMG-PEG2000.

In some aspects, including in the LNP compositions put forth in Tables 1A-1C, the structural lipid can be cholesterol, the phospholipid can be DSPC and the PEGylated lipid can be DMG-PEG2000.

In some aspects, including in the LNP compositions put forth in Tables 1A-1C, the structural lipid can be cholesterol, the phospholipid can be DPPC and the PEGylated lipid can be DMG-PEG2000.

In some aspects, including in the LNP compositions put forth in Tables 1A-1C, the structural lipid can be cholesterol, the phospholipid can be a mixture of DSPC and DOPC, and the PEGylated lipid can be DMG-PEG2000. In some aspects, the mixture of DSPC and DOPC can comprise DSPC and DOPC at a 1:1 ratio (e.g. a LNP that comprises 10% phospholipid can comprise 5% DOPC and 5% DSPC).

### Targeting Ligand

In some aspects, an LNP, including those put forth in Table 1A, can further comprise at least one targeting ligand.

In some aspects, an LNP of the present disclosure can further comprise at least about 0.05%, or at least about 0.1%, or at least about 0.15%, or at least about 0.2%, or at least about 0.25%, or at least about 0.3%, or at least about 0.35%, or at least about 0.4%, or at least about 0.45%, or at least about 0.5%, or at least about 0.55%, or at least about 0.6%, or at least about 0.65%, or at least about 0.7%, or at least about 0.75%, or at least about 0.8%, or at least about 0.85%, or at least about 0.9%, or at least about 0.95%, or at least about 1.0%, or at least about 1.1%, or at least about 1.2%, or at least about 1.3%, or at least about 1.4%, or at least about 1.5%, or at least about 1.6%, or at least about 1.7%, or at least about 1.8%, or at least about 1.9%, or at least about 2.0% of at least one targeting ligand by moles.

A targeting ligand may be any ligand that provides an enhanced affinity for a selected target, e.g., molecule, cell or cell type, e.g., a cellular or organ compartment, tissue, organ or region of the body, as, e.g., compared to a species absent such a ligand.

In some aspects, a composition comprising a targeting lipid is well-tolerated and provides an adequate therapeutic index, such that patient treatment with an effective dose of the composition is associated with an improved toxicity and/or risk profile to the patient, compared to patient treatment with an effective dose of a composition that does not comprise a targeting ligand.

In some aspects, a targeting ligand provides an enhanced affinity for the liver or liver cells, such as hepatocytes. A non-limiting example of a targeting ligand with enhanced affinity for the liver or liver cells is GalNac (n-acetyl-galactosamine). Thus, in some embodiments, the invention provides LNP compositions comprising a targeting ligand comprising GalNac.

In some aspects, a targeting ligand comprising GalNac can be a pegylated GalNac molecule. In some aspects, a pegylated GalNac molecule can be Tri-GalNac-PEG2000-DESPE (referred to herein as “GalNac-PEG”), and which structure is shown below:

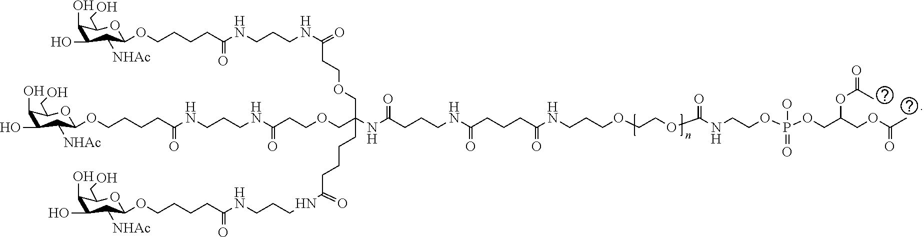

Thus, in some aspects, the present disclosure provides LNPS comprising GalNac-PEG

In some aspects, a targeting ligand can also include targeting groups, for example a group of tissue targeting agents. A non-limiting example of a targeting group can be multivalent GalNac molecule. Thus, in some embodiments, the invention provides LNP compositions comprising a targeting ligand comprising multivalent GalNac. A non-limiting example of a multivalent GalNac molecule is GalNac-PEG.

Table 1B shows exemplary LNP compositions of the present disclosure comprising at least one compound of Formula (I) and/or Formula (II), at least one structural lipid, at least one PEGylated lipid and at least one phospholipid, and at least one targeting ligand comprising GalNac.

TABLE 1B

| | At least one | | | | |
|---|---|---|---|---|---|
| | compound of | | | | Targeting |
| | Formula (I) or | | | | ligand |
| | Formula (II) | Structural lipid | Phospholipid | PEGylated lipid | comprising |
| LNP # | (% by moles) | (% by moles) | (% by moles) | (% by moles) | GalNac |
| 129 | 50 | 38.5 | 10 | 1.25 | 0.25 |
| 130 | about 40 to | about 28.5 to | about 0.1 to | about 0.1 to | about 0.1 to |
| | about 60 | about 48.5 | about 20 | about 11.25 | about 10.25 |
| 131 | about 45 to | about 33.5 to | about 5 to | about 0.2 to | about 0.2 to |
| | about 55 | about 43.5 | about 15 | about 6.25 | about 5.25 |
| 132 | about 47.5 to | about 36 to | about 7.5 to | about 0.5 to | about 0.5 to |
| | about 52.5 | about 41 | about 12.5 | about 3.75 | about 2.75 |
| 133 | 50 | 38.5 | 10 | 1 | 0.5 |
| 134 | about 40 to | about 28.5 to | about 0.1 to | about 0.1 to | about 0.1 to |
| | about 60 | about 48.5 | about 20 | about 11 | about 10.5 |
| 135 | about 45 to | about 33.5 to | about 5 to | about 0.2 to | about 0.2 to |
| | about 55 | about 43.5 | about 15 | about 6 | about 5.5 |
| 136 | about 47.5 to | about 36 to | about 7.5 to | about 0.5 to | about 0.3 to |
| | about 52.5 | about 41 | about 12.5 | about 3.5 | about 3 |
| 137 | 45 | 42.5 | 10 | 2.25 | 0.25 |
| 138 | about 35 to | about 32.5 to | about 0.1 to | about 0.1 to | about 0.1 to |
| | about 55 | about 52.5 | about 20 | about 12.25 | about 10.25 |
| 139 | about 40 to | about 37.5 to | about 5 to | about 0.2 to | about 0.2 to |
| | about 50 | about 47.5 | about 15 | about 7.25 | about 5.25 |
| 140 | about 42.5 to | about 40 to | about 7.5 to | about 1 to | about 0.5 to |
| | about 47.5 | about 45 | about 12.5 | about 4.75 | about 2.75 |
| 141 | 45 | 42.5 | 10 | 2 | 0.5 |
| 142 | about 35 to | about 32.5 to | about 0.1 to | about 0.1 to | about 0.1 to |
| | about 55 | about 52.5 | about 20 | about 12 | about 10.5 |
| 143 | about 40 to | about 37.5 to | about 5 to | about 0.2 to | about 0.2 to |
| | about 50 | about 47.5 | about 15 | about 7 | about 5.5 |
| 144 | about 42.5 to | about 40 to | about 7.5 to | about 1 to | about 0.3 to |
| | about 47.5 | about 45 | about 12.5 | about 4.5 | about 3 |
| 145 | 50 | 38.5 | 10 | 1.2 | 0.3 |
| 146 | about 40 to | about 28.5 to | about 0.1 to | about 0.1 to | about 0.1 to |
| | about 60 | about 48.5 | about 20 | about 11.2 | about 10.3 |
| 147 | about 45 to | about 33.5 to | about 5 to | about 0.2 to | about 0.2 to |
| | about 55 | about 43.5 | about 15 | about 6.2 | about 5.3 |
| 148 | about 47.5 to | about 36 to | about 7.5 to | about 0.5 to | about 0.5 to |
| | about 52.5 | about 41 | about 12.5 | about 3.7 | about 2.8 |
| 149 | 50 | 38 | 10 | 1.7 | 0.3 |
| 150 | about 40 to | about 28 to | about 0.1 to | about 0.1 to | about 0.1 to |
| | about 60 | about 48 | about 20 | about 11.7 | about 10.3 |
| 151 | about 45 to | about 33 to | about 5 to | about 0.2 to | about 0.2 to |
| | about 55 | about 43 | about 15 | about 6.7 | about 5.3 |
| 152 | about 47.5 to | about 35.5 to | about 7.5 to | about 1 to | about 0.5 to |
| | about 52.5 | about 40.5 | about 12.5 | about 4.2 | about 2.8 |
| 153 | 50 | 38 | 10 | 1.5 | 0.5 |
| 154 | about 40 to | about 28 to | about 0.1 to | about 0.1 to | about 0.1 to |
| | about 60 | about 48 | about 20 | about 11.5 | about 10.5 |
| 155 | about 45 to | about 33 to | about 5 to | about 0.2 to | about 0.2 to |
| | about 55 | about 43 | about 15 | about 6.5 | about 5.5 |
| 156 | about 47.5 to | about 35.5 to | about 7.5 to | about 0.5 to | about 0.3 to |
| | about 52.5 | about 40.5 | about 12.5 | about 4 | about 3 |
| 157 | 50 | 42.5 | 5 | 2 | 0.5 |
| 158 | about 40 to | about 32.5 to | about 0.1 to | about 0.1 to | about 0.1 to |
| | about 60 | about 52.5 | about 15 | about 12 | about 10.5 |
| 159 | about 45 to | about 37.5 to | about 0.5 to | about 0.2 to | about 0.2 to |
| | about 55 | about 47.5 | about 10 | about 7 | about 5.5 |
| 160 | about 47.5 to | about 40 to | about 2.5 to | about 1 to | about 0.3 to |
| | about 52.5 | about 45 | about 7.5 | about 4.5 | about 3 |
| 161 | 50 | 41 | 7.5 | 1 | 0.5 |
| 162 | about 40 to | about 31 to | about 0.1 to | about 0.1 to | about 0.1 to |
| | about 60 | about 51 | about 17.5 | about 11 | about 10.5 |
| 163 | about 45 to | about 36 to | about 2.5 to | about 0.2 to | about 0.2 to |
| | about 55 | about 46 | about 12.5 | about 6 | about 5.5 |
| 164 | about 47.5 to | about 38.5 to | about 5 to | about 0.5 to | about 0.3 to |
| | about 52.5 | about 43.5 | about 10 | about 3.5 | about 3 |
| 165 | 50 | 38 | 10 | 1.5 | 0.5 |
| 166 | about 40 to | about 28 to | about 0.1 to | about 0.1 to | about 0.1 to |
| | about 60 | about 48 | about 20 | about 11.5 | about 10.5 |
| 167 | about 45 to | about 33 to | about 5 to | about 0.2 to | about 0.2 to |
| | about 55 | about 43 | about 15 | about 6.5 | about 5.5 |
| 168 | about 47.5 to | about 35.5 to | about 7.5 to | about 0.5 to | about 0.3 to |
| | about 52.5 | about 40.5 | about 12.5 | about 4 | about 3 |
| 169 | 45 | 45.75 | 7.5 | 1.5 | 0.25 |
| 170 | about 35 to | about 35.75 to | about 0.1 to | about 0.1 to | about 0.1 to |
| | about 55 | about 55.75 | about 17.5 | about 11.5 | about 10.25 |
| 171 | about 40 to | about 40.75 to | about 2.5 to | about 0.2 to | about 0.2 to |
| | about 50 | about 50.75 | about 12.5 | about 6.5 | about 5.25 |
| 172 | about 42.5 to | about 43.25 to | about 5 to | about 0.5 to | about 0.5 to |
| | about 47.5 | about 48.25 | about 10 | about 4 | about 2.75 |
| 173 | 40 | 53.5 | 5 | 1 | 0.5 |
| 174 | about 30 to | about 43.5 to | about 0.1 to | about 0.1 to | about 0.1 to |
| | about 50 | about 63.5 | about 15 | about 11 | about 10.5 |
| 175 | about 35 to | about 48.5 to | about 0.5 to | about 0.2 to | about 0.2 to |
| | about 45 | about 58.5 | about 10 | about 6 | about 5.5 |
| 176 | about 37.5 to | about 51 to | about 2.5 to | about 0.5 to | about 0.3 to |
| | about 42.5 | about 56 | about 7.5 | about 3.5 | about 3 |
| 177 | 40 | 48 | 10 | 1.5 | 0.5 |
| 178 | about 30 to | about 38 to | about 0.1 to | about 0.1 to | about 0.1 to |
| | about 50 | about 58 | about 20 | about 11.5 | about 10.5 |
| 179 | about 35 to | about 43 to | about 5 to | about 0.2 to | about 0.2 to |
| | about 45 | about 53 | about 15 | about 6.5 | about 5.5 |
| 180 | about 37.5 to | about 45.5 to | about 7.5 to | about 0.5 to | about 0.3 to |
| | about 42.5 | about 50.5 | about 12.5 | about 4 | about 3 |
| 181 | 40 | 52.75 | 5 | 2 | 0.25 |
| 182 | about 30 to | about 42.75 to | about 0.1 to | about 0.1 to | about 0.1 to |
| | about 50 | about 62.75 | about 15 | about 12 | about 10.25 |
| 183 | about 35 to | about 47.75 to | about 0.5 to | about 0.2 to | about 0.2 to |
| | about 45 | about 57.75 | about 10 | about 7 | about 5.25 |
| 184 | about 37.5 to | about 50.25 to | about 2.5 to | about 1 to | about 0.5 to |
| | about 42.5 | about 55.25 | about 7.5 | about 4.5 | about 2.75 |
| 185 | 50 | 38.75 | 10 | 1 | 0.25 |
| 186 | about 40 to | about 28.75 to | about 0.1 to | about 0.1 to | about 0.1 to |
| | about 60 | about 48.75 | about 20 | about 11 | about 10.25 |
| 187 | about 45 to | about 33.75 to | about 5 to | about 0.2 to | about 0.2 to |
| | about 55 | about 43.75 | about 15 | about 6 | about 5.25 |
| 188 | about 47.5 to | about 36.25 to | about 7.5 to | about 0.5 to | about 0.5 to |
| | about 52.5 | about 41.25 | about 12.5 | about 3.5 | about 2.75 |
| 189 | 50 | 43.25 | 5 | 1.5 | 0.25 |
| 190 | about 40 to | about 33.25 to | about 0.1 to | about 0.1 to | about 0.1 to |
| | about 60 | about 53.25 | about 15 | about 11.5 | about 10.25 |
| 191 | about 45 to | about 38.25 to | about 0.5 to | about 0.2 to | about 0.2 to |
| | about 55 | about 48.25 | about 10 | about 6.5 | about 5.25 |
| 192 | about 47.5 to | about 40.75 to | about 2.5 to | about 0.5 to | about 0.5 to |
| | about 52.5 | about 45.75 | about 7.5 | about 4 | about 2.75 |

In some aspects, including in the LNPS of Table 1B, the targeting ligand comprising GalNac is GalNac-PEG.

In some aspects, a targeting ligand can comprise DSPE (1, 2-Distearoyl-sn-glycero-3-phosphoethanolamine). Thus, in some embodiments, the invention provides LNP compositions comprising a targeting ligand comprising DSPE. In some aspects, the DSPE can be pegylated. In some aspects, a targeting ligand comprising DSPE can be 1,2-distearoyl-sn-glycero-3-phosphoethanolamine-N-[methoxy (polyethylene glycol)-2000], also referred to herein as DSPE-PEG2000 or DSPE-PEG, and whose structure is shown below:

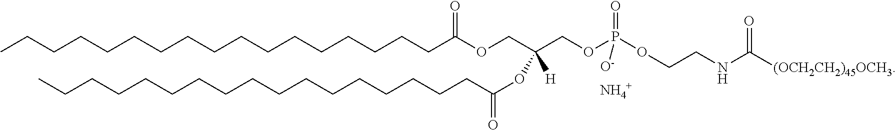

Table 1C shows exemplary LNP compositions of the present disclosure comprising at least one compound of Formula (I) and/or Formula (II), at least one structural lipid, at least one PEGylated lipid and at least one phospholipid, and at least one targeting ligand comprising DSPE.

TABLE 1C

| | At least one | | | | |
|---|---|---|---|---|---|
| | compound of | | | | Targeting |
| | Formula (I) or | | | | ligand |
| | Formula (II) | Structural lipid | Phospholipid | PEGylated lipid | comprising |
| LNP # | (% by moles) | (% by moles) | (% by moles) | (% by moles) | DSPE |
| 193 | 50 | 38.5 | 10 | 1 | 0.5 |
| 194 | about 40 to | about 28.5 to | about 0.1 to | about 0.1 to | about 0.1 to |
| | about 60 | about 48.5 | about 20 | about 11 | about 10.5 |
| 195 | about 45 to | about 33.5 to | about 5 to | about 0.2 to | about 0.2 to |
| | about 55 | about 43.5 | about 15 | about 6 | about 5.5 |
| 196 | about 47.5 to | about 36 to | about 7.5 to | about 0.5 to | about 0.3 to |
| | about 52.5 | about 41 | about 12.5 | about 3.5 | about 3 |

### Nucleic Acid Molecules

In some aspects, a lipid nanoparticle of the present disclosure, including those put forth in Tables 1A-1C, can further comprise at least one nucleic acid. In some aspects, a lipid nanoparticle can comprise a plurality of nucleic acid molecules. In some aspects, the at least one nucleic acid or the plurality of nucleic acid molecules can be formulated in a lipid nanoparticle.

Accordingly, a lipid nanoparticle can comprise at least one nucleic acid, at least one compound of the present disclosure, at least one structural lipid, at least one phospholipid, and at least one PEGylated lipid. In some aspects, the lipid nanoparticle can further comprise at least one targeting ligand.

In some aspects, the at least one nucleic acid is a DNA molecule. In one aspect, the at least one DNA molecule is a DoggyBone DNA molecule. In some aspects, the at least one DNA molecule is a DNA nanoplasmid.

the at least one nucleic acid is an RNA molecule. In some aspects, the RNA molecule is an mRNA molecule. In some aspects, the mRNA molecule further comprises a 5′-CAP. In some aspects, all of the cytidine residues in an mRNA molecule can be 5-methylcytidine.

In some aspects, the at least one RNA molecules is a guide RNA (gRNA) molecule.

In some aspects, an at least one nucleic acid can comprise both mRNA molecules and guide RNA (gRNA) molecules. That is, the LNPs of the present disclosure can comprise both mRNA molecules and gRNA molecules. In some aspects wherein the LNPs comprise both mRNA molecules and gRNA molecules, the mRNA molecules comprise at least one nucleic acid sequence that encodes a fusion protein, wherein the fusion protein comprises: (i) an inactivated Cas9 (dCas9) protein or an inactivated nuclease domain thereof; and (ii) a Clo051 protein or a nuclease domain thereof, and wherein the gRNA molecules encode guide RNA sequence targeting one or more specific genomic loci. In some aspects, the fusion protein can be a Cas-CLOVER protein. In some aspects, the gRNA molecules can target the psk9 gene.

In some aspects wherein the LNPs comprise both mRNA molecules and gRNA molecules, the ratio of mRNA: gRNA can be about 1:2, or about 1:3, or about 1:4, or about 1:5, or about 1:6, or about 1:7, or about 1:8, or about 1:9, or about 1:10 or about 1:1, or about 2:1, or about 3:1, or about 4:1, or about 5:1, or about 6:1, or about 7:1, or about 8:1, or about 9:1 or about 10:1.

In some aspects, an at least one nucleic acid can comprise at least one RNA molecule and at least one DNA molecule. That is, the LNPs of the present disclosure can comprise both RNA molecules and DNA molecules.

In some aspects, the LNPs of the present disclosure can comprise both RNA molecules and DNA molecules, wherein the RNA molecules comprise at least one nucleic acid sequence that encodes a transposase and wherein the DNA molecules comprise at least one nucleic acid sequence that comprises a transposon. In some aspects, the transposase can be any of the transposases described herein. In some aspects, the transposon can be a transposon comprising at least one nucleic acid sequence encoding a FVIII polypeptide. In some aspects, the transposon can be a transposon comprising at least one nucleic acid sequence encoding a human propionyl-CoA carboxylase subunit alpha (PCCA) polypeptide.

In some aspects wherein the LNPs of the present disclosure comprise both RNA (e.g. mRNA) and DNA, the ratio of RNA to DNA (RNA: DNA) in the LNPs can be about 1:2, or about 1:3, or about 1:4, or about 1:1, or about 2:1, or about 3:1, or about 4:1, or about 5:1, or about 6:1, or about 7:1, or about 8:1, or about 9:1 or about 10:1.

In some aspects, a lipid nanoparticle can comprise lipid and nucleic acid at a specified ratio (weight/weight).

In some aspects, a lipid nanoparticle comprising at least one nucleic acid can comprise lipid and nucleic acid at a ratio of about 5:1 to about 15:1, or about 10:1 to about 20:1, or about 15:1 to about 25:1, or about 20:1 to about 30:1, or about 25:1 to about 35:1 or about 30:1 to about 40:1, or about 35:1 to about 45:1, or about 40:1 to about 50:1, or about 45:1 to about 55:1, or about 50:1 to about 60:1, or about 55:1 to about 65:1, or about 60:1 to about 70:1, or about 65:1 to about 75:1, or about 70:1 to about 80:1, or about 75:1 to about 85:1, or about 80:1 to about 90:1, or about 85:1 to about 95:1, or about 90:1 to about 100:1, or about 95:1 to about 105:1, or about 100:1 to about 110:1, or about 105:1 to about 115:1, or about 110:1 to about 120:1, or about 115:1 to about 125:1, or about 120:1 to about 130:1, or about 125:1 to about 135:1, or about 130:1 to about 140:1, or about 135:1 to about 145:1, or about 140:1 to about 150:1, lipid: nucleic acid, weight/weight.

In some aspects, a lipid nanoparticle comprising at least one nucleic acid can comprise lipid and nucleic acid at a ratio of about 5:1, or about 10:1, or about 15:1, or about 20:1, or about 25:1, or about 30:1, or about 35:1, or about 40:1, or about 45:1, or about 50:1, or about 55:1, or about 60:1, or about 65:1, or about 70:1, or about 75:1, or about 80:1, or about 85:1, or about 90:1, or about 95:1, or about 100:1, or about 105:1, or about 110:1, or about 115:1, or about 120:1, or about 125:1, or about 130:1, or about 135:1, or about 140:1, or about 145:1, or about 150:1, lipid: nucleic acid, weight/weight.

In some aspects, a lipid nanoparticle comprising at least one nucleic acid can comprise lipid and nucleic acid at a ratio of about 10:1, or about 25:1, or about 40:1, lipid: nucleic acid, weight/weight.

In some aspects, a lipid nanoparticle comprising at least one nucleic acid can comprise lipid and nucleic acid at a ratio of about 20:1, or about 40:1, or about 60:1, or about 80:1, or about 120:1 lipid: nucleic acid, weight/weight.

In some aspects, including in the LNPs put forth in Tables 1A-1C, the ratio of lipid to nucleic acid in the nanoparticle can be about 30:1 to about 50:1 (w/w), or about 35:1 to about 45:1 (w/w). In some aspects, the ratio of lipid to nucleic acid in the nanoparticle can be about 40:1 (w/w).

In some aspects, including in the LNPs put forth in Tables 1A-1C, the ratio of lipid to nucleic acid in the nanoparticle can be about 40:1 to about 60:1 (w/w), or about 45:1 to about 55:1 (w/w). In some aspects, the ratio of lipid to nucleic acid in the nanoparticle can be about 50:1 (w/w).

In some aspects, including in the LNPs put forth in Tables 1A-1C, the ratio of lipid to nucleic acid in the nanoparticle can be about 50:1 to about 70:1 (w/w), or about 55:1 to about 65:1 (w/w). In some aspects, the ratio of lipid to nucleic acid in the nanoparticle can be about 60:1 (w/w).

In some aspects, including in the LNPs put forth in Tables 1A-1C, the ratio of lipid to nucleic acid in the nanoparticle can be about 70:1 to about 90:1 (w/w), or about 75:1 to about 85:1 (w/w). In some aspects, the ratio of lipid to nucleic acid in the nanoparticle can be about 80:1 (w/w).

In some aspects, including in the LNPs put forth in Tables 1A-1C, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, including in the LNPs put forth in Tables 1A-1C, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

Further characteristics of the nucleic acid molecules of the present disclosure are provided herein.

### Polyphenol Additives

In some aspects, a lipid nanoparticle of the present disclosure, including those put forth in Tables 1A-1C, can further comprise at least one polyphenol (also referred to herein as a “polyphenol additive”).

As used herein, the term “polyphenol” is used to refer to any compound that has at least two phenol subunits, wherein a phenol is an aromatic ring, as defined herein, that has at least one hydroxyl substituent. Polyphenols include compounds that have at least two phenol subunits, for example flavonoids, catechins, anthocyanins, stilbenes and ellagic acid. Polyphenols also include compounds that have at least three phenol subunits, for example proanthocyanins, tannins and punicalagin.

Accordingly, a lipid nanoparticle can comprise at least one compound of the present disclosure, at least one structural lipid, at least one phospholipid, at least one PEGylated lipid, and at least one polyphenol. In some aspects, the lipid nanoparticle can further comprise, at least one nucleic acid, at least one targeting ligand, or any combination thereof.

A non-limiting example of a polyphenol is tannic acid. Thus, the present disclosure provides LNPs comprising tannic acid.

Another non-limiting example of a polyphenol is proanthocyanin. Thus, the present disclosure provides LNPs comprising proanthocyanin.

Another non-limiting example of a polyphenol is punicalagin. Thus, the present disclosure provides LNPs comprising punicalagin.

Another non-limiting example of a polyphenol is ellagic acid. Thus, the present disclosure provides LNPs comprising ellagic acid.

In some aspects, a lipid nanoparticle can comprise a polyphenol and nucleic acid at a specified ratio (weight/weight).

In some aspects, a lipid nanoparticle comprising a polyphenol and at least one nucleic acid can comprise a polyphenol and nucleic acid at a ratio of about 0.1:1, or about 0.15:1, or about 0.2:1, or about 0.25:1, or about 0.3:1, or about 0.35:1, or about 0.4:1, or about 0.45:1, or about 0.5:1, or about 1:1, or about 1.5:1, or about 2:1, or about 2.5:1, or about 3:1, or about 3.5:1, or about 4:1, or about 4.5:1, or about 5:1, or about 5.5:1, or about 6:1, or about 6.5:1, or about 7:1, or about 7.5:1, or about 8:1, or about 8.5:1, or about 9:1, or about 9.5:1, or about 10:1, or about 10.5:1, or about 11:1, or about 11.5:1, or about 12:1, or about 12.5:1, or about 13:1, or about 13.5:1, or about 14:1, or about 14.5:1, or about 15:1, or about 15.5:1, or about 16:1, or about 16.5:1, or about 17:1, or about 17.5:1, or about 18:1, or about 18.5:1, or about 19:1, or about 19.5:1, or about 20:1 polyphenol: nucleic acid.

In some aspects, a lipid nanoparticle comprising tannic acid and at least one nucleic acid can comprise tannic acid and nucleic acid at a ratio of about 0.15:1, or about 0.2:1, or about 5:1, or about 7:1, or about 7.5:1, or about 10:1, or about 12.5:1, or about 15:1 tannic acid: nucleic acid, weight/weight. In some aspects, the at least one nucleic acid can comprise DNA.

In some aspects, a lipid nanoparticle comprising proanthocyanidin and at least one nucleic acid can comprise proanthocyanidin and nucleic acid at a ratio of about 2.5:1, or about 5:1, or about 7.5:1, or about 10:1, or about 12.5:1, or about 15:1, or about 20:1 proanthocyanidin: nucleic acid, weight/weight. In some aspects, the at least one nucleic acid can comprise DNA.

In some aspects, a lipid nanoparticle comprising ellagic acid and at least one nucleic acid can comprise ellagic acid and nucleic acid at a ratio of about 2.5:1, about 5:1, or about 10:1 ellagic acid: nucleic acid, weight/weight. In some aspects, the at least one nucleic acid can comprise DNA.

In some aspects, a lipid nanoparticle comprising punicalagin and at least one DNA molecule can comprise punicalagin and DNA at a ratio of about 2.5:1, about 5:1, or about 10:1 punicalagin: DNA, weight/weight. In some aspects, the at least one nucleic acid can comprise DNA.

In some aspects, a lipid nanoparticle can comprise a polyphenol and lipid at a specified ratio (weight/weight).

In some aspects, a lipid nanoparticle can comprise a polyphenol and lipid at a ratio of about 0.08:1, 0.1:1, or about 0.17:1, or about 0.2:1 or about 0.25:1, or about 0.3:1 polyphenol: lipid, weight/weight.

### Exemplary LNPs of the Present Disclosure

The following are exemplary LNPs of the present disclosure.

In some aspects, a lipid nanoparticle is provided comprising about 40.75% of at least one compound of Formula (I) by moles, about 51.75% of at least one structural lipid by moles, about 5% of at least one phospholipid by moles, and about 2.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 30.75% to about 50.75% of at least one compound of Formula (I) by moles, about 41.75% to about 61.75% of at least one structural lipid by moles, about 0.1% to about 15% of at least one phospholipid by moles, and about 0.1% to about 12.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 35.75% to about 45.75% of at least one compound of Formula (I) by moles, about 46.75% to about 56.75% of at least one structural lipid by moles, about 1% to about 10% of at least one phospholipid by moles, and about 1% to about 7.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w). In some aspects, the ratio of lipid to nucleic acid in the nanoparticle can be about 30:1 (w/w) to about 50:1 (w/w), or about 35:1 (w/w) to about 45:1 (w/w). In some aspects, the ratio of lipid to nucleic acid in the nanoparticle can be about 40:1 (w/w).

In some aspects, a lipid nanoparticle is provided comprising about 40.8% to about 45.9% of at least one compound of Formula (I) by moles, about 45.9% to about 53.8% of at least one structural lipid by moles, about 0% to about 6.2% of at least one phospholipid by moles, and about 2% to about 2.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 30.8% to about 55.9% of at least one compound of Formula (I) by moles, about 35.9% to about 63.8% of at least one structural lipid by moles, about 0% to about 16.2% of at least one phospholipid by moles, and about 0.1% to about 12.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 35.8% to about 50.9% of at least one compound of Formula (I) by moles, about 40.9% to about 58.8% of at least one structural lipid by moles, about 0% to about 11.2% of at least one phospholipid by moles, and about 1% to about 7.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 30:1 to about 60:1 (w/w), or about 35:1 to about 55:1 (w/w). In some aspects, the ratio of lipid to nucleic acid in the nanoparticle can be about 40:1 to about 50:1 (w/w). In some aspects, the ratio of lipid to nucleic acid in the nanoparticle can be about 40:1 (w/w). In some aspects, the ratio of lipid to nucleic acid in the nanoparticle can be about 50:1 (w/w).

In some aspects, a lipid nanoparticle is provided comprising about 54.2% to about 60% of at least one compound of Formula (I) by moles, about 38% to about 39.5% of at least one structural lipid by moles, about 0% to about 3.9% of at least one phospholipid by moles, and about 2% to about 2.4% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 44.2% to about 70% of at least one compound of Formula (I) by moles, about 28% to about 49.5% of at least one structural lipid by moles, about 0% to about 13.9% of at least one phospholipid by moles, and about 0.1% to about 12.4% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 49.2% to about 65% of at least one compound of Formula (I) by moles, about 33% to about 44.5% of at least one structural lipid by moles, about 1% to about 8.9% of at least one phospholipid by moles, and about 1% to about 7.4% of at least one PEGylated lipid by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 40:1 to about 60:1 (w/w), or about 45:1 to about 55:1 (w/w). In some aspects, the ratio of lipid to nucleic acid in the nanoparticle can be about 50:1 (w/w).

In some aspects, a lipid nanoparticle is provided comprising about 40.75% of at least one compound of Formula (II) by moles, about 51.75% of at least one structural lipid by moles, about 5% of at least one phospholipid by moles, and about 2.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 30.75% to about 50.75% of at least one compound of Formula (II) by moles, about 41.75% to about 61.75% of at least one structural lipid by moles, about 0.1% to about 15% of at least one phospholipid by moles, and about 0.1% to about 12.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 35.75% to about 45.75% of at least one compound of Formula (II) by moles, about 46.75% to about 56.75% of at least one structural lipid by moles, about 1% to about 10% of at least one phospholipid by moles, and about 1% to about 7.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 43.17% of at least one compound of Formula (II) by moles, about 43.17% of at least one structural lipid by moles, about 11.96% of at least one phospholipid by moles, and about 1.7% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 33.17% to about 53.17% of at least one compound of Formula (II) by moles, about 33.17% to about 53.17% of at least one structural lipid by moles, about 1.96% to about 21.96% of at least one phospholipid by moles, and about 0.1% to about 11.7% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 38.17% to about 48.17% of at least one compound of Formula (II) by moles, about 38.17% to about 48.17% of at least one structural lipid by moles, about 6.96% to about 16.96% of at least one phospholipid by moles, and about 1% to about 6.7% of at least one PEGylated lipid by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 50% of at least one compound of Formula (II) by moles, about 38.5% of at least one structural lipid by moles, about 10% of at least one phospholipid by moles, and about 1.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 40% to about 60% of at least one compound of Formula (II) by moles, about 28.5% to about 48.5% of at least one structural lipid by moles, about 0.1% to about 20% of at least one phospholipid by moles, and about 0.1% to about 11.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 45% to about 55% of at least one compound of Formula (II) by moles, about 33.5% to about 43.5% of at least one structural lipid by moles, about 5% to about 15% of at least one phospholipid by moles, and about 0.5% to about 6.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 30:1 to about 90:1 (w/w), about 70:1 to about 90:1 (w/w), about 75:1 to about 85:1 (w/w), or about 35:1 to about 85:1 (w/w). In some aspects, the ratio of lipid to nucleic acid in the nanoparticle can be about 40:1 (w/w), about 50:1 (w/w), about 60:1 (w/w), or about 80:1 (w/w).

In some aspects, a lipid nanoparticle is provided comprising about 50% of at least one compound of Formula (II) by moles, about 38% of at least one structural lipid by moles, about 10% of at least one phospholipid by moles, and about 2% of at least one PEGylated lipid by moles, wherein the lipid nanoparticle comprises at least one nucleic acid, wherein the at least one nucleic acid comprises at least one RNA molecule (e.g. mRNA molecule. In some aspects, the present disclosure provides a lipid nanoparticle comprising about 40% to about 60% of at least one compound of Formula (II) by moles, about 28% to about 48% of at least one structural lipid by moles, about 0.1% to about 20% of at least one phospholipid by moles, and about 0.1% to about 12% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 45% to about 55% of at least one compound of Formula (II) by moles, about 33% to about 43% of at least one structural lipid by moles, about 5% to about 15% of at least one phospholipid by moles, and about 0.5% to about 7% of at least one PEGylated lipid by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 30:1 to about 60:1 (w/w), or about 35:1 to about 55:1 (w/w). In some aspects, the ratio of lipid to nucleic acid in the nanoparticle can be about 40:1 (w/w) or about 50:1 (w/w).

In some aspects, a lipid nanoparticle is provided comprising about 54% of at least one compound of Formula (II) by moles, about 35% of at least one structural lipid by moles, about 10% of at least one phospholipid by moles, and about 1% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 44% to about 64% of at least one compound of Formula (II) by moles, about 25% to about 45% of at least one structural lipid by moles, about 0.1% to about 20% of at least one phospholipid by moles, and about 0.1% to about 10% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 49% to about 59% of at least one compound of Formula (II) by moles, about 30% to about 40% of at least one structural lipid by moles, about 5% to about 15% of at least one phospholipid by moles, and about 0.5% to about 5% of at least one PEGylated lipid by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 40.8% to about 54% of at least one compound of Formula (II) by moles, about 35% to about 51.8% of at least one structural lipid by moles, about 5% to about 12% of at least one phospholipid by moles, and about 1% to about 2.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 30.8% to about 64% of at least one compound of Formula (II) by moles, about 25% to about 61.8% of at least one structural lipid by moles, about 0.1% to about 22% of at least one phospholipid by moles, and about 0.1% to about 12.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 35.8% to about 59% of at least one compound of Formula (II) by moles, about 30% to about 56.8% of at least one structural lipid by moles, about 1% to about 17% of at least one phospholipid by moles, and about 0.5% to about 7.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 50% of at least one compound of Formula (II) by moles, about 38.5% of at least one structural lipid by moles, about 10% of at least one phospholipid by moles, and about 1.5% of at least one PEGylated lipid by moles, wherein the lipid nanoparticle comprises at least one nucleic acid, wherein the at least one nucleic acid comprises at least one RNA molecule (e.g. mRNA molecule). In some aspects, a lipid nanoparticle is provided comprising about 50% of at least one compound of Formula (II) by moles, about 42.5% of at least one structural lipid by moles, about 5% of at least one phospholipid by moles, and about 2.5% of at least one PEGylated lipid by moles, wherein the lipid nanoparticle comprises at least one nucleic acid, wherein the at least one nucleic acid comprises at least one RNA molecule (e.g. mRNA molecule). In some aspects, a lipid nanoparticle is provided comprising about 50% of at least one compound of Formula (II) by moles, about 37.5% of at least one structural lipid by moles, about 10% of at least one phospholipid by moles, and about 2.5% of at least one PEGylated lipid by moles, wherein the lipid nanoparticle comprises at least one nucleic acid, wherein the at least one nucleic acid comprises at least one RNA molecule (e.g. mRNA molecule). In some aspects, a lipid nanoparticle is provided comprising about 50% of at least one compound of Formula (II) by moles, about 41% of at least one structural lipid by moles, about 7.5% of at least one phospholipid by moles, and about 1.5% of at least one PEGylated lipid by moles, wherein the lipid nanoparticle comprises at least one nucleic acid, wherein the at least one nucleic acid comprises at least one RNA molecule (e.g. mRNA molecule). In some aspects, a lipid nanoparticle is provided comprising about 50% of at least one compound of Formula (II) by moles, about 43% of at least one structural lipid by moles, about 5% of at least one phospholipid by moles, and about 2% of at least one PEGylated lipid by moles, wherein the lipid nanoparticle comprises at least one nucleic acid, wherein the at least one nucleic acid comprises at least one RNA molecule (e.g. mRNA molecule). In some aspects, a lipid nanoparticle is provided comprising about 45% of at least one compound of Formula (II) by moles, about 48.5% of at least one structural lipid by moles, about 5% of at least one phospholipid by moles, and about 1.5% of at least one PEGylated lipid by moles, wherein the lipid nanoparticle comprises at least one nucleic acid, wherein the at least one nucleic acid comprises at least one RNA molecule (e.g. mRNA molecule). In some aspects, a lipid nanoparticle is provided comprising about 45% of at least one compound of Formula (II) by moles, about 45.5% of at least one structural lipid by moles, about 7.5% of at least one phospholipid by moles, and about 2% of at least one PEGylated lipid by moles, wherein the lipid nanoparticle comprises at least one nucleic acid, wherein the at least one nucleic acid comprises at least one RNA molecule (e.g. mRNA molecule). In some aspects, a lipid nanoparticle is provided comprising about 40% of at least one compound of Formula (II) by moles, about 52.5% of at least one structural lipid by moles, about 5% of at least one phospholipid by moles, and about 2.5% of at least one PEGylated lipid by moles, wherein the lipid nanoparticle comprises at least one nucleic acid, wherein the at least one nucleic acid comprises at least one RNA molecule (e.g. mRNA molecule). In some aspects, a lipid nanoparticle is provided comprising about 40% of at least one compound of Formula (II) by moles, about 50% of at least one structural lipid by moles, about 7.5% of at least one phospholipid by moles, and about 2.5% of at least one PEGylated lipid by moles, wherein the lipid nanoparticle comprises at least one nucleic acid, wherein the at least one nucleic acid comprises at least one RNA molecule (e.g. mRNA molecule). In some aspects, a lipid nanoparticle is provided comprising about 40% of at least one compound of Formula (II) by moles, about 48% of at least one structural lipid by moles, about 10% of at least one phospholipid by moles, and about 2% of at least one PEGylated lipid by moles, wherein the lipid nanoparticle comprises at least one nucleic acid, wherein the at least one nucleic acid comprises at least one RNA molecule (e.g. mRNA molecule). In some aspects, a lipid nanoparticle is provided comprising about 40% of at least one compound of Formula (II) by moles, about 53.5% of at least one structural lipid by moles, about 5% of at least one phospholipid by moles, and about 1.5% of at least one PEGylated lipid by moles, wherein the lipid nanoparticle comprises at least one nucleic acid, wherein the at least one nucleic acid comprises at least one RNA molecule (e.g. mRNA molecule). In some aspects, a lipid nanoparticle is provided comprising about 40% of at least one compound of Formula (II) by moles, about 48.5% of at least one structural lipid by moles, about 10% of at least one phospholipid by moles, and about 1.5% of at least one PEGylated lipid by moles, wherein the lipid nanoparticle comprises at least one nucleic acid, wherein the at least one nucleic acid comprises at least one RNA molecule (e.g. mRNA molecule).

In some aspects, a lipid nanoparticle is provided comprising about 50% of at least one compound of Formula (II) by moles, about 38.5% of at least one structural lipid by moles, about 10% of at least one phospholipid by moles, about 1.25% of at least one PEGylated lipid by moles, and about 0.25% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 40% to about 60% of at least one compound of Formula (II) by moles, about 28.5% to about 48.5% of at least one structural lipid by moles, about 0.1% to about 20% of at least one phospholipid by moles, about 0.1% to about 11.25% of at least one PEGylated lipid by moles, and about 0.1% to about 10.25% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 45% to about 55% of at least one compound of Formula (II) by moles, about 33.5% to about 43.5% of at least one structural lipid by moles, about 5% to about 15% of at least one phospholipid by moles, about 0.5% to about 6.25% of at least one PEGylated lipid by moles, and about 0.1% to about 5.25% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 70:1 to about 90:1 (w/w), or about 75:1 to about 85:1 (w/w). In some aspects, the ratio of lipid to nucleic acid in the nanoparticle can be about 80:1 (w/w).

In some aspects, a lipid nanoparticle is provided comprising about 50% of at least one compound of Formula (II) by moles, about 38.5% of at least one structural lipid by moles, about 10% of at least one phospholipid by moles, about 1.2% of at least one PEGylated lipid by moles, and about 0.3% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 40% to about 60% of at least one compound of Formula (II) by moles, about 28.5% to about 48.5% of at least one structural lipid by moles, about 0.1% to about 20% of at least one phospholipid by moles, about 0.1% to about 11.2% of at least one PEGylated lipid by moles, and about 0.1% to about 10.3% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 45% to about 55% of at least one compound of Formula (II) by moles, about 33.5% to about 43.5% of at least one structural lipid by moles, about 5% to about 15% of at least one phospholipid by moles, about 0.5% to about 6.2% of at least one PEGylated lipid by moles, and about 0.1% to about 5.3% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 40:1 to about 60:1 (w/w), or about 45:1 to about 55:1 (w/w). In some aspects, the ratio of lipid to nucleic acid in the nanoparticle can be about 50:1 (w/w).

In some aspects, a lipid nanoparticle is provided comprising about 50% of at least one compound of Formula (II) by moles, about 38.5% of at least one structural lipid by moles, about 10% of at least one phospholipid by moles, about 1% of at least one PEGylated lipid by moles, and about 0.5% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 40% to about 60% of at least one compound of Formula (II) by moles, about 28.5% to about 48.5% of at least one structural lipid by moles, about 0.1% to about 20% of at least one phospholipid by moles, about 0.1% to about 11% of at least one PEGylated lipid by moles, and about 0.1% to about 10.5% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 45% to about 55% of at least one compound of Formula (II) by moles, about 33.5% to about 43.5% of at least one structural lipid by moles, about 5% to about 15% of at least one phospholipid by moles, about 0.5% to about 6% of at least one PEGylated lipid by moles, and about 0.1% to about 5.5% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 70:1 to about 90:1 (w/w), or about 75:1 to about 85:1 (w/w). In some aspects, the ratio of lipid to nucleic acid in the nanoparticle can be about 80:1 (w/w). In some aspects, the ratio of lipid to nucleic acid in the nanoparticle can be about 40:1 to about 60:1 (w/w), or about 45:1 to about 55:1 (w/w). In some aspects, the ratio of lipid to nucleic acid in the nanoparticle can be about 50:1 (w/w).

In some aspects, a lipid nanoparticle is provided comprising about 45% of at least one compound of Formula (II) by moles, about 42.5% of at least one structural lipid by moles, about 10% of at least one phospholipid by moles, and about 2.5% of at least one PEGylated lipid by moles, wherein the lipid nanoparticle comprises at least one nucleic acid, wherein the at least one nucleic acid comprises at least one RNA molecule (e.g. mRNA molecule In some aspects, the present disclosure provides a lipid nanoparticle comprising about 35% to about 55% of at least one compound of Formula (II) by moles, about 32.5% to about 52.5% of at least one structural lipid by moles, about 0.1% to about 20% of at least one phospholipid by moles, and about 0.1% to about 12.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 40% to about 50% of at least one compound of Formula (II) by moles, about 37.5% to about 47.5% of at least one structural lipid by moles, about 5% to about 15% of at least one phospholipid by moles, and about 0.5% to about 7.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 50:1 to about 70:1 (w/w), or about 55:1 to about 65:1 (w/w). In some aspects, the ratio of lipid to nucleic acid in the nanoparticle can be about 60:1 (w/w).

In some aspects, a lipid nanoparticle is provided comprising about 45% of at least one compound of Formula (II) by moles, about 42.5% of at least one structural lipid by moles, about 10% of at least one phospholipid by moles, about 2.25% of at least one PEGylated lipid by moles, and about 0.25% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 35% to about 55% of at least one compound of Formula (II) by moles, about 32.5% to about 52.5% of at least one structural lipid by moles, about 0.1% to about 20% of at least one phospholipid by moles, about 0.1% to about 12.25% of at least one PEGylated lipid by moles, and about 0.1% to about 10.25% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 40% to about 50% of at least one compound of Formula (II) by moles, about 37.5% to about 47.5% of at least one structural lipid by moles, about 5% to about 15% of at least one phospholipid by moles, about 0.5% to about 7.25% of at least one PEGylated lipid by moles, and about 0.1% to about 5.25% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 50:1 to about 70:1 (w/w), or about 55:1 to about 65:1 (w/w). In some aspects, the ratio of lipid to nucleic acid in the nanoparticle can be about 60:1 (w/w).

In some aspects, a lipid nanoparticle is provided comprising about 45% of at least one compound of Formula (II) by moles, about 42.5% of at least one structural lipid by moles, about 10% of at least one phospholipid by moles, about 2% of at least one PEGylated lipid by moles, and about 0.5% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 35% to about 55% of at least one compound of Formula (II) by moles, about 32.5% to about 52.5% of at least one structural lipid by moles, about 0.1% to about 20% of at least one phospholipid by moles, about 0.1% to about 12% of at least one PEGylated lipid by moles, and about 0.1% to about 10.5% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 40% to about 50% of at least one compound of Formula (II) by moles, about 37.5% to about 47.5% of at least one structural lipid by moles, about 5% to about 15% of at least one phospholipid by moles, about 0.5% to about 7% of at least one PEGylated lipid by moles, and about 0.1% to about 5.5% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 50:1 to about 70:1 (w/w), or about 55:1 to about 65:1 (w/w). In some aspects, the ratio of lipid to nucleic acid in the nanoparticle can be about 60:1 (w/w).

In some aspects, a lipid nanoparticle is provided comprising about 50% of at least one compound of Formula (II) by moles, about 38% of at least one structural lipid by moles, about 10% of at least one phospholipid by moles, about 1.7% of at least one PEGylated lipid by moles, and about 0.3% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 40% to about 60% of at least one compound of Formula (II) by moles, about 28% to about 48% of at least one structural lipid by moles, about 0.1% to about 20% of at least one phospholipid by moles, about 0.1% to about 11.7% of at least one PEGylated lipid by moles, and about 0.1% to about 10.3% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 45% to about 55% of at least one compound of Formula (II) by moles, about 33% to about 43% of at least one structural lipid by moles, about 5% to about 15% of at least one phospholipid by moles, about 0.5% to about 6.7% of at least one PEGylated lipid by moles, and about 0.1% to about 5.3% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 40:1 to about 60:1 (w/w), or about 45:1 to about 55:1 (w/w). In some aspects, the ratio of lipid to nucleic acid in the nanoparticle can be about 50:1 (w/w).

In some aspects, a lipid nanoparticle is provided comprising about 50% of at least one compound of Formula (II) by moles, about 38% of at least one structural lipid by moles, about 10% of at least one phospholipid by moles, about 1.5% of at least one PEGylated lipid by moles, and about 0.5% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 40% to about 60% of at least one compound of Formula (II) by moles, about 28% to about 48% of at least one structural lipid by moles, about 0.1% to about 20% of at least one phospholipid by moles, about 0.1% to about 11.5% of at least one PEGylated lipid by moles, and about 0.1% to about 10.5% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 45% to about 55% of at least one compound of Formula (II) by moles, about 33% to about 43% of at least one structural lipid by moles, about 5% to about 15% of at least one phospholipid by moles, about 0.5% to about 6.5% of at least one PEGylated lipid by moles, and about 0.1% to about 5.5% of a targeting ligand comprising GalNac by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 40:1 to about 60:1 (w/w), or about 45:1 to about 55:1 (w/w). In some aspects, the ratio of lipid to nucleic acid in the nanoparticle can be about 50:1 (w/w).

In some aspects, the nucleic acid molecule is a DNA molecule. Thus, the present disclosure provides a lipid nanoparticle is provided comprising about 40.75% of at least one compound of Formula (I) by moles, about 51.75% of at least one structural lipid by moles, about 5% of at least one phospholipid by moles, and about 2.5% of at least one PEGylated lipid by moles, wherein the lipid nanoparticle comprises at least one nucleic acid, wherein the at least one nucleic acid comprises at least one DNA molecule. In some aspects, the present disclosure provides a lipid nanoparticle comprising about 30.75% to about 50.75% of at least one compound of Formula (I) by moles, about 41.75% to about 61.75% of at least one structural lipid by moles, about 0.1% to about 15% of at least one phospholipid by moles, and about 0.1% to about 12.5% of at least one PEGylated lipid by moles, wherein the lipid nanoparticle comprises at least one nucleic acid, wherein the at least one nucleic acid comprises at least one DNA molecule. In some aspects, the present disclosure provides a lipid nanoparticle comprising about 35.75% to about 45.75% of at least one compound of Formula (I) by moles, about 46.75% to about 56.75% of at least one structural lipid by moles, about 1% to about 10% of at least one phospholipid by moles, and about 1% to about 7.5% of at least one PEGylated lipid by moles, wherein the lipid nanoparticle comprises at least one nucleic acid, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, the nucleic acid molecule is a DNA molecule. Thus, the present disclosure provides a lipid nanoparticle is provided comprising about 40.75% of at least one compound of Formula (II) by moles, about 51.75% of at least one structural lipid by moles, about 5% of at least one phospholipid by moles, and about 2.5% of at least one PEGylated lipid by moles, wherein the lipid nanoparticle comprises at least one nucleic acid, wherein the at least one nucleic acid comprises at least one DNA molecule. In some aspects, the present disclosure provides a lipid nanoparticle comprising about 30.75% to about 50.75% of at least one compound of Formula (II) by moles, about 41.75% to about 61.75% of at least one structural lipid by moles, about 0.1% to about 15% of at least one phospholipid by moles, and about 0.1% to about 12.5% of at least one PEGylated lipid by moles, wherein the lipid nanoparticle comprises at least one nucleic acid, wherein the at least one nucleic acid comprises at least one DNA molecule. In some aspects, the present disclosure provides a lipid nanoparticle comprising about 35.75% to about 45.75% of at least one compound of Formula (II) by moles, about 46.75% to about 56.75% of at least one structural lipid by moles, about 1% to about 10% of at least one phospholipid by moles, and about 1% to about 7.5% of at least one PEGylated lipid by moles, wherein the lipid nanoparticle comprises at least one nucleic acid, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 40% to about 46% of at least one compound of Formula (I) by moles, about 45.9% to about 51.8% of at least one structural lipid by moles, about 4.9% to about 7% of at least one phospholipid by moles, and about 2% to about 3% of at least one PEGylated lipid by moles, wherein the lipid nanoparticle comprises at least one nucleic acid, wherein the at least one nucleic acid comprises at least one DNA molecule. In some aspects, the present disclosure provides a lipid nanoparticle comprising about 30% to about 56% of at least one compound of Formula (I) by moles, about 35.9% to about 61.8% of at least one structural lipid by moles, about 0.1% to about 17% of at least one phospholipid by moles, and about 0.1% to about 13% of at least one PEGylated lipid by moles, wherein the lipid nanoparticle comprises at least one nucleic acid, wherein the at least one nucleic acid comprises at least one DNA molecule. In some aspects, the present disclosure provides a lipid nanoparticle comprising about 35% to about 51% of at least one compound of Formula (I) by moles, about 40.9% to about 56.8% of at least one structural lipid by moles, about 1% to about 12% of at least one phospholipid by moles, and about 1% to about 8% of at least one PEGylated lipid by moles, wherein the lipid nanoparticle comprises at least one nucleic acid, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 70:1 to about 130:1 (w/w), or about 75:1 to about 125:1 (w/w). In some aspects, the ratio of lipid to nucleic acid in the nanoparticle can be about 80:1 (w/w) to about 120:1 (w/w). In some aspects, the ratio of lipid to nucleic acid in the nanoparticle can be about 80:1 (w/w). In some aspects, the ratio of lipid to nucleic acid in the nanoparticle can be about 120:1 (w/w).

In some aspects, a lipid nanoparticle is provided comprising about 54% to about 60% of at least one compound of Formula (I) by moles, about 30% to about 36% of at least one structural lipid by moles, about 2.8% to about 7% of at least one phospholipid by moles, and about 3% of at least one PEGylated lipid by moles, wherein the lipid nanoparticle comprises at least one nucleic acid, wherein the at least one nucleic acid comprises at least one DNA molecule. In some aspects, the present disclosure provides a lipid nanoparticle comprising about 44% to about 70% of at least one compound of Formula (I) by moles, about 20% to about 46% of at least one structural lipid by moles, about 0.1% to about 17% of at least one phospholipid by moles, and about 0.1% to about 13% of at least one PEGylated lipid by moles, wherein the lipid nanoparticle comprises at least one nucleic acid, wherein the at least one nucleic acid comprises at least one DNA molecule. In some aspects, the present disclosure provides a lipid nanoparticle comprising about 49% to about 65% of at least one compound of Formula (I) by moles, about 25% to about 41% of at least one structural lipid by moles, about 0.1% to about 12% of at least one phospholipid by moles, and about 0.1% to about 8% of at least one PEGylated lipid by moles, wherein the lipid nanoparticle comprises at least one nucleic acid, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 50:1 to about 110:1 (w/w), or about 55:1 to about 105:1 (w/w). In some aspects, the ratio of lipid to nucleic acid in the nanoparticle can be about 60:1 (w/w) to about 100:1 (w/w). In some aspects, the ratio of lipid to nucleic acid in the nanoparticle can be about 60:1 (w/w). In some aspects, the ratio of lipid to nucleic acid in the nanoparticle can be about 100:1 (w/w).

In some aspects, a lipid nanoparticle is provided comprising about 50% of at least one compound of Formula (II) by moles, about 38.5% of at least one structural lipid by moles, about 10% of at least one phospholipid by moles, and about 1.5% of at least one PEGylated lipid by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 50:1 (w/w).

In some aspects, a lipid nanoparticle is provided comprising about 50% of at least one compound of Formula (II) by moles, about 38.5% of at least one structural lipid by moles, about 10% of at least one phospholipid by moles, about 1% of at least one PEGylated lipid by moles, and about 0.5% of at least one targeting ligand comprising GalNac by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 50:1 (w/w).

In some aspects, a lipid nanoparticle is provided comprising about 50% of at least one compound of Formula (II) by moles, about 38% of at least one structural lipid by moles, about 10% of at least one phospholipid by moles, and about 2% of at least one PEGylated lipid by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 50:1 (w/w).

In some aspects, a lipid nanoparticle is provided comprising about 50% of at least one compound of Formula (II) by moles, about 38.5% of at least one structural lipid by moles, about 10% of at least one phospholipid by moles, and about 1.5% of at least one PEGylated lipid by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 80:1 (w/w).

In some aspects, a lipid nanoparticle is provided comprising about 45% of at least one compound of Formula (II) by moles, about 42.5% of at least one structural lipid by moles, about 10% of at least one phospholipid by moles, about 2% of at least one PEGylated lipid by moles, and about 0.5% of at least one targeting ligand comprising GalNac by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 60:1 (w/w).

In some aspects, a lipid nanoparticle is provided comprising about 50% of at least one compound of Formula (II) by moles, about 41% of at least one structural lipid by moles, about 7.5% of at least one phospholipid by moles, about 1% of at least one PEGylated lipid by moles, and about 0.5% of at least one targeting ligand comprising GalNac by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 60:1 (w/w).

In some aspects, a lipid nanoparticle is provided comprising about 45% of at least one compound of Formula (II) by moles, about 45.75% of at least one structural lipid by moles, about 7.5% of at least one phospholipid by moles, about 1.5% of at least one PEGylated lipid by moles, and about 0.25% of at least one targeting ligand comprising GalNac by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 50:1 (w/w).

In some aspects, a lipid nanoparticle is provided comprising about 40% of at least one compound of Formula (II) by moles, about 53.5% of at least one structural lipid by moles, about 5% of at least one phospholipid by moles, about 1% of at least one PEGylated lipid by moles, and about 0.5% of at least one targeting ligand comprising GalNac by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 50:1 (w/w).

In some aspects, a lipid nanoparticle is provided comprising about 40% of at least one compound of Formula (II) by moles, about 48% of at least one structural lipid by moles, about 10% of at least one phospholipid by moles, about 1.5% of at least one PEGylated lipid by moles, and about 0.5% of at least one targeting ligand comprising GalNac by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 40:1 (w/w).

In some aspects, a lipid nanoparticle is provided comprising about 40% of at least one compound of Formula (II) by moles, about 52.75% of at least one structural lipid by moles, about 5% of at least one phospholipid by moles, about 2% of at least one PEGylated lipid by moles, and about 0.25% of at least one targeting ligand comprising GalNac by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 60:1 (w/w).

In some aspects, a lipid nanoparticle is provided comprising about 45% of at least one compound of Formula (II) by moles, about 42.5% of at least one structural lipid by moles, about 10% of at least one phospholipid by moles, about 2% of at least one PEGylated lipid by moles, and about 0.5% of at least one targeting ligand comprising GalNac by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 60:1 (w/w).

In some aspects, a lipid nanoparticle is provided comprising about 50% of at least one compound of Formula (II) by moles, about 38% of at least one structural lipid by moles, about 10% of at least one phospholipid by moles, about 1.7% of at least one PEGylated lipid by moles, and about 0.3% of at least one targeting ligand comprising GalNac by moles, wherein the lipid nanoparticle comprises at least one nucleic acid, wherein the at least one nucleic acid comprises at least one DNA molecule and at least one RNA molecule. In some aspects, the ratio of lipid to nucleic acid in the nanoparticle can be about 50:1 (w/w).

In some aspects, a lipid nanoparticle is provided comprising about 50% of at least one compound of Formula (II) by moles, about 38.5% of at least one structural lipid by moles, about 10% of at least one phospholipid by moles, and about 1.5% of at least one PEGylated lipid by moles, wherein the at least one nucleic acid comprises at least one DNA molecule. In some aspects, the present disclosure provides a lipid nanoparticle comprising about 40% to about 60% of at least one compound of Formula (II) by moles, about 28.5% to about 48.5% of at least one structural lipid by moles, about 0.1% to about 20% of at least one phospholipid by moles, and about 0.1% to about 11.5% of at least one PEGylated lipid by moles, wherein the at least one nucleic acid comprises at least one DNA molecule. In some aspects, the present disclosure provides a lipid nanoparticle comprising about 45% to about 55% of at least one compound of Formula (II) by moles, about 33.5% to about 43.5% of at least one structural lipid by moles, about 5% to about 15% of at least one phospholipid by moles, and about 0.5% to about 6.5% of at least one PEGylated lipid by moles, wherein the at least one nucleic acid comprises at least one DNA molecule. In some aspects, the ratio of lipid to nucleic acid in the nanoparticle can be about 30:1 to about 90:1 (w/w), about 70:1 to about 90:1 (w/w), about 75:1 to about 85:1 (w/w), or about 35:1 to about 85:1 (w/w). In some aspects, the ratio of lipid to nucleic acid in the nanoparticle can be about 40:1 (w/w), about 50:1 (w/w), about 60:1 (w/w), or about 80:1 (w/w). In some aspects of the preceding LNPs, the lipid nanoparticle further comprises tannic acid in a ratio of tannic acid to nucleic acid of about 7.5:1, or about 10:1, or about 12.5:1, or about 15:1.

In some aspects, a lipid nanoparticle is provided comprising about 50% of at least one compound of Formula (II) by moles, about 38.5% of at least one structural lipid by moles, about 10% of at least one phospholipid by moles, about 1% of at least one PEGylated lipid by moles, and about 0.5% of a targeting ligand comprising GalNac by moles, wherein the at least one nucleic acid comprises at least one DNA molecule. In some aspects, the present disclosure provides a lipid nanoparticle comprising about 40% to about 60% of at least one compound of Formula (II) by moles, about 28.5% to about 48.5% of at least one structural lipid by moles, about 0.1% to about 20% of at least one phospholipid by moles, about 0.1% to about 11% of at least one PEGylated lipid by moles, and about 0.1% to about 10.5% of a targeting ligand comprising GalNac by moles, wherein the at least one nucleic acid comprises at least one DNA molecule. In some aspects, the present disclosure provides a lipid nanoparticle comprising about 45% to about 55% of at least one compound of Formula (II) by moles, about 33.5% to about 43.5% of at least one structural lipid by moles, about 5% to about 15% of at least one phospholipid by moles, about 0.5% to about 6% of at least one PEGylated lipid by moles, and about 0.1% to about 5.5% of a targeting ligand comprising GalNac by moles, wherein the at least one nucleic acid comprises at least one DNA molecule. In some aspects, the ratio of lipid to nucleic acid in the nanoparticle can be about 70:1 to about 90:1 (w/w), or about 75:1 to about 85:1 (w/w). In some aspects, the ratio of lipid to nucleic acid in the nanoparticle can be about 80:1 (w/w). In some aspects of the preceding LNPs, the lipid nanoparticle further comprises tannic acid in a ratio of tannic acid to nucleic acid of about 7.5:1, or about 10:1, or about 12.5:1, or about 15:1.

In some aspects, a lipid nanoparticle is provided comprising about 45% of at least one compound of Formula (II) by moles, about 45.75% of at least one structural lipid by moles, about 7.5% of at least one phospholipid by moles, about 1.5% of at least one PEGylated lipid by moles, and about 0.25% of a targeting ligand comprising GalNac by moles, wherein the at least one nucleic acid comprises at least one DNA molecule and at least one RNA molecule. In some aspects, the ratio of lipid to nucleic acid in the nanoparticle can be about 50:1 (w/w). In some aspects, the lipid nanoparticle further comprises tannic acid in a ratio of tannic acid to nucleic acid of about 7:1.

In some aspects, a lipid nanoparticle is provided comprising about 50% of at least one compound of Formula (II) by moles, about 41% of at least one structural lipid by moles, about 7.5% of at least one phospholipid by moles, about 1% of at least one PEGylated lipid by moles, and about 0.5% of a targeting ligand comprising GalNac by moles, wherein the at least one nucleic acid comprises at least one DNA molecule and at least one RNA molecule. In some aspects, the ratio of lipid to nucleic acid in the nanoparticle can be about 50:1 (w/w). In some aspects, the ratio of lipid to nucleic acid in the nanoparticle can be about 60:1 (w/w). In some aspects, the lipid nanoparticle further comprises tannic acid in a ratio of tannic acid to nucleic acid of about 5:1, about 10:1, or about 15:1. In some aspects, the lipid nanoparticle further comprises tannic acid in a ratio of tannic acid to total lipid of about 0.2 or about 0.25.

In some aspects, a lipid nanoparticle is provided comprising about 45% of at least one compound of Formula (II) by moles, about 45.75% of at least one structural lipid by moles, about 7.5% of at least one phospholipid by moles, about 1.5% of at least one PEGylated lipid by moles, and about 0.25% of a targeting ligand comprising GalNac by moles, wherein the at least one nucleic acid comprises at least one DNA molecule and at least one RNA molecule. In some aspects, the ratio of lipid to nucleic acid in the nanoparticle can be about 50:1 (w/w). In some aspects, the lipid nanoparticle further comprises tannic acid in a ratio of tannic acid to nucleic acid of about 5:1, about 10:1, or about 15:1. In some aspects, the lipid nanoparticle further comprises tannic acid in a ratio of tannic acid to total lipid of about 0.2 or about 0.25.

In some aspects, a lipid nanoparticle is provided comprising about 50% of at least one compound of Formula (II) by moles, about 38.5% of at least one structural lipid by moles, about 10% of at least one phospholipid by moles, and about 1.5% of at least one PEGylated lipid by moles, wherein the at least one nucleic acid comprises at least one DNA molecule. In some aspects, the ratio of lipid to nucleic acid in the nanoparticle can be about 80:1 (w/w). In some aspects of the preceding LNPs, the lipid nanoparticle further comprises proanthocyanidin in a ratio of proanthocyanidin to nucleic acid of about 2.5:1, or about 5:1, or about 7.5:1, or about 10:1, or about 15:1, or about 20:1.

In some aspects, a lipid nanoparticle is provided comprising about 50% of at least one compound of Formula (II) by moles, about 38.5% of at least one structural lipid by moles, about 10% of at least one phospholipid by moles, and about 1.5% of at least one PEGylated lipid by moles, wherein the at least one nucleic acid comprises at least one DNA molecule. In some aspects, the ratio of lipid to nucleic acid in the nanoparticle can be about 80:1 (w/w). In some aspects of the preceding LNPs, the lipid nanoparticle further comprises ellagic acid in a ratio of ellagic acid to nucleic acid of about 2:5:1, or about 5:1, or about 10:1.

In some aspects, a lipid nanoparticle is provided comprising about 50% of at least one compound of Formula (II) by moles, about 38.5% of at least one structural lipid by moles, about 10% of at least one phospholipid by moles, and about 1.5% of at least one PEGylated lipid by moles, wherein the at least one nucleic acid comprises at least one DNA molecule. In some aspects, the ratio of lipid to nucleic acid in the nanoparticle can be about 80:1 (w/w). In some aspects of the preceding LNPs, the lipid nanoparticle further comprises punicalagin in a ratio of punicalagin to nucleic acid of about 2:5:1, or about 5:1, or about 10:1.

### Pharmaceutical Compositions of the Present Disclosure

In some aspects, the present disclosure provides a pharmaceutical composition comprising at least one lipid nanoparticle of the present disclosure. In some aspects, the present disclosure provides a pharmaceutical composition comprising at least one first nanoparticle of the present disclosure and at least one second nanoparticle of the present disclosure, wherein the at least one first nanoparticle comprises at least one nucleic acid molecule encoding at least one transposase, wherein the at least one second nanoparticle comprises at least one nucleic acid molecule encoding at least one transposon. In some aspects, the at least one nucleic acid molecule encoding at least one transposase can be an RNA molecule (e.g. mRNA molecule) and the at least one nucleic acid molecule encoding at least one transposon can be a DNA molecule (e.g. a DoggyBone DNA molecule or a DNA nanoplasmid).

In some aspects, the present disclosure provides a composition comprising at least one cell that has been contacted by at least one nanoparticle of the present disclosure. In some aspects, the present disclosure provides a composition comprising at least one cell that has been genetically modified using at least one nanoparticle of the present disclosure. In some aspects, the present disclosure provides a composition comprising at least one cell that has been genetically modified using any method of the present disclosure.

In some aspects, the present disclosure provides a pharmaceutical composition comprising at least one cell that has been contacted by at least one nanoparticle of the present disclosure. In some aspects, the present disclosure provides a pharmaceutical composition comprising at least one cell that has been genetically modified using at least one nanoparticle of the present disclosure. In some aspects, the present disclosure provides a pharmaceutical composition comprising at least one cell that has been genetically modified using any method of the present disclosure.

### Methods of the Present Disclosure

The present disclosure provides a method of delivering at least one nucleic acid to at least one cell comprising contacting the at least one cell with at least one composition of the present disclosure. The present disclosure provides a method of delivering at least one nucleic acid to at least one cell comprising contacting the at least one cell with at least one nanoparticle of the present disclosure.

In all methods, compositions and kits of the present disclosure, at least one cell can be a liver cell. A liver cell can include, but is not limited to, a hepatocyte, a hepatic stellate cell, Kupffer cell or a liver sinusoidal endothelial cell.

In some aspects of any methods of the present disclosure, a cell can be in vivo, ex vivo or in vitro. In some aspects, any of the methods of the present disclosure can be applied in vivo, ex vivo or in vitro.

The present disclosure provides a method of genetically modifying at least one cell comprising contacting the at least one cell with at least one composition of the present disclosure. The present disclosure provides a method of genetically modifying at least one cell comprising contacting the at least one cell with at least one nanoparticle of the present disclosure.

In some aspects, genetically modifying a cell can comprise delivering at least one exogenous nucleic acid to the cell such that the cell expresses at least one protein that the cell otherwise would not normally express, or such that the at least one cell expresses at least one protein at a level that is higher than the level that the cell would otherwise normally express the at least one protein, or such that the cell expresses at least one protein at a level that is lower than the level that the cell would otherwise normally express. In some aspects, genetically modifying a cell can comprise delivering at least one exogenous nucleic to the cell such that at least one exogenous nucleic acid is integrated into the genome of the at least one cell.

In some aspects, the methods of the present disclosure can yield a plurality of cells, wherein at least about 1%, or at least about 2%, or at least about 3%, or at least about 4%, or at least about 5%, or at least 10%, or at least 15%, or at least 20%, or at least 25%, or at least 30%, or at least 35%, or at least 40%, or at least 45%, or at least 50%, or at least 55%, or at least 60%, or at least about 65%, or at least about 70%, or at least about 75%, or at least about 80%, or at least about 85%, or at least about 90%, or at least about 95%, or at least about 99% of the cell in the plurality express at least one protein that was encoded in at least one nucleic acid that was delivered to the plurality of cells via a nanoparticle of the present disclosure.

The present disclosure provides a method of treating at least one disease in a subject, the method comprising administering to the subject at least one therapeutically effective amount of at least one composition of the present disclosure comprising at least one nucleic acid encoding a therapeutic protein.

The present disclosure provides a method of treating at least one disease in a subject, the method comprising administering a therapeutically effective amount of at least one nanoparticle of the present disclosure comprising at least one nucleic acid encoding a therapeutic protein.

The present disclosure provides a method of treating at least one disease in a subject, the method comprising administering a therapeutically effective amount of cells, wherein the cells have been contacted by at least one nanoparticle of the present disclosure comprising at least one nucleic acid encoding a therapeutic protein. The present disclosure provides a method of treating at least one disease in a subject, the method comprising administering a therapeutically effective amount of cells, wherein the cells have been genetically modified using the compositions and/or methods of the present disclosure.

The disclosure provides methods for the treatment of a disease or disorder in a cell, tissue, organ, animal, or subject, comprising administering or contacting the cell, tissue, organ, animal, or subject with a therapeutic effective amount of a composition disclosed herein. In one aspect, the subject is a mammal. Preferably, the subject is human. The terms “subject” and “patient” are used interchangeably herein.

The disclosure provides methods of treating at least one disease or disorder in a subject, comprising administering to the subject at least one therapeutically effective amount of at least one composition disclosed herein comprising at least one nucleic acid encoding a therapeutic protein.

Without wishing to be bound by theory, it is hypothesized that the LNP compositions of the present disclosure target liver cells more effectively than other cells, thus reducing off-target effects associated with other delivery compositions.

In some embodiments, the LNP compositions provided herein that comprise a targeting ligand result in less cytokine release than the same LNP composition not comprising the targeting ligand. Cytokine release may be measured using any suitable method know in the art or described herein. For example, cytokine levels may be determined in the blood of a subject receiving the LNP composition comprising the targeting ligand using enzyme-linked immunosorbent assays (ELISAs). The cytokine levels may then be compared to pre-treatment baseline levels.

The disclosure provides a method for modulating or treating at least one malignant disease or disorder in a cell, tissue, organ, animal or subject. In some aspects, the at least one disease can be a malignant disease, including, but not limited to, cancer. In some aspects, the at least one disease can be Hemophilia A or Hemophilia B. In some aspects, the at least one disease can be a metabolic liver disorder (MLD). In some aspects, the at least one disease can be a urea cycle disorder (UCD). An MLD and/or UCD can include, but is not limited to, N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome), methylmalonic acidemia (MMA) or any combination thereof.

Methods of the disclosure may be used to treat a disease or disorder by use of a therapeutic transgene encoding for an exogenous nucleic acid sequence or exogenous amino acid sequence. In such methods, the transgene is delivered to a target cell to replace or repair a mutated gene. Diseases that may be treated with such methods are generally caused by a mutation in a gene that results in no protein being expressed or non-functional proteins being expressed. Examples of therapeutic transgenes that can be delivered using the compositions disclosed herein include: Beta-Thalassemia (HBB T87Q, BCL11A shRNA, IGF2BP1), Sickle Cell Disease (HBB T87Q, BCL11A shRNA, IGF2BP1), Hemophilia A (Factor VIII), Hemophilia B (Factor IX), X-linked Severe Combined Immunodeficiency (Interleukin 2 receptor gamma (IL2RG)), Hypophosphatasia (Tissue Non-specific Alkaline Phosphatase (TNAP)), Osteopetrosis (TCIRG1), Glycogen Storage Disease Type II (Pompe Disease) (Alpha Glucosidase (GAA)), Alpha-Galactosidase A Deficiency (Fabry disease) (Alpha-galactosidase A (GLA)), Mucopolysaccharidosis Type I (MPS I) (Alpha-L-iduronidase (IDUA)), Mucopolysaccharidosis Type II (MPS II) (Iduronate 2-sulfatase (IDS)), Mucopolysaccharidosis Type IIIA (MPS IIIA) (sulfoglycosamine-sulfohydrolase (SGSH)), Mucopolysaccharidosis Type IIIB (MPS IIIB) (N-alpha-acetylglucosaminidase (NAGLU)), Mucopolysaccharidosis Type IV A (MPS IVA) (Morquio) (N-acetylgalactosamine-6-sulfate sulfatase (GALNS)), Mucopolysaccharidosis Type IV B (MPS IVB) Beta-galactosidase (GLB1 (Beta-galactosidase (GLB1)), Cholesteryl Ester Storage Disease (CESD) (Lysosomal acid lipase (LIPA)), Cystinosis (Cystinosin lysosomal cystine transporter (CTNS)), X-linked chronic granulomatous disease (X-CGD) (CYBB), Wiskott-Aldrich Syndrome (WAS) (WAS), X-linked Adrenoleukodystrophy (X-ALD) (ABCD1), Metachromatic leukopdystrophy (MLD) (ARSA), Phenylketonuria (PAH), Methylmalonic academia (MMUT), Propionic Acidemia (PCCA, PCCB), Retinitis Pigmentosa (RPE65), Usher Syndrome (MYO7A), and Gaucher Disease (GBA).

Methods of the present disclosure can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, an a mitotic inhibitor, a radiopharmaceutical).

### Nucleic Acid Molecules

In some aspects, a nucleic acid molecule can be a synthetic nucleic acid molecule. In some aspects, a nucleic acid molecule can be a non-naturally occurring nucleic acid molecule. In some aspects, a non-naturally occurring nucleic acid molecule can comprise at least one non-naturally occurring nucleotide. The at least one non-naturally occurring nucleotide can be any non-naturally occurring nucleotide known in the art. In some aspects, a nucleic acid molecule can be a modified nucleic acid molecule. In some aspects, a modified nucleic acid molecule can comprise at least one modified nucleotide. The at least one modified nucleotide can be any modified nucleic acid known in the art.

In some aspects, an mRNA molecule can be capped using any method and/or capping moiety known in the art. An mRNA molecule can be capped with m7G (5′) ppp (5′) G moiety. A m7G (5′) ppp (5′) G moiety is also referred to herein as a “Cap0”. An mRNA molecule can be capped with a CleanCap® moiety. A CleanCap® moiety can comprise a m7G (5′) ppp (5′) (2′OMeA) (CleanCap® AG) moiety. A CleanCap® moiety can comprise a m7G (5′) ppp (5′) (2′OMeG) (CleanCap® GG) moiety. An mRNA molecule can be capped with an anti-reverse cap analog (ARCAR) moiety. An ARCA® moiety can comprise a m7 (3′-O-methyl) G (5′) ppp (5′) G moiety. An mRNA molecule can be capped with a CleanCap® 3′OMe moiety (CleanCap®+ARCA®).

In some aspects, an mRNA molecule can comprise at least one modified nucleic acid.

The at least one modified nucleic acid can comprise 5-methoxyuridine (5moU). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, at least about 90%, or at least about 95%, or at least about 99% of the uridine bases in an mRNA molecule are 5-methoxyuridine bases. In some aspects, all of the uridine bases in an mRNA molecule are 5-methoxyuridine bases. Without wishing to be bound by theory, 5-methoxyuridine can improve protein expression and reduce immunogenicity (see Li et al., Bioconjugate Chem. 2016, 27, 3, 849-853 and Vaidyanathan et al. Molecular Therapy—Nucleic Acids, 2018, 12, 530-542).

In some aspects, an mRNA molecule can comprise at least one modified nucleic acid.

The at least one modified nucleic acid can comprise N₁-methylpseudouridine (me¹ψ). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, at least about 90%, or at least about 95%, or at least about 99% of the uridine bases in an mRNA N₁-methylpseudouridine bases. In some aspects, all of the uridine bases in an mRNA molecule are N₁-methylpseudouridine bases. Without wishing to be bound by theory, N₁-methylpseudouridine can improve protein expression (see Li et al., Bioconjugate Chem. 2016, 27, 3, 849-853).

In some aspects, an mRNA molecule can comprise at least one modified nucleic acid.

The at least one modified nucleic acid can comprise pseudouridine (Y). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, at least about 90%, or at least about 95%, or at least about 99% of the uridine bases in an mRNA pseudouridine bases. In some aspects, all of the uridine bases in an mRNA molecule are pseudouridine bases. Without wishing to be bound by theory, pseudouridine can improve protein expression and reduce immunogenicity (see Li et al., Bioconjugate Chem. 2016, 27, 3, 849-853 and Vaidyanathan et al. Molecular Therapy—Nucleic Acids, 2018, 12, 530-542).

In some aspects, an mRNA molecule can comprise at least one modified nucleic acid.

The at least one modified nucleic acid can comprise 5-methylcytidine (5-MeC). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, at least about 90%, or at least about 95%, or at least about 99% of the cytidine bases in an mRNA 5-MeC bases. In some aspects, all of the cytidine bases in an mRNA molecule are 5-MeC bases.

In some aspects, a nucleic acid molecule can comprise a DNA molecule. Thus, in some aspects, a lipid nanoparticle can comprise a DNA molecule. In some aspects, the DNA molecule can be a circular DNA molecule, such as, but not limited to, a DNA plasmid or DNA nanoplasmid. Thus, in some aspects, a lipid nanoparticle can comprise a circular DNA molecule. In some aspects, a lipid nanoparticle can comprise a Doggybone DNA molecule. In some aspects, a lipid nanoparticle can comprise a DNA plasmid. In some aspects, a lipid nanoparticle can comprise a DNA nanoplasmid. In some aspects, a DNA molecule can be a linearized DNA molecule, such as, but not limited to, a linearized DNA plasmid or a linearized DNA nanoplasmid.

A DNA plasmid or DNA nanoplasmid can comprise can be at least about 0.25 kb, or at least about 0.5 kb, or at least about 0.75 kb, or at least about 1.0 kb, or at least about 1.25 kb, or at least about 1.5 kb, or at least about 1.75 kb, or at least about 2.0 kb, or at least about 2.25 kb, or at least about 2.5 kb, or at least about 2.75 kb, or at least about 3.0 kb, or at least about 3.25 kb, or at least about 3.5 kb, or at least about 3.75 kb, or at least about 4.0 kb, or at least about 4.25 kb, or at least about 4.5 kb, or at least about 4.75 kb, or at least about 5.0 kb, or at least about 5.25 kb, or at least about 5.5 kb, or at least about 5.75 kb, or at least about 6.0 kb, or at least about 6.25 kb, or at least about 6.5 kb, or at least about 6.75 kb, or at least about 7.0 kb, or at least about 7.25 kb, or at least about 7.5 kb, or at least about 7.75 kb, or at least about 8.0 kb, or at least about 8.25 kb, or at least about 8.5 kb, or at least about 8.75 kb, or at least about 9.0 kb, or at least about 9.25 kb, or at least about 9.5 kb, or at least about 9.75 kb, or at least about 10.0 kb, or at least about 10.25 kb, or at least about 10.5 kb, or at least about 10.75 kb, or at least about 11.0 kb, or at least about 11.25 kb, or at least about 11.5 kb, or at least about 11.75 kb, or at least about 12 kb, or at least about 12.25 kb, or at least about 12.5 kb, or at least about 12.75 kb, or at least about 13.0 kb, or at least about 13.25 kb, or at least about 13.5 kb, or at least about 13.75 kb, or at least about 14.0 kb, or at least about 14.25 kb, or at least about 14.5 kb, or at least about 14.75 kb or at least about 15.0 kb in length.

In some aspects, a nucleic acid molecule formulated in a lipid nanoparticle of the present disclosure can comprise at least one transgene sequence. In some aspects, a transgene sequence can comprise a nucleotide sequence encoding at least one therapeutic protein. In some aspects, a transgene sequence can comprise a nucleotide sequence encoding at least one transposase. In some aspects, a transgene sequence can comprise a nucleotide sequence encoding at least one transposon. In some aspects, a transposon can comprise a nucleotide sequence encoding at least one therapeutic protein. In some aspects, a transposon can comprise a nucleotide sequence encoding at least one therapeutic protein and at least one protomer sequence, wherein the at least one therapeutic protein is operatively linked to the at least one promoter sequence.

In some aspects, the lipid nanoparticles of the present disclosure can be produced using a microfluidic-mixing platform. In some aspects, the microfluidic-mixing platform can be a non-turbulent microfluidic mixing platform.

In some aspects, a microfluidic-mixing platform can produce the lipid nanoparticles of the present invention by combining a miscible solvent phase comprising the lipid components of the nanoparticle and an aqueous phase comprising the lipid nanoparticle cargo (e.g. nucleic acid, DNA, mRNA, etc.) using a microfluidic device. In some aspects, the miscible solvent phase and the aqueous phase are mixed in the microfluidic device under laminar flow conditions that do not allow for immediate mixing of the two phases. As the two phases move under laminar flow in a microfluidic channel, microscopic features in the channel can allow for controlled, homogenous mixing to produce the lipid nanoparticles of the present disclosure.

In some aspects, the microfluidic-mixing platform can include, but are not limited to the NanoAssemblr® Spark (Precision NanoSystems), the NanoAssemblr® Ignite™ (Precision NanoSystems), the NanoAssemblr® Benchtop (Precision NanoSystems), the NanoAssemblr® Blaze (Precision NanoSystems) or the NanoAssemblr® GMP System (Precision NanoSystems).

In some aspects, the lipid nanoparticles of the present disclosure can be produced using a microfluidic-mixing platform, wherein the microfluidic mixing platform mixes at a rate of at least about 2.5 ml/min, or at least about 5 ml/min, or at least about 7.5 ml/min, or at least about 10 ml/min, or at least about 12.5 ml/min, or at least about 15 ml/min, or at least about 17.5 ml/min, or at least about 20 ml/min, or at least about 22.5 ml/min, or at least about 25 ml/min, or at least about 27.5 ml/min, or at least about 30 ml/min.

In some aspects, the lipid nanoparticles of the present disclosure can be produced using a microfluidic-mixing platform, wherein the microfluidic mixing platform mixes a miscible solvent phase and an aqueous phase at a ratio of about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, solvent: aqueous, v: v.

piggyBac ITR sequences

In some aspects, a nucleic acid can comprise a piggyBac ITR sequence. In some aspects, a nucleic acid can comprise a first piggyBac ITR sequence and a second piggyBac ITR sequence.

In some aspects, a piggyBac ITR sequence can comprise any piggyBac ITR sequence known in the art.

In some aspects of the methods of the present disclosure, a piggyBac ITR sequence, such as a first piggyBac ITR sequence and/or a second piggyBac ITR sequence in an AAV piggyBac transposon can comprise, consist essentially of, or consist of a Sleeping Beauty transposon ITR, a Helraiser transposon ITR, a Tol2 transposon ITR, a TcBuster transposon ITR or any combination thereof.

Transposition systems

In some aspects, a nucleic acid can comprise a transposon or a nanotransposon comprising: a first nucleic acid sequence comprising: (a) a first inverted terminal repeat (ITR) or a sequence encoding a first ITR, (b) a second ITR or a sequence encoding a second ITR, and (c) an intra-ITR sequence or a sequence encoding an intra-ITR, wherein the intra-ITR sequence comprises a transposon sequence or a sequence encoding a transposon.

In some aspects, a nucleic acid can comprise a transposon or a nanotransposon comprising: a first nucleic acid sequence comprising: (a) a first inverted terminal repeat (ITR) or a sequence encoding a first ITR, (b) a second ITR or a sequence encoding a second ITR, and (c) an intra-ITR sequence or a sequence encoding an intra-ITR, wherein the intra-ITR sequence comprises a transposon sequence or a sequence encoding a transposon, and a second nucleic acid sequence comprising an inter-ITR sequence or a sequence encoding an inter-ITR, wherein the length of the inter-ITR sequence is equal to or less than 700 nucleotides.

The transposon or nanotransposon of the present disclosure can be a piggyBac™ (PB) transposon. In some aspects when the transposon is a PB transposon, the transposase is a piggyBac™ (PB) transposase a piggyBac-like (PBL) transposase or a Super piggyBac™ (SPB) transposase. Preferably, the sequence encoding the SPB transposase is an mRNA sequence.

Non-limiting examples of PB transposons and PB, PBL and SPB transposases are described in detail in U.S. Pat. Nos. 6,218,182; 6,962,810; 8,399,643 and PCT Publication No. WO 2010/099296.

The PB, PBL and SPB transposases recognize transposon-specific inverted terminal repeat sequences (ITRs) on the ends of the transposon, and inserts the contents between the ITRs at the sequence 5′-TTAT-3′ within a chromosomal site (a TTAT target sequence) or at the sequence 5′-TTAA-3′ within a chromosomal site (a TTAA target sequence). The target sequence of the PB or PBL transposon can comprise or consist of 5′-CTAA-3′,5′-TTAG-3′,5′-ATAA-3′,5′-TCAA-3′,5′AGTT-3′,5′-ATTA-3′,5′-GTTA-3′,5′-TTGA-3′,5′-TTTA-3′,5′-TTAC-3′,5′-ACTA-3′,5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. The PB or PBL transposon system has no payload limit for the genes of interest that can be included between the ITRs.

Exemplary amino acid sequences for one or more PB, PBL and SPB transposases are disclosed in U.S. Pat. Nos. 6,218,185; 6,962,810 and 8,399,643, each of which is incorporated herein by reference in its entirety for examples of transposases that may be used in conjunction with the compositions and methods described herein. In some embodiments, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 1. In some embodiments, the PB transposases comprises the amino acid sequence of SEQ ID NO: 1.

The PB or PBL transposase can comprise or consist of an amino acid sequence having an amino acid substitution at two or more, at three or more or at each of positions 30, 165, 282, and/or 538 of the sequence of SEQ ID NO: 1. The transposase can be a SPB transposase that comprises or consists of the amino acid sequence of the sequence of SEQ ID NO: 1 wherein the amino acid substitution at position 30 can be a substitution of a valine (V) for an isoleucine (I), the amino acid substitution at position 165 can be a substitution of a serine(S) for a glycine (G), the amino acid substitution at position 282 can be a substitution of a valine (V) for a methionine (M), and the amino acid substitution at position 538 can be a substitution of a lysine (K) for an asparagine (N). In some embodiments, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 2. In some embodiments, the SPB transposase comprises the amino sequence set forth in SEQ ID NO: 2.

In certain aspects wherein the transposase comprises the above-described mutations at positions 30, 165, 282 and/or 538, the PB, PBL and SPB transposases can further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 1 or SEQ ID NO: 2 are described in more detail in PCT Publications No. WO 2019/173636 and No. WO 2020/051374, each of which is incorporated herein by reference in its entirety for examples of transposases that may be used in conjunction with the compositions and methods described herein.

In some embodiments, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 3. In some embodiments, the PB transposase comprises the amino acid sequence set forth in SEQ ID NO: 3.

The PB or PBL transposase can comprise or consist of an amino acid sequence having an amino acid substitution at two or more, at three or more or at each of positions 29, 164, 281, and/or 537 of the sequence of SEQ ID NO: 3. The transposase can be a SPB transposase that comprises or consists of the amino acid sequence of the sequence of SEQ ID NO: 3 wherein the amino acid substitution at position 29 can be a substitution of a valine (V) for an isoleucine (I), the amino acid substitution at position 164 can be a substitution of a serine(S) for a glycine (G), the amino acid substitution at position 281 can be a substitution of a valine (V) for a methionine (M), and the amino acid substitution at position 537 can be a substitution of a lysine (K) for an asparagine (N). In some embodiments, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 4. In some embodiments, the SPB transposase comprises the amino acid sequence set forth in SEQ ID NO: 4.

In certain aspects wherein the transposase comprises the above-described mutations at positions 29, 164, 281, and/or 537, the PB, PBL and SPB transposases can further comprise an amino acid substitution at one or more of positions 2, 45, 81, 102, 118, 124, 176, 179, 184, 186, 199, 206, 208, 225, 234, 239, 240, 242, 257, 295, 297, 310, 314, 318, 326, 327, 339, 420, 435, 455, 469, 485, 502, 551, 569 and 590 of the sequence of SEQ ID NO: 3 or SEQ ID NO: 4 are described in more detail in PCT Publication No. WO 2019/173636 and No. WO 2020/051374, each of which is incorporated herein by reference in its entirety for examples of transposases that may be used in conjunction with the compositions and methods described herein.

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is isolated or derived from the insect Trichoplusia ni (GenBank Accession No. AAA87375) or Bombyx mori (GenBank Accession No. BAD11135).

A hyperactive PB or PBL transposase is a transposase that is more active than the endogenous transposase from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636, each of which is incorporated herein by reference in its entirety for examples of transposases that may be used in conjunction with the compositions and methods described herein. A list of hyperactive amino acid substitutions is disclosed in U.S. Pat. No. 10,041,077, which is incorporated herein by reference in its entirety for examples of amino acid substitutions that may be introduced into the transposases described herein. A transposon or nanotransposon of the present disclosure can be a Sleeping Beauty transposon. In some aspects, when the transposon is a Sleeping Beauty transposon, the transposase is a Sleeping Beauty transposase (for example as disclosed in U.S. Pat. No. 9,228,180, which is incorporated herein by reference in its entirety for examples of transposases that may be used in conjunction with the compositions and methods described herein) or a hyperactive Sleeping Beauty (SB100X) transposase.

In some aspects, the PB or PBL transposase is integration deficient. An integration deficient PB or PBL transposase is a transposase that can excise its corresponding transposon, but that integrates the excised transposon at a lower frequency than a corresponding wild type transposase. Examples of integration deficient PB or PBL transposases are disclosed in U.S. Pat. Nos. 6,218,185; 6,962,810, 8,399,643 and WO 2019/173636, each of which is incorporated herein by reference in its entirety for examples of transposases that may be used in conjunction with the compositions and methods described herein. A list of integration deficient amino acid substitutions is disclosed in U.S. Pat. No. 10,041,077, which is incorporated herein by reference in its entirety for examples of amino acid substitutions that may be introduced into transposases described herein.

In some aspects, the PB or PBL transposase is fused to a nuclear localization signal. Examples of PB or PBL transposases fused to a nuclear localization signal are disclosed in U.S. Pat. Nos. 6,218,185; 6,962,810, 8,399,643 and WO 2019/173636, each of which is incorporated herein by reference in its entirety for examples of transposases that may be used in conjunction with the compositions and methods described herein.

A transposon or nanotransposon of the present disclosure can be a Sleeping Beauty transposon. In some aspects, when the transposon is a Sleeping Beauty transposon, the transposase is a Sleeping Beauty transposase (for example as disclosed in U.S. Pat. No. 9,228,180, which is incorporated herein by reference in its entirety for examples of transposases that may be used in conjunction with the compositions and methods described herein) or a hyperactive Sleeping Beauty (SB100X) transposase.

A transposon or nanotransposon of the present disclosure can be a Helraiser transposon. An exemplary Helraiser transposon includes Helibat1. In some aspects, when the transposon is a Helraiser transposon, the transposase is a Helitron transposase (for example, as disclosed in WO 2019/173636, which is incorporated herein by reference in its entirety for examples of transposases that may be used in conjunction with the compositions and methods described herein).

A transposon or nanotransposon of the present disclosure can be a Tol2 transposon. In some aspects, when the transposon is a Tol2 transposon, the transposase is a Tol2 transposase (for example, as disclosed in WO 2019/173636).

A transposon or nanotransposon of the present disclosure can be a TcBuster transposon. In some aspects, when the transposon is a TcBuster transposon, the transposase is a TcBuster transposase or a hyperactive TcBuster transposase (for example, as disclosed in WO 2019/173636, which is incorporated herein by reference in its entirety for examples of transposases that may be used in conjunction with the compositions and methods described herein). The TcBuster transposase can comprise or consist of a naturally occurring amino acid sequence or a non-naturally occurring amino acid sequence. The polynucleotide encoding a TcBuster transposase can comprise or consist of a naturally occurring nucleic acid sequence or a non-naturally occurring nucleic acid sequence.

In some aspects, a mutant TcBuster transposase comprises one or more sequence variations when compared to a wild type TcBuster transposase as described in more detail in PCT Publications No. WO 2019/173636 and No. WO 2020/051374, each of which is incorporated herein by reference in its entirety for examples of transposases that may be used in conjunction with the compositions and methods described herein.

The cell delivery compositions (e.g., transposons) disclosed herein can comprise a nucleic acid molecule encoding a therapeutic protein or therapeutic agent. Examples of therapeutic proteins include those disclosed in PCT Publications No. WO 2019/173636 and No. WO 2020/051374, each of which is incorporated herein by reference in its entirety for examples of transposases that may be used in conjunction with the compositions and methods described herein.

In some aspects, a therapeutic protein can comprise a FVIII polypeptide. An exemplary nanoplasmid encoding an FVIII polypeptide is provided in SEQ ID NO: 9. Accordingly, a nucleic acid formulated in a nanoparticle of the present disclosure can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 9.

In some aspects, a therapeutic protein can comprise a propionyl-CoA carboxylase subunit alpha (PCCA) polypeptide. An exemplary transposon encoding a PCCA polypeptide is provided in SEQ ID NO: 10. Accordingly, a nucleic acid formulated in a nanoparticle of the present disclosure can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 10.

### Gene Editing Systems

The present disclosure provides a gene editing composition and/or a cell comprising the gene editing composition. The gene editing composition can comprise a nanoparticle comprising a nucleic acid, wherein the nucleic acid comprises a sequence encoding a DNA binding domain and a sequence encoding a nuclease protein or a nuclease domain thereof. The sequence encoding a nuclease protein or the sequence encoding a nuclease domain thereof can comprise a DNA sequence, an RNA sequence, or a combination thereof. The nuclease or the nuclease domain thereof can comprise one or more of a CRISPR/Cas protein, a Transcription Activator-Like Effector Nuclease (TALEN), a Zinc Finger Nuclease (ZFN), and an endonuclease.

The nuclease or the nuclease domain thereof can comprise a nuclease-inactivated Cas (dCas) protein and an endonuclease. The endonuclease can comprise a Clo051 nuclease or a nuclease domain thereof. The gene editing composition can comprise a fusion protein. The fusion protein can comprise a nuclease-inactivated Cas9 (dCas9) protein and a Clo051 nuclease or a Clo051 nuclease domain. In some aspects, the fusion protein can further comprise at least one nuclear localization signal (NLS). In some aspects, the fusion protein can further comprise at least two NLSs. The gene editing composition can further comprise a guide sequence. The guide sequence can comprise an RNA sequence.

A transgene can comprise a nucleic sequence encoding a small, Cas9 (Cas9) operatively-linked to an effector. The disclosure provides a fusion protein comprising, consisting essentially of or consisting of a DNA localization component and an effector molecule, wherein the effector comprises a small, Cas9 (Cas9). A small Cas9 construct of the disclosure can comprise an effector comprising a type IIS endonuclease.

A transgene can comprise a nucleic sequence encoding an inactivated, small, Cas9 (dSaCas9) operatively-linked to an effector. A transgene can comprise a nucleic sequence encoding a fusion protein comprising, consisting essentially of or consisting of a DNA localization component and an effector molecule, wherein the effector comprises a small, inactivated Cas9 (dSaCas9). A small, inactivated Cas9 (dSaCas9) construct of the disclosure can comprise an effector comprising a type IIS endonuclease.

A transgene can comprise a nucleic sequence encoding an inactivated Cas9 (dCas9) operatively-linked to an effector. A transgene can comprise a nucleic sequence encoding a fusion protein comprising, consisting essentially of or consisting of a DNA localization component and an effector molecule, wherein the effector comprises an inactivated Cas9 (dCas9). An inactivated Cas9 (dCas9) construct of the disclosure can comprise an effector comprising a type IIS endonuclease.

The dCas9 can be isolated or derived from Streptoccocus pyogenes. The dCas9 can comprise a dCas9 with substitutions at amino acid positions 10 and 840, which inactivate the catalytic site. In some aspects, these substitutions are D10A and H840A.

A cell comprising the gene editing composition can express the gene editing composition stably or transiently. Preferably, the gene editing composition is expressed transiently. The guide RNA can comprise a sequence complementary to a target sequence within a genomic DNA sequence. The target sequence within a genomic DNA sequence can be a target sequence within a safe harbor site of a genomic DNA sequence.

Gene editing compositions, including Cas-CLOVER, and methods of using these compositions for gene editing are described in detail in U.S. Patent Publication Nos. 2017/0107541, 2017/0114149, 2018/0187185 and U.S. Pat. No. 10,415,024, each of which is incorporated herein by reference in its entirety for examples of gene editing systems that may be used in conjunction with the compositions and methods described herein. In some aspects, a Cas-CLOVER protein can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 11. In some embodiments, the Cas-CLOVER protein comprises the amino acid sequence set forth in SEQ ID NO: 11.

Accordingly, the present disclosure provides any of the lipid nanoparticle compositions described herein, wherein the lipid nanoparticle comprises at least one genomic editing composition, wherein the at least one genomic editing composition comprises: a) a nucleic acid molecule comprising a nucleic acid sequence encoding a fusion protein, wherein the fusion protein comprises (i) an inactivated Cas9 (dCas9) protein or an inactivated nuclease domain thereof, (ii) a Clo051 protein or a nuclease domain thereof; and b) at least one gRNA molecule. In some aspects, the fusion protein can further comprise at least one NLS. In some aspects, the at least one genomic editing composition can comprise at least two species of gRNA molecules.

Exemplary nucleic acid sequence encoding a fusion protein are presented in SEQ ID NO: 5. Accordingly, a nucleic acid molecule formulated in a lipid nanoparticle of the present disclosure can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 5.

Exemplary gRNA sequences are presented in SEQ ID NOs: 6 and 7. Accordingly, gRNA molecules formulated in a lipid nanoparticle of the present disclosure can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 6 or SEQ ID NO: 7.

### Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the composition as well known in the art or as described herein.

For example, the disclosed LNP compositions of the present invention can further comprise a diluent. In some compositions, the diluent can be phosphate buffered saline (“PBS”).

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate. In some aspects, the buffer can include sucrose.

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994,), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446, each of which is incorporated herein by reference in its entirety for examples of injection devices that may be used in conjunction with the compositions and methods described herein.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g., jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol. A more detailed description of pulmonary administration, formulations and related devices is disclosed in PCT Publication No. WO 2019/049816, which is incorporated herein by reference in its entirety for examples of transposases that may be used in conjunction with the compositions and methods described herein.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (see, e.g., U.S. Pat. No. 5,514,670, which is incorporated herein by reference in its entirety for examples). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (see, e.g., U.S. Pat. No. 5,849,695, which is incorporated herein by reference in its entirety for examples). A more detailed description of mucosal administration and formulations is disclosed in PCT Publication No. WO 2019/049816, each of which is incorporated herein by reference in its entirety for examples of formulations that may be used in conjunction with the compositions and methods described herein.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (see, e.g., U.S. Pat. No. 5,814,599, each of which is incorporated herein by reference in its entirety for examples). A more detailed description of transdermal administration, formulations and suitable devices is disclosed in PCT Publication No. WO 2019/049816, which is incorporated herein by reference in its entirety for examples of formulations and devices that may be used in conjunction with the compositions and methods described herein.

It can be desirable to deliver the disclosed compounds to the subject over prolonged periods of time, for example, for periods of one week to one year from a single administration. Various slow release, depot or implant dosage forms can be utilized.

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired.

As a non-limiting example, treatment of humans or animals can be provided as a one-time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

In aspects where the compositions to be administered to a subject in need thereof are modified cells as disclosed herein, the cells can be administered between about 1×10³ and 1×10¹⁵ cells; 1×10³ and 1×10¹⁵ cells, about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In an aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/04981, which is incorporated herein by reference in its entirety for examples of transposases that may be used in conjunction with the compositions and methods described herein.

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In an aspect, the subject is a mammal. Preferably, the subject is human. The terms “subject” and “patient” are used interchangeably herein.

The disclosure provides a method for modulating or treating at least one malignant disease or disorder in a cell, tissue, organ, animal or subject. Non-limiting examples of a malignant disease or disorder include cancer and liver diseases or disorders.

Any method can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, an a mitotic inhibitor, a radiopharmaceutical).

In some aspects, the subject does not develop graft vs. host (GvH) and/or host vs. graft (HvG) following administration. In an aspect, the administration is systemic. Systemic administration can be any means known in the art and described in detail herein. Preferably, systemic administration is by an intravenous injection or an intravenous infusion. In an aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously. In some aspects, where the composition is autologous cells or allogeneic cells, the dose is an amount sufficient for the cells to engraft and/or persist for a sufficient time to treat the disease or disorder.

In some aspects of the methods of treatment described herein, the treatment can be modified or terminated. Specifically, in aspects where the composition used for treatment comprises an inducible proapoptotic polypeptide, apoptosis may be selectively induced in the cell by contacting the cell with an induction agent. A treatment may be modified or terminated in response to, for example, a sign of recovery or a sign of decreasing disease severity/progression, a sign of disease remission/cessation, and/or the occurrence of an adverse event. In some aspects, the method comprises the step of administering an inhibitor of the induction agent to inhibit modification of the cell therapy, thereby restoring the function and/or efficacy of the cell therapy (for example, when a sign or symptom of the disease reappear or increase in severity and/or an adverse event is resolved).

### Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

### Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art. (See, e.g., Ausubel, supra; or Sambrook, supra).

### Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The disclosure further provides recombinant expression cassettes comprising a nucleic acid of the disclosure. A nucleic acid sequence of the disclosure can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

### Expression Vectors and Host Cells

The disclosure also relates to vectors that include isolated nucleic acid molecules of the disclosure and host cells that are genetically engineered with the recombinant vectors, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124 (8): 1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCE, RAD51C, GCS, MDR1, ALDH1, NKX2.2, or any combination thereof.

Those of ordinary skill in the art are knowledgeable in the numerous expression systems available for expression of a nucleic acid molecule encoding a protein of the disclosure.

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

In the chemical formulas shown herein, the marking indicates the position where a functional group bonds to another portion of a molecule. Definitions of specific functional groups and chemical terms are described in more detail below.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

One of ordinary skill in the art will appreciate that the synthetic methods, as described herein, utilize a variety of protecting groups. By the term “protecting group,” as used herein, it is meant that a particular functional moiety, e.g., O, S, or N, is temporarily blocked so that a reaction can be carried out selectively at another reactive site in a multifunctional compound. In certain embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the invention contain 1-18 aliphatic carbon atoms. In certain embodiments, the alkyl, alkenyl, and alkynyl groups employed in the invention contain 1-15 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkyl” as used herein refers to saturated, straight- (e.g., unbranched) or branched-chain aliphatic groups having from 1 to 18 carbon atoms, As such, “alkyl” encompasses C₁, C₂, C₃, C₄, C₅, C₆, C₇, C₈, C₉, C₁₀, C₁₁ and C₁₂ groups. Examples of alkyl groups include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

The term “alkylene” refers to a divalent alkyl radical. Any of the above mentioned monovalent alkyl groups may be an alkylene by abstraction of a second hydrogen atom from the alkyl. As herein defined, alkylene may also be a C₁-C₁₈ alkylene. An alkylene may further be a C₁-C₁₂ alkylene. Typical alkylene groups include, but are not limited to, —CH₂—, —CH(CH₃)—, —C(CH₃)₂—, —CH₂CH₂—, —CH₂CH(CH₃)—, —CH₂C (CH₃)₂—, —CH₂CH₂CH₂—, —CH₂CH₂CH₂CH₂—, and the like.

The term “alkenyl” refers to an unsaturated straight or, when applicable, branched chain aliphatic group with one or more carbon-carbon double bonds, having from 2 to 18 carbon atoms. As such, “alkenyl” encompasses C₂, C₃, C₄, C₅, C₆, C₇, C₈, C₉, C₁₀, C₁₁ and C₁₂ groups. Alkenyl groups include, for example, ethenyl, propenyl, butenyl, 1-methyl-2-buten-1-yl, and the like.

The term “alkynyl” refers to an unsaturated straight or, when applicable, branched chain aliphatic group with one or more carbon-carbon triple bonds, having from 2 to 18 carbon atoms. As such, “alkynyl” encompasses C₂, C₃, C₄, C₅, C₆, C₇, C₈, C₉, C₁₀, C₁₁ and C₁₂ groups. Representative alkynyl groups include ethynyl, 2-propynyl (propargyl), 1-propynyl, and the like.

As used herein, the term “aryl” group is a C₆-C₁₄ aromatic moiety comprising one to three aromatic rings, which is optionally substituted. As such, “aryl” includes C₆, C₇, C₈, C₉, C₁₀, C₁₁, C₁₂ C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl.

As used herein, the term “cycloalkyl” as employed herein includes saturated and partially unsaturated cyclic hydrocarbon groups having 3 to 12 carbons. As such, “cycloalkyl” includes C₃, C₄, C₅, C₆, C₇, C₈, C₉, C₁₀, C₁₁ and C₁₂ cyclic hydrocarbon groups.

Representative cycloalkyl groups include, without limitation, cyclopropyl, cyclobutyl, cyclopentyl, cyclopentenyl, cyclohexyl, cyclohexenyl, cycloheptyl, and cyclooctyl.

As used herein, the term “hydroxyalkyl” refers to -alkyl-OH or an alkyl chain substituted with at least one-OH.

As used herein, the term “halo” or “halogen” refers to fluoro, chloro, bromo and iodo.

It will be understood that the compounds of any one of the Formulae disclosed herein and any pharmaceutically acceptable salts thereof, comprise stereoisomers, mixtures of stereoisomers, polymorphs of all isomeric forms of said compounds.

The term “independently selected” is used herein to indicate that the R groups can be identical or different.

The term “substituted,” whether preceded by the term “optionally” or not, and “substituent,” as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions).

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “antibody” is used in the broadest sense and specifically covers single monoclonal antibodies (including agonist and antagonist antibodies) and antibody compositions with polyepitopic specificity. It is also within the scope hereof to use natural or synthetic analogs, mutants, variants, alleles, homologs and orthologs (herein collectively referred to as “analogs”) of the antibodies hereof as defined herein. Thus, according to an aspect hereof, the term “antibody hereof” in its broadest sense also covers such analogs. Generally, in such analogs, one or more amino acid residues may have been replaced, deleted and/or added, compared to the antibodies hereof as defined herein.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, tRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 2.

TABLE 1
Conservative Substitutions I

| Side chain characteristics | | Amino Acid |
|---|---|---|
| Aliphatic | Non-polar | GAPILVF |
| | Polar-uncharged | CSTMNQ |
| | Polar-charged | DEKR |
| Aromatic | | HFWY |
| Other | | NQDE |

Alternately, conservative amino acids can be grouped as described in Lehninger, (Biochemistry, Second Edition; Worth Publishers, Inc. NY, N.Y. (1975), pp. 71-77) as set forth in Table 3.

TABLE 2
Conservative Substitutions II

| Side Chain Characteristic | | Amino Acid |
|---|---|---|
| Non-polar (hydrophobic) | Aliphatic: | ALIVP |
| | Aromatic: | FWY |
| | Sulfur-containing: | M |
| | Borderline: | GY |
| Uncharged-polar | Hydroxyl: | STY |
| | Amides: | NQ |
| | Sulfhydryl: | C |
| | Borderline: | GY |

| | |
|---|---|
| Positively Charged (Basic): | KRH |
| Negatively Charged (Acidic): | DE |

Alternately, exemplary conservative substitutions are set out in Table 4.

TABLE 3
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (b12seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

For the following Examples, Compound Numbers are assigned to compounds of Formula (I) and (II) of the present disclosure according to the following:

| COMPOUND | |
|---|---|
| NO. | Structure |
| 1 | 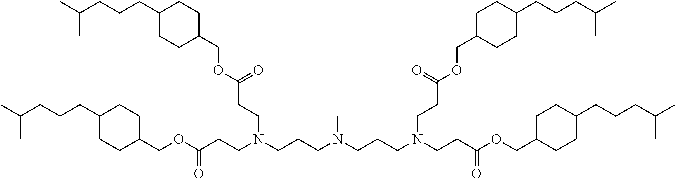 [SMILES: CC(C)CCCC1CCC(COC(=O)CCN(CCCN(C)CCCN(CCC(=O)OCC2CCC(CCCC(C)C)CC2)CCC(=O)OCC2CCC(CCCC(C)C)CC2)CCC(=O)OCC2CCC(CCCC(C)C)CC2)CC1] |
| 2 | 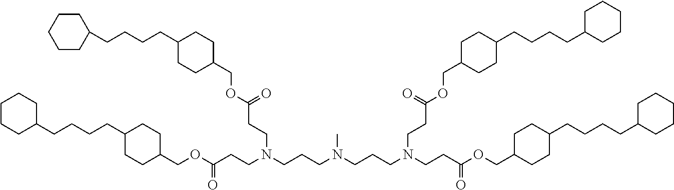 [SMILES: CN(CCCN(CCC(=O)OCC1CCC(CCCCC2CCCCC2)CC1)CCC(=O)OCC1CCC(CCCCC2CCCCC2)CC1)CCCN(CCC(=O)OCC1CCC(CCCCC2CCCCC2)CC1)CCC(=O)OCC1CCC(CCCCC2CCCCC2)CC1] |
| 3 | 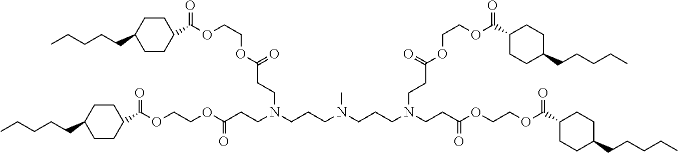 |
| 4 | 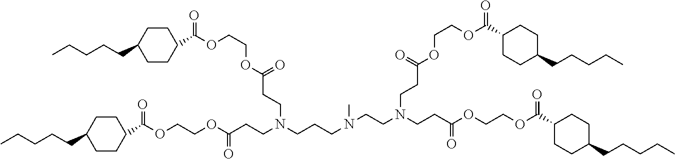 |
| 5 | 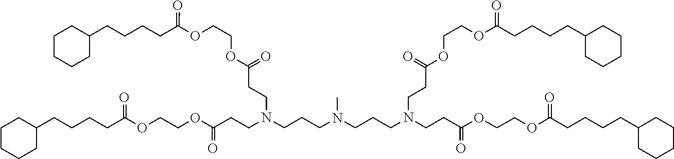 [SMILES: CN(CCCN(CCC(=O)OCCOC(=O)CCCCC1CCCCC1)CCC(=O)OCCOC(=O)CCCCC1CCCCC1)CCCN(CCC(=O)OCCOC(=O)CCCCC1CCCCC1)CCC(=O)OCCOC(=O)CCCCC1CCCCC1] |
| 6 | 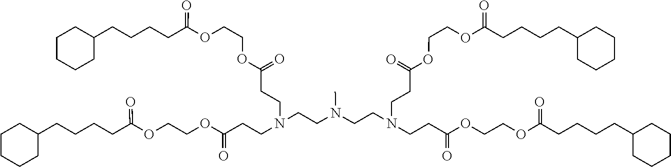 [SMILES: CN(CCN(CCC(=O)OCCOC(=O)CCCCC1CCCCC1)CCC(=O)OCCOC(=O)CCCCC1CCCCC1)CCN(CCC(=O)OCCOC(=O)CCCCC1CCCCC1)CCC(=O)OCCOC(=O)CCCCC1CCCCC1] |
| 7 | 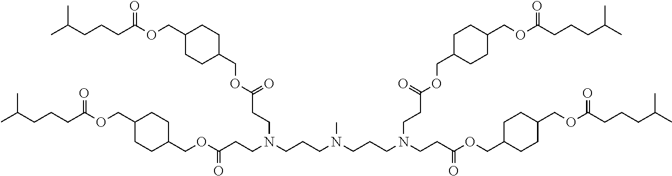 [SMILES: CC(C)CCCC(=O)OCC1CCC(COC(=O)CCN(CCCN(C)CCCN(CCC(=O)OCC2CCC(COC(=O)CCCC(C)C)CC2)CCC(=O)OCC2CCC(COC(=O)CCCC(C)C)CC2)CCC(=O)OCC2CCC(COC(=O)CCCC(C)C)CC2)CC1] |
| 8 | 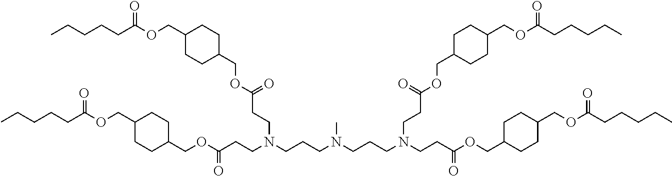 [SMILES: CCCCCC(=O)OCC1CCC(COC(=O)CCN(CCCN(C)CCCN(CCC(=O)OCC2CCC(COC(=O)CCCCC)CC2)CCC(=O)OCC2CCC(COC(=O)CCCCC)CC2)CCC(=O)OCC2CCC(COC(=O)CCCCC)CC2)CC1] |
| 9 | 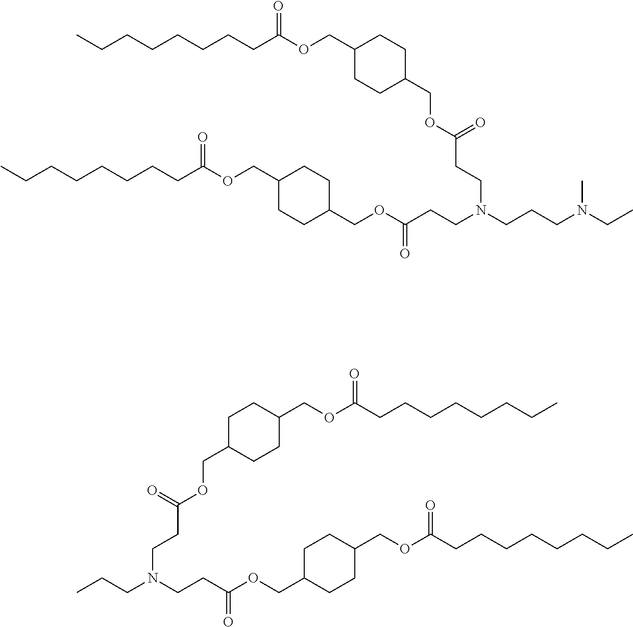 |
| 10 | 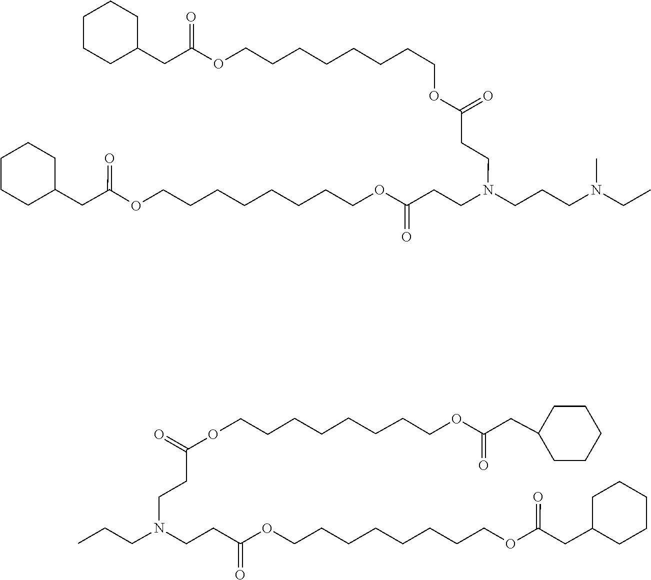 |
| 11 | 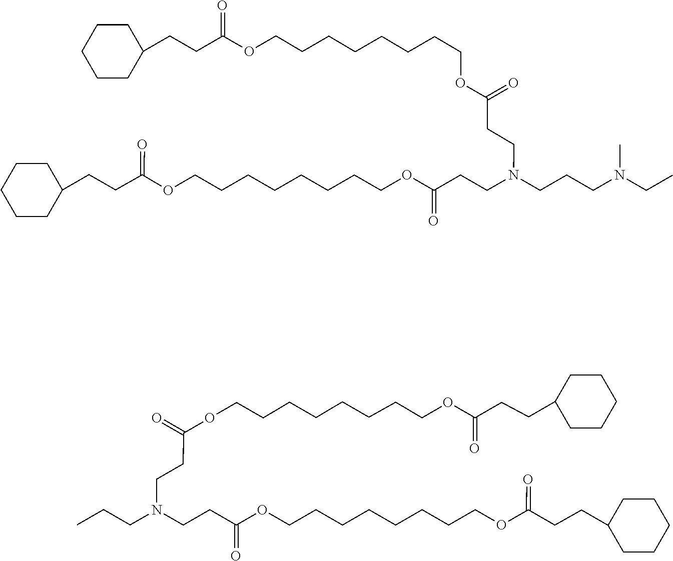 |
| 12 | 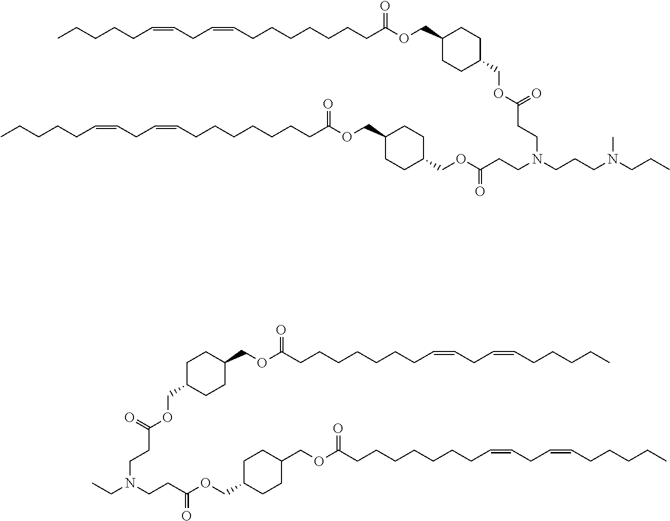 |
| 13 | 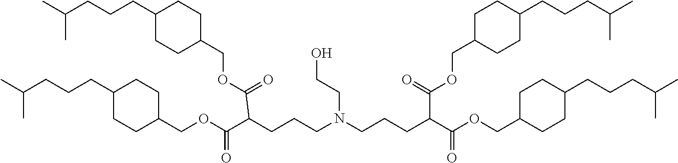 [SMILES: CC(C)CCCC1CCC(COC(=O)C(CCCN(CCO)CCCC(C(=O)OCC2CCC(CCCC(C)C)CC2)C(=O)OCC2CCC(CCCC(C)C)CC2)C(=O)OCC2CCC(CCCC(C)C)CC2)CC1] |
| 14 | 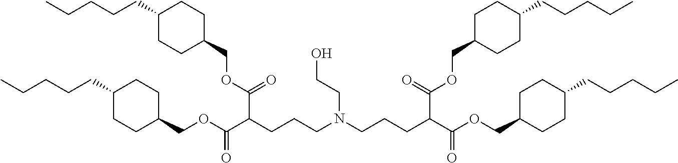 [SMILES: CCCCC[C@H]1CC[C@H](COC(=O)C(CCCN(CCO)CCCC(C(=O)OC[C@H]2CC[C@H](CCCCC)CC2)C(=O)OC[C@H]2CC[C@H](CCCCC)CC2)C(=O)OC[C@H]2CC[C@H](CCCCC)CC2)CC1] |
| 15 | 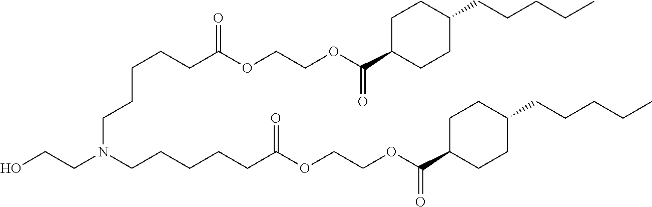 [SMILES: CCCCC[C@H]1CC[C@H](C(=O)OCCOC(=O)CCCCCN(CCO)CCCCCC(=O)OCCOC(=O)[C@H]2CC[C@H](CCCCC)CC2)CC1] |
| 16 | 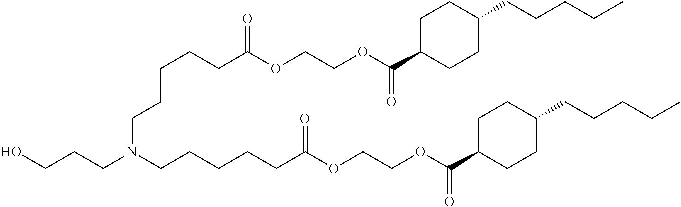 [SMILES: CCCCC[C@H]1CC[C@H](C(=O)OCCOC(=O)CCCCCN(CCCO)CCCCCC(=O)OCCOC(=O)[C@H]2CC[C@H](CCCCC)CC2)CC1] |
| 17 | 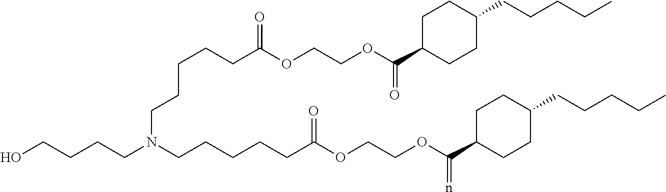 |
| 18 | 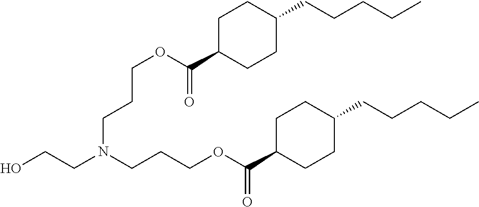 [SMILES: CCCCC[C@H]1CC[C@H](C(=O)OCCCN(CCO)CCCOC(=O)[C@H]2CC[C@H](CCCCC)CC2)CC1] |
| 19 | 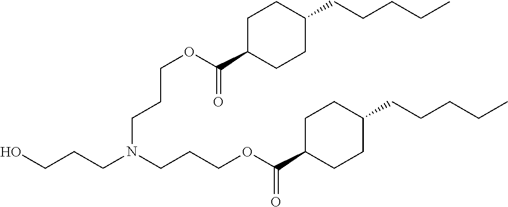 [SMILES: CCCCC[C@H]1CC[C@H](C(=O)OCCCN(CCCO)CCCOC(=O)[C@H]2CC[C@H](CCCCC)CC2)CC1] |
| 20 | 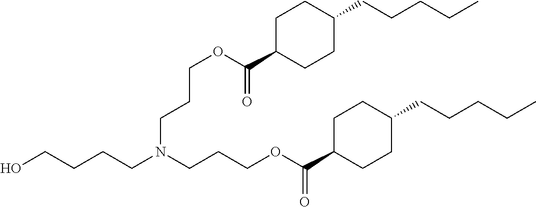 [SMILES: CCCCC[C@H]1CC[C@H](C(=O)OCCCN(CCCCO)CCCOC(=O)[C@H]2CC[C@H](CCCCC)CC2)CC1] |
| 21 | 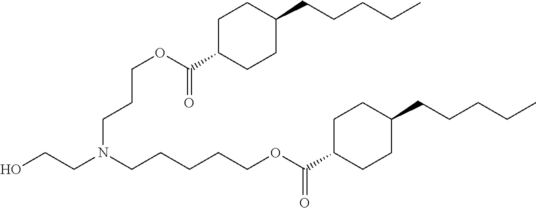 [SMILES: CCCCC[C@H]1CC[C@H](C(=O)OCCCCCN(CCO)CCCOC(=O)[C@H]2CC[C@H](CCCCC)CC2)CC1] |
| 22 | 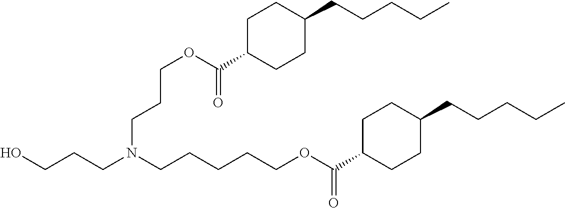 [SMILES: CCCCC[C@H]1CC[C@H](C(=O)OCCCCCN(CCCO)CCCOC(=O)[C@H]2CC[C@H](CCCCC)CC2)CC1] |
| 23 | 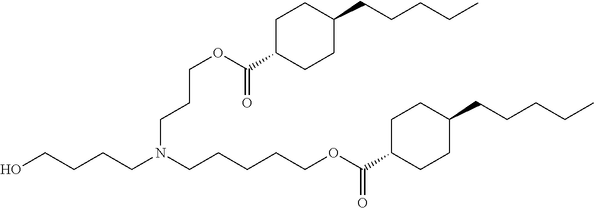 [SMILES: CCCCC[C@H]1CC[C@H](C(=O)OCCCCCN(CCCCO)CCCOC(=O)[C@H]2CC[C@H](CCCCC)CC2)CC1] |
| 24 | 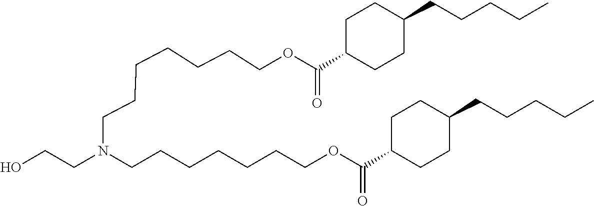 [SMILES: CCCCC[C@H]1CC[C@H](C(=O)OCCCCCCCN(CCO)CCCCCCCOC(=O)[C@H]2CC[C@H](CCCCC)CC2)CC1] |
| 25 | 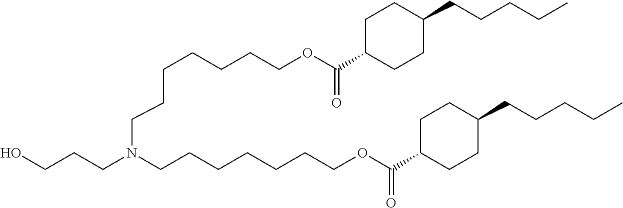 [SMILES: CCCCC[C@H]1CC[C@H](C(=O)OCCCCCCCN(CCCO)CCCCCCCOC(=O)[C@H]2CC[C@H](CCCCC)CC2)CC1] |
| 26 | 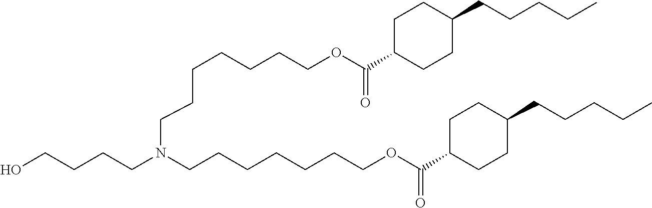 [SMILES: CCCCC[C@H]1CC[C@H](C(=O)OCCCCCCCN(CCCCO)CCCCCCCOC(=O)[C@H]2CC[C@H](CCCCC)CC2)CC1] |
| 27 | 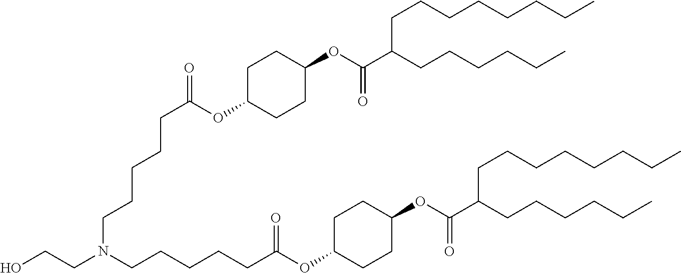 [SMILES: CCCCCCCCC(CCCCCC)C(=O)O[C@H]1CC[C@H](OC(=O)CCCCCN(CCO)CCCCCC(=O)O[C@H]2CC[C@H](OC(=O)C(CCCCCC)CCCCCCCC)CC2)CC1] |
| 28 | 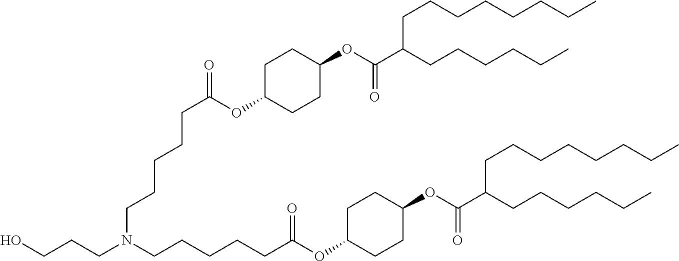 [SMILES: CCCCCCCCC(CCCCCC)C(=O)O[C@H]1CC[C@H](OC(=O)CCCCCN(CCCO)CCCCCC(=O)O[C@H]2CC[C@H](OC(=O)C(CCCCCC)CCCCCCCC)CC2)CC1] |
| 29 | 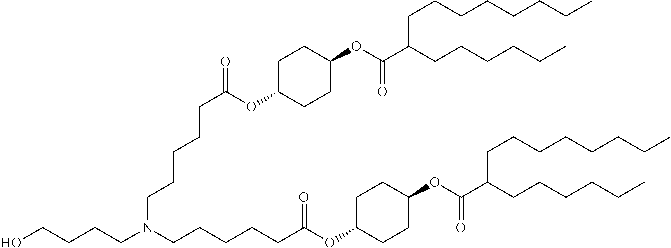 [SMILES: CCCCCCCCC(CCCCCC)C(=O)O[C@H]1CC[C@H](OC(=O)CCCCCN(CCCCO)CCCCCC(=O)O[C@H]2CC[C@H](OC(=O)C(CCCCCC)CCCCCCCC)CC2)CC1] |
| 30 | 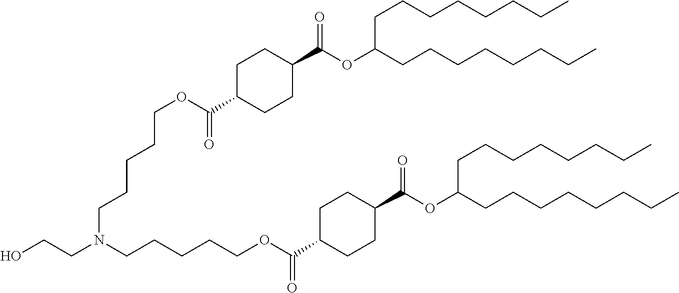 [SMILES: CCCCCCCCC(CCCCCCCC)OC(=O)[C@H]1CC[C@H](C(=O)OCCCCCN(CCO)CCCCCOC(=O)[C@H]2CC[C@H](C(=O)OC(CCCCCCCC)CCCCCCCC)CC2)CC1] |
| 31 | 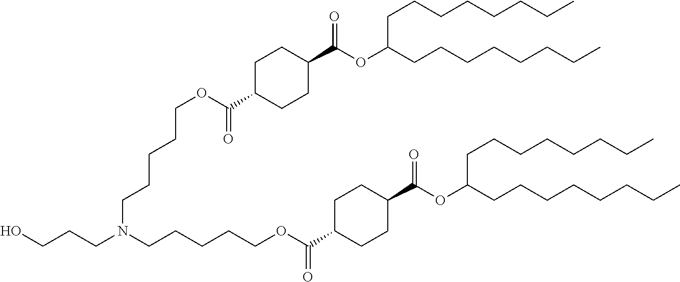 [SMILES: CCCCCCCCC(CCCCCCCC)OC(=O)[C@H]1CC[C@H](C(=O)OCCCCCN(CCCO)CCCCCOC(=O)[C@H]2CC[C@H](C(=O)OC(CCCCCCCC)CCCCCCCC)CC2)CC1] |
| 32 | 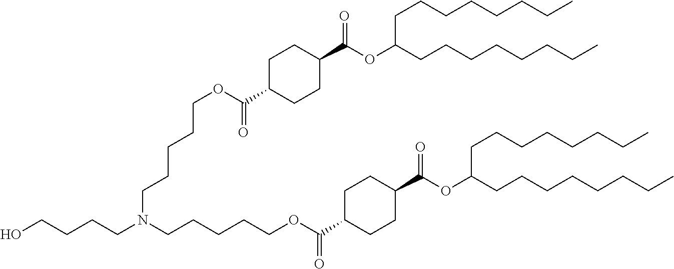 [SMILES: CCCCCCCCC(CCCCCCCC)OC(=O)[C@H]1CC[C@H](C(=O)OCCCCCN(CCCCO)CCCCCOC(=O)[C@H]2CC[C@H](C(=O)OC(CCCCCCCC)CCCCCCCC)CC2)CC1] |
| 33 | 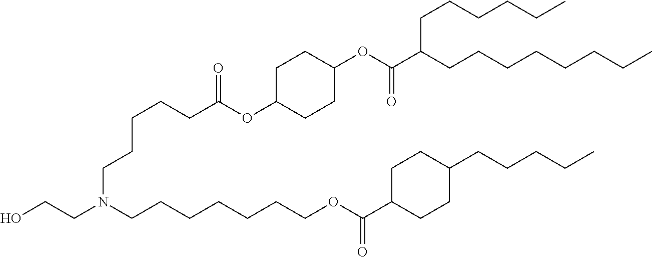 [SMILES: CCCCCCCCC(CCCCCC)C(=O)OC1CCC(OC(=O)CCCCCN(CCO)CCCCCCCOC(=O)C2CCC(CCCCC)CC2)CC1] |
| 34 | 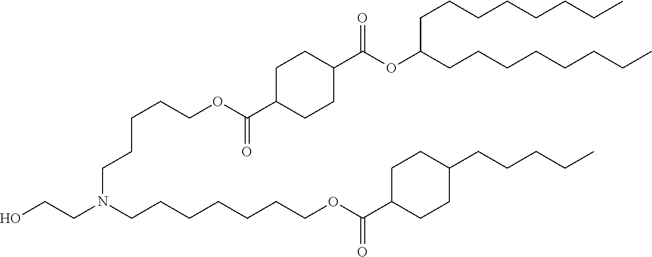 [SMILES: CCCCCCCCC(CCCCCCCC)OC(=O)C1CCC(C(=O)OCCCCCN(CCO)CCCCCCCOC(=O)C2CCC(CCCCC)CC2)CC1] |
| 35 | 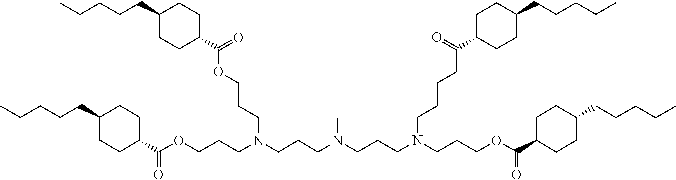 [SMILES: CCCCC[C@H]1CC[C@H](C(=O)CCCCN(CCCOC(=O)[C@H]2CC[C@H](CCCCC)CC2)CCCN(C)CCCN(CCCOC(=O)[C@H]2CC[C@H](CCCCC)CC2)CCCOC(=O)[C@H]2CC[C@H](CCCCC)CC2)CC1] |
| 36 | 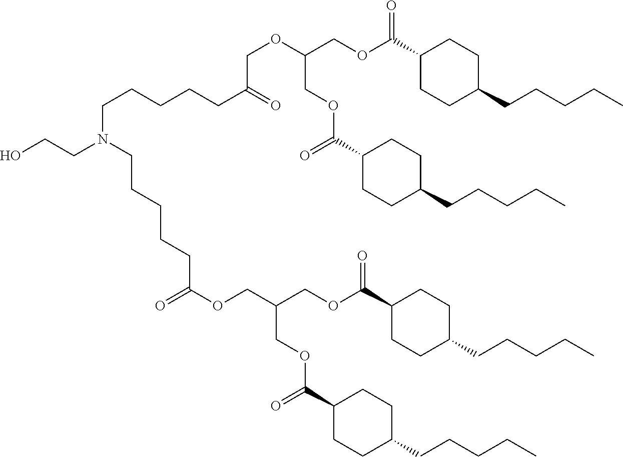 [SMILES: CCCCC[C@H]1CC[C@H](C(=O)OCC(COC(=O)CCCCCN(CCO)CCCCCC(=O)COC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1] |
| 37 | 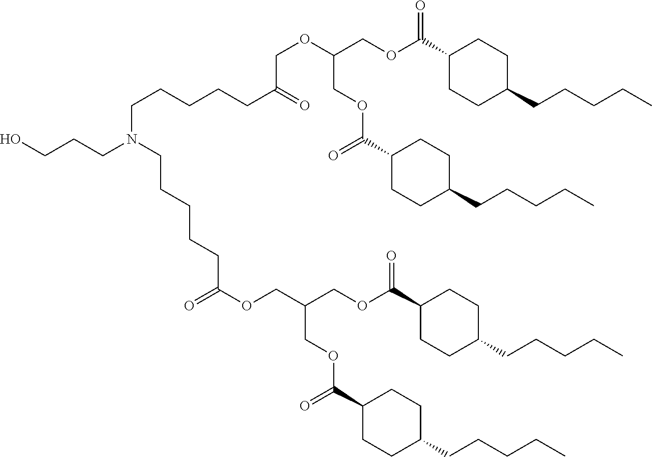 [SMILES: CCCCC[C@H]1CC[C@H](C(=O)OCC(COC(=O)CCCCCN(CCCO)CCCCCC(=O)COC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1] |
| 38 | 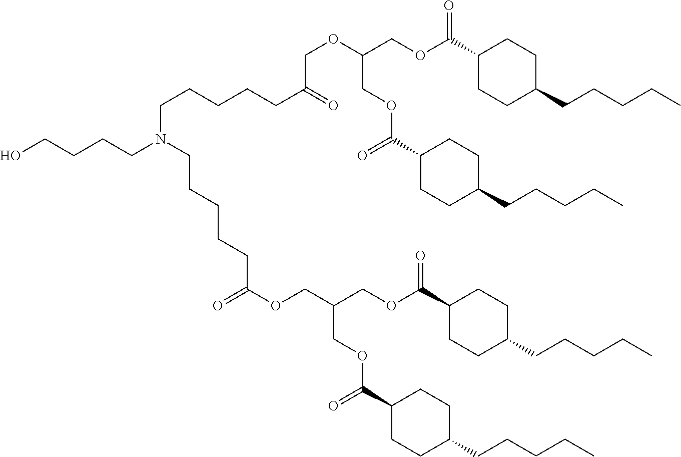 [SMILES: CCCCC[C@H]1CC[C@H](C(=O)OCC(COC(=O)CCCCCN(CCCCO)CCCCCC(=O)COC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1] |
| 39 | 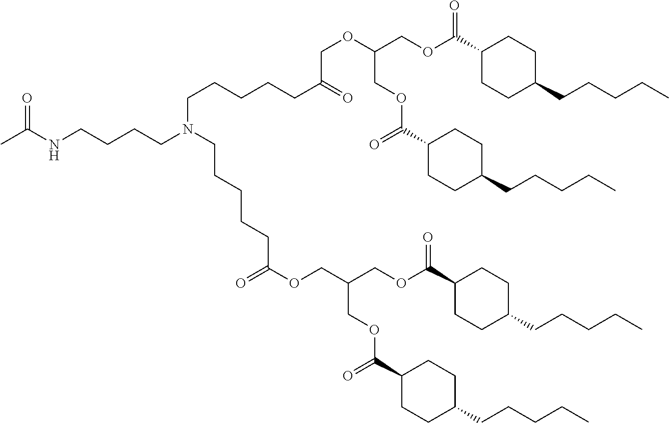 [SMILES: CCCCC[C@H]1CC[C@H](C(=O)OCC(COC(=O)CCCCCN(CCCCCC(=O)COC(COC(=O)[C@H]2CC[C@H](CCCCC)CC2)COC(=O)[C@H]2CC[C@H](CCCCC)CC2)CCCCNC(C)=O)COC(=O)[C@H]2CC[C@H](CCCCC)CC2)CC1] |
| 40 | 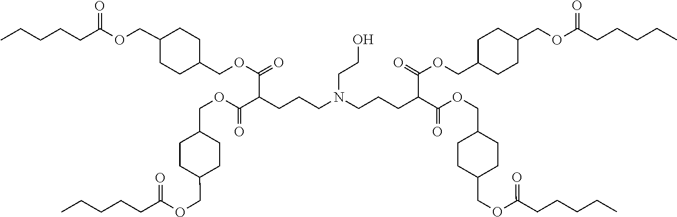 [SMILES: CCCCCC(=O)OCC1CCC(COC(=O)C(CCCN(CCO)CCCC(C(=O)OCC2CCC(COC(=O)CCCCC)CC2)C(=O)OCC2CCC(COC(=O)CCCCC)CC2)C(=O)OCC2CCC(COC(=O)CCCCC)CC2)CC1] |
| 41 | 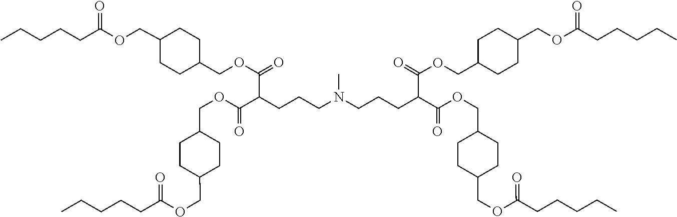 [SMILES: CCCCCC(=O)OCC1CCC(COC(=O)C(CCCN(C)CCCC(C(=O)OCC2CCC(COC(=O)CCCCC)CC2)C(=O)OCC2CCC(COC(=O)CCCCC)CC2)C(=O)OCC2CCC(COC(=O)CCCCC)CC2)CC1] |
| 42 | 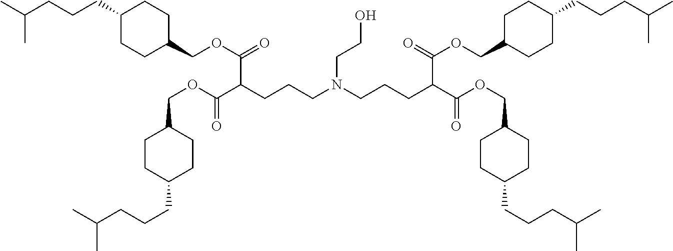 |
| 43 | 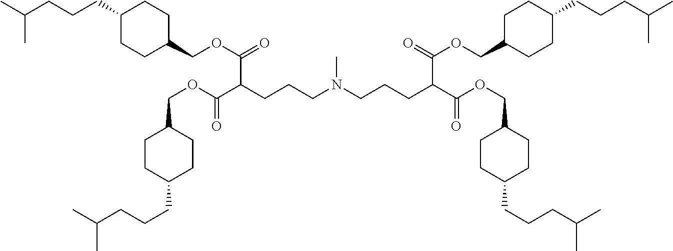 |
| 44 | 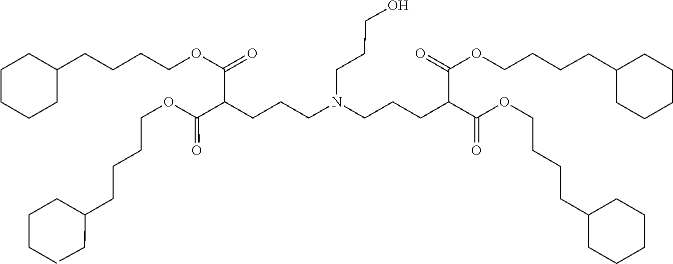 [SMILES: CCCCC(CC)CCCCOC(=O)C(CCCN(CCCO)CCCC(C(=O)OCCCCC1CCCCC1)C(=O)OCCCCC1CCCCC1)C(=O)OCCCCC1CCCCC1] |
| 45 | 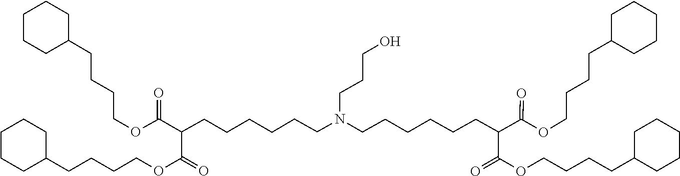 [SMILES: O=C(OCCCCC1CCCCC1)C(CCCCCCN(CCCO)CCCCCCC(C(=O)OCCCCC1CCCCC1)C(=O)OCCCCC1CCCCC1)C(=O)OCCCCC1CCCCC1] |
| 46 | 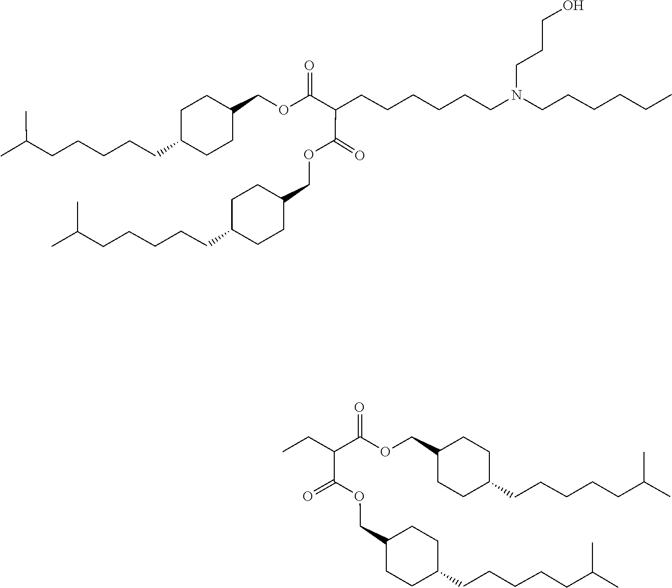 |
| 47 | 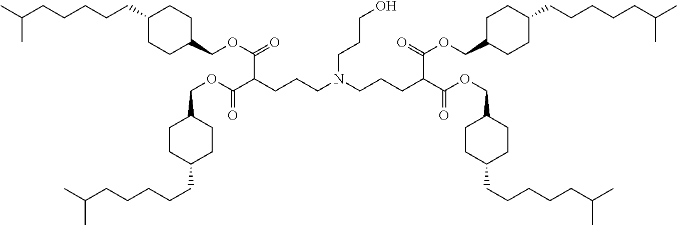 |
| 48 | 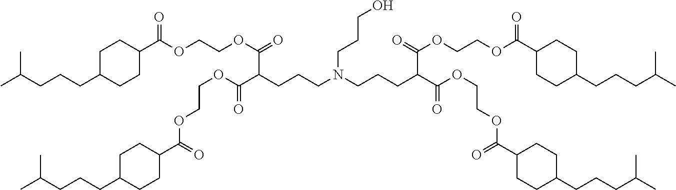 [SMILES: CC(C)CCCC1CCC(C(=O)OCCOC(=O)C(CCCN(CCCO)CCCC(C(=O)OCCOC(=O)C2CCC(CCCC(C)C)CC2)C(=O)OCCOC(=O)C2CCC(CCCC(C)C)CC2)C(=O)OCCOC(=O)C2CCC(CCCC(C)C)CC2)CC1] |
| 49 | 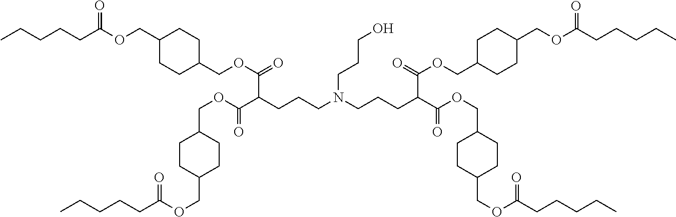 [SMILES: CCCCCC(=O)OCC1CCC(COC(=O)C(CCCN(CCCO)CCCC(C(=O)OCC2CCC(COC(=O)CCCCC)CC2)C(=O)OCC2CCC(COC(=O)CCCCC)CC2)C(=O)OCC2CCC(COC(=O)CCCCC)CC2)CC1] |
| 50 | 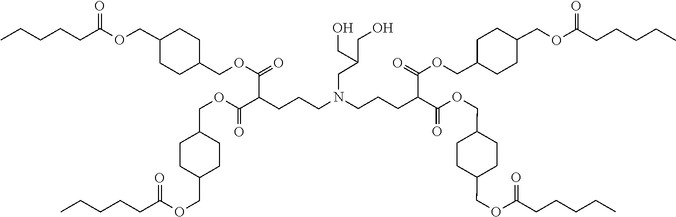 [SMILES: CCCCCC(=O)OCC1CCC(COC(=O)C(CCCN(CCCC(C(=O)OCC2CCC(COC(=O)CCCCC)CC2)C(=O)OCC2CCC(COC(=O)CCCCC)CC2)CC(CO)CO)C(=O)OCC2CCC(COC(=O)CCCCC)CC2)CC1] |
| 51 | 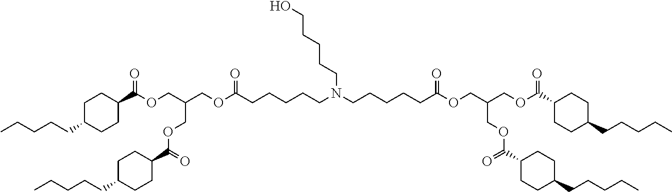 [SMILES: CCCCC[C@H]1CC[C@H](C(=O)OCC(COC(=O)CCCCCN(CCCCCO)CCCCCC(=O)OCC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1] |
| 52 | 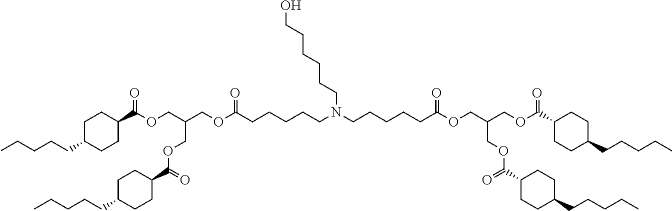 [SMILES: CCCCC[C@H]1CC[C@H](C(=O)OCC(COC(=O)CCCCCN(CCCCCCO)CCCCCC(=O)OCC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1] |
| 53 | 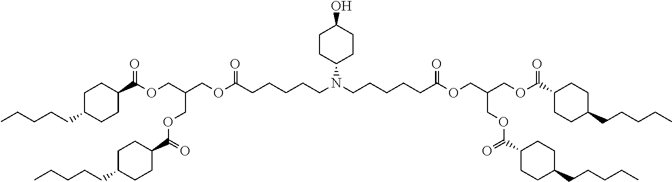 [SMILES: CCCCC[C@H]1CC[C@H](C(=O)OCC(COC(=O)CCCCCN(CCCCCC(=O)OCC(COC(=O)[C@H]2CC[C@H](CCCCC)CC2)COC(=O)[C@H]2CC[C@H](CCCCC)CC2)[C@H]2CC[C@H](O)CC2)COC(=O)[C@H]2CC[C@H](CCCCC)CC2)CC1] |
| 54 | 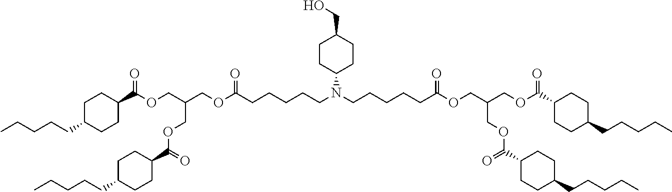 [SMILES: CCCCC[C@H]1CC[C@H](C(=O)OCC(COC(=O)CCCCCN(CCCCCC(=O)OCC(COC(=O)[C@H]2CC[C@H](CCCCC)CC2)COC(=O)[C@H]2CC[C@H](CCCCC)CC2)[C@H]2CC[C@H](CO)CC2)COC(=O)[C@H]2CC[C@H](CCCCC)CC2)CC1] |
| 55 | 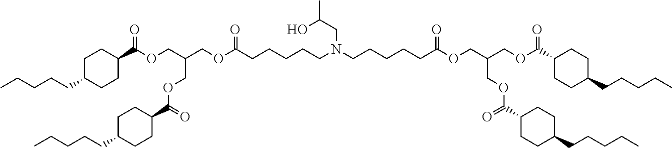 [SMILES: CCCCC[C@H]1CC[C@H](C(=O)OCC(COC(=O)CCCCCN(CCCCCC(=O)OCC(COC(=O)[C@H]2CC[C@H](CCCCC)CC2)COC(=O)[C@H]2CC[C@H](CCCCC)CC2)CC(C)O)COC(=O)[C@H]2CC[C@H](CCCCC)CC2)CC1] |
| 56 | 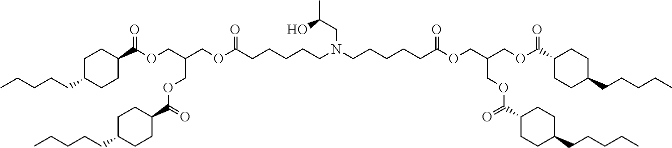 [SMILES: CCCCC[C@H]1CC[C@H](C(=O)OCC(COC(=O)CCCCCN(CCCCCC(=O)OCC(COC(=O)[C@H]2CC[C@H](CCCCC)CC2)COC(=O)[C@H]2CC[C@H](CCCCC)CC2)C[C@H](C)O)COC(=O)[C@H]2CC[C@H](CCCCC)CC2)CC1] |
| 57 | 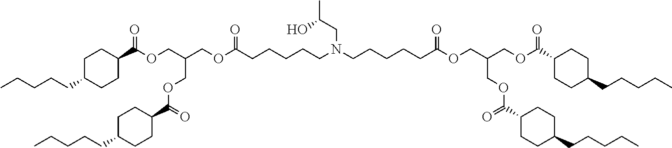 [SMILES: CCCCC[C@H]1CC[C@H](C(=O)OCC(COC(=O)CCCCCN(CCCCCC(=O)OCC(COC(=O)[C@H]2CC[C@H](CCCCC)CC2)COC(=O)[C@H]2CC[C@H](CCCCC)CC2)C[C@@H](C)O)COC(=O)[C@H]2CC[C@H](CCCCC)CC2)CC1] |
| 58 | 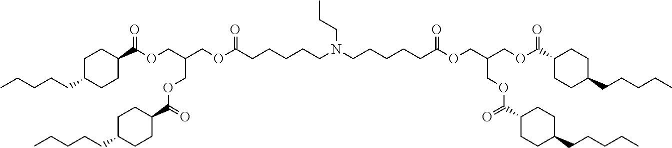 [SMILES: CCCCC[C@H]1CC[C@H](C(=O)OCC(COC(=O)CCCCCN(CCC)CCCCCC(=O)OCC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1] |
| 59 | 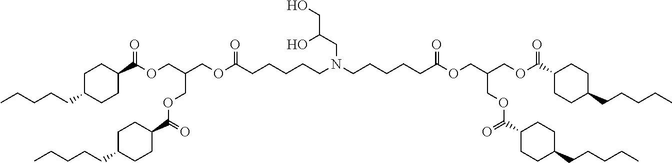 [SMILES: CCCCC[C@H]1CC[C@H](C(=O)OCC(COC(=O)CCCCCN(CCCCCC(=O)OCC(COC(=O)[C@H]2CC[C@H](CCCCC)CC2)COC(=O)[C@H]2CC[C@H](CCCCC)CC2)CC(O)CO)COC(=O)[C@H]2CC[C@H](CCCCC)CC2)CC1] |
| 60 | 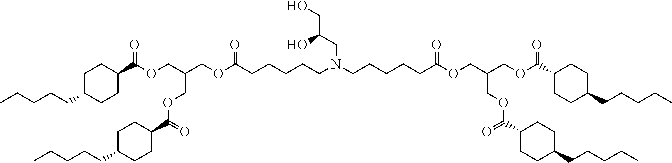 [SMILES: CCCCC[C@H]1CC[C@H](C(=O)OCC(COC(=O)CCCCCN(CCCCCC(=O)OCC(COC(=O)[C@H]2CC[C@H](CCCCC)CC2)COC(=O)[C@H]2CC[C@H](CCCCC)CC2)C[C@@H](O)CO)COC(=O)[C@H]2CC[C@H](CCCCC)CC2)CC1] |
| 61 | 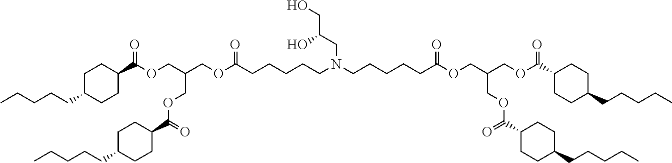 [SMILES: CCCCC[C@H]1CC[C@H](C(=O)OCC(COC(=O)CCCCCN(CCCCCC(=O)OCC(COC(=O)[C@H]2CC[C@H](CCCCC)CC2)COC(=O)[C@H]2CC[C@H](CCCCC)CC2)C[C@H](O)CO)COC(=O)[C@H]2CC[C@H](CCCCC)CC2)CC1] |
| 62 | 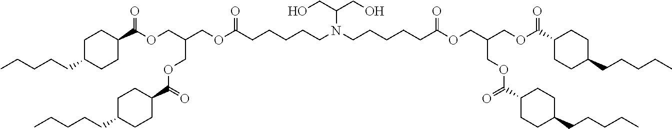 [SMILES: CCCCC[C@H]1CC[C@H](C(=O)OCC(COC(=O)CCCCCN(CCCCCC(=O)OCC(COC(=O)[C@H]2CC[C@H](CCCCC)CC2)COC(=O)[C@H]2CC[C@H](CCCCC)CC2)C(CO)CO)COC(=O)[C@H]2CC[C@H](CCCCC)CC2)CC1] |
| 63 | 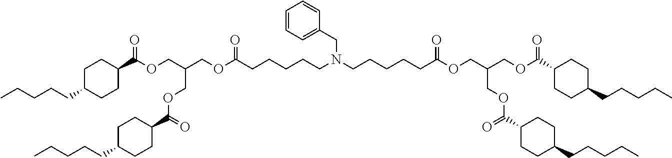 [SMILES: CCCCC[C@H]1CC[C@H](C(=O)OCC(COC(=O)CCCCCN(CCCCCC(=O)OCC(COC(=O)[C@H]2CC[C@H](CCCCC)CC2)COC(=O)[C@H]2CC[C@H](CCCCC)CC2)Cc2ccccc2)COC(=O)[C@H]2CC[C@H](CCCCC)CC2)CC1] |
| 64 | 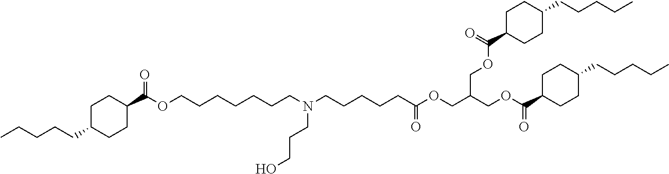 [SMILES: CCCCC[C@H]1CC[C@H](C(=O)OCCCCCCCN(CCCO)CCCCCC(=O)OCC(COC(=O)[C@H]2CC[C@H](CCCCC)CC2)COC(=O)[C@H]2CC[C@H](CCCCC)CC2)CC1] |
| 65 | 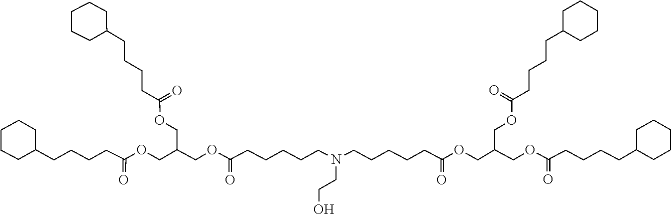 [SMILES: O=C(CCCCCN(CCO)CCCCCC(=O)OCC(COC(=O)CCCCC1CCCCC1)COC(=O)CCCCC1CCCCC1)OCC(COC(=O)CCCCC1CCCCC1)COC(=O)CCCCC1CCCCC1] |
| 66 | 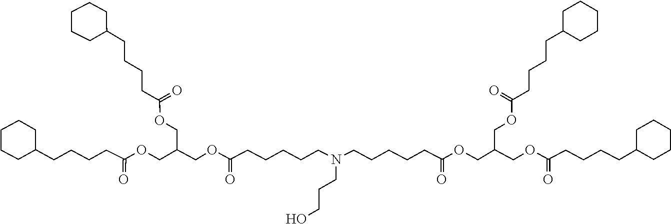 [SMILES: O=C(CCCCCN(CCCO)CCCCCC(=O)OCC(COC(=O)CCCCC1CCCCC1)COC(=O)CCCCC1CCCCC1)OCC(COC(=O)CCCCC1CCCCC1)COC(=O)CCCCC1CCCCC1] |
| 67 | 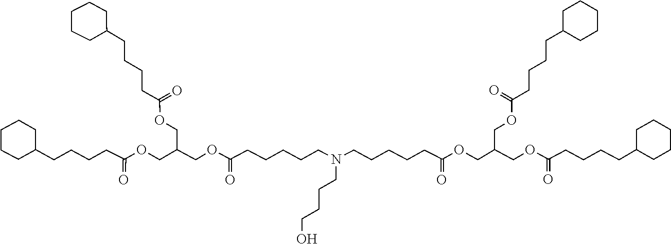 [SMILES: O=C(CCCCCN(CCCCO)CCCCCC(=O)OCC(COC(=O)CCCCC1CCCCC1)COC(=O)CCCCC1CCCCC1)OCC(COC(=O)CCCCC1CCCCC1)COC(=O)CCCCC1CCCCC1] |
| 68 | 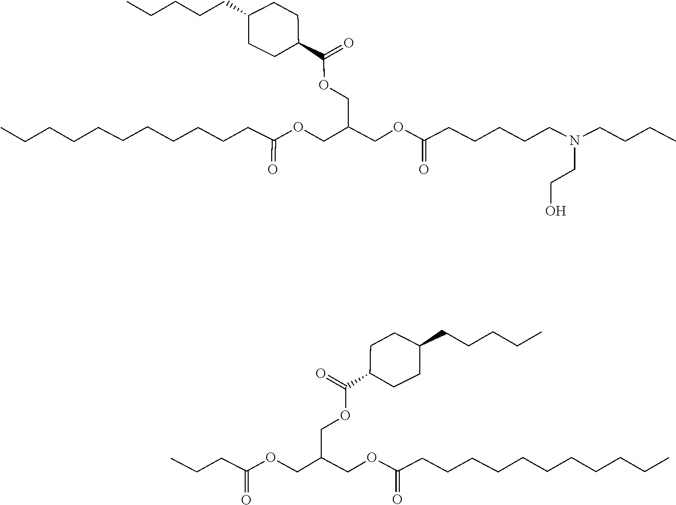 |
| 69 | 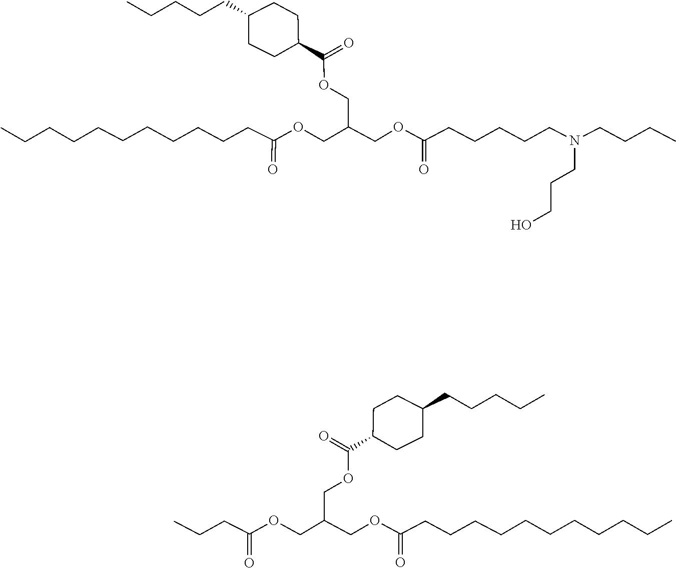 |
| 70 | 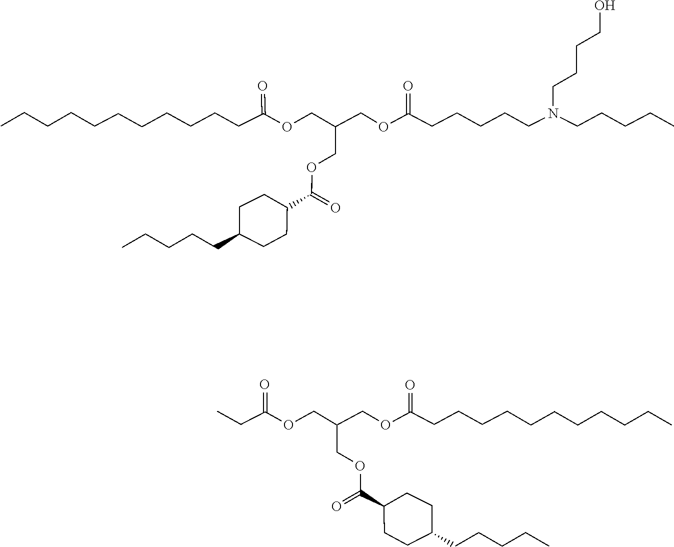 |
| 71 | 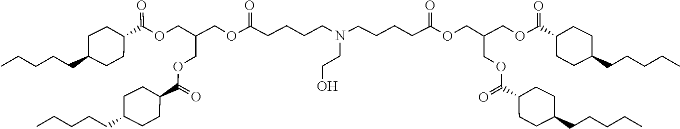 [SMILES: CCCCC[C@H]1CC[C@H](C(=O)OCC(COC(=O)CCCCN(CCO)CCCCC(=O)OCC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1] |
| 72 | 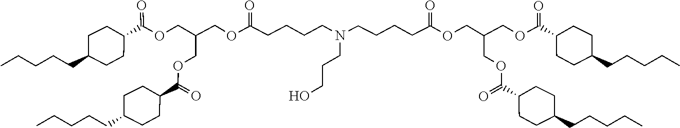 [SMILES: CCCCC[C@H]1CC[C@H](C(=O)OCC(COC(=O)CCCCN(CCCO)CCCCC(=O)OCC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1] |
| 73 | 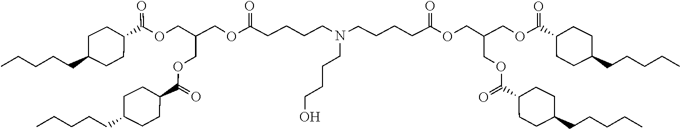 [SMILES: CCCCC[C@H]1CC[C@H](C(=O)OCC(COC(=O)CCCCN(CCCCO)CCCCC(=O)OCC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1] |
| 74 | 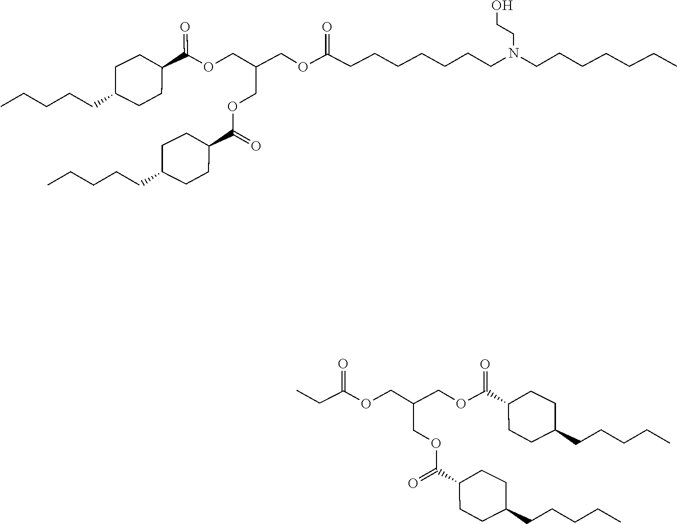 |
| 75 | 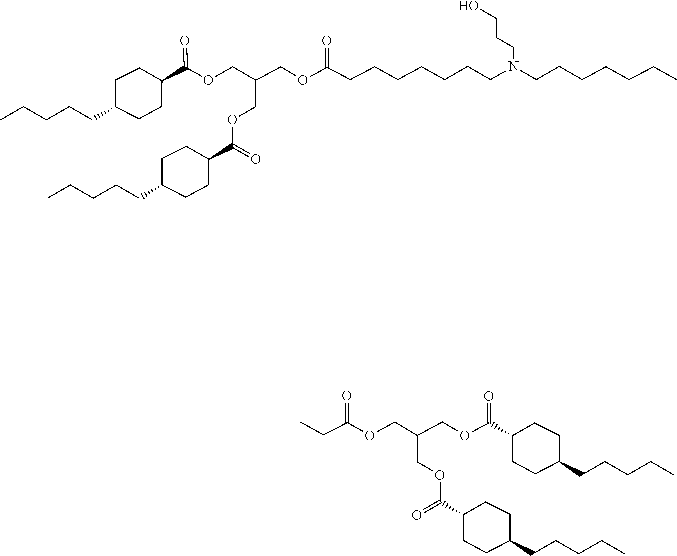 |
| 76 | 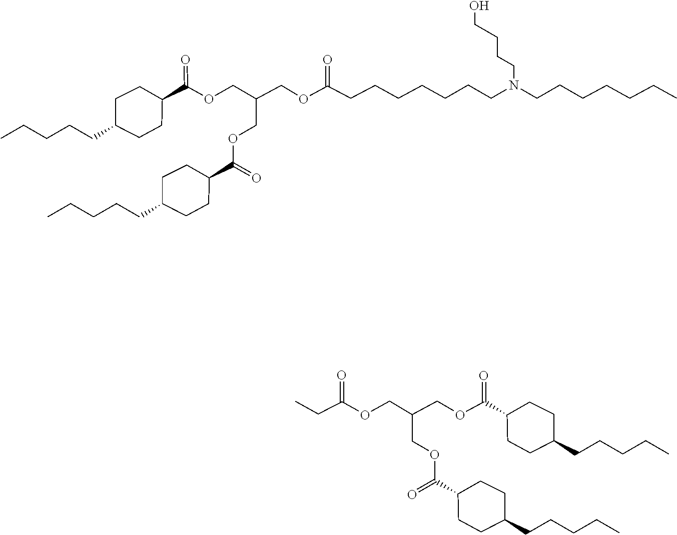 |

### Example 1—Preparation of Compound No. 1

COMPOUND NO. 1 was prepared in accordance with the General Scheme (A). The crude was purified by silica gel flash column chromatography with DCM/EtOAC. ¹H NMR (500 MHz, CDCl₃): δ 3.87 (d, 8H, J=9.8 Hz), 2.77 (t, 8H, J=9.8 Hz), 2.43 (t, 12H, J=4.8 Hz), 2.28 (m, 4H), 2.18 (s, 3H), 1.78-1.74 (m, 14H), 1.58-1.49 (m, 20H), 1.31-1.22 (m, 16H), 1.16-1.11 (m, 14H), 0.97-0.88 (m, 8H), 0.86 (d, 24H, J=9.8 Hz). MS: m/z 1155.2 (M+H).

### Example 2—Preparation of Compound No. 2

COMPOUND NO. 2 was prepared in accordance with the General Scheme (A). The crude was purified by silica gel flash column chromatography with DCM/EtOAC. ¹H NMR (500 MHz, CDCl₃): δ 3.87 (d, 8H, J=6 Hz), 2.77 (t, 8H, J=6 Hz), 2.43 (t, 12H, J=4.8 Hz), 2.19 (m, 4H), 2.18 (s, 3H), 1.77-1.61 (m, 30H), 1.25-1.19 (m, 20H), 1.18-1.12 (m, 32H), 0.98-0.82 (m, 24H). MS: m/z 1371.2 (M+H).

### Example 3—Preparation of Compound No. 3

COMPOUND NO. 3 was prepared in accordance with the General Scheme (C). The crude was purified by silica gel flash column chromatography with 4% MeOH/CH₂Cl₂. Pale brown oil, 0.91 g; Yield: 85%; ¹H NMR (499 MHz, CDCl₃) δ 4.25 (s, 16H), 2.76 (t, J=7.2 Hz, 8H), 2.46 (t, J=7.3 Hz, 11H), 2.35-2.12 (m, 10H), 1.98-1.92 (m, 8H), 1.83-1.77 (m, 8H), 1.66-1.53 (m, 6H), 1.40 (qd, J=13.3, 3.4 Hz, 8H), 1.32-1.14 (m, 36H), 0.96-0.84 (m, 20H).

### Example 4—Preparation of Compound No. 4

COMPOUND NO. 4 was prepared in accordance with the General Scheme (C). The crude was purified by silica gel flash column chromatography with 4% MeOH/CH₂Cl₂. Pale brown oil, 0.80 g; Yield: 67%; ¹H NMR (499 MHz, CDCl₃) δ 4.25 (s, 16H), 2.79 (t, J=7.2 Hz, 8H), 2.59-2.38 (m, 15H), 2.30-2.15 (m, 7H), 1.99-1.91 (m, 8H), 1.84-1.77 (m, 8H), 1.40 (qd, J=13.1, 3.5 Hz, 8H), 1.34-1.12 (m, 37H), 0.97-0.82 (m, 20H).

### Example 5—Preparation of Compound No. 5

COMPOUND NO. 5 was prepared in accordance with the General Scheme (C). The crude was purified by silica gel flash column chromatography with 4% MeOH/CH₂Cl₂. Pale brown oil, 0.136 mg; Yield: 60%; ¹H NMR (499 MHz, CDCl₃) δ 4.26 (s, 16H), 2.77 (t, J=7.2 Hz, 8H), 2.50-2.40 (m, 12H), 2.36-2.25 (m, 12H), 2.18 (s, 3H), 1.71-1.55 (m, 33H), 1.35-1.28 (m, 8H), 1.23-1.12 (m, 23H), 0.89-0.80 (m, 8H).

### Example 6—Preparation of Compound No. 6

COMPOUND NO. 6 was prepared in accordance with the General Scheme (C). The crude was purified by silica gel flash column chromatography with 4% MeOH/CH₂Cl₂. Pale brown oil, 0.101 mg; Yield: 45%; ¹H NMR (499 MHz, CDCl₃) δ 4.26 (s, 16H), 2.79 (t, J=7.2 Hz, 8H), 2.58-2.40 (m, 15H), 2.32 (t, J=7.5 Hz, 8H), 2.22 (s, 3H), 1.71-1.56 (m, 29H), 1.34-1.10 (m, 32H), 0.89-0.80 (m, 8H).

### Example 7—Preparation of Compound No. 7

COMPOUND NO. 7 was prepared in accordance with the General Scheme (D). The crude was purified by column chromatography (MeOH/DCM). ¹H NMR (499 MHz, CDCl₃) δ 3.98 (d, J=7.2 Hz, 5H), 3.89 (dd, J=6.6, 2.2 Hz, 11H), 2.77 (t, J=7.4 Hz, 8H), 2.43 (t, J=7.3 Hz, 12H), 2.28 (t, J=7.5 Hz, 12H), 2.17 (s, 3H), 1.85-1.78 (m, 2H), 1.67-1.49 (m, 34H), 1.46-1.36 (m, 6H), 1.23-1.15 (m, 10H), 1.06-0.94 (m, 12H), 0.88 (d, J=6.6 Hz, 24H). MS found 1386.8 [M+H]⁺, calcd for [C79H139N3O16=1386.02].

### Example 8—Preparation of Compound No. 8

COMPOUND NO. 8 was prepared in accordance with the General Scheme (D). The crude was purified by column chromatography (MeOH/DCM). ¹H NMR (500 MHz, CDCl₃) δ 3.98 (d, J=7.2 Hz, 5H), 3.89 (dd, J=6.6, 1.8 Hz, 11H), 2.77 (t, J=7.3 Hz, 8H), 2.43 (t, J=7.3 Hz, 12H), 2.29 (q, J=7.1 Hz, 12H), 2.17 (s, 3H), 1.85-1.75 (m, 14H), 1.61 (h, J=7.6 Hz, 18H), 1.58-1.49 (m, 4H), 1.45-1.37 (m, 4H), 1.31 (ddt, J=11.2, 8.0, 5.3 Hz, 16H), 1.06-0.93 (m, 12H), 0.89 (t, J=6.9 Hz, 12H). MS found 1330.5 [M+H]⁺, calcd for [C75H131N3O16=1329.95].

### Example 9—Preparation of Compound No. 9

COMPOUND NO. 9 was prepared in accordance with the General Scheme (D). The crude was purified by column chromatography (MeOH/DCM). ¹H NMR (500 MHz, CDCl₃) δ 3.98 (d, J=7.2 Hz, 5H), 3.89 (dd, J=6.5, 1.8 Hz, 11H), 2.76 (t, J=7.3 Hz, 8H), 2.43 (t, J=7.2 Hz, 12H), 2.30 (q, J=9.8 Hz, 12H), 1.94 (s, 3H), 1.83-1.76 (m, 12H), 1.64-1.56 (m, 18H), 1.59-1.50 (m, 5H, 1.45-1.37 (m, 5H), 1.34-1.22 (m, 40H), 1.06-0.93 (m, 12H), 0.88 (t, J=6.9 Hz, 12H). MS found 1498.6 [M+H]⁺, calcd for [C87H155N3O16=1498.14].

### Example 10—Preparation of Compound No. 10

COMPOUND NO. 10 was prepared in accordance with the General Scheme (D). The crude was purified by column chromatography (MeOH/DCM). ¹H NMR (500 MHz, CDCl₃) δ 4.05 (td, J=6.8, 2.1 Hz, 16H), 2.76 (t, J=7.3 Hz, 8H), 2.43 (td, J=7.3, 3.0 Hz, 12H), 2.28 (dd, J=8.9, 5.8 Hz, 4H), 2.17 (d, J=7.0 Hz, 12H), 1.80-1.73 (m, 3H), 1.73-1.65 (m, 16H), 1.64-1.55 (m, 24H), 1.32 (s, 32H), 1.37-1.20 (m, 8H), 1.20-1.08 (m, 4H), 1.02-0.90 (m, 8H). MS found 1442.5 [M+H]⁺, calcd for [C83H147N3O16=1442.08].

### Example 11—Preparation of Compound No. 11

COMPOUND NO. 11 was prepared in accordance with the General Scheme (D). The crude was purified by column chromatography (MeOH/DCM). ¹H NMR (499 MHz, CDCl₃) δ 4.05 (td, J=6.8, 1.6 Hz, 16H), 2.77 (t, J=7.3 Hz, 8H), 2.43 (td, J=7.0, 2.2 Hz, 12H), 2.29 (q, J=7.0 Hz, 12H), 2.18 (s, 3H), 1.74-1.63 (m, 16H), 1.61-1.54 (m, 32H), 1.52 (dt, J=8.7, 7.0 Hz, 8H), 1.32 (m, 24H), 0.94-0.83 (m, 8H). MS found 1498.6 [M+H]⁺, calcd for [C87H155N3O16=1498.1].

### Example 12—Preparation of Compound No. 12

COMPOUND NO. 12 was prepared in accordance with the General Scheme (D). The crude was purified by column chromatography (MeOH/DCM). ¹H NMR (500 MHz, CDCl₃): δ 5.27-5.32 (m, 8H), 3.88 (d, 16H, J=9.8 Hz), 2.77 (t, 8H, J=12 Hz), 2.43 (t, 8H, J=9.8 Hz), 2.23 (t, 8H, J=9.8 Hz), 2.18 (s, 3H), 2.06-2.02 (dd, 8H, J=3 Hz, 9.8 Hz), 1.80-1.82 (m, 16H), 1.62-1.60 (m, 34H), 1.36-1.26 (m, 56H), 1.01-0.97 (m, 14H), 0.89 (d, 12H, J=3 Hz). MS: m/z 1988.1 (M+H).

### Example 13—Preparation of Compound No. 13

COMPOUND NO. 13 was prepared in accordance with the General Scheme (B).

### Example 14—Preparation of Compound No. 14

COMPOUND NO. 14 was prepared in accordance with the General Scheme (B).

### Example 15—Preparation of Compound No. 15

COMPOUND NO. 15 was prepared in accordance with the General Scheme (E).

Following the general protocol for amine alkylation described in General Scheme E.1, amine H2 (17 mg) was combined with C6C25C (300 mg) and DIPEA (200 μL) in THF/CH₃CN (1:1, 1.0 mL). After the reaction, the crude was purified by 4% MeOH/DCM eluants. Brown oil, 119 mg (58%); LC-MS: Rt 7.867 min, m/z calculated [M+H]: 738.55, found 738.4.

### Example 16—Preparation of Compound No. 16

COMPOUND NO. 16 was prepared in accordance with the General Scheme (E).

Following the general protocol for amine alkylation described in General Scheme E.1, amine H3 (17 mg) was combined with C6C25C (290 mg) and DIPEA (200 μL) in THF/CH₃CN (1:1, 1.0 mL). After the reaction, the crude was purified by 4% MeOH/DCM eluants. Brown oil, 134 mg (79%); LC-MS: Rt 7.883 min, m/z calculated [M+H]: 752.6, found 752.4.

### Example 17—Preparation of Compound No. 17

COMPOUND NO. 17 was prepared in accordance with the General Scheme (E).

Following the general protocol for amine alkylation described in General Scheme E.1, amine H4 (22 mg) was combined with C6C25C (310 mg) and DIPEA (200 μL) in THF/CH₃CN (1:1, 1.0 mL). After the reaction, the crude was purified by 4% MeOH/DCM eluants. Brown oil, 101 mg (53%); LC-MS: Rt 7.900 min, m/z calculated [M+H]: 766.58, found 766.4.

### Example 18—Preparation of Compound No. 18

COMPOUND NO. 18 was prepared in accordance with the General Scheme (E).

Following the general protocol for amine alkylation described in General Scheme E.1, amine H2 (16.7 mg) was combined with 5CC3 (192 mg) and DIPEA (110 μL) in THF/CH₃CN (1:1, 1.0 mL). After the reaction, the crude was purified by 4% MeOH/DCM eluants. Brown oil, 88 mg (60%); ¹H NMR (499 MHz, CDCl₃) δ 4.10 (t, J=6.3 Hz, 4H), 3.55 (t, J=5.3 Hz, 2H), 2.64-2.48 (m, 6H), 2.21 (tt, J=12.2, 3.6 Hz, 2H), 1.98-1.91 (m, 4H), 1.83-1.74 (m, 8H), 1.40 (qd, J=13.1, 3.4 Hz, 4H), 1.33-1.13 (m, 19H), 0.95-0.85 (m, 10H); LC-MS: Rt 7.617 min, m/z calculated [M+H]: 538.45, found 538.2.

### Example 19—Preparation of Compound No. 19

COMPOUND NO. 19 was prepared in accordance with the General Scheme (E).

Following the general protocol for amine alkylation described in General Scheme E.1, amine H3 (25.7 mg) was combined with 5CC3 (322 mg) and DIPEA (175 μL) in THF/CH₃CN (1:1, 1.0 mL). After the reaction, the crude was purified by 4% MeOH/DCM eluants. Brown oil, 108 mg (57%); LC-MS: Rt 7.583 min, m/z calculated [M+H]: 552.46, found 552.2.

### Example 20—Preparation of Compound No. 20

COMPOUND NO. 20 was prepared in accordance with the General Scheme (E).

Following the general protocol for amine alkylation described in General Scheme E.1, amine H4 (30.7 mg) was combined with 5CC3 (334 mg) and DIPEA (180 μL) in THF/CH₃CN (1:1, 1.0 mL). After the reaction, the crude was purified by 4% MeOH/DCM eluants. Brown oil, 134 mg (71%); LC-MS: Rt 7.583 min, m/z calculated [M+H]: 566.48, found 566.2.

### Example 21—Preparation of Compound No. 21

COMPOUND NO. 21 was prepared in accordance with the General Scheme (E).

Following the general protocol for amine alkylation described in General Scheme E.1, amine H2 (23 mg) was combined with 5CC7 (372 mg) and DIPEA (200 μL) in THF/CH₃CN (1:1, 1.0 mL). After the reaction, the crude was purified by 4% MeOH/DCM eluants. Brown oil, 161 mg (72%); LC-MS: Rt 7.983 min, m/z calculated [M+H]: 594.51, found 594.4.

### Example 22—Preparation of Compound No. 22

COMPOUND NO. 22 was prepared in accordance with the General Scheme (E).

Following the general protocol for amine alkylation described in General Scheme E.1, amine H3 (29 mg) was combined with 5CC7 (413 mg) and DIPEA (200 μL) in THF/CH₃CN (1:1, 1.0 mL). After the reaction, the crude was purified by 4% MeOH/DCM eluants. Brown oil, 154 mg (66%); LC-MS: Rt 8.000 min, m/z calculated [M+H]: 608.52, found 608.2.

### Example 23—Preparation of Compound No. 23

COMPOUND NO. 23 was prepared in accordance with the General Scheme (E).

Following the general protocol for amine alkylation described in General Scheme E.1, amine H4 (28 mg) was combined with 5CC7 (363 mg) and DIPEA (200 μL) in THF/CH₃CN (1:1, 1.0 mL). After the reaction, the crude was purified by 4% MeOH/DCM eluants. Brown oil, 122 mg (62%); LC-MS: Rt 8.000 min, m/z calculated [M+H]: 622.54, found 622.2.

### Example 24—Preparation of Compound No. 24

COMPOUND NO. 24 was prepared in accordance with the General Scheme (E).

Following the general protocol for amine alkylation described in General Scheme E.1, amine H2 (25 mg) was combined with 5CC7 (370 mg) and DIPEA (200 μL) in THF/CH₃CN (1:1, 1.0 mL). After the reaction, the crude was purified by 4% MeOH/DCM eluants. Brown oil, 161 mg (61%); LC-MS: Rt 8.283 min, m/z calculated [M+H]: 650.57, found 650.4.

### Example 25—Preparation of Compound No. 25

COMPOUND NO. 25 was prepared in accordance with the General Scheme (E).

Following the general protocol for amine alkylation described in General Scheme E.1, amine H3 (28 mg) was combined with 5CC7 (399 mg) and DIPEA (200 μL) in THF/CH₃CN (1:1, 1.0 mL). After the reaction, the crude was purified by 4% MeOH/DCM eluants. Brown oil, 180 mg (73%); LC-MS: Rt 8.267 min, m/z calculated [M+H]: 664.59, found 664.4.

### Example 26—Preparation of Compound No. 26

COMPOUND NO. 26 was prepared in accordance with the General Scheme (E).

Following the general protocol for amine alkylation described in General Scheme E.1, amine H4 (27 mg) was combined with 5CC7 (332 mg) and DIPEA (200 μL) in THF/CH₃CN (1:1, 1.0 mL). After the reaction, the crude was purified by 4% MeOH/DCM eluants. Brown oil, 142 mg (69%); LC-MS: Rt 8.267 min, m/z calculated [M+H]: 678.6, found 679.0.

### Example 27—Preparation of Compound No. 27

COMPOUND NO. 27 was prepared in accordance with the General Scheme (E).

Following the general protocol for amine alkylation described in General Scheme E.1, amine H2 (19 mg) was combined with C6CyO_{6,10} (470 mg) and DIPEA (200 μL) in THF/CH₃CN (1:1, 1.0 mL). After the reaction, the crude was purified by 4% MeOH/DCM eluants. Brown oil, 210 mg (70%); LC-MS: Rt 9.383 min, m/z calculated [M+H]: 962.80, found 963.0.

### Example 28—Preparation of Compound No. 28

COMPOUND NO. 28 was prepared in accordance with the General Scheme (E).

Following the general protocol for amine alkylation described in General Scheme E.1, amine H3 (15 mg) was combined with C6CyO_{6,10} (304 mg) and DIPEA (200 μL) in THF/CH₃CN (1:1, 1.0 mL). After the reaction, the crude was purified by 4% MeOH/DCM eluants. Brown oil, 125 mg (64%); LC-MS: Rt 9.383 min, m/z calculated [M+H]: 976.82, found 977.0.

### Example 29—Preparation of Compound No. 29

COMPOUND NO. 29 was prepared in accordance with the General Scheme (E).

Following the general protocol for amine alkylation described in General Scheme E.1, amine H4 (16 mg) was combined with C6CyO_{6,10} (275 mg) and DIPEA (200 μL) in THF/CH₃CN (1:1, 1.0 mL). After the reaction, the crude was purified by 4% MeOH/DCM eluants. Brown oil, 92 mg (52%); LC-MS: Rt 9.583 min, m/z calculated [M+H]: 990.83, found 991.0.

### Example 30—Preparation of Compound No. 30

COMPOUND NO. 30 was prepared in accordance with the General Scheme (E).

Following the general protocol for amine alkylation described in General Scheme E.1, amine H2 (11 mg) was combined with C5Cy_{8,9} (289 mg) and DIPEA (150 μL) in THF/CH₃CN (1:1, 0.8 mL). After the reaction, the crude was purified by 4% MeOH/DCM eluants. Brown oil, 92 mg (50%); LC-MS: Rt 9.583 min, m/z calculated [M+H]: 1018.86, found 1019.0.

### Example 31—Preparation of Compound No. 31

COMPOUND NO. 31 was prepared in accordance with the General Scheme (E).

Following the general protocol for amine alkylation described in General Scheme E.1, amine H3 (10 mg) was combined with C5Cy_{8,9} (254 mg) and DIPEA (150 μL) in THF/CH₃CN (1:1, 0.8 mL). After the reaction, the crude was purified by 4% MeOH/DCM eluants. Brown oil, 71 mg (52%); LC-MS: Rt 9.533 min, m/z calculated [M+H]: 1032.88, found 1033.0.

### Example 32—Preparation of Compound No. 32

COMPOUND NO. 32 was prepared in accordance with the General Scheme (E).

Following the general protocol for amine alkylation described in General Scheme E.1, amine H4 (16 mg) was combined with C5Cy_{8,9} (283 mg) and DIPEA (150 μL) in THF/CH₃CN (1:1, 0.8 mL). After the reaction, the crude was purified by 4% MeOH/DCM eluants. Brown oil, 124 mg (65%); LC-MS: Rt 9.567 min, m/z calculated [M+H]: 1046.89, found 1047.0.

### Example 33—Preparation of Compound No. 33

COMPOUND NO. 33 was prepared in accordance with the General Scheme (E).

Following the general protocol for amine alkylation described in General Scheme E.1, H2NHC75C (70 mg) was combined with C6CyO_{6,10} (160 mg) and DIPEA (150 μL) in THF/CH₃CN (1:1, 0.8 mL). After the reaction, the crude was purified by 4% MeOH/DCM eluants. Brown oil, 96 mg (62%); LC-MS: Rt 9.000 min, m/z calculated [M+H]: 806.69, found 807.0.

### Example 34—Preparation of Compound No. 34

COMPOUND NO. 34 was prepared in accordance with the General Scheme (E).

Following the general protocol for amine alkylation described in General Scheme E.1, H2NHC75C (71 mg) was combined with C5CyO_{8,9} (162 mg) and DIPEA (150 μL) in THF/CH₃CN (1:1, 0.8 mL). After the reaction, the crude was purified by 4% MeOH/DCM eluants. Brown oil, 102 mg (65%); LC-MS: Rt 9.133 min, m/z calculated [M+H]: 835.73, found 835.8.

### Example 35—Preparation of Compound No. 35

COMPOUND NO. 35 was prepared in accordance with the General Scheme (E).

Following the general protocol for amine alkylation described in General Scheme E.1, amine 404 (19 mg) was combined with 5CC3 (264 mg) and DIPEA (120 μL) in THF/CH₃CN (1:1, 1.0 mL). After the reaction, the crude was purified by 4% MeOH/DCM eluants. Colorless oil, 23 mg (16%); LC-MS: Rt 7.583 min, m/z calculated [M+H]: 1098.95, found 1099.

### Example 36—Preparation of Compound No. 36

COMPOUND NO. 36 was prepared in accordance with the General Scheme (E).

Following the general protocol for amine alkylation described in General Scheme E.1, amine H2 (12 mg) was combined with BC6B5C (292 mg) and DIPEA (200 μL) in THF/CH₃CN (1:1, 0.8 mL). After the reaction, the crude was purified by 6% MeOH/DCM eluants. Brown oil, 142 mg (61%); ¹H NMR (499 MHz, CDCl₃) δ 4.12 (t, J=5.5 Hz, 12H), 3.55 (s, 2H), 2.61 (s, 2H), 2.49 (s, 3H), 2.39 (p, J=6.0 Hz, 2H), 2.32 (t, J=7.5 Hz, 4H), 2.22 (tt, J=12.2, 3.6 Hz, 4H), 1.98-1.91 (m, 8H), 1.80 (dt, J=15.1, 3.2 Hz, 8H), 1.64 (q, J=7.6 Hz, 4H), 1.53-1.13 (m, 53H), 0.88 (q, J=6.8 Hz, 20H); LC-MS: Rt 9.625 min, m/z calculated [M+H]: 1186.91, found 1186.80.

### Example 37—Preparation of Compound No. 37

COMPOUND NO. 37 was prepared in accordance with the General Scheme (E).

Following the general protocol for amine alkylation described in General Scheme E.1, amine H3 (22 mg) was combined with BC6B5C (442 mg) and DIPEA (200 μL) in THF/CH₃CN (1:1, 0.8 mL). After the reaction, the crude was purified by 6% MeOH/DCM eluants. Brown oil, 127 mg (36%); ¹H NMR (499 MHz, CDCl₃) δ 4.12 (t, J=4.8 Hz, 12H), 3.81-3.75 (m, 2H), 2.65 (s, 2H), 2.39 (td, J=12.8, 6.8 Hz, 5H), 2.32 (t, J=7.5 Hz, 4H), 2.22 (tt, J=12.2, 3.6 Hz, 4H), 1.98-1.91 (m, 8H), 1.84-1.76 (m, 8H), 1.72-1.60 (m, 7H), 1.50 (s, 4H), 1.44-1.13 (m, 49H), 0.95-0.84 (m, 20H); LC-MS: Rt 9.504 min, m/z calculated [M+H]: 1200.92, found 1200.75.

### Example 38—Preparation of Compound No. 38

COMPOUND NO. 38 was prepared in accordance with the General Scheme (E).

Following the general protocol for amine alkylation described in General Scheme E.1, amine H4 (10 mg) was combined with BC6B5C (179 mg) and DIPEA (100 μL) in THF/CH₃CN (1:1, 0.8 mL). After the reaction, the crude was purified by 6% MeOH/DCM eluants. Brown oil, 27 mg (20%); LC-MS: Rt 9.481 min, m/z calculated [M+H]: 1214.94, found 1214.75.

### Example 39—Preparation of Compound No. 39

COMPOUND NO. 39 was prepared in accordance with the General Scheme (E).

Following the general protocol for amine alkylation described in General Scheme E.1, amine N-(4-Aminobutyl) acetamide A4 (10 mg) was combined with B6B5C (117 mg) and DIPEA (60 μL) in THF/CH₃CN (1:1, 0.8 mL). After the reaction, the crude was purified by 8% MeOH/DCM eluants. Brown oil, 35 mg (36%); LC-MS: Rt 9.469 min, m/z calculated [M+H]: 1255.96, found 1255.75.

### Example 40—Preparation of Compound No. 40

COMPOUND NO. 40 was prepared in accordance with the General Scheme (B). ¹H NMR (500 MHz, CDCl₃) δ 4.12-3.91 (m, 13H), 3.89 (d, J=6.5 Hz, 6H), 3.43-3.33 (m, 2H), 3.13 (d, J=16.1 Hz, 6H), 2.29 (t, J=7.6 Hz, 8H), 1.96-1.71 (m, 22H), 1.68-1.47 (m, 19H), 1.46-1.24 (m, 22H), 1.06-0.94 (m, 11H), 0.89 (t, J=6.8 Hz, 12H). MS (ESI): calcd. for C₇₀H₁₁₉NO₁₇ [M+H]⁺ 1246.8, found 1247.0.

### Example 41—Preparation of Compound No. 41

COMPOUND NO. 41 was prepared in accordance with the General Scheme (B). ¹H NMR (500 MHz, CDCl₃) δ 4.13-3.92 (m, 11H), 3.89 (d, J=6.5 Hz, 6H), 3.43-3.33 (m, 3H), 3.14 (s, 3H), 2.94 (s, 2H), 2.76 (s, 3H), 2.29 (t, J=7.6 Hz, 8H), 1.97-1.73 (m, 23H), 1.67-1.57 (m, 14H), 1.53 (tq, J=8.3, 4.4 Hz, 6H), 1.41 (q, J=7.3 Hz, 7H), 1.37-1.25 (m, 17H), 1.06-0.94 (m, 12H), 0.93-0.85 (m, 12H). MS (ESI): calcd. for C₆₉H₁₁₇NO₁₆ [M+H]⁺ 1216.8, found 1216.6.

### Example 42—Preparation of Compound No. 42

COMPOUND NO. 42 was prepared in accordance with the General Scheme (B). ¹H NMR (500 MHz, CDCl₃) δ 4.05-3.83 (m, 9H), 3.38 (t, J=7.1 Hz, 2H), 3.22-2.99 (m, 5H), 1.91 (dd, J=10.4, 5.3 Hz, 4H), 1.89-1.66 (m, 19H), 1.54 (ddq, J=26.6, 13.3, 6.7 Hz, 9H), 1.39-1.22 (m, 10H), 1.24-1.08 (m, 19H), 1.01-0.88 (m, 12H), 0.86 (d, J=6.6 Hz, 26H). MS (ESI): calcd. for C₆₆H₁₁₉NO₉ [M+H]⁺ 1070.9, found 1071.1.

### Example 43—Preparation of Compound No. 43

COMPOUND NO. 43 was prepared in accordance with the General Scheme (B). ¹H NMR (500 MHz, CDCl₃) δ 3.99-3.86 (m, 6H), 3.35 (t, J=7.6 Hz, 2H), 2.38-2.29 (m, 4H), 2.15 (s, 3H), 1.88 (q, J=7.6 Hz, 4H), 1.81-1.69 (m, 11H), 1.64-1.43 (m, 29H), 1.26 (t, J=5.2 Hz, 8H), 1.21-1.08 (m, 14H), 1.02-0.88 (m, 8H), 0.86 (d, J=6.6 Hz, 18H). MS (ESI): calcd. for C₆₅H₁₁₇NO₈ [M+H]⁺ 1040.9, found 1040.9.

### Example 44—Preparation of Compound No. 44

COMPOUND NO. 44 was prepared in accordance with the General Scheme (B). ¹H NMR (500 MHz, CDCl₃) δ 4.12 (qt, J=10.8, 6.8 Hz, 4H), 3.08 (d, J=9.5 Hz, 2H), 1.91 (q, J=7.3 Hz, 11H), 1.79 (d, J=7.1 Hz, 2H), 1.73-1.55 (m, 14H), 1.33 (tdd, J=9.6, 7.1, 5.3 Hz, 4H), 1.28-1.07 (m, 12H), 0.86 (q, J=11.3 Hz, 4H). MS (ESI): calcd. for C₅₅H₉₇NO₉ [M+H]⁺ 916.7, found 917.0. MS (ESI): calcd. for C₅₅H₉₇NO₉ [M+H]⁺ 916.7, found 917.0.

### Example 45—Preparation of Compound No. 45

COMPOUND NO. 45 was prepared in accordance with the General Scheme (B). ¹H NMR (500 MHz, CDCl₃) δ 4.18-4.03 (m, 7H), 3.78 (s, 2H), 3.31 (t, J=7.5 Hz, 2H), 3.22 (s, 2H), 3.03 (d, J=10.0 Hz, 3H), 1.92-1.84 (m, 4H), 1.82-1.54 (m, 42H), 1.43-1.27 (m, 17H), 1.26-1.07 (m, 21H), 0.92-0.76 (m, 8H). MS (ESI): calcd. for C₆₁H₁₀₉NO₉ [M+H]⁺ 1000.8, found 1001.0.

### Example 46—Preparation of Compound No. 46

COMPOUND NO. 46 was prepared in accordance with the General Scheme (B). ¹H NMR (500 MHz, CDCl₃) δ 4.00-3.86 (m, 7H), 3.32 (td, J=7.5, 3.8 Hz, 2H), 3.17 (t, J=7.0 Hz, 2H), 1.88 (t, J=7.7 Hz, 5H), 1.84-1.65 (m, 39H), 1.59 (d, J=10.0 Hz, 4H), 1.52 (dq, J=13.2, 6.6 Hz, 5H), 1.43-1.31 (m, 12H), 1.31-1.20 (m, 23H), 1.16 (tt, J=9.1, 6.2 Hz, 18H), 1.04-0.89 (m, 11H), 0.86 (d, J=6.6 Hz, 25H). MS (ESI): calcd. for C₈₁H₁₄₉NO₉ [M+H]⁺ 1281.1, found 1281.5.

### Example 47—Preparation of Compound No. 47

COMPOUND NO. 47 was prepared in accordance with the General Scheme (B). ¹H NMR (500 MHz, CDCl₃) δ 4.01-3.87 (m, 7H), 3.79 (s, 1H), 3.42-3.30 (m, 2H), 3.24 (s, 1H), 3.18 (t, J=6.9 Hz, 1H), 3.10 (s, 2H), 2.54 (t, J=8.0 Hz, 2H), 1.89 (s, 26H), 1.75 (t, J=16.4 Hz, 17H), 1.63-1.45 (m, 8H), 1.32-1.10 (m, 36H), 1.04-0.89 (m, 10H), 0.86 (d, J=6.6 Hz, 24H). MS (ESI): calcd. for C₇₅H₁₃₇NO₃ [M+H]⁺ 1197.0, found 1197.5.

### Example 48—Preparation of Compound No. 48

COMPOUND NO. 48 was prepared in accordance with the General Scheme (B). MS (ESI): calcd. for C₇₅H₁₂₉NO₁₇ [M+H]⁺ 1316.9, found 1317.0.

### Example 49—Preparation of Compound No. 49

COMPOUND NO. 49 was prepared in accordance with the General Scheme (B). ¹H NMR (500 MHz, CDCl₃) δ 4.10-4.00 (m, 2H), 4.00-3.91 (m, 7H), 3.89 (d, J=6.5 Hz, 5H), 3.75 (t, J=5.2 Hz, 2H), 3.35 (t, J=7.4 Hz, 2H), 2.59 (s, 2H), 2.43 (t, J=7.6 Hz, 4H), 2.29 (t, J=7.5 Hz, 7H), 1.86 (q, J=7.7 Hz, 6H), 1.80 (dt, J=8.8, 4.4 Hz, 10H), 1.70-1.45 (m, 37H), 1.42 (dd, J=11.4, 4.7 Hz, 5H), 1.35-1.24 (m, 15H), 1.05-0.94 (m, 10H), 0.93-0.86 (m, 10H). MS (ESI): calcd. for C₇₁H₁₂₁NO₁₇ [M+H]⁺ 1261.7, found 1261.4.

### Example 50—Preparation of Compound No. 50

COMPOUND NO. 50 was prepared in accordance with the General Scheme (B). ¹H NMR (500 MHz, CDCl₃) δ 4.13-4.01 (m, 3H), 4.00-3.91 (m, 8H), 3.89 (d, J=6.5 Hz, 6H), 3.67 (s, 2H), 3.60 (dd, J=10.7, 6.7 Hz, 2H), 3.35 (t, J=7.5 Hz, 3H), 2.54 (s, 2H), 2.45 (s, 3H), 2.30 (t, J=7.6 Hz, 8H), 1.87 (d, J=8.3 Hz, 6H), 1.83-1.75 (m, 11H), 1.62 (p, J=7.4 Hz, 13H), 1.54 (d, J=16.6 Hz, 20H), 1.42 (dd, J=12.8, 6.6 Hz, 6H), 1.30 (qd, J=8.3, 4.6 Hz, 17H), 1.07-0.94 (m, 11H), 0.89 (t, J=6.9 Hz, 13H). MS (ESI): calcd. for C₇₂H₁₂₃ NO₁₉ [M+H]⁺ 1290.9, found 1291.2.

### Example 51—Preparation of Compound No. 51

COMPOUND NO. 51 was prepared in accordance with the General Scheme (E).

Following the general protocol for amine alkylation described in General Scheme E.1, 5-amino-1-pentanol (19 mg, 1.0 eq), BC6B5C (272 mg, 2.2 eq), K₂CO₃ (165 mg, 4.4 eq) and KI (62 mg, 1.0 eq) were combined and poured in CH₃CN/THF (1:1, 3 mL). The crude was purified by silica gel flash chromatography and the desired product was eluted with 4-5% MeOH/DCM eluants. Pale yellow oil, 27 mg (20%); LC-MS: Rt 9.481 min, m/z calculated [M+H]: 1228.95, found 1229.

### Example 52—Preparation of Compound No. 52

COMPOUND NO. 52 was prepared in accordance with the General Scheme (E).

Following the general protocol for amine alkylation described in General Scheme E.1, 6-amino-1-hexanol (20 mg, 1.0 eq), 65C (258 mg, 2.2 eq), K₂CO₃ (160 mg, 4.4 eq) and KI (65 mg, 1.0 eq) were combined and poured in CH₃CN/THF (1:1, 3 mL). Pale yellow oil, 42 mg (20%); LC-MS: Rt 9.4 min, m/z calculated [M+H]: 1242.97, found 1243.

### Example 53—Preparation of Compound No. 53

COMPOUND NO. 53 was prepared in accordance with the General Scheme (E).

Following the general protocol for amine alkylation described in General Scheme E.1, trans-4-aminocyclohexanol (13 mg, 1.0 eq), 65C (172, 2.2 eq), K₂CO₃ (172 mg, 4.4 eq) and KI (56 mg, 1.0 eq) were combined and poured in CH₃CN/THF (1:1, 3 mL). Pale brown oil, 45 mg (32%); LC-MS: Rt 9.4 min, m/z calculated [M+H]: 1240.95, found 1241.

### Example 54—Preparation of Compound No. 54

COMPOUND NO. 54 was prepared in accordance with the General Scheme (E).

Following the general protocol for amine alkylation described in General Scheme E.1, trans 4-aminocyclohexanemethanol (19 mg, 1.0 eq), 65C (207 mg, 2.2 eq), K₂CO₃ (164 mg, 4.4 eq) and KI (52 mg, 1.0 eq) were combined and poured in CH₃CN/THF (1:1, 3 mL). Pale brown oil, 68 mg (39%); LC-MS: Rt 9.4 min, m/z calculated [M+H]: 1254.97, found 1253.

### Example 55—Preparation of Compound No. 55

COMPOUND NO. 55 was prepared in accordance with the General Scheme (E).

Following the general protocol for amine alkylation described in General Scheme E.1, racemic 1-amino-2-propanol (15.5 mg, 1.0 eq), 65C (304 mg, 2.2 eq), K₂CO₃ (152 mg, 4.4 eq) and KI (44 mg, 1.0 eq) were combined and poured in CH₃CN/THF (1:1, 3 mL). Pale brown oil, 88 mg (35%); LC-MS: Rt 9.4 min, m/z calculated [M+H]: 1200.92, found 1201.

### Example 56—Preparation of Compound No. 56

COMPOUND NO. 56 was prepared in accordance with the General Scheme (E).

Following the general protocol for amine alkylation described in General Scheme E.1, S-1-amino-2-propanol (13 mg, 1.0 eq), 65C (271 mg, 2.2 eq), K₂CO₃ (163 mg, 4.4 eq) and KI (42 mg, 1.0 eq) were combined and poured in CH₃CN/THF (1:1, 3 mL). Pale brown oil, 61 mg (30%); LC-MS: Rt 9.4 min, m/z calculated [M+H]: 1200.92, found 1201.

### Example 57—Preparation of Compound No. 57

COMPOUND NO. 57 was prepared in accordance with the General Scheme (E).

Following the general protocol for amine alkylation described in General Scheme E.1, R-1-amino-2-propanol (17.2 mg, 1.0 eq), 65C (330 mg, 2.2 eq), K₂CO₃ (198 mg, 4.4 eq) and KI (66 mg, 1.0 eq) were combined and poured in CH₃CN/THF (1:1, 3 mL). Pale brown oil, 72 mg (26%); LC-MS: Rt 9.4 min, m/z calculated [M+H]: 1200.92, found 1201.

### Example 58—Preparation of Compound No. 58

COMPOUND NO. 58 was prepared in accordance with the General Scheme (E).

Following the general protocol for amine alkylation described in General Scheme E.1, n-propylamine (10.1 mg, 1.0 eq), 65C (247 mg, 2.2 eq), K₂CO₃ (158 mg, 4.4 eq) and KI (44 mg, 1.0 eq) were combined and poured in CH₃CN/THF (1:1, 3 mL). Pale yellow oil, 100 mg (50%); LC-MS: Rt 9.6 min, m/z calculated [M+H]: 1184.93, found 1185.

### Example 59—Preparation of Compound No. 59

COMPOUND NO. 59 was prepared in accordance with the General Scheme (E).

Following the general protocol for amine alkylation described in General Scheme E.1, racemic 3-amino-1,2-propanediol (15.6 mg, 1.0 eq), 65C (260 mg, 2.2 eq), K₂CO₃ (168 mg, 4.4 eq) and KI (55 mg, 1.0 eq) were combined and poured in CH₃CN/THF (1:1, 3 mL). Pale yellow oil, 74 mg (36%); LC-MS: Rt 9.3 min, m/z calculated [M+H]: 1216.92, found 1217.

### Example 60—Preparation of Compound No. 60

COMPOUND NO. 60 was prepared in accordance with the General Scheme (E).

Following the general protocol for amine alkylation described in General Scheme E.1, S-3-amino-1,2-propanediol (16 mg, 1.0 eq), 65C (269 mg, 2.2 eq), K₂CO₃ (155 mg, 4.4 eq) and KI (63 mg, 1.0 eq) were combined and poured in CH₃CN/THF (1:1, 3 mL). Pale yellow oil, 51 mg (24%); LC-MS: Rt 9.3 min, m/z calculated [M+H]: 1216.92, found 1217.

### Example 61—Preparation of Compound No. 61

COMPOUND NO. 61 was prepared in accordance with the General Scheme (E).

Following the general protocol for amine alkylation described in General Scheme E.1, R-3-amino-1,2-propanediol (16 mg, 1.0 eq), 65C (261 mg, 2.2 eq), K₂CO₃ (177 mg, 4.4 eq) and KI (66 mg, 1.0 eq) were combined and poured in CH₃CN/THF (1:1, 3 mL). Pale yellow oil, 74 mg (36%); LC-MS: Rt 9.3 min, m/z calculated [M+H]: 1216.92, found 1217.

### Example 62—Preparation of Compound No. 62

COMPOUND NO. 62 was prepared in accordance with the General Scheme (E).

Following the general protocol for amine alkylation described in General Scheme E.1, serinol (29 mg, 1.0 eq), 65C (474 mg, 2.2 eq), K₂CO₃ (227 mg, 4.4 eq) and KI (109 mg, 1.0 eq) were combined and poured in CH₃CN/THF (1:1, 3 mL). Pale yellow oil, 53 mg (14%); LC-MS: Rt 9.4 min, m/z calculated [M+H]: 1216.92, found 1217.

### Example 63—Preparation of Compound No. 63

COMPOUND NO. 63 was prepared in accordance with the General Scheme (E).

Following the general protocol for amine alkylation described in General Scheme E.1, benzylamine (91 mg, 1.0 eq), 65C (1.15 g, 2.2 eq), K₂CO₃ (593 mg, 4.4 eq) and KI (282 mg, 1.0 eq) were combined and poured in CH₃CN/THF (1:1, 3 mL). Pale yellow oil, 252 mg (24%); LC-MS: Rt 9.4 min, m/z calculated [M+H]: 1232.93, found 1233.

### Example 64—Preparation of Compound No. 64

COMPOUND NO. 64 was prepared in accordance with the General Scheme (E). Pale yellow oil. 62 mg (20%): ¹H NMR (499 MHz. CDCl₃) δ 4.12 (t. J=5.5 Hz. 6H), 4.04 (t. J=6.7 Hz. 2H). 3.81-3.76 (m, 2H), 2.65 (t. J=5.7 Hz, 2H). 2.47-2.36 (m. 5H). 2.32 (t. J=7.6 Hz, 2H). 2.27-2.16 (m, 3H). 1.99-1.90 (m. 6H). 1.84-1.76 (m. 6H). 1.72-1.57 (m. 7H), 1.54-1.12 (m. 45H). 0.94-0.84 (m. 14H). LC-MS: Rt 9.4 min, m/z calculated [M+H]: 932.75. found 933.

### Example 65—Preparation of Compound No. 65

COMPOUND NO. 65 was prepared in accordance with the General Scheme (E). Pale yellow oil, 77 mg (25%); LC-MS: Rt 9.4 min, m/z calculated [M+H]: 1130.84, found 1131.

### Example 66—Preparation of Compound No. 66

COMPOUND NO. 66 was prepared in accordance with the General Scheme (E). In a 20 mL scintillation glass vial, H3 (18.7 mg, 1.0 eq), 6C4 (350 mg, 2.2 eq), K₂CO₃ (184 mg, 4.4 eq) and KI (88 mg, 1.0 eq) were combined and poured in CH₃CN/THF (1:1, 3 mL). Pale yellow oil, 37 mg (14%); LC-MS: Rt 9.4 min, m/z calculated [M+H]: 1144.86, found 1145.

### Example 67—Preparation of Compound No. 67

COMPOUND NO. 67 was prepared in accordance with the General Scheme (E). In a 20 mL scintillation glass vial, H4 (15.4 mg, 1.0 eq), 6C4 (237 mg, 2.2 eq), K₂CO₃ (128 mg, 4.4 eq) and KI (60 mg, 1.0 eq) were combined and poured in CH₃CN/THF (1:1, 3 mL). Pale yellow oil, 63 mg (32%); LC-MS: Rt 9.4 min, m/z calculated [M+H]: 1158.88, found 1159.

### Example 68—Preparation of Compound No. 68

COMPOUND NO. 68 was prepared in accordance with the General Scheme (E). Pale yellow oil, 59 mg (27%); LC-MS: Rt 9.4 min, m/z calculated [M+H]: 1190.94, found 1191.

### Example 69—Preparation of Compound No. 69

COMPOUND NO. 69 was prepared in accordance with the General Scheme (E). In a 20 mL scintillation glass vial, H3 (16 mg, 1.0 eq), 6C125C (295 mg, 2.2 eq), K₂CO₃ (213 mg, 4.4 eq) and KI (85 mg, 1.0 eq) were combined and poured in CH₃CN/THF (1:1, 3 mL). Pale yellow oil, 47 mg (18%); LC-MS: Rt 9.4 min, m/z calculated [M+H]: 1204.95, found 1205.

### Example 70—Preparation of Compound No. 70

COMPOUND NO. 70 was prepared in accordance with the General Scheme (E). In a 20 mL scintillation glass vial, H4 (15.6 mg, 1.0 eq), 6C125C (246 mg, 2.2 eq), K₂CO₃ (160 mg, 4.4 eq) and KI (83 mg, 1.0 eq) were combined and poured in CH₃CN/THF (1:1, 3 mL). Pale yellow oil, 90 mg (42%); LC-MS: Rt 9.4 min, m/z calculated [M+H]: 1218.97, found 1219.

### Example 71—Preparation of Compound No. 71

COMPOUND NO. 71 was prepared in accordance with the General Scheme (E). Pale yellow oil, 71 mg (26%); LC-MS: Rt 9.4 min, m/z calculated [M+H]: 1158.88, found 1159.

### Example 72—Preparation of Compound No. 72

COMPOUND NO. 72 was prepared in accordance with the General Scheme (E). In a 20 mL scintillation glass vial, H3 (16.9 mg, 1.0 eq), 55C (300 mg, 2.2 eq), K₂CO₃ (156 mg, 4.4 eq) and KI (38 mg, 1.0 eq) were combined and poured in CH₃CN/THF (1:1, 3 mL). Pale yellow oil, 79 mg (30%); LC-MS: Rt 9.4 min, m/z calculated [M+H]: 1172.89, found 1173.

### Example 73—Preparation of Compound No. 73

COMPOUND NO. 73 was prepared in accordance with the General Scheme (E). In a 20 mL scintillation glass vial, H4 (19.7 mg, 1.0 eq), 55C (302 mg, 2.2 eq), K₂CO₃ (155 mg, 4.4 eq) and KI (37 mg, 1.0 eq) were combined and poured in CH₃CN/THF (1:1, 3 mL). Pale yellow oil, 102 mg (38%); LC-MS: Rt 9.4 min, m/z calculated [M+H]: 1186.91, found 1187.

### Example 74—Preparation of Compound No. 74

COMPOUND NO. 74 was prepared in accordance with the General Scheme (E). Pale yellow oil, 22 mg (10%); LC-MS: Rt 9.4 min, m/z calculated [M+H]: 1242.97, found 1243.

### Example 75—Preparation of Compound No. 75

COMPOUND NO. 75 was prepared in accordance with the General Scheme (E). In a 20 mL scintillation glass vial, H3 (15.6 mg, 1.0 eq), 85C (313 mg, 2.2 eq), K₂CO₃ (147 mg, 4.4 eq) and KI (35 mg, 1.0 eq) were combined and poured in CH₃CN/THF (1:1, 3 mL). Pale yellow oil, 42 mg (18%); LC-MS: Rt 9.4 min, m/z calculated [M+H]: 1256.98, found 1257.

### Example 76—Preparation of Compound No. 76

COMPOUND NO. 76 was prepared in accordance with the General Scheme (E). In a 20 mL scintillation glass vial, H4 (18.1 mg, 1.0 eq), 85C (311 mg, 2.2 eq), K₂CO₃ (158 mg, 4.4 eq) and KI (37 mg, 1.0 eq) were combined and poured in CH₃CN/THF (1:1, 3 mL). Pale yellow oil, 83 mg (32%); LC-MS: Rt 9.4 min, m/z calculated [M+H]: 1271.00, found 1271.

### Example 77—Preparation of LNPs of Present Disclosure Comprising DNA and In Vivo Screening

The following is a nonlimiting example that provides exemplary methods for formulating a plurality of multi-component LNP compositions comprising exemplary compounds of Formula (I) and DNA.

To formulate the LNPs, one of COMPOUND NOS. 1-14, the phospholipid DOPC, the structural lipid cholesterol (Chol) and 1,2-dimyristoyl-sn-glycerol methoxypolyethylene glycol (DMG-PEG2000; Avanti Polar Lipids, Alabaster, Alabama, USA) were combined to prepare LNP compositions.

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol percentages. The LNP compositions are shown in Table 5.

TABLE 4

| LNPs | One of | | | | | |
|---|---|---|---|---|---|---|
| comprising | COMPOUND | | | | Total | |
| COMPOUND | NOS. 1-14 | DOPC | Chol | PEG-DMG | Lipid | Lipid:DNA |
| NO. | (% moles) | (% moles) | (% moles) | (% moles) | (mM) | (w/w) |
| 3 | 40.75 | 5.0 | 51.75 | 2.5 | 25 | 120 |
| 4 | 40.75 | 5.0 | 51.75 | 2.5 | 25 | 120 |

A 1 mg/ml solution of the desired DNA to be incorporated into the LNPs was added to 150 mM sodium acetate buffer (pH 5.2) to form a stock solution and kept on ice. The ethanol phase was vigorously mixed with the nucleic acid in sodium acetate phase using the Precision Nanoassemblr instrument.

The resultant LNP compositions were then transferred to a Repligen Float-A-Lyzer dialysis device-having a molecular weight cut off (MWCO) of 8-10 kDa (Spectrum Chemical Mfg. Corp, CA, USA) and processed by dialysis against phosphate buffered saline (PBS) (dialysate: dialysis buffer volume at least 1:200 v/v), pH 7.4 overnight at 4° C. (or alternatively room temperature for at least 4 hours), to remove the 25% ethanol and achieve a complete buffer exchange. In some experiments the LNPs were further concentrated by in an Amicon® Ultra-4 centrifugal filter unit, MWCO-30 kDa (Millipore Sigma, USA) spun at ˜4100×g in an ultracentrifuge. The LNPs were then stored at 4° C. until further use.

Adult BALB/C mice (n=3) were administered 0.5 mg/kg of DNA encoding firefly luciferase (Nature Technology Corp) formulated in a LNP composition listed in Table 4. One group of mice was treated with vehicle (PBS, Thermo Fisher Scientific, USA) as a negative control.

The location and extent of luciferase expression in treated and control mice were determined at 48 hr for DNA delivery by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 6.

TABLE 5

| LNPs comprising | Mean Luciferase |
|---|---|
| COMPOUND NO. | Flux (p/s) |
| 3 | 2.12E+08 |
| 4 | 1.36E+09 |
| Vehicle | 3.29E+05 |

Visual analysis of the BLI images revealed that the BLI signal was predominantly located in the live. Accordingly, as shown in Table 5, LNP compositions of the present disclosure successfully delivered DNA to liver cells and the encoded transgene was expressed in the liver cells. In addition, administration of LNP compositions of Table 4 were well tolerated in the mice. Most of the mice lost some weight 24 hours after administration but then recovered.

### Example 78—Preparation of LNPs of Present Disclosure Comprising mRNA or DNA and In Vivo Screening

The following is a nonlimiting example that provides exemplary methods for formulating a plurality of multi-component LNP compositions comprising exemplary compounds of Formula (I) and mRNA or DNA.

LNP compositions of the present disclosure comprising COMPOUND NO. 3 and RNA encoding firefly luciferase (TriLink BioTechnologies) were prepared as described in Example 77. The LNP compositions are shown in Table 7.

TABLE 6

| | COMPOUND | | | | Total | |
|---|---|---|---|---|---|---|
| | NO. 3 | DOPC | Chol | PEG-DMG | Lipid | Lipid:RNA |
| LNP ID | (% moles) | (% moles) | (% moles) | (% moles) | (mM) | (w/w) |
| 42 | 40.75 | 5.0 | 51.75 | 2.5 | 25 | 40 |
| m-D1-9 | 45.89 | 6.21 | 45.89 | 2 | 25 | 50 |
| m-D1-12 | 60 | 0 | 38.02 | 1.98 | 25 | 50 |
| m-D2-1 | 54.19 | 3.89 | 39.5 | 2.41 | 25 | 50 |
| m-D2-2 | 43.82 | 0 | 53.83 | 2.33 | 25 | 50 |

Adult female BALB/C mice (n=3/group) were intravenously administered 0.5 mg/kg of 5′-CleanCap—fLuciferase mRNA (TriLink Biotech) formulated with the LNP compositions shown in Table 6. One group of mice was treated with vehicle (PBS, Thermo Fisher Scientific, USA) as a negative control.

In another experiment, LNP compositions of the present disclosure comprising COMPOUND NO. 3 and DNA encoding firefly luciferase (Nature Technology Corporation) were prepared as described in Example 77. The LNP compositions are shown in Table 8.

TABLE 7

| | COMPOUND | | | | Total | |
|---|---|---|---|---|---|---|
| | NO. 3 | DOPC | Chol | PEG-DMG | Lipid | Lipid:DNA |
| LNP ID | (% moles) | (% moles) | (% moles) | (% moles) | (mM) | (w/w) |
| 42 | 40.75 | 5.0 | 51.75 | 2.5 | 25 | 120 |
| d-D1-8 | 54 | 7 | 36 | 3 | 25 | 80 |
| d-D1-9 | 60 | 7 | 30 | 3 | 25 | 60 |
| d-D1-14 | 46 | 4.9 | 46.1 | 3 | 25 | 100 |
| d-D1-15 | 60 | 2.8 | 34.2 | 3 | 25 | 100 |
| d-D1-17 | 40 | 7 | 51 | 2 | 25 | 80 |
| d-D1-20 | 45.3 | 4.9 | 47.8 | 2 | 25 | 80 |
| d-D1-27 | 45.89 | 6.21 | 45.89 | 2 | 25 | 100 |

The location and extent of luciferase expression in treated and control mice were determined at 4 hr for mRNA and 48 hr for DNA by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results for mRNA are shown in Table 9 and DNA are shown in Table 10.

TABLE 8

| LNP ID. | Mean Luciferase Flux (p/s) |
|---|---|
| 42 | 2.19E+11 |
| m-D1-9 | 7.25E+10 |
| m-D1-12 | 1.17E+11 |
| m-D2-1 | 1.32E+11 |
| m-D2-2 | 4.33E+10 |
| Vehicle | 3.16E+05 |

TABLE 9

| LNP ID. | Mean Luciferase Flux (p/s) |
|---|---|
| 42 | 1.95E+08 |
| d-D1-8 | 8.09E+07 |
| d-D1-9 | 3.07E+07 |
| d-D1-14 | 4.57E+07 |
| d-D1-15 | 1.45E+08 |
| d-D1-17 | 5.95E+07 |
| d-D1-20 | 9.31E+07 |
| d-D1-27 | 4.15E+08 |
| Vehicle | 3.09E+05 |

Visual analysis of the BLI images revealed that the BLI signal was predominantly located in the live. Accordingly, as shown in Table 9 and Table 10, respectively, LNP compositions of the present disclosure successfully delivered mRNA or DNA in vivo, predominantly to cells in the liver, and the encoded transgene was subsequently expressed by the cells.

### Example 79—Preparation of LNPs of Present Disclosure Comprising mRNA and In Vivo Screening

The following is a nonlimiting example that provides exemplary methods for formulating a plurality of multi-component LNP compositions comprising exemplary compounds of Formula (II) and mRNA.

LNP compositions of the present disclosure comprising one of COMPOUND NOS. 1-14 and RNA encoding firefly luciferase (TriLink BioTechnologies) were prepared as described in Example 77. The LNP compositions are shown in Table 11.

TABLE 10

| LNPs | One of | | | | | |
|---|---|---|---|---|---|---|
| comprising | COMPOUND | | | | Total | |
| COMPOUND | NOS. 1-14 | DOPC | Chol | PEG-DMG | Lipid | Lipid:RNA |
| NO. | (% moles) | (% moles) | (% moles) | (% moles) | (mM) | (w/w) |
| 1 | 40.75 | 5.0 | 51.75 | 2.5 | 25 | 100 |
| 1 | 43.17 | 11.96 | 43.17 | 1.7 | 25 | 100 |
| 1 | 54.0 | 10.0 | 35.0 | 1.0 | 25 | 100 |
| 2 | 40.75 | 5.0 | 51.75 | 2.5 | 25 | 100 |
| 5 | 40.75 | 5.0 | 51.75 | 2.5 | 25 | 100 |
| 5 | 43.17 | 11.96 | 43.17 | 1.7 | 25 | 100 |
| 5 | 54.0 | 10.0 | 35.0 | 1.0 | 25 | 100 |
| 6 | 40.75 | 5.0 | 51.75 | 2.5 | 25 | 100 |
| 6 | 43.17 | 11.96 | 43.17 | 1.7 | 25 | 100 |
| 6 | 54.0 | 10.0 | 35.0 | 1.0 | 25 | 100 |
| 7 | 40.75 | 5.0 | 51.75 | 2.5 | 25 | 100 |
| 7 | 43.17 | 11.96 | 43.17 | 1.7 | 25 | 100 |
| 7 | 54.0 | 10.0 | 35.0 | 1.0 | 25 | 100 |
| 8 | 40.75 | 5.0 | 51.75 | 2.5 | 25 | 100 |
| 8 | 43.17 | 11.96 | 43.17 | 1.7 | 25 | 100 |
| 8 | 54.0 | 10.0 | 35.0 | 1.0 | 25 | 100 |
| 9 | 40.75 | 5.0 | 51.75 | 2.5 | 25 | 100 |
| 9 | 43.17 | 11.96 | 43.17 | 1.7 | 25 | 100 |
| 9 | 54.0 | 10.0 | 35.0 | 1.0 | 25 | 100 |
| 10 | 40.75 | 5.0 | 51.75 | 2.5 | 25 | 100 |
| 10 | 43.17 | 11.96 | 43.17 | 1.7 | 25 | 100 |
| 10 | 54.0 | 10.0 | 35.0 | 1.0 | 25 | 100 |
| 11 | 40.75 | 5.0 | 51.75 | 2.5 | 25 | 100 |
| 11 | 43.17 | 11.96 | 43.17 | 1.7 | 25 | 100 |
| 11 | 54.0 | 10.0 | 35.0 | 1.0 | 25 | 100 |
| 12 | 40.75 | 5.0 | 51.75 | 2.5 | 23 | 100 |
| 12 | 43.17 | 11.96 | 43.17 | 1.7 | 22 | 100 |

Adult female BALB/C mice (n-3/group) were intravenously administered 0.5 mg/kg of 5′-CleanCap—fLuciferase mRNA (TriLink Biotech) formulated with the LNP compositions shown in Table 11. One group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 12.

TABLE 11

| LNPs comprising | One of COMPOUND | Mean Luciferase |
|---|---|---|
| COMPOUND NO. | NOS. 1-14 (% moles) | Flux (p/s) |
| 1 | 40.75 | 8.79E+09 |
| 1 | 43.17 | 1.01E+10 |
| 1 | 54.0 | 6.15E+08 |
| 2 | 40.74 | 2.53E+07 |
| 5 | 40.75 | 2.84E+11 |
| 5 | 43.17 | 7.16E+10 |
| 5 | 54.0 | 7.25E+08 |
| 6 | 40.75 | 1.78E+11 |
| 6 | 43.17 | 3.23E+10 |
| 6 | 54.0 | 4.58E+10 |
| 7 | 40.75 | 6.41E+10 |
| 7 | 43.17 | 1.84E+11 |
| 7 | 54.0 | 1.55E+11 |
| 8 | 40.75 | 1.27E+11 |
| 8 | 43.17 | 3.31E+11 |
| 8 | 54.0 | 2.33E+11 |
| 9 | 40.75 | 4.11E+10 |
| 9 | 43.17 | 2.17E+11 |
| 9 | 54.0 | 1.31E+11 |
| 10 | 40.75 | 7.56E+10 |
| 10 | 43.17 | 1.77E+11 |
| 10 | 54.0 | 1.44E+11 |
| 11 | 40.75 | 1.10E+11 |
| 11 | 43.17 | 2.24E+11 |
| 11 | 54.0 | 2.12E+11 |
| 12 | 40.75 | 9.37E+10 |
| 12 | 43.17 | 8.35E+10 |
| Vehicle | | 5.15E+05 |

Visual analysis of the BLI images revealed that the BLI signal was predominantly located in the live. Accordingly, as shown in Table 12, LNP compositions of the present disclosure successfully delivered mRNA in vivo, predominantly to cells in the liver, and the encoded transgene was subsequently expressed by the cells.

### Example 80—Preparation of LNPs of Present Disclosure Comprising mRNA and In Vivo Screening

The following is a nonlimiting example that provides exemplary methods for formulating a plurality of multi-component LNP compositions comprising exemplary compounds of Formula (II) and mRNA.

LNP compositions of the present disclosure comprising one of COMPOUND NOS. 21-43 and RNA encoding firefly luciferase (TriLink BioTechnologies) were prepared as described in Example 77. The LNP compositions are shown in Table 13.

TABLE 12

| LNPs | One of | | | | | |
|---|---|---|---|---|---|---|
| comprising | COMPOUND | | | | Total | |
| COMPOUND | NOS. 21-43 | DOPC | Chol | PEG-DMG | Lipid | Lipid:RNA |
| NO. | (% moles) | (% moles) | (% moles) | (% moles) | (mM) | (w/w) |
| 21 | 50 | 10 | 38.5 | 1.5 | 25 | 80 |
| 22 | 50 | 10 | 38.5 | 1.5 | 25 | 80 |
| 24 | 50 | 10 | 38.5 | 1.5 | 25 | 80 |
| 27 | 50 | 10 | 38.5 | 1.5 | 25 | 80 |
| 28 | 50 | 10 | 38.5 | 1.5 | 25 | 80 |
| 30 | 50 | 10 | 38.5 | 1.5 | 25 | 80 |
| 31 | 50 | 10 | 38.5 | 1.5 | 25 | 80 |
| 32 | 50 | 10 | 38.5 | 1.5 | 25 | 80 |
| 33 | 50 | 10 | 38.5 | 1.5 | 25 | 80 |
| 34 | 50 | 10 | 38.5 | 1.5 | 25 | 80 |
| 35 | 40.75 | 5 | 51.75 | 2.5 | 25 | 80 |
| 35 | 40.75 | 5 | 51.75 | 2.5 | 25 | 40 |
| 36 | 50 | 10 | 38.5 | 1.5 | 25 | 80 |
| 36 | 43.17 | 11.96 | 43.17 | 1.7 | 25 | 80 |
| 37 | 50 | 10 | 38.5 | 1.5 | 25 | 80 |
| 37 | 43.17 | 11.96 | 43.17 | 1.7 | 25 | 80 |
| 40 | 50 | 10 | 38.5 | 1.5 | 25 | 80 |
| 40 | 40.75 | 5 | 51.75 | 2.5 | 25 | 80 |
| 41 | 50 | 10 | 38.5 | 1.5 | 25 | 80 |
| 41 | 40.75 | 5 | 51.75 | 2.5 | 25 | 80 |
| 42 | 50 | 10 | 38.5 | 1.5 | 25 | 80 |
| 42 | 40.75 | 5 | 51.75 | 2.5 | 25 | 80 |
| 43 | 50 | 10 | 38.5 | 1.5 | 25 | 80 |
| 43 | 40.75 | 5 | 51.75 | 2.5 | 25 | 40 |

In three different experiments, adult female BALB/C mice (n=3/group) were intravenously administered 0.5 mg/kg of 5′-CleanCap—fLuciferase mRNA (TriLink Biotech) formulated with the LNP compositions shown in Table 12. In each experiment, one group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results for the three different experiments are shown in Table 14, Table 15, and Table 16.

TABLE 13

| LNPs comprising | One of COMPOUND | Mean Luciferase |
|---|---|---|
| COMPOUND NO. | NOS. 21-43 (% moles) | Flux (p/s) |
| 21 | 50 | 1.27E+08 |
| 22 | 50 | 2.31E+07 |
| 24 | 50 | 3.46E+06 |
| 27 | 50 | 1.10E+10 |
| 28 | 50 | 3.28E+10 |
| 30 | 50 | 2.54E+10 |
| 31 | 50 | 1.00E+10 |
| 32 | 50 | 7.76E+09 |
| 33 | 50 | 1.73E+10 |
| 34 | 50 | 1.87E+10 |
| 35 | 50 | 2.35E+09 |
| 35 | 50 | 3.13E+09 |
| vehicle | | 4.83E+05 |

TABLE 14

| LNPs comprising | One of COMPOUND | Mean Luciferase |
|---|---|---|
| COMPOUND NO. | NOS. 21-43 (% moles) | Flux (p/s) |
| 36 | 50 | 1.19E+11 |
| 36 | 43.17 | 1.31E+11 |
| 37 | 50 | 3.55E+11 |
| 37 | 43.17 | 3.49E+11 |
| 43 | 50 | 1.10E+09 |
| 43 | 40.75 | 1.12E+09 |
| vehicle | | 4.30E+05 |

TABLE 15

| LNPs | One of | Mean |
|---|---|---|
| comprising | COMPOUND | Luciferase |
| COMPOUND | NOS. 21-43 | Flux |
| NO. | (% moles) | (p/s) |
| 40 | 50 | 1.02E+11 |
| 40 | 40.75 | 2.20E+10 |
| 41 | 50 | 5.26E+09 |
| 41 | 40.75 | 3.79E+09 |
| 42 | 50 | 8.27E+09 |
| 42 | 40.75 | 1.23E+09 |
| vehicle | | 3.29E+05 |

Visual analysis of the BLI images revealed that the BLI signal was predominantly located in the live. Accordingly, as shown in Table 14, Table 15, and Table 16, LNP compositions of the present disclosure successfully delivered mRNA in vivo, predominantly to cells in the liver, and the encoded transgene was subsequently expressed by the cells.

### Example 81—In Vivo LNP Delivery of mRNA to Liver

The following is a non-limiting example demonstrating that the lipid nanoparticle compositions of the present disclosure can be used to deliver mRNA to liver cells in vivo and subsequently express the encoded proteins with good tolerability.

LNP compositions of the present disclosure comprising Compound No. 40 were prepared as described in Example 80. The LNP compositions are shown in Table 17.

TABLE 16

| Compound | | | PEG- | Total | |
|---|---|---|---|---|---|
| No. 40 | DOPC | Chol | DMG | Lipid | Lipid:RNA |
| (% moles) | (% moles) | (% moles) | (% moles) | (mM) | (w/w) |
| 50 | 10 | 38.5 | 1.5 | 25 | 80 |
| 40.8 | 5.0 | 51.8 | 2.5 | 25 | 80 |
| (40.75) | | (51.75) | | | |

Adult female BALB/C mice (n=3/group) were intravenously administered 0.5 mg/kg of 5′-CleanCap—fLuciferase mRNA (TriLink Biotech) formulated with the LNP compositions shown in Table 17. In each experiment, one group of mice was treated with vehicle (PBS, Thermo Fisher Scientific, USA) as a negative control.

The levels of five proinflammatory cytokines present in serum were evaluated at 4 hours after LNP administration for each of the tested concentrations. Briefly, serum samples were prepared as described for liver enzyme analysis and the serum concentration of each cytokine was determined using commercially available ELISA kits (e.g., R&D Systems Quantikine ELISA kits). The levels of the proinflammatory cytokines interleukin-6 (IL-6), interferon gamma (INF-G), tumor necrosis factor alpha (TNF-α), monocyte chemoattractant protein-1 (MCP-1), macrophage inflammatory protein-1 alpha (MIP-1a) in mice treated with LNPs of the present disclosure compared to vehicle treated mice at 4 hours are shown in Table 18.

TABLE 17
cytokine fold changes in treated mice relative to untreated mice

| Compound | | | | | |
|---|---|---|---|---|---|
| No. 40 | | | | | |
| (% moles) | IL-6 | INF-G | TNF-a | MCP-1 | MIP-1a |
| 50 | 0.6 | 2.0 | 1.1 | 5.2 | 3.8 |
| 40.8 | 0.4 | 1.5 | 1.0 | 1.6 | 1.4 |
| (40.75) | | | | | |
| vehicle | 1.0 | 1.0 | 1.0 | 1.0 | 1.0 |

In addition, the body weight of mice treated with LNP compositions of the present disclosure prepared as described in Example 77 was assessed prior to intravenous administration and 24 hours post-administration and the body weights at baseline and post-treatment were compared. The average percentage of body weight change for each group of mice treated with each LNP composition is shown in Table 19.

TABLE 18
% Body weight change 24 hr

| LNPs | One of | |
|---|---|---|
| comprising | COMPOUND | |
| COMPOUND | NOS. 21-43 | % body |
| NO. | (% moles) | weight change |
| 40 | 50 | −2.0 |
| 40 | 40.75 | −5.5 |
| 41 | 50 | −2.6 |
| 41 | 40.75 | −1.8 |
| 42 | 50 | −4.0 |
| 42 | 40.75 | −2.3 |
| vehicle | | −1.1 |

The results of this example show that LNP compositions of the present disclosure successfully delivered mRNA in vivo, predominantly to cells in the liver, and the encoded protein was subsequently expressed by the cells. The LNP compositions exhibited similar toxicity profiles compared to the negative control. Administration of LNP compositions were also well tolerated in the mice. Most of the mice lost some weight 24 hours after administration but then recovered.

### Example 82—Preparation of LNPs of Present Disclosure Comprising DNA and In Vivo Screening

The following is a nonlimiting example that provides exemplary methods for formulating a plurality of multi-component LNP compositions comprising exemplary compounds of Formula (I) and DNA.

LNP compositions of the present disclosure comprising either COMPOUND NO. 36 or COMPOUND NO. 37 and DNA encoding firefly luciferase (Nature Technology Corporation) were prepared as described in Example 77. The LNP compositions are shown in Table 20.

TABLE 19

| LNPs | | | | | | |
|---|---|---|---|---|---|---|
| comprising | COMPOUND | | | | Total | |
| COMPOUND | NO. 36 or 37 | DOPC | Chol | PEG-DMG | Lipid | Lipid:DNA |
| NO. | (% moles) | (% moles) | (% moles) | (% moles) | (mM) | (w/w) |
| 36 | 50 | 10 | 38.5 | 1.5 | 25 | 80 |
| 36 | 43.17 | 11.96 | 43.17 | 1.7 | 25 | 80 |
| 37 | 50 | 10 | 38.5 | 1.5 | 25 | 80 |
| 37 | 43.17 | 11.96 | 43.17 | 1.7 | 25 | 80 |

The location and extent of luciferase expression in treated and control mice were determined at 48 hr for DNA by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 21. Visual analysis of the BLI images revealed that the BLI signal was predominantly located in the live.

TABLE 20

| LNPs | | Mean |
|---|---|---|
| comprising | COMPOUND | Luciferase |
| COMPOUND | NO. 36 or 37 | Flux |
| NO. | (% moles) | (p/s) |
| 36 | 50 | 1.82E+08 |
| 36 | 43.17 | 7.99E+07 |
| 37 | 50 | 1.28E+08 |
| 37 | 43.17 | 5.80E+07 |
| Vehicle | | 2.71E+05 |

In addition, the body weight of mice treated with LNP compositions of the present disclosure was assessed prior to intravenous administration and 24 hours post-administration and the body weights at baseline and post-treatment were compared. The average percentage of body weight change for each group of mice treated with each LNP composition is shown in Table 22.

TABLE 21
% Body weight change 24 hr

| LNPs | | |
|---|---|---|
| comprising | COMPOUND | |
| COMPOUND | NO. 36 or 37 | % body |
| NO. | (% moles) | weight change |
| 36 | 50 | −6.7 |
| 36 | 43.17 | −4.9 |
| 37 | 50 | −10.7 |
| 37 | 43.17 | −6.0 |
| vehicle | | 2.8 |

The results of this example show that LNP compositions of the present disclosure successfully delivered DNA in vivo, predominantly to cells in the liver, and the encoded protein was subsequently expressed by the cells. The LNP compositions exhibited similar toxicity profiles compared to the negative control. Administration of LNP compositions were also well tolerated in the mice. Most of the mice lost some weight 24 hours after administration but then recovered.

### Example 83—LNP Compositions of Present Disclosure Deliver RNA with High Specificity to the Liver In Vivo

This example shows the ability of LNP compositions of the present disclosure to deliver Cas-CLOVER mRNA to the liver, targeted by a pair of gRNAs to the psk9 gene, resulting in subsequent in vivo gene editing of the psk9 gene. Pcsk9 protein is secreted by hepatocytes and binds to the LDL receptor, inducing its internalization and lysosomal degradation, resulting in increased circulating levels of LDL-cholesterol.

In two separate experiments, each group of adult female BALB/C mice (n=2/group) was intravenously co-administered mRNA encoding 5′-CleanCap-5MeC-Cas-CLOVER (SEQ ID NO: 5) and a pair of gRNAs (SEQ ID NOs: 6 and 7) targeted to the first exon of the mouse pcsk9 gene.

In the first experiment, the mRNA and gRNA molecules were formulated within LNP compositions of the present disclosure comprising COMPOUND NO. 37, DOPC, Cholesterol and DMG-PEG2000 at the following molar ratio: 50:10:38.5:1.5. All cytidine residues in the mRNA were 5-methylcytidine (5-MeC). The lipid: RNA (w/w) ratio in the LNP compositions was 80:1.

LNP compositions of the present disclosure (0.5 mg/kg or 1 mg/kg) were administered to the mice from each group. One group of mice was administered a dose of Cas-CLOVER mRNA and a pair of pcsk9 gRNA, both co-encapsulated in a LNP composition of the present disclosure. One group of mice was treated with vehicle (PBS, Thermo Fisher Scientific, USA) as a negative control.

In the second experiment, the mRNA and gRNA molecules were formulated within LNP compositions of the present disclosure comprising COMPOUND NO. 37, DOPC and/or DSPC, Cholesterol and DMG-PEG2000 at the following molar ratio: 50:10:38.5:1.5. All cytidine residues in the mRNA were 5-methylcytidine (5-MeC). The lipid: RNA (w/w) ratio in the LNP compositions was 80:1.

LNP compositions of the present disclosure (0.5 mg/kg) were administered to the mice from each group. One group of mice was administered a dose of Cas-CLOVER mRNA and a pair of pcsk9 gRNA, both co-encapsulated in a LNP composition of the present disclosure. One group of mice was treated with vehicle (PBS, Thermo Fisher Scientific, USA) as a negative control.

After seven days post-administration, blood samples were drawn from the subjects. Briefly, 500 μL of blood was collected after euthanasia via cardiac puncture using 2 ml syringes and 25G needles, transferred to microcentrifuge tubes, incubated at room temperature for 1 hour, and centrifuged at 1500 g for 15 minutes to separate the cellular fraction from serum. Serum fraction (200 μL) was transferred to a new tube and stored at −80C until further analysis.

Serum levels of the pcsk9 protein in the mice were measured 7 days after administration. Results are shown in Table 23 for the first experiment and Table 24 for the second experiment. Briefly, a mouse Pcsk9 ELISA kit (Biolegend) was used to determine Pcsk9 in each serum sample following manufacturer's instructions. All serum samples were assayed in triplicate and results were expressed as percentage in Pcsk9 levels compared with Pcsk9 levels of PBS-treated mice.

TABLE 22

| | % Pcsk9 |
|---|---|
| | compared to |
| LNP Dose | control |
| PBS control | 100 |
| 0.5 mg/kg | 40.6 |
| 1.0 mg/kg | 23.8 |

TABLE 23

| DSPC:DOPC | |
|---|---|
| molar ratio in | |
| LNPs | |
| comprising | % Pcsk9 |
| COMPOUND | compared to |
| NO. 37 | control |
| PBS control | 100 |
| 5%:5% | 50.4 |
| 10%:0% | 39.7 |

The results of these experiments show that Cas-CLOVER mRNA delivered by LNP compositions of the present disclosure is effective at editing the pcsk9 gene in the liver in vivo, even at the lower dose.

### Example 84—Preparation of LNPs of Present Disclosure Comprising mRNA and In Vivo Screening

The following is a nonlimiting example that provides exemplary methods for formulating a plurality of multi-component LNP compositions comprising exemplary compounds of Formula (II) and mRNA.

LNP compositions of the present disclosure comprising COMPOUND NO. 37 and RNA encoding firefly luciferase (TriLink BioTechnologies) were prepared as described in Example 77. The LNP compositions are shown in Table 25.

TABLE 24

| | COMPOUND | | | | Total | |
|---|---|---|---|---|---|---|
| LNP | NO. 37 | phospholipid | Chol | PEG-DMG | Lipid | Lipid:RNA |
| ID | (% moles) | (% moles) | (% moles) | (% moles) | (mM) | (w/w) |
| 1.1 | 50 | 5% DOPC | 42.5 | 2.5 | 20 | 60 |
| 1.2 | 45 | 5% DOPC | 48.5 | 1.5 | 20 | 40 |
| 1.3 | 50 | 10% DOPC | 37.5 | 2.5 | 20 | 40 |
| 1.4 | 50 | 7.5% DOPC | 41 | 1.5 | 20 | 60 |
| 1.5 | 40 | 5% DOPC | 52.5 | 2.5 | 20 | 50 |
| 1.6 | 40 | 7.5% DOPC | 50 | 2.5 | 20 | 40 |
| 1.7 | 50 | 5% DOPC | 43 | 2 | 20 | 40 |
| 1.8 | 50 | 10% DOPC | 38.5 | 1.5 | 20 | 50 |
| 1.9 | 45 | 7.5% DOPC | 45.5 | 2 | 20 | 50 |
| 1.10 | 40 | 10% DOPC | 48 | 2 | 20 | 60 |
| 1.11 | 40 | 5% DOPC | 53.5 | 1.5 | 20 | 60 |
| 1.12 | 45 | 10% DOPC | 42.5 | 2.5 | 20 | 60 |
| 1.13 | 40 | 10% DOPC | 48.5 | 1.5 | 20 | 40 |
| 1.14 | 50 | 10% DOPC | 38.5 | 1.5 | 20 | 40 |
| 1.15 | 50 | 10% DOPC | 38.5 | 1.5 | 20 | 80 |
| 1.16 | 50 | 5% DOPC/ | 38.5 | 1.5 | 20 | 80 |
| | | 5% DSPC | | | | |
| 1.17 | 50 | 10% DSPC | 38.5 | 1.5 | 20 | 80 |

In the same experiment, LNP compositions of the present disclosure comprising COMPOUND NO. 38 or 39 and RNA encoding firefly luciferase (TriLink BioTechnologies) were prepared as described in Example 77. The LNP compositions are shown in Table 26.

TABLE 25

| LNPs | | | | | | |
|---|---|---|---|---|---|---|
| comprising | COMPOUND | | | | Total | |
| COMPOUND | NO. 38 or 39 | DOPC | Chol | PEG-DMG | Lipid | Lipid:RNA |
| NO. | (% moles) | (% moles) | (% moles) | (% moles) | (mM) | (w/w) |
| 38 | 50 | 10 | 38.5 | 1.5 | 25 | 80 |
| 39 | 50 | 10 | 38.5 | 1.5 | 25 | 80 |

Adult female BALB/C mice (n-3/group) were intravenously administered 0.5 mg/kg of 5′-CleanCap—fLuciferase mRNA (TriLink Biotech) formulated with the LNP compositions shown in Table 25 and Table 26. One group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions.

Briefly, mice were anesthetized using isoflurane in oxygen, and placed supine on a heated stage. Mice were then administered D-luciferin (Perkin-Elmer #122799) IP, and BLI was performed. The results are shown in Table 27 and Table 28.

TABLE 26

| | Mean Luciferase |
|---|---|
| LNP ID | Flux (p/s) |
| 1.1 | 2.08E+11 |
| 1.2 | 2.17E+11 |
| 1.3 | 1.89E+11 |
| 1.4 | 2.38E+11 |
| 1.5 | 1.67E+11 |
| 1.6 | 1.45E+11 |
| 1.7 | 2.36E+11 |
| 1.8 | 2.01E+11 |
| 1.9 | 2.07E+11 |
| 1.10 | 2.03E+11 |
| 1.11 | 2.66E+11 |
| 1.12 | 2.31E+11 |
| 1.13 | 1.58E+11 |
| 1.14 | 1.41E+11 |
| 1.15 | 2.52E+11 |
| 1.16 | 2.24E+11 |
| 1.17 | 3.93E+11 |
| vehicle | 3.83E+05 |

TABLE 27

| LNPs | Mean |
|---|---|
| comprising | Luciferase |
| COMPOUND | Flux |
| NO. | (p/s) |
| 38 | 1.17E+11 |
| 39 | 9.49E+10 |
| Vehicle | 3.83E+05 |

Visual analysis of the BLI images revealed that the BLI signal was predominantly located in the live. Accordingly, as shown in Table 27 and Table 28, LNP compositions of the present disclosure successfully delivered mRNA in vivo, predominantly to cells in the liver, and the encoded transgene was subsequently expressed by the cells.

### Example 85—LNP Compositions of Present Disclosure Deliver RNA with High Specificity to the Liver In Vivo

As described in Example 83, mRNA and gRNA molecules were formulated within LNP compositions of the present disclosure comprising COMPOUND NO. 37, a phospholipid, Cholesterol and DMG-PEG2000 at the following molar ratio: 50:10:38.5:1.5 as shown in Table 29. All uridine residues in the mRNA were N1-methylpsuedouridine.

TABLE 28

| | | | Lipid:RNA |
|---|---|---|---|
| LNP ID | phospholipid | mRNA:gRNA | (w/w) |
| 2.1 | DSPC | 3:1 | 40 |
| 2.2 | DSPC | 2:1 | 40 |
| 2.3 | DSPC | 1:1 | 40 |
| 2.4 | DSPC | 3:1 | 50 |
| 2.5 | DSPC | 2:1 | 50 |
| 2.6 | DSPC | 1:1 | 50 |
| 2.7 | DSPC | 3:1 | 60 |
| 2.8 | DSPC | 2:1 | 60 |
| 2.9 | DSPC | 1:1 | 60 |
| 2.10 | DSPC | 3:1 | 80 |
| 2.11 | DSPC | 2:1 | 80 |
| 2.12 | DSPC | 1:1 | 80 |
| 2.13 | DOPC | 3:1 | 80 |
| 2.14 | DPPC | 3:1 | 80 |

Mice from each group were administered a dose (0.5 mg/kg) of Cas-CLOVER mRNA and a pair of pcsk9 gRNA, both co-encapsulated in a LNP composition of the present disclosure. One group of mice was treated with vehicle (PBS, Thermo Fisher Scientific, USA) as a negative control.

Serum levels of the pcsk9 protein in the mice were measured as described in Example 83. The results are shown in Table 30.

TABLE 29

| | % Pcsk9 |
|---|---|
| | compared to |
| LNP ID | control |
| PBS control | 100 |
| 2.1 | 54.0 |
| 2.2 | 52.0 |
| 2.3 | 45.4 |
| 2.4 | 64.4 |
| 2.5 | 31.3 |
| 2.6 | 23.7 |
| 2.7 | 26.8 |
| 2.8 | 47.3 |
| 2.9 | 31.8 |
| 2.10 | 42.5 |
| 2.11 | 41.4 |
| 2.12 | 26.8 |
| 2.13 | 41.3 |
| 2.14 | 25.3 |

The results of these experiments show that Cas-CLOVER mRNA delivered by LNP compositions of the present disclosure is effective at editing the pcsk9 gene in the liver in vivo, even at the lower lipid to payload ratios (e.g., 40:1 and 50:1).

### Example 86—LNP Compositions of Present Disclosure Deliver RNA with High Specificity to the Liver In Vivo

As described in Example 83, mRNA and gRNA molecules were formulated within LNP compositions of the present disclosure comprising COMPOUND NO. 37, a phospholipid, Cholesterol and DMG-PEG2000 at the following molar ratios: 50:10:38.5:1.5 or 50:10:38:2 as shown in Table 31. All uridine residues in the mRNA were N1-methylpsuedouridine.

TABLE 30

| | | | | Lipid:RNA |
|---|---|---|---|---|
| LNP ID | phospholipid | cholesterol | % PEG | (w/w) |
| 3.1 | DSPC | 38.5 | 1.5 | 40 |
| 3.2 | DSPC | 38.5 | 1.5 | 50 |
| 3.3 | DPPC | 38.5 | 1.5 | 40 |
| 3.4 | DPPC | 38.5 | 1.5 | 50 |
| 3.5 | DSPC | 38 | 2 | 40 |
| 3.6 | DSPC | 38 | 2 | 50 |
| 3.7 | DPPC | 38 | 2 | 40 |
| 3.8 | DPPC | 38 | 2 | 50 |

Mice from each group were administered a dose (0.5 mg/kg) of Cas-CLOVER mRNA and a pair of pcsk9 gRNA, both co-encapsulated in an LNP composition of the present disclosure. One group of mice was treated with vehicle (PBS, Thermo Fisher Scientific, USA) as a negative control. Another group of mice was treated with a benchmark LNP composition comprising the cationic lipid ssPalmO-Ph-P4C2, as described in Akita et al., (2020) Biol. Phar. Bull. 43:1617-1625.

Serum levels of the pcsk9 protein in the mice were measured as described in Example 83. The results are shown in Table 32.

TABLE 31

| | % Pcsk9 |
|---|---|
| | compared to |
| LNP ID | control |
| PBS control | 100 |
| 3.1 | 52.4 |
| 3.2 | 28.0 |
| 3.3 | 41.3 |
| 3.4 | 30.0 |
| 3.5 | 41.2 |
| 3.6 | 32.0 |
| 3.7 | 44.5 |
| 3.8 | 39.8 |
| Benchmark LNP | 75.4 |

The results of these experiments show that Cas-CLOVER mRNA delivered by LNP compositions of the present disclosure is effective at editing the pcsk9 gene in the liver in vivo. Additionally, gene editing resulting from Cas-CLOVER mRNA delivered by LNP compositions of the present disclosure was markedly more effective than gene editing resulting from Cas-CLOVER mRNA delivered by a benchmark LNP composition.

### Example 87—In Vivo LNP Delivery of mRNA to Liver with Improved Tolerability

The following is a non-limiting example demonstrating that lipid nanoparticle compositions comprising GalNac or DSPE (1, 2-Distearoyl-sn-glycero-3-phosphoethanolamine) can be used to deliver mRNA to liver cells in vivo with improved tolerability compared to LNP compositions not comprising GalNac or DSPE.

LNP compositions of the present disclosure comprising COMPOUND NO. 37 and RNA encoding firefly luciferase (TriLink BioTechnologies) were prepared as described in Example 77. The LNP compositions are shown in Table 33.

TABLE 32

| | COMPOUND | | | PEG- | GalNac- | DSPE- | Lipid:Nucleic |
|---|---|---|---|---|---|---|---|
| LNP | NO. 37 | DSPC | Chol | DMG | PEG | PEG | Acid |
| ID | (% moles) | (% moles) | (% moles) | (% moles) | (% moles) | (% moles) | (w/w) |
| 4.1 | 50 | 10 | 38.5 | 1.5 | 0 | 0 | 80 |
| 4.2 | 50 | 10 | 38.5 | 1.25 | 0.25 | 0 | 80 |
| 4.3 | 50 | 10 | 38.5 | 1 | 0.5 | 0 | 80 |
| 4.4 | 50 | 10 | 38.5 | 1 | 0 | 0.5 | 80 |
| 4.5 | 45 | 10 | 42.5 | 2.5 | 0 | 0 | 60 |
| 4.6 | 45 | 10 | 42.5 | 2.25 | 0.25 | 0 | 60 |
| 4.7 | 45 | 10 | 42.5 | 2 | 0.5 | 0 | 60 |

Adult female BALB/C mice (n=3/group) were intravenously administered 0.5 mg/kg of 5′-CleanCap—fLuciferase mRNA (TriLink Biotech) formulated with the LNP compositions shown in Table 30. One group of mice was treated with vehicle (PBS, Thermo Fisher Scientific, USA) as a negative control.

The location and extent of luciferase expression in treated and control mice were determined as described in Example 77 and the results are shown in Table 34. Visual analysis of the BLI images revealed that the BLI signal was predominantly located in the live.

TABLE 33

| | Mean Luciferase |
|---|---|
| LNP ID. | Flux (p/s) |
| 4.1 | 3.38E+11 |
| 4.2 | 4.26E+11 |
| 4.3 | 2.55E+11 |
| 4.4 | 2.29E+11 |
| 4.5 | 2.38E+11 |
| 4.6 | 1.67E+11 |
| 4.7 | 1.17E+11 |
| vehicle | 5.46E+05 |

The levels of five proinflammatory cytokines present in serum were evaluated at 4 hours after LNP administration as described in Example 81. The levels of the proinflammatory cytokines interleukin-6 (IL-6), interferon gamma (INF-G), tumor necrosis factor alpha (TNF-a), monocyte chemoattractant protein-1 (MCP-1), macrophage inflammatory protein-1 beta (MIP-1b) in mice treated with LNPs of the present disclosure are shown in Table 35.

TABLE 34
cytokine concentration (pg/mL)

| LNP ID | IL-6 | INF-G | TNF-a | MCP-1 | MIP-1b |
|---|---|---|---|---|---|
| 4.1 | 1243.68 | 180.12 | 147.07 | 7010.41 | 8282.94 |
| 4.2 | 1324.85 | 29.95 | 127.18 | 5292.95 | 7310.97 |
| 4.3 | 631.27 | 20.15 | 60.24 | 2315.19 | 3830.57 |
| 4.4 | 868.97 | 30.92 | 53.80 | 2754.62 | 3703.88 |
| 4.5 | 709.00 | 33.11 | 47.89 | 2449.59 | 3397.22 |
| 4.6 | 454.32 | 16.06 | 23.32 | 1164.99 | 2022.46 |
| 4.7 | 282.93 | 9.80 | 14.83 | 1020.35 | 1589.41 |

The results of this example show that LNP compositions of the present disclosure successfully delivered mRNA in vivo, predominantly to cells in the liver, and the encoded transgene was subsequently expressed by the cells.

The results of this example also show that the addition of GalNac or DSPE to LNP compositions for the administration of mRNA to liver cells in vivo improved tolerability compared to LNP compositions that did not comprise GalNac or DSPE. The LNP compositions with GalNac or DSPE exhibited significantly lower cytokine induction relative to LNP compositions without GalNac.

### Example 88—In Vivo LNP Delivery of mRNA to Liver with Improved Tolerability

The following is a non-limiting example demonstrating that lipid nanoparticle compositions comprising GalNac can be used to deliver mRNA to liver cells in vivo with improved tolerability compared to LNP compositions not comprising GalNac.

As described in Example 83, mRNA and gRNA molecules were formulated within LNP compositions of the present disclosure comprising COMPOUND NO. 37, a phospholipid, Cholesterol and DMG-PEG2000 as shown in Table 36. All uridine residues in the mRNA were N1-methylpsuedouridine.

TABLE 35

| | COMPOUND | | | PEG- | GalNac- | Lipid:Nucleic |
|---|---|---|---|---|---|---|
| | NO. 37 | DSPC | Chol | DMG | PEG | Acid |
| LNP ID | (% moles) | (% moles) | (% moles) | (% moles) | (% moles) | (w/w) |
| 5.1 | 50 | 10 | 38.5 | 1.5 | 0 | 50 |
| 5.2 | 50 | 10 | 38.5 | 1.2 | 0.3 | 50 |
| 5.3 | 50 | 10 | 38.5 | 1 | 0.5 | 50 |
| 5.4 | 50 | 10 | 38 | 2 | 0 | 50 |
| 5.5 | 50 | 10 | 38 | 1.7 | 0.3 | 50 |
| 5.6 | 50 | 10 | 38 | 1.5 | 0.5 | 50 |

Adult BALB/C mice (n=2/group) were administered a dose (0.5 mg/kg or 1.0 mg/kg) of Cas-CLOVER mRNA and a pair of pcsk9 gRNA, both co-encapsulated in a LNP composition of the present disclosure. One group of mice was treated with vehicle (PBS, Thermo Fisher Scientific, USA) as a negative control.

Serum levels of the pcsk9 protein in the mice were measured as described in Example 83. The results are shown in Table 37.

TABLE 36

| | % Pcsk9 | % Pcsk9 |
|---|---|---|
| | compared to | compared to |
| | control | control |
| LNP ID | (0.5 mpk dose) | (1.0 mpk dose) |
| PBS control | 100 | 100 |
| 5.1 | 36.3 | 11.4 |
| 5.2 | 22.0 | 12.5 |
| 5.3 | 19.8 | 11.8 |
| 5.4 | 41.9 | 10.4 |
| 5.5 | 30.8 | 12.9 |
| 5.6 | 36.5 | 11.1 |

The levels of five proinflammatory cytokines present in serum were evaluated at 4 hours after LNP administration as described in Example 81. The levels of the proinflammatory cytokines interleukin-6 (IL-6), interferon gamma (INF-G), tumor necrosis factor alpha (TNF-a), monocyte chemoattractant protein-1 (MCP-1), macrophage inflammatory protein-1 beta (MIP-1b) in mice treated with LNPs of the present disclosure are shown in Table 38 and Table 39.

TABLE 37
cytokine concentration (pg/mL), 0.5 mpk dose

| LNP ID | IL-6 | INF-G | TNF-a | MCP-1 | MIP-1b |
|---|---|---|---|---|---|
| 5.1 | 329 | 19 | 46 | 1705 | 2013 |
| 5.2 | 88 | 5 | 45 | 754 | 1960 |
| 5.3 | 19 | 5 | 37 | 835 | 1566 |
| 5.4 | 128 | 6 | 40 | 1100 | 1878 |
| 5.5 | 0 | 3 | 14 | 346 | 791 |
| 5.6 | 0 | 5 | 22 | 443 | 900 |

TABLE 38
cytokine concentration (pg/mL), 1.0 mpk dose

| LNP ID | IL-6 | INF-G | TNF-a | MCP-1 | MIP-1b |
|---|---|---|---|---|---|
| 5.1 | 2480 | 20 | 98 | 3512 | 4978 |
| 5.2 | 420 | 7 | 141 | 3345 | 6271 |
| 5.3 | 110 | 5 | 55 | 949 | 1963 |
| 5.4 | 746 | 16 | 199 | 5072 | 8904 |
| 5.5 | 244 | 7 | 90 | 1784 | 4429 |
| 5.6 | 413 | 17 | 168 | 3802 | 6338 |

The results of these experiments show that Cas-CLOVER mRNA delivered by LNP compositions of the present disclosure is effective at editing the pcsk9 gene in the liver in vivo.

Additionally, the addition of GalNac to LNP compositions for the administration of mRNA to liver cells in vivo improved tolerability compared to LNP compositions that did not comprise GalNac. The LNP compositions with GalNac exhibited significantly lower cytokine induction relative to LNP compositions without GalNac.

### Example 89—In Vivo LNP Delivery of Co-Encapsulated mRNA and DNA to Liver

The following is a nonlimiting example that provides exemplary methods for formulating a plurality of multi-component LNP compositions comprising exemplary compounds of Formula (I) and co-encapsulated mRNA and DNA.

In these experiments, luciferase expression indicates successful delivery by LNP compositions of a two-component, DNA/RNA system to liver cells, resulting in transposition of the luciferase transgene facilitated by SPB.

Adult BALB/C mice (n=3) were administered a single co-encapsulated LNP encapsulating both firefly luciferase transposon and SPB. The compositions of the co-encapsulated LNPs are shown in Table 40 and were prepared as described in Example 77, incorporating mRNA encoding active SPB and nanoplasmid DNA (SEQ ID NO: 8). All cytidine residues in the mRNA were 5-methylcytidine (5-MeC).

TABLE 39

| | | | | | PEG- | GalNac- | |
|---|---|---|---|---|---|---|---|
| | COMPOUND | DOPC | DSPC | Chol | DMG | PEG | Lipid:Nucleic Acid |
| LNP | NO. 37 | (% | (% | (% | (% | (% | (Lipid:RNA + DNA) |
| ID | (% moles) | moles) | moles) | moles) | moles) | moles) | (w/w) |
| 6.1 | 50 | 0 | 10 | 38.5 | 1.5 | 0 | 50 |
| 6.2 | 50 | 0 | 10 | 38.5 | 1 | 0.5 | 50 |
| 6.3 | 50 | 0 | 10 | 38 | 2 | 0 | 50 |
| 6.4 | 50 | 10 | 0 | 38.5 | 1.5 | 0 | 50 |
| 6.5 | 50 | 10 | 0 | 38.5 | 1.5 | 0 | 80 |

Mice received either 0.5 mg/kg or 1.0 mg/kg of co-encapsulated LNP encapsulating mRNA and DNA at 1:2 mRNA: DNA ratio. One group of mice was treated with vehicle (PBS, Thermo Fisher Scientific, USA) as a negative control.

The location and extent of luciferase expression in treated and control mice were determined as described in Example 77 and the results are shown in Table 41.

TABLE 40

| | | Mean |
|---|---|---|
| | LNP | Luciferase |
| | Dose | Flux |
| LNP ID. | (mg/kg) | (p/s) |
| 6.1 | 1.0 | 1.09E+10 |
| 6.2 | 1.0 | 1.27E+10 |
| 6.3 | 1.0 | 6.84E+09 |
| 6.4 | 1.0 | 9.34E+09 |
| 6.5 | 0.5 | 1.52E+09 |
| vehicle | | 1.50E+05 |

Visual analysis of the BLI images revealed that the BLI signal was predominantly located in the live. Accordingly, this example shows that LNP compositions of the present disclosure successfully delivered a two-component, DNA/RNA system to liver cells and the desired transgene was stably integrated into the genome and not episomal.

### Example 90—In Vivo LNP Delivery of Co-Encapsulated mRNA and DNA to Liver with Improved Tolerability

The following is a non-limiting example demonstrating that lipid nanoparticle compositions comprising GalNac can be used to deliver co-encapsulated mRNA and DNA to liver cells in vivo with improved tolerability compared to a benchmark LNP composition comprising GalNac or LNP compositions of the present disclosure that did not comprise GalNac.

As described in Example 89, luciferase expression in this Example indicates successful delivery by LNP compositions of a two-component, DNA/RNA system to liver cells, resulting in transposition of the luciferase transgene facilitated by SPB.

Adult BALB/C mice (n-3) were administered a single co-encapsulated LNP encapsulating both firefly luciferase transposon and SPB. The compositions of the co-encapsulated LNPs are shown in Table 42 and were prepared as described in Example 77, incorporating mRNA encoding active SPB and nanoplasmid DNA (SEQ ID NO: 8). All cytidine residues in the mRNA were 5-methylcytidine (5-MeC).

TABLE 41

| | | | | PEG- | GalNac- | |
|---|---|---|---|---|---|---|
| | COMPOUND | DOPC | Chol | DMG | PEG | Lipid:Nucleic Acid |
| LNP | NO. 37 | (% | (% | (% | (% | (Lipid:RNA + DNA) |
| ID | (% moles) | moles) | moles) | moles) | moles) | (w/w) |
| 7.1 | 45 | 10 | 42.5 | 2 | 0.5 | 60 |
| 7.2 | 40 | 7.5 | 50.5 | 2 | 0 | 40 |
| 7.3 | 50 | 5 | 42.5 | 2 | 0.5 | 40 |
| 7.4 | 40 | 10 | 49 | 1 | 0 | 60 |
| 7.5 | 50 | 7.5 | 41 | 1 | 0.5 | 60 |
| 7.6 | 50 | 10 | 38 | 1.5 | 0.5 | 50 |
| 7.7 | 45 | 5 | 49 | 1 | 0 | 40 |
| 7.8 | 45 | 7.5 | 45.75 | 1.5 | 0.25 | 50 |
| 7.9 | 40 | 5 | 53.5 | 1 | 0.5 | 50 |
| 7.10 | 40 | 10 | 48 | 1.5 | 0.5 | 40 |
| 7.11 | 40 | 5 | 52.75 | 2 | 0.25 | 60 |
| 7.12 | 50 | 10 | 38.75 | 1 | 0.25 | 40 |
| 7.13 | 50 | 5 | 43.25 | 1.5 | 0.25 | 60 |
| 7.14 | 50 | 10 | 38.5 | 1.5 | 0 | 50 |
| 7.15 | 50 | 10 | 38.5 | 1 | 0.5 | 50 |
| 7.16 | 50 | 10 | 38 | 2 | 0 | 50 |
| 7.17 | 50 | 10 | 38 | 1.7 | 0.3 | 50 |

Mice received 1.0 mg/kg of co-encapsulated LNP encapsulating mRNA and DNA at 1:2 mRNA: DNA ratio. One group of mice was treated with vehicle (PBS, Thermo Fisher Scientific, USA) as a negative control. Another group of mice was treated with a benchmark LNP composition comprising the cationic lipid HMA-404 with the following structure:

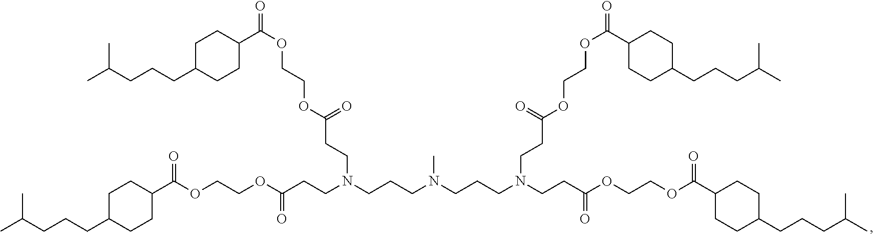 [SMILES: CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]

prepared as described in PCT Application No. PCT/US2023/061005.

The location and extent of luciferase expression in treated and control mice were determined as described in Example 77 and the results are shown in Table 43. LNP compositions of the present disclosure successfully delivered a two-component, DNA/RNA system to liver cells, as indicated by the luciferase expression levels compared to vehicle as shown in Table 43.

TABLE 42

| | Mean |
|---|---|
| | Luciferase |
| | Flux |
| LNP ID. | (p/s) |
| 7.1 | 7.8e9 |
| 7.2 | 5.2e9 |
| 7.3 | 4.2e9 |
| 7.4 | 1.2e10 |
| 7.5 | 1.2e10 |
| 7.6 | 8.0e9 |
| 7.7 | 1.3e10 |
| 7.8 | 1.6e10 |
| 7.9 | 1.0e10 |
| 7.10 | 7.3e9 |
| 7.11 | 1.1e10 |
| 7.12 | 5.5e9 |
| 7.13 | 1.5e10 |
| 7.14 | 7.4e9 |
| 7.15 | 1.5e10 |
| 7.16 | 1.2e10 |
| 7.17 | 7.8e9 |
| vehicle | 1.4e5 |
| Benchmark | 6.3e9 |
| LNP | |

The levels of five proinflammatory cytokines present in serum were evaluated at 4 hours after LNP administration as described in Example 81. The levels of the proinflammatory cytokines interleukin-6 (IL-6), interferon gamma (INF-G), tumor necrosis factor alpha (TNF-α), monocyte chemoattractant protein-1 (MCP-1), macrophage inflammatory protein-1 beta (MIP-1b) in mice treated with LNPs of the present disclosure are shown in Table 44.

TABLE 43
cytokine concentration (pg/ml)

| LNP ID | IL-6 | INF-G | TNF-a | MCP-1 | MIP-1b |
|---|---|---|---|---|---|
| 7.1 | 194 | 120 | 104 | 3845 | 7243 |
| 7.2 | 3322 | 346 | 301 | 9701 | 12619 |
| 7.3 | 2246 | 134 | 162 | 6577 | 9186 |
| 7.4 | 44817 | 3584 | 871 | 18629 | 20143 |
| 7.5 | 7779 | 687 | 346 | 13486 | 17031 |
| 7.6 | 7722 | 1381 | 426 | 12547 | 15716 |
| 7.7 | 71045 | 7002 | 1707 | 28081 | 35488 |
| 7.8 | 1419 | 78 | 222 | 7955 | 13158 |
| 7.9 | 3932 | 893 | 336 | 9148 | 16790 |
| 7.10 | 6 | 19 | 37 | 2811 | 5895 |
| 7.11 | 340 | 6 | 118 | 4521 | 10916 |
| 7.12 | 65125 | 6194 | 1068 | 21932 | 24840 |
| 7.13 | 25147 | 1297 | 1498 | 19819 | 27431 |
| 7.14 | 57905 | 5404 | 1533 | 23009 | 28408 |
| 7.15 | 14011 | 1079 | 696 | 13399 | 23506 |
| 7.16 | 52095 | 2037 | 1916 | 20072 | 35240 |
| 7.17 | 12014 | 693 | 439 | 11789 | 19682 |
| vehicle | 0 | 0 | 0 | 0 | 0 |
| Benchmark | 4719 | 204 | 337 | 9074 | 16998 |
| LNP | | | | | |

In addition, the body weight of mice treated with LNP compositions of the present disclosure was assessed prior to intravenous administration and 24 hours post-administration and the body weights at baseline and post-treatment were compared. The average percentage of body weight change for each group of mice treated with each LNP composition is shown in Table 45.

TABLE 44

| | Body weight |
|---|---|
| LNP ID. | change |
| 7.1 | −5.7% |
| 7.2 | −7.9% |
| 7.3 | −7.8% |
| 7.4 | −11.9% |
| 7.5 | −10.4% |
| 7.6 | −9.0% |
| 7.7 | −10.7% |
| 7.8 | −6.9% |
| 7.9 | −12.5% |
| 7.10 | −3.1% |
| 7.11 | −6.2% |
| 7.12 | −10.4% |
| 7.13 | −9.5% |
| 7.14 | −9.8% |
| 7.15 | −10.6% |
| 7.16 | −11.2% |
| 7.17 | −9.2% |
| vehicle | −1.1% |
| Benchmark | −6.4% |
| LNP | |

The results of this example show that the addition of GalNac to LNP compositions of the present disclosure for the administration of co-encapsulated mRNA and DNA to liver cells in vivo improved tolerability compared to a benchmark LNP composition comprising GalNac or LNP compositions of the present disclosure that did not comprise GalNac. LNP compositions of the present disclosure with GalNac (e.g., LNP IDs 7.1, 7.5, 7.8, 7.9, 7.10, 7.11) exhibited comparable or higher luciferase expression and comparable, or even reduced, cytokine induction relative to a benchmark LNP composition, also with GalNac, that is known to induce lower cytokine levels when delivered to liver cells without GalNac. Further, the addition of GalNac to certain LNP compositions of the present disclosure resulted in significantly lower cytokine levels compared to the same LNP compositions without GalNac (e.g., compare LNP ID. 7.14 to LNP ID. 7.15 and LNP ID. 7.16 to LNP ID 7.17). Administration of LNP compositions of the present disclosure with GalNac was also well tolerated in the mice, with body weight loss at 24 hours in treated mice comparable to body weight loss at 24 hours in mice treated with the benchmark LNP composition. Most of the mice lost some weight 24 hours after administration but then recovered.

### Example 91—LNP Compositions Comprising Tannic Acid Enhance Delivery of DNA to HepG2 Liver Cells In Vitro

This experiment shows the ability of LNP compositions of the present disclosure that comprise tannic acid to enhance delivery of DNA to liver HepG2 cells in vitro.

A series of LNP compositions of the present disclosure were prepared comprising varying tannic acid: DNA weight ratio (7.5, 10, 12.5, 15) and a DNA nanoplasmid comprising a gene encoding GFP operably associated with an EF1-a promoter. The compositions and lipid: DNA ratios of the LNP compositions are listed in Table 46.

TABLE 45

| | | | | PEG- | | |
|---|---|---|---|---|---|---|
| | COMPOUND | DOPC | Chol | DMG | Tannic | |
| LNP | NO. 37 | (% | (% | (% | Acid:DNA | Lipid:DNA |
| ID | (% moles) | moles) | moles) | moles) | (w/w) | (w/w) |
| A.1 | 50 | 10 | 38.5 | 1.5 | 0 | 80 |
| A.2 | 50 | 10 | 38.5 | 1.5 | 7.5 | 80 |
| A.3 | 50 | 10 | 38.5 | 1.5 | 10 | 80 |
| A.4 | 50 | 10 | 38.5 | 1.5 | 12.5 | 80 |
| A.5 | 50 | 10 | 38.5 | 1.5 | 15 | 80 |

LNP compositions A.1-A.5 exhibit similar particle size (70.4-113.2 μM), pdi (0.07-0.11), zeta potential (−1.12-−1.5,), and percent DNA encapsulation efficiencies (98.8%-99.5%). These LNP compositions were used to transfect cultured HepG2 liver cells in vitro.

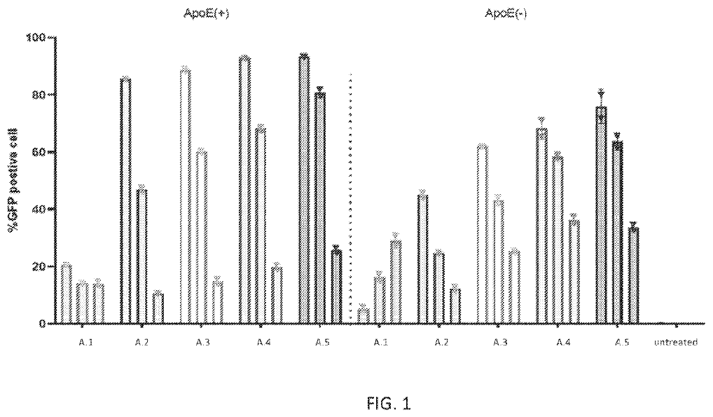

HepG2 cells were detached from a T75 flask by washing the monolayer with 1×DPBS, then incubating with TrypLE 1× buffer for 5 min, and then quenched with EMEM+10% media. The cells were pelleted by centrifugation and resuspended in EMEM+10% FBS, and approximately 60,000 HepG2 cells were dispensed in 96-well plates. The cells were allowed to attach and grow in the 96 well plates overnight prior to treatment. The following day, each of the LNP A.1-A.5 compositions was titrated in 1×DPBS to deliver the DNA nanoplasmid at a concentration of 0.06 μg/well, 0.17 μg/well or 0.5 μg/well +/−recombinant ApoE4. For the ApoE4 delivery conditions, a second 96-well LNP dilution plate was made with the aforementioned concentrations to include 1 ug of recombinant ApoE4 added to each well. These formulations were then added to the HepG2 cells. After 48 hours, the percentage of GFP positive HepG2 cells was determined by detaching the cells from plates using TrypLE as described above and running flow cytometry evaluating % GFP+ cells. The results are shown in FIG. 1.

As shown in FIG. 1, the calculated percentage of GFP positive cells demonstrated a dose-dependent response with increasing concentrations of DNA nanoplasmid encapsulated into the LNP composition resulting in increasing percentages of GFP positive cells. LNP formulations, in the presence or absence of recombinant ApoE4, demonstrated increasing percentages of GFP positive cells with increasing tannic acid concentrations, though LNP samples containing ApoE demonstrated the higher percentages of GFP positive cells at higher tannic acid concentrations. These results demonstrate that the addition of tannic acid to LNP compositions of the present invention results in enhanced DNA delivery and expression in HepG2 cells in the absence or presence of ApoE.

### Example 92—LNP Compositions Comprising Tannic Acid Enhance Delivery of DNA to Liver Cells In Vivo

This experiment shows the ability of LNP compositions of the present disclosure that comprise tannic acid to enhance delivery of DNA to liver cells in vivo.

In this experiment, each group of adult female BALB/C mice (n=3/group) was intravenously administered via tail vein injection a DNA nanoplasmid encoding the fluc gene operably associated with the constitutive CMV promoter. The DNA molecules were formulated within LNP compositions A. 1 and A.3 of the present disclosure (Table 46). The LNP compositions of the present disclosure (0.25 mg/kg) were administered to the mice from each of the two groups.

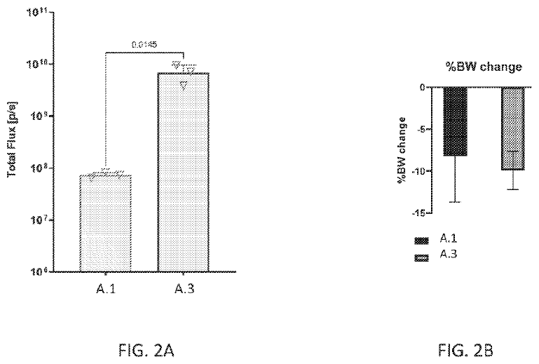

Body weight loss (BWL) measurements were taken at 24 hrs post administration and luciferase expression was measured by whole body luminescence imaging (BLI) at 48 hours post-administration. The results of BLI measurements (total flux [p/s]) are shown in FIGS. 2A and 2B.

As shown in FIG. 2A, the addition of tannic acid to the LNP composition (A.3) resulted in an increase in BLI of over 100-fold compared to LNP composition lacking tannic acid (A.1).

BLW measurements at 24 hours showed a similar degree of loss amongst the tested groups suggesting tannic acid does not directly increase BLW (FIG. 2B).

### Example 93—Addition of GalNac to LNP Compositions Comprising Tannic Acid Enhances Delivery of DNA to Liver Cells and Reduces Body Weight Loss in Treated Animals

This experiment shows that GalNac added to LNP compositions of the present disclosure that comprise tannic acid enhances delivery of DNA to liver cells in vivo, while reducing body weight loss in treated animals.

In this experiment, each group of adult female BALB/C mice (n=3/group) was intravenously administered via tail vein injection a DNA nanoplasmid encoding the fluc gene operably associated with the constitutive CMV promoter. The DNA molecule was formulated within LNP compositions of the present disclosure listed in Table 47.

TABLE 46

| | | | | PEG- | | | |
|---|---|---|---|---|---|---|---|
| | COMPOUND | DOPC | Chol | DMG | GalNac | Tannic | |
| LNP | NO. 37 | (% | (% | (% | (% | Acid:DNA | Lipid:DNA |
| ID | (% moles) | moles) | moles) | moles) | moles) | (w/w) | (w/w) |
| A.1 | 50 | 10 | 38.5 | 1.5 | 0 | 0 | 80 |
| A.3 | 50 | 10 | 38.5 | 1.5 | 0 | 10 | 80 |
| A.6 | 50 | 10 | 38.5 | 1.0 | 0.5 | 10 | 80 |

The LNP compositions of the present disclosure (0.5 mg/kg) were administered to the mice from each of the three groups. One group of mice was treated with vehicle (PBS, Thermo Fisher Scientific, USA) as a negative control.

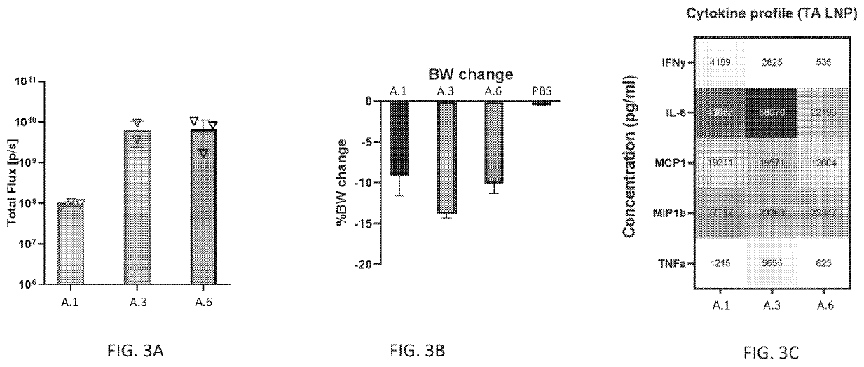

Blood was drawn from control and treated mice 4 hrs post-administration for cytokine analysis, body weight loss (BWL) measurements were taken at 24 hrs post administration and luciferase expression was measured by whole body luminescence imaging (BLI) at 48 hours post-administration. The results of BLI measurements (total flux [p/s]), BWL measurements, and cytokine analysis are shown in FIGS. 3A-3C.

As shown in FIG. 3A, the addition of GalNac (LNP A.6) to the tannic acid comprising LNP composition (A.3) resulted in a similar increase in increase in BLI of over 100-fold compared to the LNP composition A.3 comprising tannic acid (FIG. 3A).

The cytokine profile of the LNP compositions lacking tannic acid (LNP A.1), comprising tannic acid (LNP A.3) and comprising tannic acid and GalNac (LNP A.6) is shown in FIG. 3C. As shown in FIG. 3C, the addition of GalNac to LNP compositions of the present disclosure resulted in decreased cytokine elevation for all tested cytokines compared to LNP compositions comprising or lacking tannic acid. The addition of tannic acid to the LNP composition (LNP A.3) demonstrated some elevation in cytokine levels compared to those lacking tannic acid (LNP A.1); however, GalNac addition was capable of suppressing and further reducing cytokine levels for several cytokines (compare A.6 v A.3).

The addition of GalNac to tannic acid comprising LNP composition (LNP A.6) resulted in a reduction in BWL of about 30% compared to LNP compositions comprising tannic acid (A.3) but slightly greater than LNP compositions lacking tannic acid (FIG. 3B).

### Example 94—LNP Compositions Comprising Tannic Acid Enhance Delivery of Co-Encapsulated DNA and RNA to Liver Cells In Vivo

This experiment shows the ability of LNP compositions of the present disclosure that comprise tannic acid to enhance delivery of co-encapsulated DNA and RNA to liver cells in vivo.

In this experiment, FVIII expression indicates successful delivery by LNP compositions of a two-component, DNA/RNA system to liver cells, resulting in transposition of the FVIII transgene facilitated by SPB.

Wild-type C57BL/6 adult mice (N=4) were dosed with a single co-encapsulated LNP encapsulating both FVIII transposon and SPB. The compositions of the co-encapsulated LNPs are shown in Table 48, incorporating mRNA encoding active SPB and TTR-FVIII nanoplasmid DNA (SEQ ID NO: 9). All cytidine residues in the mRNA were 5-methylcytidine (5-MeC).

TABLE 47

| | | | | PEG- | | | |
|---|---|---|---|---|---|---|---|
| | COMPOUND | DOPC | Chol | DMG | GalNac | Tannic | |
| LNP | NO. 37 | (% | (% | (% | (% | Acid:DNA | Lipid:DNA + |
| ID | (% moles) | moles) | moles) | moles) | moles) | (w/w) | RNA (w/w) |
| B.1 | 45 | 7.5 | 45.75 | 1.5 | 0.25 | 0 | 50 |
| B.2 | 45 | 7.5 | 45.75 | 1.5 | 0.25 | 7 | 50 |

Each group of mice received 1 mg/kg of co-encapsulated LNP encapsulating mRNA and DNA at a 1:2 mRNA: DNA ratio. One group of mice was treated with vehicle (PBS, Thermo Fisher Scientific, USA) as a negative control.

Prior to administration, mice were placed under anesthesia induced by isoflurane. For delivery, 50-80 μL of co-encapsulated LNP was drawn into a single 29 gauge insulin syringe, and delivered via intravenous (IV) through the retro-orbital sinus. On Day 6 post-treatment, plasma was collected from treated mice. For plasma collection, treated mice were put under anesthesia with isoflurane, approximately 150 μL whole blood was retro-orbitally collected, whole blood was mixed with 10% volume of 3.2% sodium citrate, centrifuged at 15,000 g for 15 minutes at 20° C., and plasma supernatant was collected. hFVIII antigen levels were measured using the Visualize™ Factor VIII Antigen Plus Kit (Affinity Biologicals™ Inc.).

The results of FVIII antigen expression are shown in Table 49.

TABLE 48

| | % of Normal |
|---|---|
| | hFVIII |
| LNP ID | Plasma Ag |
| B.1 | 66 |
| B.2 | 182 |
| vehicle | 0 |

As shown in Table 49, the addition of tannic acid to the LNP composition (B.2) resulted in an increase in FVIII antigen expression of about 3-fold compared to the LNP composition lacking tannic acid (B.2).

### Example 95—In Vivo LNP Delivery of Co-Encapsulated mRNA and DNA to Liver is Enhanced by Addition of Tannic Acid

The following is a non-limiting example demonstrating that lipid nanoparticle compositions comprising GalNac can be used to deliver co-encapsulated mRNA and DNA to liver cells in vivo and this delivery is enhanced by the addition of tannic acid to the LNPs.

In this experiment, FVIII expression indicates successful delivery by LNP compositions of a two-component, DNA/RNA system to liver cells, resulting in transposition of the FVIII transgene facilitated by SPB.

Wild-type C57BL/6 adult mice (N=4) were dosed with a single co-encapsulated LNP encapsulating both FVIII transposon and SPB. The compositions of the co-encapsulated LNPs are shown in Table 50, incorporating mRNA encoding active SPB and TTR-FVIII nanoplasmid DNA (SEQ ID NO: 9). All cytidine residues in the mRNA were 5-methylcytidine (5-MeC).

TABLE 49

| | | | | PEG- | | | |
|---|---|---|---|---|---|---|---|
| | COMPOUND | DOPC | Chol | DMG | GalNac | Tannic | Lipid:DNA + |
| LNP | NO. 37 | (% | (% | (% | (% | Acid:DN | RNA |
| ID | (% moles) | moles) | moles) | moles | moles) | A (w/w) | (w/w) |
| C.1 | 45 | 10 | 42.5 | 2 | 0.5 | 0 | 60 |
| C.2 | 50 | 7.5 | 41 | 1 | 0.5 | 0 | 60 |
| C.3 | 45 | 7.5 | 45.75 | 1.5 | 0.25 | 0 | 50 |
| C.4 | 45 | 7.5 | 45.75 | 1.5 | 0.25 | 10:1 | 50 |
| C.5 | 40 | 5 | 53.5 | 1 | 0.5 | 0 | 50 |
| C.6 | 40 | 10 | 48 | 1.5 | 0.5 | 0 | 40 |
| C.7 | 40 | 5 | 52.75 | 2 | 0.25 | 0 | 60 |
| C.8 | 50 | 10 | 38.5 | 1 | 0.5 | 0 | 50 |

Each group of mice received 1 mg/kg of co-encapsulated LNP encapsulating mRNA and DNA at a 1:2 mRNA: DNA ratio. One group of mice was treated with vehicle (PBS, Thermo Fisher Scientific, USA) as a negative control.

As described in Example 94, hFVIII antigen levels were measured and the results are shown in Table 51.

TABLE 51

| | % of Normal |
|---|---|
| | hFVIII |
| LNP ID | Plasma Ag |
| C.1 | 41.3 |
| C.2 | 139.7 |
| C.3 | 66.2 |
| C.4 | 242.9 |
| C.5 | 28.1 |
| C.6 | 11.1 |
| C.7 | 24.9 |
| C.8 | 21.8 |
| vehicle | 0 |

As shown in Table 51, LNP compositions comprising GalNac successfully delivered co-encapsulated mRNA and DNA to liver cells in vivo and the addition of tannic acid to the LNP composition (C.4) resulted in an increase in FVIII antigen expression of about 3.5-fold compared to the LNP composition lacking tannic acid (C.3).

### Example 96—In Vivo LNP Delivery of Co-Encapsulated mRNA and DNA to Liver is Enhanced by Addition of Tannic Acid

This experiment shows the ability of LNP compositions of the present disclosure that comprise varying tannic acid: DNA weight ratios to enhance delivery of co-encapsulated DNA and RNA to liver cells in vivo.

In this experiment, adult BALB/C mice (n=3) were dosed with either (a) a DNA nanoplasmid encoding the fluc gene or (b) a single co-encapsulated LNP encapsulating both firefly luciferase transposon and SPB.

The compositions of the LNPs are shown in Table 52, incorporating either the DNA fluc nanoplasmid (SEQ ID NO: 8) or the mRNA encoding active SPB and the nanoplasmid DNA (SEQ ID NO: 8). All cytidine residues in the mRNA were 5-methylcytidine (5-MeC).

TABLE 50

| | | | | PEG- | | | | Tannic |
|---|---|---|---|---|---|---|---|---|
| | COMPOUND | DOPC | Chol | DMG | GalNac | Tannic | Lipid: | acid:total |
| LNP | NO. 37 | (% | (% | (% | (% | Acid:DNA | DNA + RNA | lipid |
| ID | (% moles) | moles) | moles) | moles) | moles) | (w/w) | (w/w) | (w/w) |
| D.1 | 50 | 7.5 | 41 | 1 | 0.5 | 0 | 60 | 0 |
| D.2 | 50 | 7.5 | 41 | 1 | 0.5 | 5:1 | 60 | 0.08 |
| D.3 | 50 | 7.5 | 41 | 1 | 0.5 | 10:1 | 60 | 0.17 |
| D.4 | 50 | 7.5 | 41 | 1 | 0.5 | 15:1 | 60 | 0.25 |
| D.5 | 45 | 7.5 | 45.75 | 1.5 | 0.25 | 0 | 50 | 0 |
| D.6 | 45 | 7.5 | 45.75 | 1.5 | 0.25 | 5:1 | 50 | 0.10 |
| D.7 | 45 | 7.5 | 45.75 | 1.5 | 0.25 | 10:1 | 50 | 0.20 |
| D.8 | 45 | 7.5 | 45.75 | 1.5 | 0.25 | 15:1 | 50 | 0.30 |

Each group of mice received either (a) 0.5 mg/kg of LNP encapsulating DNA only or (b) 0.75 mg/kg of co-encapsulated LNP encapsulating mRNA and DNA at a 1:2 mRNA: DNA ratio. One group of mice was treated with vehicle (PBS, Thermo Fisher Scientific, USA) as a negative control.

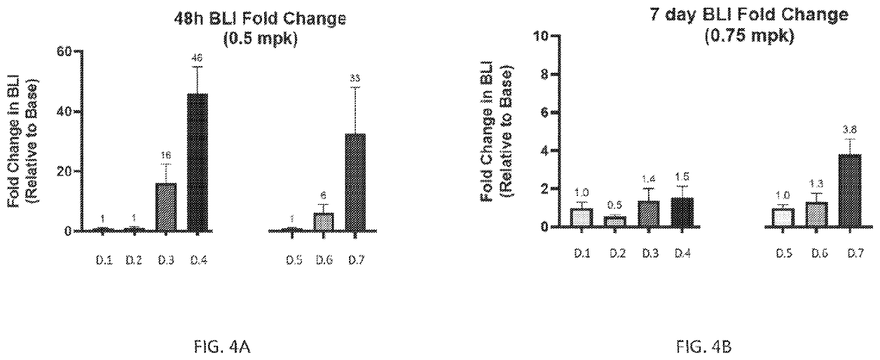

Luciferase expression was measured by whole body luminescence imaging (BLI) at 48 hours or one week post-administration. The results of BLI measurements (total flux [p/s]), are shown in FIGS. 4A and 4B.

As shown in FIG. 4A, the addition of increasing tannic acid concentration to LNP compositions resulted in an increase in BLI of up to about 46-fold in mice treated with DNA nanoplasmid alone compared to vehicle. FIG. 4B shows the addition of increasing tannic acid concentration to LNP compositions resulted in an increase in BLI of up to about 3.8-fold in mice treated with co-encapsulated RNA and DNA compared to vehicle.

### Example 97—In Vivo LNP Delivery of Co-Encapsulated mRNA and DNA to Liver is Enhanced by Addition of Tannic Acid

This experiment shows the ability of LNP compositions of the present disclosure that comprise varying tannic acid: total lipid weight ratios to enhance delivery of co-encapsulated DNA and RNA to liver cells in vivo.

Wild-type C57BL/6 adult mice (N=4) were dosed with a single co-encapsulated LNP encapsulating both FVIII transposon and SPB. The compositions of the co-encapsulated LNPs are shown in Table 53, incorporating mRNA encoding active SPB and TTR-FVIII nanoplasmid DNA (SEQ ID NO: 9). All cytidine residues in the mRNA were 5-methylcytidine (5-MeC).

TABLE 53

| | | | | PEG- | | | Tannic |
|---|---|---|---|---|---|---|---|
| | COMPOUND | DOPC | Chol | DMG | GalNac | Lipid: | acid:total |
| LNP | NO. 37 | (% | (% | (% | (% | DNA + RNA | lipid |
| ID | (% moles) | moles) | moles) | moles) | moles) | (w/w) | (w/w) |
| E.1 | 50 | 7.5 | 41 | 1 | 0.5 | 60 | 0 |
| E.2 | 50 | 7.5 | 41 | 1 | 0.5 | 60 | 0.2 |
| E.3 | 50 | 7.5 | 41 | 1 | 0.5 | 60 | 0.25 |
| E.4 | 45 | 7.5 | 45.75 | 1.5 | 0.25 | 50 | 0 |
| E.5 | 45 | 7.5 | 45.75 | 1.5 | 0.25 | 50 | 0.20 |
| E.6 | 45 | 7.5 | 45.75 | 1.5 | 0.25 | 50 | 0.25 |
| E.7 | 50 | 7.5 | 41 | 1 | 0.5 | 50 | 0 |
| E.8 | 50 | 7.5 | 41 | 1 | 0.5 | 50 | 0.2 |

Each group of mice received 0.75 mg/kg of co-encapsulated LNP encapsulating mRNA and DNA at a 1:2 mRNA: DNA ratio. One group of mice was treated with vehicle (PBS, Thermo Fisher Scientific, USA) as a negative control.

As described in Example 94, hFVIII antigen levels were measured and the results are shown in Table 54.

TABLE 54

| | % of Normal |
|---|---|
| | hFVIII |
| LNP ID | Plasma Ag |
| E.1 | 6.1 |
| E.2 | 64.5 |
| E.3 | 109.1 |
| E.4 | 6.5 |
| E.5 | 21.6 |
| E.6 | 69.2 |
| E.7 | 10.1 |
| E.8 | 41.0 |
| vehicle | 0 |

As shown in Table 54, the addition of increasing tannic acid concentration to LNP compositions resulted in increasing FVIII antigen expression.

### Example 98—LNP Compositions Comprising Proanthocyanidin Enhance Delivery of DNA to HepG2 Liver Cells In Vitro

This experiment shows the ability of LNP compositions of the present disclosure that comprise proanthocyanidin to enhance delivery of DNA to liver HepG2 cells in vitro.

A series of LNP compositions of the present disclosure were prepared comprising varying proanthocyanidin: DNA weight ratio (2.5, 5, 10, 20) and a DNA nanoplasmid encoding the fluc gene operably associated with the constitutive CMV promoter. LNP ID A.5 from Example 91, comprising tannic acid as the additive, was also prepared. The compositions and lipid: DNA ratios of the LNP compositions are listed in Table 55.

TABLE 55

| | | | | PEG- | | |
|---|---|---|---|---|---|---|
| | COMPOUND | DOPC | Chol | DMG | | |
| LNP | NO. 37 | (% | (% | (% | Proanthocyanidin:DNA | Lipid:DNA |
| ID | (% moles) | moles) | moles) | moles) | (w/w) | (w/w) |
| F.1 | 50 | 10 | 38.5 | 1.5 | 0 | 80 |
| F.2 | 50 | 10 | 38.5 | 1.5 | 2.5 | 80 |
| F.3 | 50 | 10 | 38.5 | 1.5 | 5 | 80 |
| F.4 | 50 | 10 | 38.5 | 1.5 | 10 | 80 |
| F.5 | 50 | 10 | 38.5 | 1.5 | 20 | 80 |
| A.5 | 50 | 10 | 38.5 | 1.5 | Tannic acid:DNA (w/w) | 80 |
| | | | | | 15 | |

LNP compositions F.1-F.5 exhibit similar particle size (76.5-123.1 μM), pdi (0.05-0.15), zeta potential (−0.73-−2.22), and percent DNA encapsulation efficiencies (99.2%-99.8%). These LNP compositions were used to transfect cultured HepG2 liver cells in vitro.

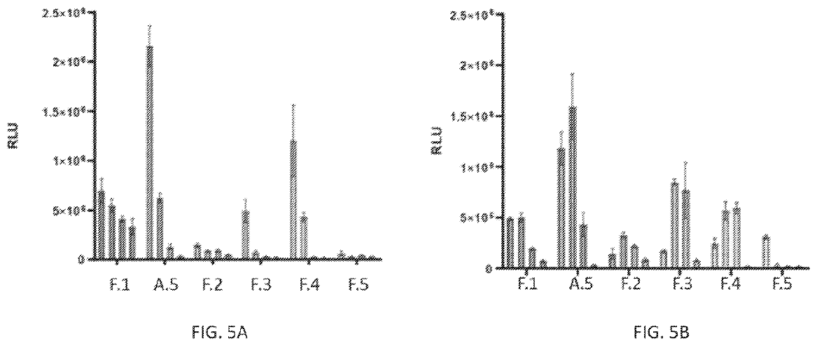

HepG2 cells were detached from a T75 flask by washing the monolayer with 1× DPBS, then incubating with TrypLE 1× buffer for 5 min, and then quenched with EMEM+10% media. The cells were pelleted by centrifugation and resuspended in EMEM+10% FBS, and approximately 60,000 HepG2 cells were dispensed in 96-well plates. The cells were allowed to attach and grow in the 96 well plates overnight prior to treatment. The following day, each of the LNP compositions was titrated in 1×DPBS to deliver the DNA nanoplasmid at a concentration of 0.02 ug/well, 0.06 μg/well, 0.17 μg/well or 0.5 μg/well+/−recombinant ApoE4. For the ApoE4 delivery conditions, a second 96-well LNP dilution plate was made with the aforementioned concentrations to include 1 ug of recombinant ApoE4 added to each well. These formulations were then added to the HepG2 cells. After 48 hours, the luciferase expression by the HepG2 cells was determined after detaching the cells from plates using TrypLE as described above. The results are shown in FIG. 5A and FIG. 5B.

As shown in FIG. 5A and FIG. 5B, luciferase expression by the HepG2 cells generally demonstrated a dose-dependent response with increasing concentrations of DNA nanoplasmid encapsulated into the LNP composition resulting in increasing luciferase expression. LNP formulations, in the presence (FIG. 5B) or absence (FIG. 5A) of recombinant ApoE4, generally demonstrated increasing luciferase expression with increasing proanthocyanidin concentrations up to 10:1 proanthocyanidin: DNA (w/w). FIG. 5 also shows that the LNP composition A.5 comprising tannic acid, in the presence or absence of recombinant ApoE4, demonstrated higher luciferase expression than the LNP compositions comprising proanthocyanidin. These results demonstrate that the addition of proanthocyanidin or tannic acid to LNP compositions of the present invention results in enhanced DNA delivery and expression in HepG2 cells in the absence or presence of ApoE.

### Example 99—LNP Compositions Comprising Proanthocyanidin Enhance Delivery of DNA to HepG2 Liver Cells In Vitro

This experiment shows the ability of LNP compositions of the present disclosure that comprise proanthocyanidin to enhance delivery of DNA to liver HepG2 cells in vitro.

A series of LNP compositions of the present disclosure were prepared comprising varying proanthocyanidin: DNA weight ratio (7.5, 10, 12.5, 15) and a DNA nanoplasmid encoding the fluc gene operably associated with the constitutive CMV promoter. The compositions and lipid: DNA ratios of the LNP compositions are listed in Table 56.

TABLE 56

| | | | | PEG- | | |
|---|---|---|---|---|---|---|
| | COMPOUND | DOPC | Chol | DMG | | |
| LNP | NO. 37 | (% | (% | (% | Proanthocyanidin:DNA | Lipid:DNA |
| ID | (% moles) | moles) | moles) | moles) | (w/w) | (w/w) |
| G.1 | 50 | 10 | 38.5 | 1.5 | 0 | 80 |
| G.2 | 50 | 10 | 38.5 | 1.5 | 7.5 | 80 |
| G.3 | 50 | 10 | 38.5 | 1.5 | 10 | 80 |
| G.4 | 50 | 10 | 38.5 | 1.5 | 12.5 | 80 |
| G.5 | 50 | 10 | 38.5 | 1.5 | 15 | 80 |

LNP compositions G.1-G.5 exhibit similar particle size (73.8-90.1 μM), pdi (0.09-0.12), zeta potential (−0.03-−1.61), and percent DNA encapsulation efficiencies (98.6%-100%). These LNP compositions were used to transfect cultured HepG2 liver cells in vitro.

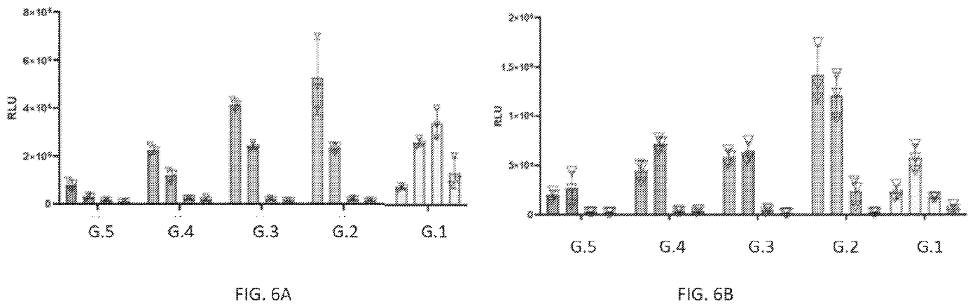

As described in Example 65, luciferase expression after 48 hours by the HepG2 cells was determined after detaching the cells from plates using TrypLE. The results are shown in FIG. 6A and FIG. 6B.

As shown in FIG. 6A and FIG. 6B, both in the presence (FIG. 6B) or absence (FIG. 6A) of recombinant ApoE4, the highest luciferase expression was observed in cells transfected with the LNP formulation with 7.5:1 proanthocyanidin: DNA (w/w). These results demonstrate that the addition of proanthocyanidin to LNP compositions of the present invention results in enhanced DNA delivery and expression in HepG2 cells in the absence or presence of ApoE.

### Example 100—LNP Compositions Comprising Ellagic Acid or Punicalagin Enhance Delivery of DNA to HepG2 Liver Cells In Vitro

This experiment shows the ability of LNP compositions of the present disclosure that comprise ellagic acid or punicalagin to enhance delivery of DNA to liver HepG2 cells in vitro.

A series of LNP compositions of the present disclosure were prepared comprising ellagic acid or punicalagin at additive: DNA weight ratios of 5:1 or 10:1 and a DNA nanoplasmid encoding the fluc gene operably associated with the constitutive CMV promoter. The compositions and lipid: DNA ratios of the LNP compositions are listed in Table 57.

TABLE 57

| | | | | PEG- | | |
|---|---|---|---|---|---|---|
| | COMPOUND | DOPC | Chol | DMG | | |
| LNP | NO. 37 | (% | (% | (% | | Lipid:DNA |
| ID | (% moles) | moles) | moles) | moles) | Additive:DNA (w/w) | (w/w) |
| H.1 | 50 | 10 | 38.5 | 1.5 | 0 | 80 |
| H.2 | 50 | 10 | 38.5 | 1.5 | ellagic acid:DNA | 80 |
| | | | | | 5:1 | |
| H.3 | 50 | 10 | 38.5 | 1.5 | ellagic acid:DNA | 80 |
| | | | | | 10:1 | |
| H.4 | 50 | 10 | 38.5 | 1.5 | punicalagin:DNA | 80 |
| | | | | | 5:1 | |
| H.5 | 50 | 10 | 38.5 | 1.5 | punicalagin:DNA | 80 |
| | | | | | 10:1 | |

LNP compositions H.1-G.5 exhibit similar particle size (73.3-90.1 μM), pdi (0.04-0.22), zeta potential (−0.518-−1.76), and percent DNA encapsulation efficiencies (99.4%-99.7%). These LNP compositions were used to transfect cultured HepG2 liver cells in vitro.

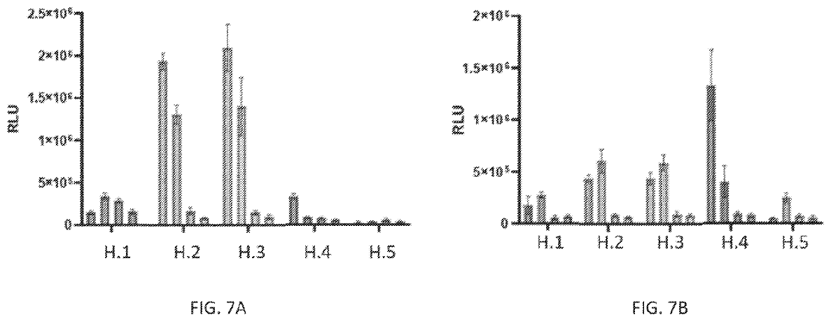

As described in Example 98, luciferase expression after 48 hours by the HepG2 cells was determined after detaching the cells from plates using TrypLE. The results are shown in FIG. 7A and FIG. 7B.

As shown in FIG. 7A and FIG. 7B, in the absence of recombinant ApoE4 (FIG. 7A), the highest luciferase expression was observed in cells transfected with the LNP formulations H.2 and H.3 comprising ellagic acid. FIG. 7 also shows in the presence of recombinant ApoE4 (FIG. 7B), the highest luciferase expression was observed in cells transfected with the LNP formulation H.4 comprising punicalagin, though LNP formulations H.2 and H.3 comprising ellagic acid also demonstrated higher luciferase expression than the base formulation H.1 not comprising any additive. These results demonstrate that the addition of ellagic acid or punicalagin to LNP compositions of the present invention results in enhanced DNA delivery and expression in HepG2 cells in the absence or presence of ApoE.

### Example 101—LNP Compositions Comprising Proanthocyanidin, Ellagic Acid or Punicalagin to Enhance Delivery of DNA to Liver Cells In Vivo

This experiment shows the ability of LNP compositions of the present disclosure that comprise proanthocyanidin, ellagic acid or punicalagin to enhance delivery of DNA to liver cells in vivo.

In this experiment, each group of adult female BALB/C mice (n=3/group) was intravenously administered via tail vein injection a DNA nanoplasmid encoding the fluc gene operably associated with the constitutive CMV promoter. The DNA molecules were formulated within LNP compositions of the present disclosure (Table 58). The LNP compositions of the present disclosure (0.5 mg/kg) were administered to the mice from each of the groups.

TABLE 58

| | | | | PEG- | | |
|---|---|---|---|---|---|---|
| | COMPOUND | DOPC | Chol | DMG | | |
| LNP | NO.37 | (% | (% | (% | | Lipid:DNA |
| ID | (% moles) | moles) | moles) | moles) | Additive:DNA (w/w) | (w/w) |
| I.1 | 50 | 10 | 38.5 | 1.5 | 0 | 80 |
| I.2 | 50 | 10 | 38.5 | 1.5 | proanthocyanidin:DNA | 80 |
| | | | | | 2.5:1 | |
| I.3 | 50 | 10 | 38.5 | 1.5 | proanthocyanidin:DNA | 80 |
| | | | | | 5:1 | |
| I.4 | 50 | 10 | 38.5 | 1.5 | ellagic acid:DNA | 80 |
| | | | | | 2.5:1 | |
| I.5 | 50 | 10 | 38.5 | 1.5 | ellagic acid:DNA | 80 |
| | | | | | 5:1 | |
| I.6 | 50 | 10 | 38.5 | 1.5 | ellagic acid:DNA | 80 |
| | | | | | 10:1 | |
| I.7 | 50 | 10 | 38.5 | 1.5 | punicalagin:DNA | 80 |
| | | | | | 2.5:1 | |

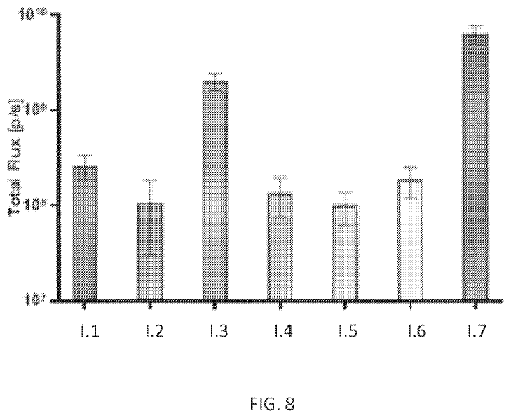

Luciferase expression was measured by whole body luminescence imaging (BLI) at 48 hours post-administration. The results of BLI measurements (total flux [p/s]) are shown in FIG. 8.

As shown in FIG. 8, the addition of proanthocyanidin to the LNP composition (I.3) resulted in an increase in BLI of about 7.8-fold, and the addition of punicalagin to the LNP composition (I.7) resulted in an increase of BLI of about 24-fold, compared to LNP composition lacking an additive (I.1).

### Example 102—LNP Compositions Comprising Tannic Acid Enhance Delivery of Co-Encapsulated mRNA and DNA to Liver Cells In Vivo

This experiment shows the ability of LNP compositions of the present disclosure that comprise tannic acid to enhance delivery of co-encapsulated mRNA and DNA to liver cells in vivo.

In this experiment, HA-tagged human propionyl-CoA carboxylase subunit alpha (PCCA) expression indicates successful delivery by LNP compositions of a two-component, DNA/RNA system to liver cells, resulting in transposition of the HA-PCCA transgene facilitated by SPB. The PCCA gene encodes for the alpha subunit of propionyl-CoA carboxylase, which is an enzyme that plays a role in the normal processing of proteins, such as several amino acids (e.g., isoleucine, methionine, threonine and valine), and certain types of lipids (fats) and cholesterol.

Wild-type C57BL/6 adult mice (N=5) were dosed with a single co-encapsulated LNP encapsulating both HA-PCCA transposon and SPB. The compositions of the co-encapsulated LNPs are shown in Table 59, incorporating mRNA encoding active SPB and HA-PCCA transposon nanoplasmid DNA (SEQ ID NO: 10). All cytidine residues in the mRNA were 5-methylcytidine (5-MeC).

TABLE 59

| | | | | PEG- | | | |
|---|---|---|---|---|---|---|---|
| | COMPOUND | DOPC | Chol | DMG | GalNac | Tannic | |
| LNP | NO. 37 | (% | (% | (% | (% | Acid:DNA | Lipid:DNA + RNA |
| ID | (% moles) | moles) | moles) | moles) | moles) | (w/w) | (w/w) |
| J.1 | 50 | 7.5 | 41 | 1 | 0.5 | 0 | 60 |
| J.2 | 50 | 7.5 | 41 | 1 | 0.5 | 0.15:1 | 60 |
| J.3 | 50 | 7.5 | 41 | 1 | 0.5 | 0.2:1 | 60 |
| J.4 | 45 | 7.5 | 45.75 | 1.5 | 0.25 | 0 | 50 |
| J.5 | 45 | 7.5 | 45.75 | 1.5 | 0.25 | 0.15:1 | 50 |
| J.6 | 45 | 7.5 | 45.75 | 1.5 | 0.25 | 0.2:1 | 50 |

Each group of mice received 0.5 mg/kg or 0.75 mg/kg of co-encapsulated LNP encapsulating mRNA and DNA at a 1:2 mRNA: DNA ratio. One group of mice was treated with vehicle (PBS, Thermo Fisher Scientific, USA) as a negative control. Another group of mice was treated with a benchmark LNP composition comprising the cationic lipid HMA-404 with the following structure:

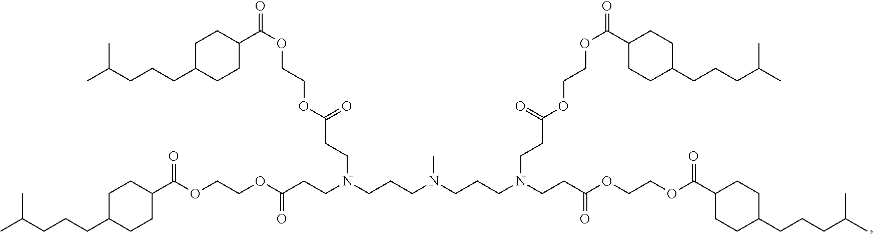 [SMILES: CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]

prepared as described in PCT Application No. PCT/US2023/061005.

Prior to administration, mice were placed under anesthesia induced by isoflurane. For delivery, 50-80 μL of co-encapsulated LNP was drawn into a single 29 gauge insulin syringe, and delivered via intravenous (IV) through the retro-orbital sinus. On Day 14 post-treatment, liver samples were obtained from the mice that underwent treatment. The snap-frozen livers were then homogenized using the Tissue Protein Extraction Reagent (catalog number 78510; Thermo Scientific, Waltham, MA, USA). HA-protein levels were assessed using an in-house developed HA-ELISA protocol, optimized the process by utilizing the Anti-HA-Tag rabbit polyclonal antibody from QED Bioscience (catalog number #18850-01) as the capture antibody and the Anti-HA-tag mAb-HRP-DirecT from MBL Life Science (catalog number #M180-7) as the detection antibody.

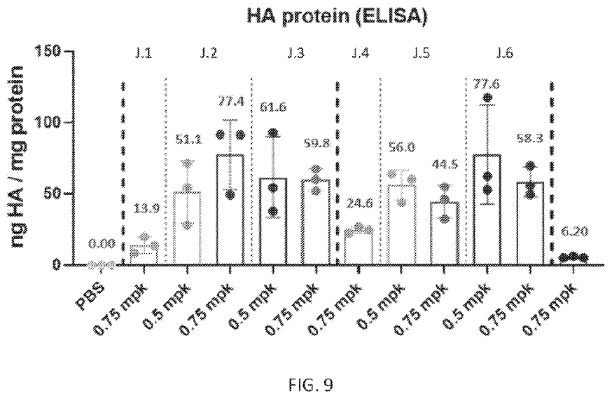

The results of PCCA-HA expression are shown in FIG. 9. As shown in FIG. 9, the addition of tannic acid to the LNP composition resulted in an increase in PCCA-HA expression compared to the LNP composition lacking tannic acid (compare J.2 and J.3 to J.1; also J.5 and J.6 to J.4). Additionally, administration of LNP compositions comprising tannic acid resulted in an increase in PCCA-HA expression of up to 10-fold compared to protein expression resulting from administration of the benchmark LNP formulation.

### Example 103—Preparation of LNPs of Present Disclosure Comprising DNA and In Vivo Screening

The following is a nonlimiting example that provides exemplary methods for formulating a plurality of multi-component LNP compositions comprising exemplary compounds of Formula (II) and DNA.

LNP compositions of the present disclosure comprising one of COMPOUND NOS. 21-76 and DNA encoding firefly luciferase (Nature Technology Corporation) were prepared as described in Example 77. The LNP compositions are shown in Table 60.

TABLE 60

| LNPs | One of | | | | PEG- | | |
|---|---|---|---|---|---|---|---|
| comprising | COMPOUND | DOPC | DSPC | Chol | DMG | Total | |
| COMPOUND | NOS. 21-76 | (% | (% | (% | (% | Lipid | Lipid:DNA |
| NO. | (% moles) | moles) | moles) | moles) | moles) | (mM) | (w/w) |
| 36 | 50 | 10 | 0 | 38.5 | 1.5 | 25 | 80 |
| 36 | 50 | 0 | 10 | 38.5 | 1.5 | 25 | 80 |
| 37 | 50 | 10 | 0 | 38.5 | 1.5 | 25 | 80 |
| 37 | 50 | 0 | 10 | 38.5 | 1.5 | 25 | 80 |
| 38 | 50 | 0 | 10 | 38.5 | 1.5 | 25 | 80 |
| 39 | 50 | 10 | 0 | 38.5 | 1.5 | 25 | 80 |
| 53 | 50 | 10 | 0 | 38.5 | 1.5 | 25 | 80 |

Adult female BALB/C mice (n=3/group) were intravenously administered 0.5 mg/kg of DNA formulated within the LNP compositions shown in Table 60. Another group of mice was treated with 0.5 mg/kg of DNA formulated within an LNP composition comprising the following compound 100:

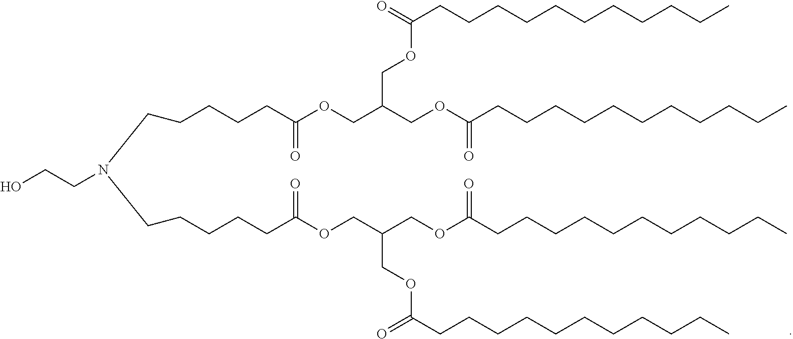 [SMILES: CCCCCCCCCCCC(=O)OCC(COC(=O)CCCCCCCCCCC)COC(=O)CCCCCN(CCO)CCCCCC(=O)OCC(COC(=O)CCCCCCCCCCC)COC(=O)CCCCCCCCCCC]

A third group of mice was treated with vehicle (PBS, Thermo Fisher Scientific, USA) as a negative control.

The location and extent of luciferase expression in treated and control mice were determined at 48 hr for DNA by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 61.

TABLE 61

| LNPs | |
|---|---|
| comprising | Total Flux |
| COMPOUND NO. | (p/s) |
| 36 (10% DOPC) | 7.52E+07 |
| 36 (10% DSPC) | 1.23E+08 |
| 37 (10% DOPC) | 7.64E+07 |
| 37 (10% DSPC) | 1.58E+08 |
| 38 | 8.29E+07 |
| 39 | 2.03E+06 |
| 53 | 4.23E+07 |
| 100 | 1.97E+07 |
| Vehicle | 1.43E+05 |

Visual analysis of the BLI images revealed that the BLI signal was predominantly located in the live. Accordingly, the results of this example show that LNP compositions of the present disclosure successfully delivered DNA in vivo, predominantly to cells in the liver, and the encoded protein was subsequently expressed by the cells. The results of this example also show that DNA delivery potency is greatly reduced when the cyclohexyl group is removed from all the tails in the lipid compound (compare LNPs comprising COMPOUND NOS. 36, 37, or 38 to LNP comprising compound 100).

### Example 104—Preparation of LNPs of Present Disclosure Comprising mRNA and In Vivo Screening

The following is a nonlimiting example that provides exemplary methods for formulating a plurality of multi-component LNP compositions comprising exemplary compounds of Formula (II) and mRNA.

LNP compositions of the present disclosure comprising one of COMPOUND NOS. 44-76 and RNA encoding firefly luciferase (TriLink BioTechnologies) were prepared as described in Example 77. The LNP compositions are shown in Table 62.

TABLE 62

| LNPs | One of | | | PEG- | | |
|---|---|---|---|---|---|---|
| comprising | COMPOUND | DOPC | Chol | DMG | Total | |
| COMPOUND | NOS. 44-76 | (% | (% | (% | Lipid | Lipid:RNA |
| NO. | (% moles) | moles) | moles) | moles) | (mM) | (w/w) |
| 44 | 50 | 10 | 38.5 | 1.5 | 25 | 80 |
| 45 | 50 | 10 | 38.5 | 1.5 | 25 | 80 |
| 46 | 50 | 10 | 38.5 | 1.5 | 25 | 80 |
| 49 | 50 | 10 | 38.5 | 1.5 | 25 | 80 |
| 51 | 50 | 10 | 38.5 | 1.5 | 25 | 80 |
| 52 | 50 | 10 | 38.5 | 1.5 | 25 | 80 |
| 54 | 50 | 10 | 38.5 | 1.5 | 25 | 80 |
| 55 | 50 | 10 | 38.5 | 1.5 | 25 | 80 |
| 56 | 50 | 10 | 38.5 | 1.5 | 25 | 80 |
| 57 | 50 | 10 | 38.5 | 1.5 | 25 | 80 |
| 58 | 50 | 10 | 38.5 | 1.5 | 25 | 80 |
| 59 | 50 | 10 | 38.5 | 1.5 | 25 | 80 |
| 60 | 50 | 10 | 38.5 | 1.5 | 25 | 80 |
| 61 | 50 | 10 | 38.5 | 1.5 | 25 | 80 |
| 62 | 50 | 10 | 38.5 | 1.5 | 25 | 80 |
| 63 | 50 | 10 | 38.5 | 1.5 | 25 | 80 |
| 65 | 50 | 10 | 38.5 | 1.5 | 25 | 80 |
| 66 | 50 | 10 | 38.5 | 1.5 | 25 | 80 |
| 67 | 50 | 10 | 38.5 | 1.5 | 25 | 80 |
| 68 | 50 | 10 | 38.5 | 1.5 | 25 | 80 |
| 69 | 50 | 10 | 38.5 | 1.5 | 25 | 80 |
| 70 | 50 | 10 | 38.5 | 1.5 | 25 | 80 |
| 71 | 50 | 10 | 38.5 | 1.5 | 25 | 80 |
| 72 | 50 | 10 | 38.5 | 1.5 | 25 | 80 |
| 73 | 50 | 10 | 38.5 | 1.5 | 25 | 80 |
| 74 | 50 | 10 | 38.5 | 1.5 | 25 | 80 |
| 75 | 50 | 10 | 38.5 | 1.5 | 25 | 80 |
| 76 | 50 | 10 | 38.5 | 1.5 | 25 | 80 |

In three different experiments, adult female BALB/C mice (n=3/group) were intravenously administered 0.5 mg/kg of 5′-CleanCap—fLuciferase mRNA (TriLink Biotech) formulated with the LNP compositions shown in Table 62. In one experiment, another group of mice was treated with 0.5 mg/kg of RNA formulated within an LNP composition comprising the following compound 100:

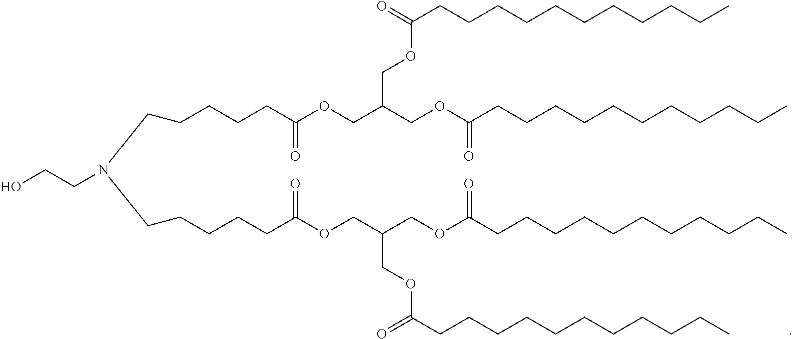 [SMILES: CCCCCCCCCCCC(=O)OCC(COC(=O)CCCCCCCCCCC)COC(=O)CCCCCN(CCO)CCCCCC(=O)OCC(COC(=O)CCCCCCCCCCC)COC(=O)CCCCCCCCCCC]

In each experiment, one group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results for the three different experiments are shown in Table 63, Table 64, and Table 65.

TABLE 63

| LNPs | |
|---|---|
| comprising | Total Flux |
| COMPOUND NO. | (p/s) |
| 44 | 5.73E+08 |
| 45 | 2.42E+10 |
| 46 | 4.97E+08 |
| 49 | 2.00E+11 |
| 51 | 2.65E+10 |
| 52 | 1.81E+11 |
| 54 | 1.30E+11 |
| 55 | 7.89E+10 |
| 56 | 9.88E+10 |
| 57 | 9.69E+10 |
| 65 | 1.30E+11 |
| 66 | 3.25E+11 |
| vehicle | 2.54E+05 |

TABLE 64

| LNPs | |
|---|---|
| comprising | Total Flux |
| COMPOUND NO. | (p/s) |
| 36 | 3.25E+11 |
| 58 | 4.38E+09 |
| 59 | 1.06E+11 |
| 60 | 6.51E+10 |
| 61 | 1.04E+11 |
| 63 | 3.48E+05 |
| 68 | 1.37E+11 |
| 69 | 1.42E+11 |
| 70 | 1.30E+11 |
| 100 | 3.24E+10 |
| vehicle | 4.76E+05 |

TABLE 65

| LNPs | |
|---|---|
| comprising | Total Flux |
| COMPOUND NO. | (p/s) |
| 62 | 2.20E+11 |
| 67 | 3.79E+11 |
| 71 | 1.78E+11 |
| 72 | 2.40E+11 |
| 73 | 1.61E+11 |
| 74 | 1.05E+11 |
| 75 | 5.17E+09 |
| 76 | 4.88E+09 |
| vehicle | 5.48E+05 |

Visual analysis of the BLI images revealed that the BLI signal was predominantly located in the live. Accordingly, as shown in Table 63, Table 64, and Table 65, LNP compositions of the present disclosure successfully delivered mRNA in vivo, predominantly to cells in the liver, and the encoded transgene was subsequently expressed by the cells. The results of this example also show that RNA delivery potency increases with increasing number of cyclohexyl-substituted tails in the lipid compound (compare LNPs comprising compound 100, COMPOUND NO. 68 and COMPOUND NO. 36).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A compound of Formula (I): 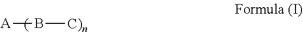
or a salt thereof, wherein:
A is: 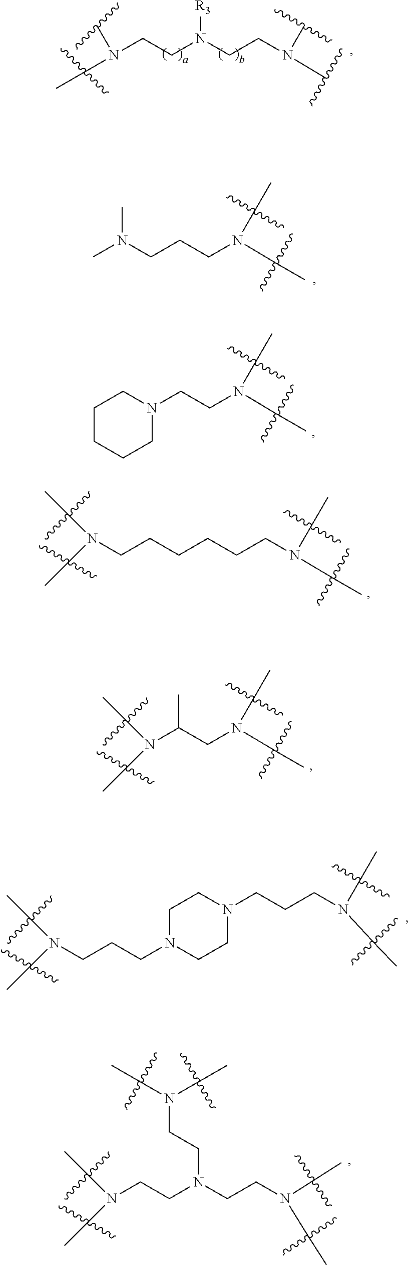 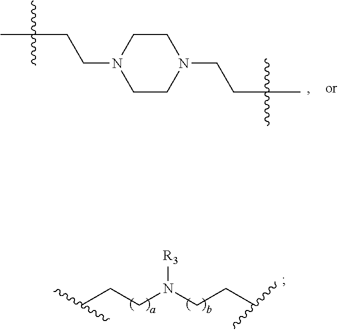
each B is independently 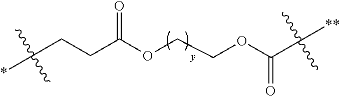
in which * indicates attachment to A and ** indicates attachment to C,
each C is independently 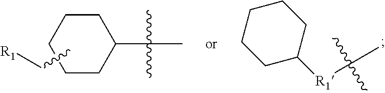
n is an integer ranging from 2 to 6;
a is an integer ranging from 1 to 5;
b is an integer ranging from 1 to 5;
each y is independently an integer ranging from 1 to 10;
each R₁ is independently unbranched C1-C18 alkyl optionally substituted with one or more C3-C12 cycloalkyl;
each R₁′ is independently unbranched C1-C18 alkylene; and
R₃ is C1-C10 alkyl optionally substituted with one or more hydroxyl or —NH—(C═O)—(C1-C6 alkyl).

2.-20. (canceled)

21. The compound of claim 1, wherein the compound is selected from the group consisting of:

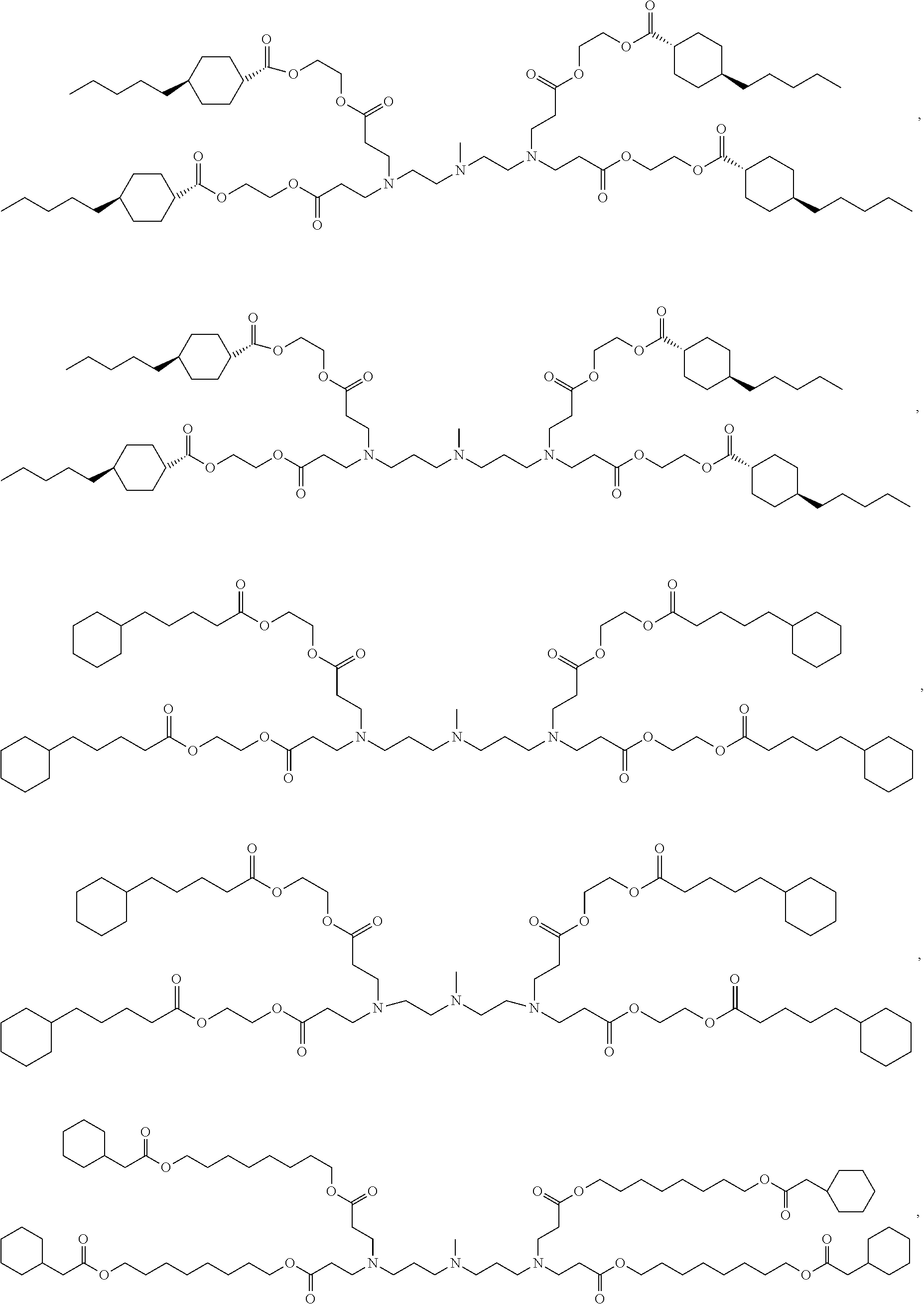

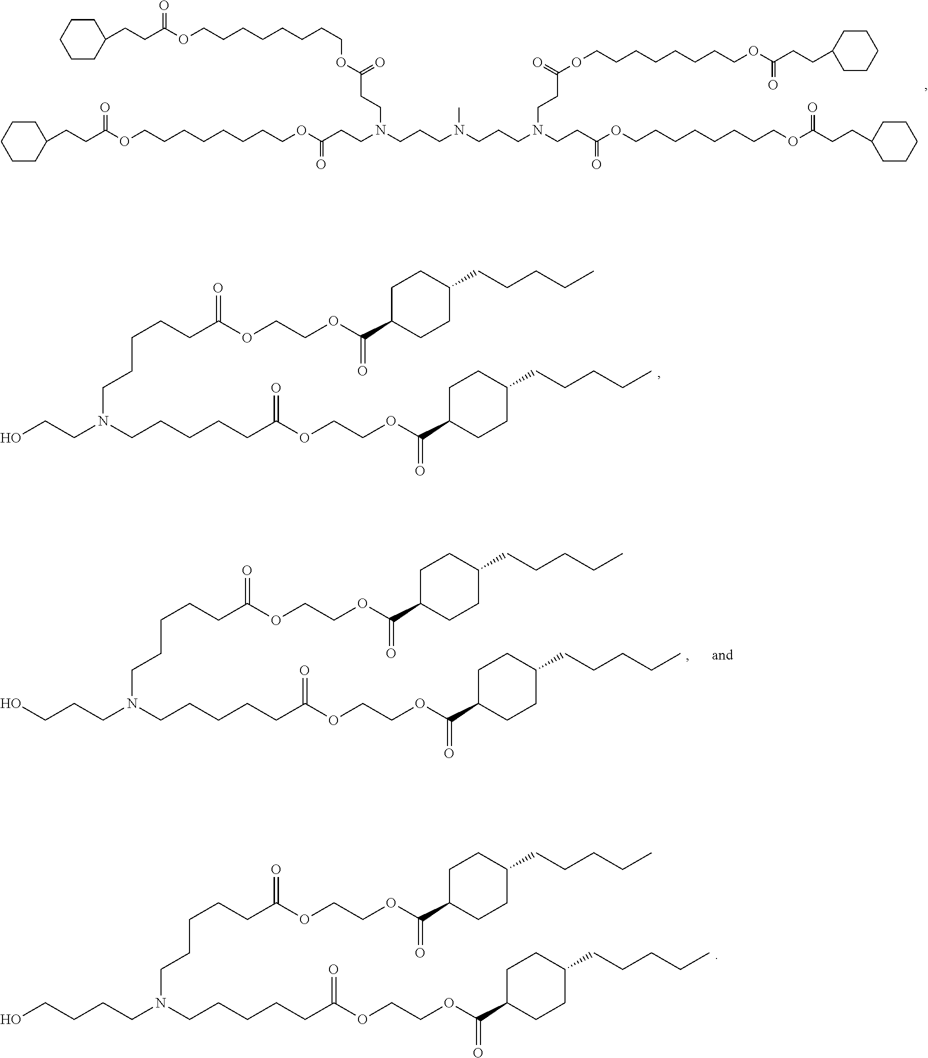 [SMILES: CN(CCCN(CCC(=O)OCCCCCCCCOC(=O)CCC1CCCCC1)CCC(=O)OCCCCCCCCOC(=O)CCC1CCCCC1)CCCN(CCC(=O)OCCCCCCCCOC(=O)CCC1CCCCC1)CCC(=O)OCCCCCCCCOC(=O)CCC1CCCCC1 ; CCCCC[C@H]1CC[C@H](C(=O)OCCOC(=O)CCCCCN(CCO)CCCCCC(=O)OCCOC(=O)[C@H]2CC[C@H](CCCCC)CC2)CC1 ; CCCCC[C@H]1CC[C@H](C(=O)OCCOC(=O)CCCCCN(CCCO)CCCCCC(=O)OCCOC(=O)[C@H]2CC[C@H](CCCCC)CC2)CC1 ; CCCCC[C@H]1CC[C@H](C(=O)OCCOC(=O)CCCCCN(CCCCO)CCCCCC(=O)OCCOC(=O)[C@H]2CC[C@H](CCCCC)CC2)CC1]

22. A compound of Formula (II): 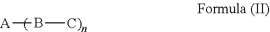
or a salt thereof,
wherein:
A is: 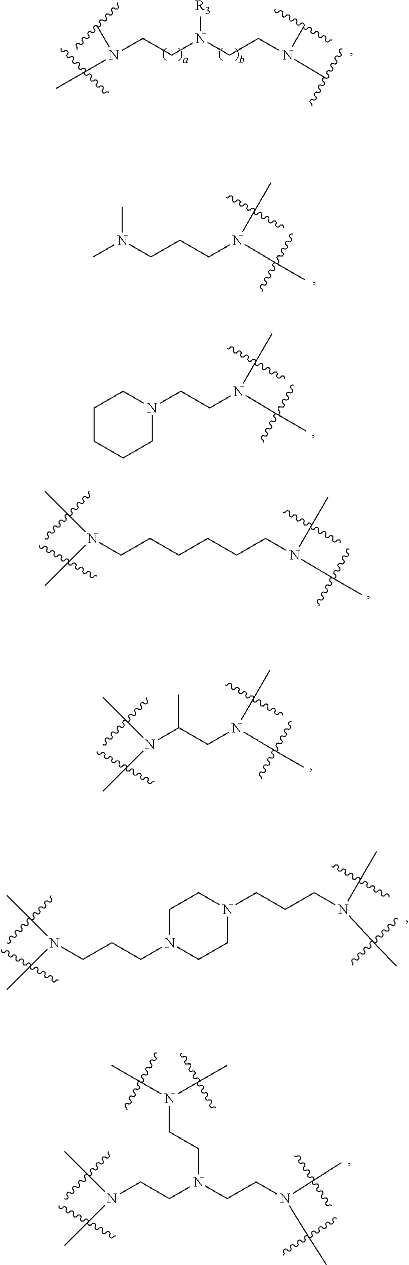 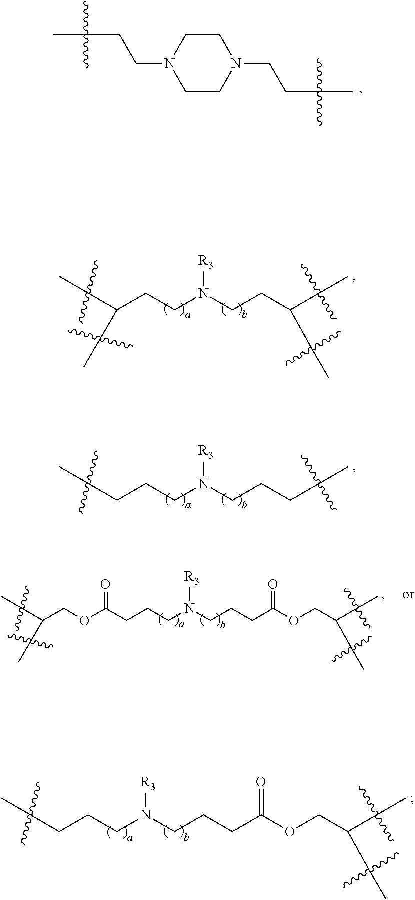
each B is independently 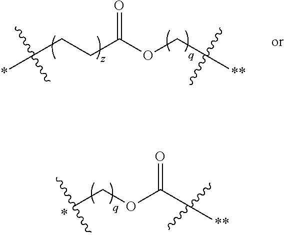
in which * indicates attachment to A and ** indicates attachment to C,
each C is independently 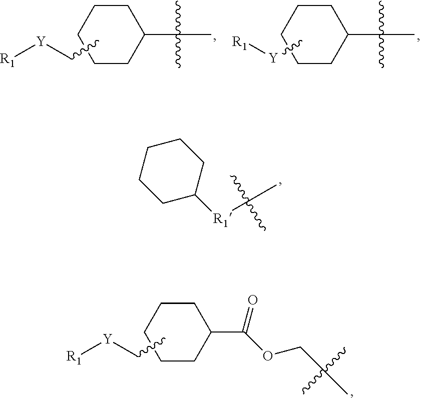
or C1-C18 alkyl;
n is an integer ranging from 2 to 6;
a is an integer ranging from 1 to 5;
b is an integer ranging from 1 to 5;
each R₁ is independently C1-C18 alkyl or C2-C18 alkenyl, wherein the C1-C18 alkyl or C2-C18 alkenyl is optionally substituted with one or more C3-C12 cycloalkyl;
each R₁′ is independently unbranched C1-C18 alkylene;
R₃ is (i) C1-C10 alkyl optionally substituted with one or more hydroxyl, —NH—(C—O)—(C1-C6 alkyl), or phenyl, or (ii) cyclohexyl optionally substituted with one or more hydroxyl or —(C1-C6 alkylene)-hydroxyl;
each Y is independently 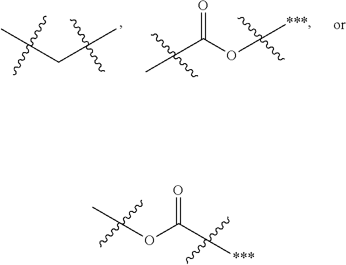
in which *** indicates attachment to R₁;
each p is independently an integer ranging from 0 to 3;
each q is independently 0 or 1; and
each z is independently 0 or 1.

23.-24. (canceled)

25. The compound of claim 22, wherein A is

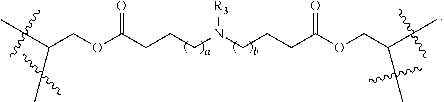

26. (canceled)

27. The compound of claim 22, wherein each B is 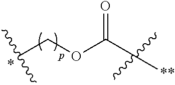
in which * indicates attachment to A and ** indicates attachment to C.

28. (canceled)

29. The compound of claim 22, wherein each C is

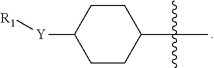

30.-34. (canceled)

35. The compound of claim 22, wherein each Y is

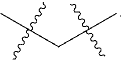 [SMILES: *C*]

36. The compound of claim 22, wherein each R₁ is C1-C18 alkyl.

37. (canceled)

38. The compound of claim 22, wherein each C is 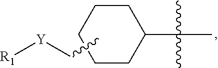
each Y is 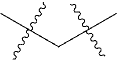 [SMILES: *C*]
and each R₁ is C1-C18 alkyl.

39.-53. (canceled)

54. The compound of claim 22, wherein a is 3; b is 3; or a is 3 and b is 3.

55.-58. (canceled)

59. The compound of claim 22, wherein p is 0, 1, or 3.

60.-62. (canceled)

63. The compound of claim 22, wherein R₃ is C1-C10 alkyl substituted with one or more hydroxyl.

64.-65. (canceled)

66. The compound of claim 22, wherein n is 4.

67. The compound of claim 22, wherein the compound is selected from the group consisting of:

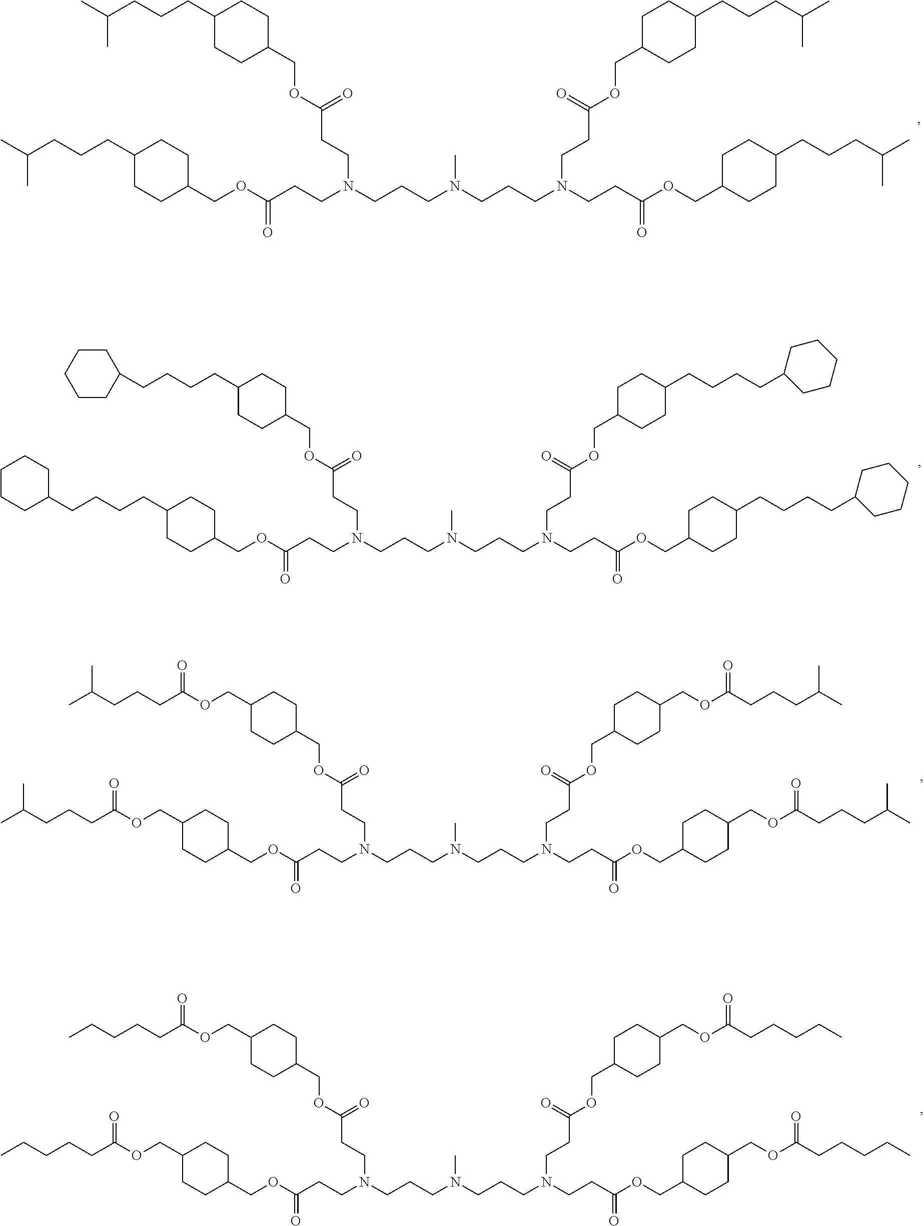

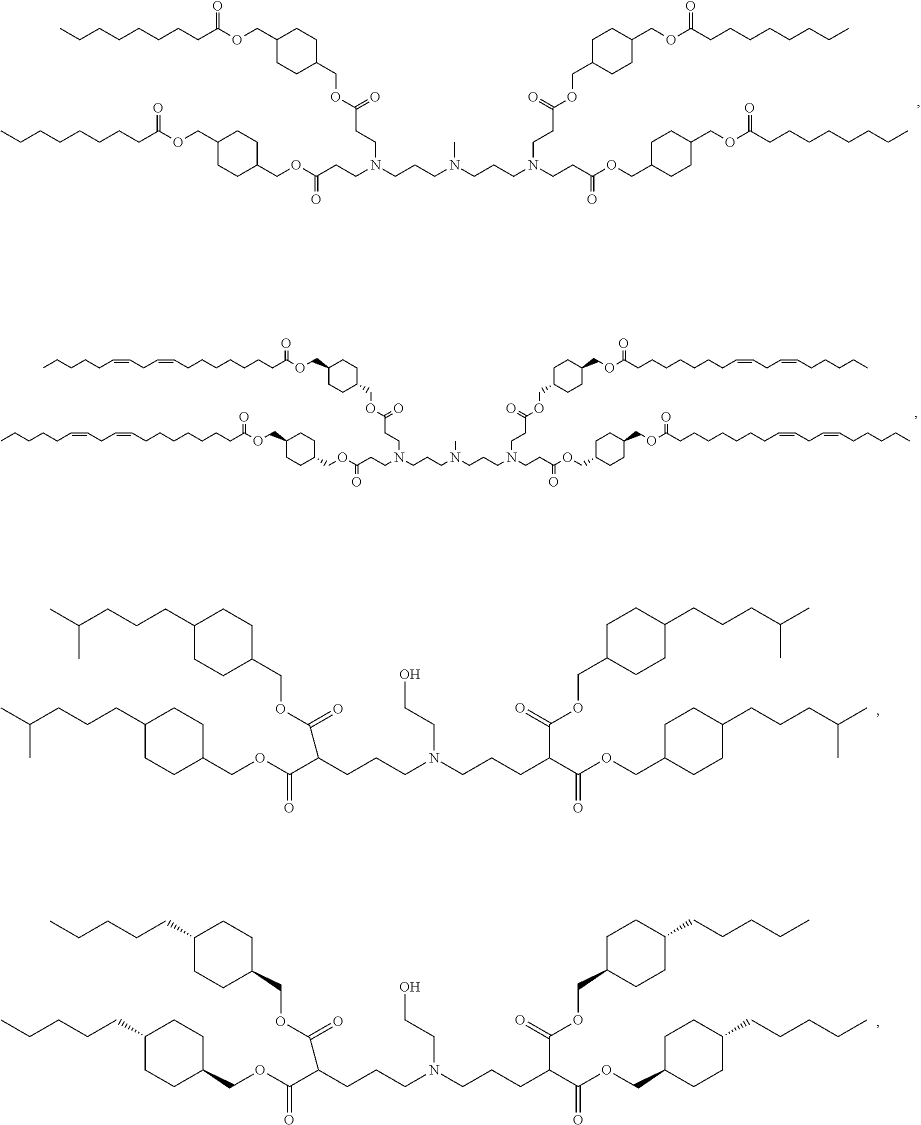

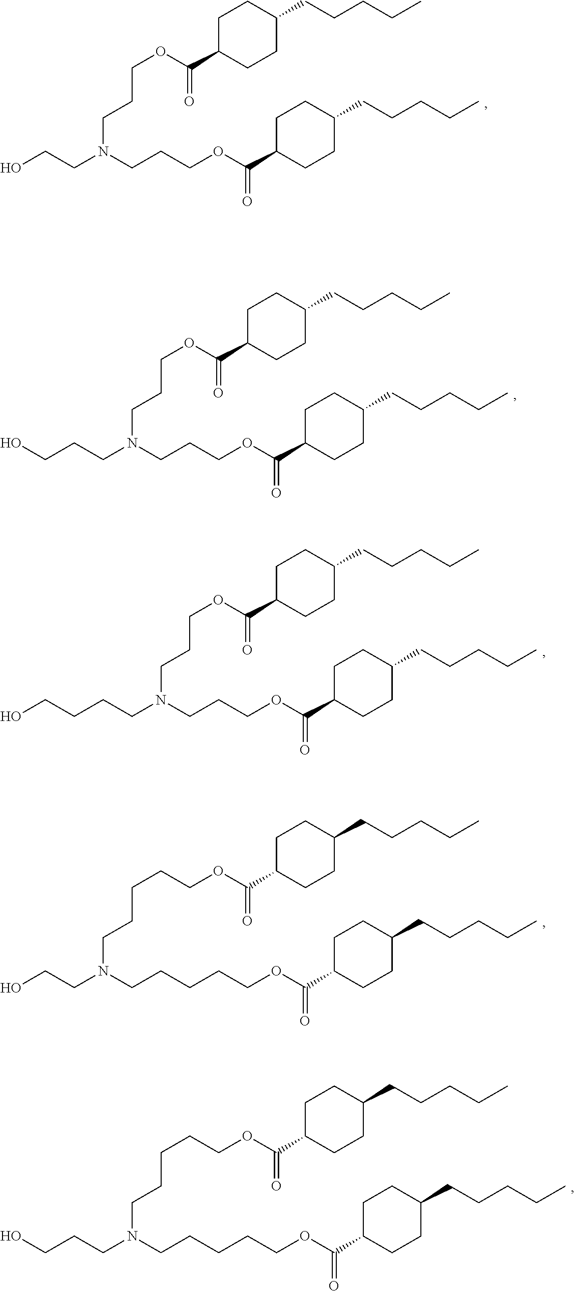 [SMILES: CCCCC[C@H]1CC[C@H](C(=O)OCCCN(CCO)CCCOC(=O)[C@H]2CC[C@H](CCCCC)CC2)CC1 ; CCCCC[C@H]1CC[C@H](C(=O)OCCCN(CCCO)CCCOC(=O)[C@H]2CC[C@H](CCCCC)CC2)CC1 ; CCCCC[C@H]1CC[C@H](C(=O)OCCCN(CCCCO)CCCOC(=O)[C@H]2CC[C@H](CCCCC)CC2)CC1 ; CCCCC[C@H]1CC[C@H](C(=O)OCCCCCN(CCO)CCCCCOC(=O)[C@H]2CC[C@H](CCCCC)CC2)CC1 ; CCCCC[C@H]1CC[C@H](C(=O)OCCCCCN(CCCO)CCCCCOC(=O)[C@H]2CC[C@H](CCCCC)CC2)CC1]

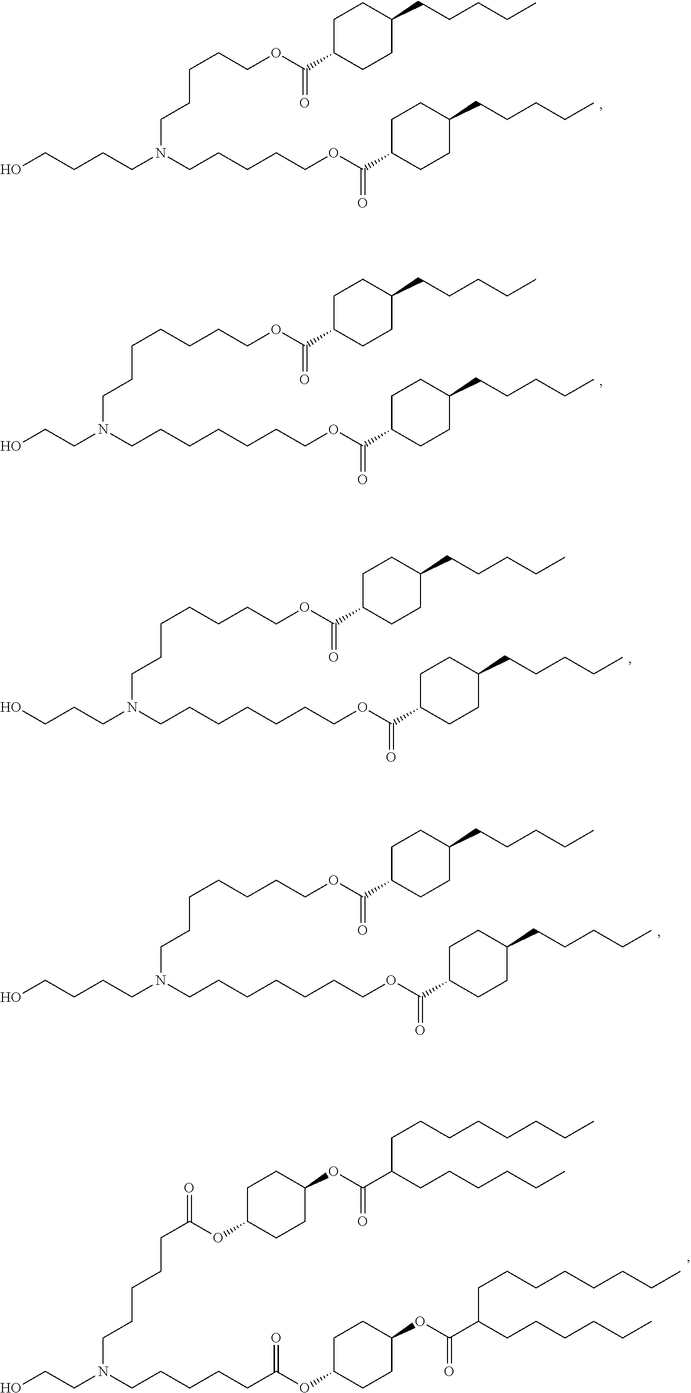 [SMILES: CCCCC[C@H]1CC[C@H](C(=O)OCCCCCN(CCCCO)CCCCCOC(=O)[C@H]2CC[C@H](CCCCC)CC2)CC1 ; CCCCC[C@H]1CC[C@H](C(=O)OCCCCCCCN(CCO)CCCCCCCOC(=O)[C@H]2CC[C@H](CCCCC)CC2)CC1 ; CCCCC[C@H]1CC[C@H](C(=O)OCCCCCCCN(CCCO)CCCCCCCOC(=O)[C@H]2CC[C@H](CCCCC)CC2)CC1 ; CCCCC[C@H]1CC[C@H](C(=O)OCCCCCCCN(CCCCO)CCCCCCCOC(=O)[C@H]2CC[C@H](CCCCC)CC2)CC1 ; CCCCCCCCC(CCCCCC)C(=O)O[C@H]1CC[C@H](OC(=O)CCCCCN(CCO)CCCCCC(=O)O[C@H]2CC[C@H](OC(=O)C(CCCCCC)CCCCCCCC)CC2)CC1]

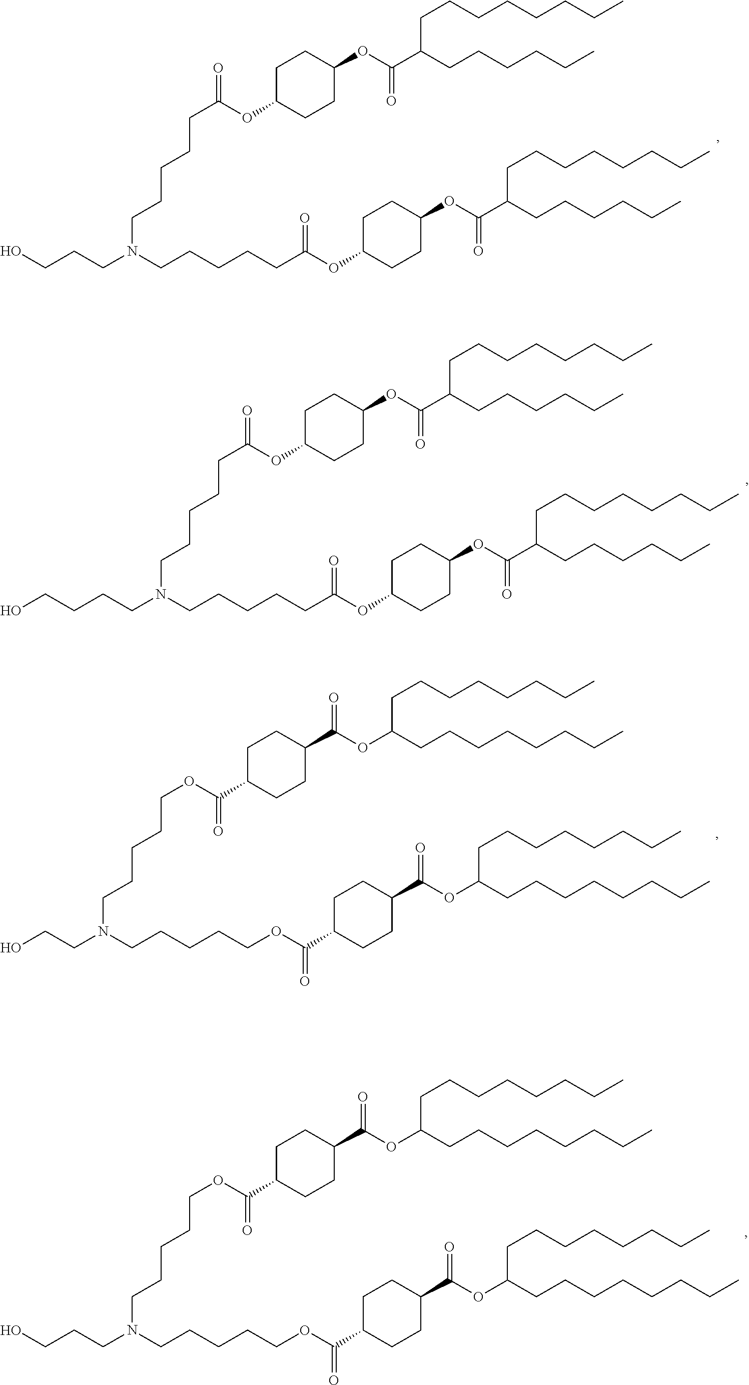 [SMILES: CCCCCCCCC(CCCCCC)C(=O)O[C@H]1CC[C@H](OC(=O)CCCCCN(CCCO)CCCCCC(=O)O[C@H]2CC[C@H](OC(=O)C(CCCCCC)CCCCCCCC)CC2)CC1 ; CCCCCCCCC(CCCCCC)C(=O)O[C@H]1CC[C@H](OC(=O)CCCCCN(CCCCO)CCCCCC(=O)O[C@H]2CC[C@H](OC(=O)C(CCCCCC)CCCCCCCC)CC2)CC1 ; CCCCCCCCC(CCCCCCCC)OC(=O)[C@H]1CC[C@H](C(=O)OCCCCCN(CCO)CCCCCOC(=O)[C@H]2CC[C@H](C(=O)OC(CCCCCCCC)CCCCCCCC)CC2)CC1 ; CCCCCCCCC(CCCCCCCC)OC(=O)[C@H]1CC[C@H](C(=O)OCCCCCN(CCCO)CCCCCOC(=O)[C@H]2CC[C@H](C(=O)OC(CCCCCCCC)CCCCCCCC)CC2)CC1]

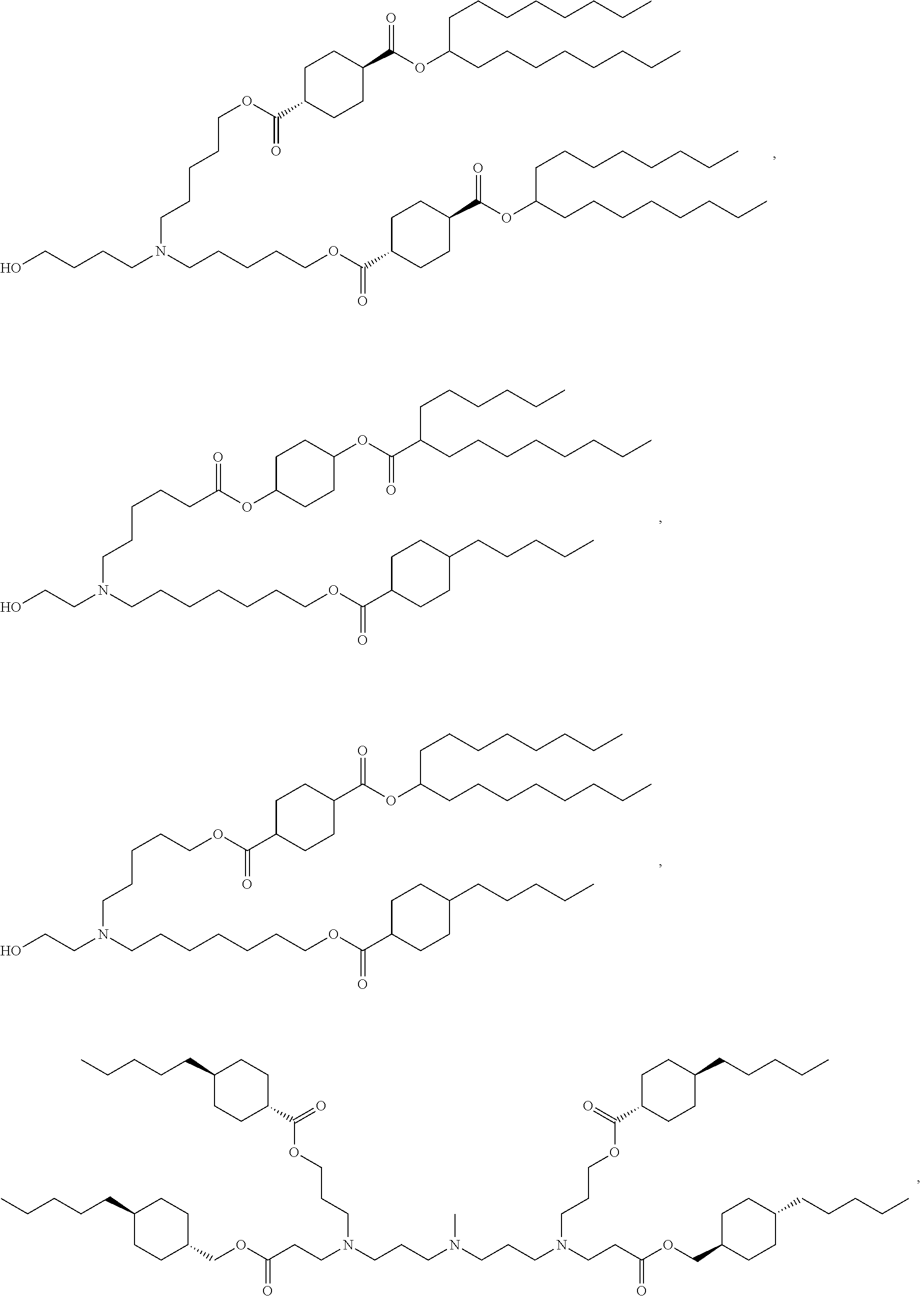

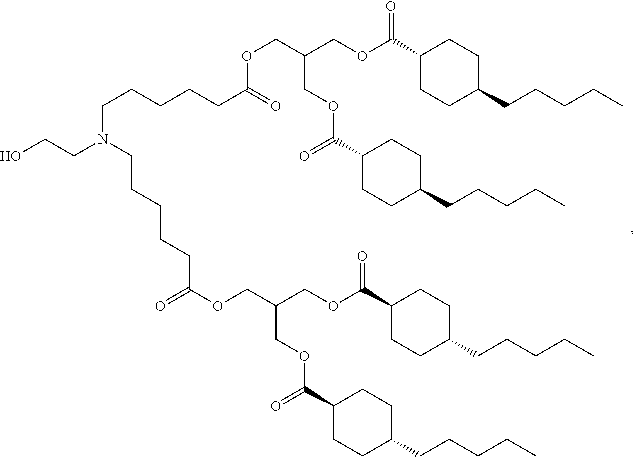 [SMILES: CCCCC[C@H]1CC[C@H](C(=O)OCC(COC(=O)CCCCCN(CCO)CCCCCC(=O)OCC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1]

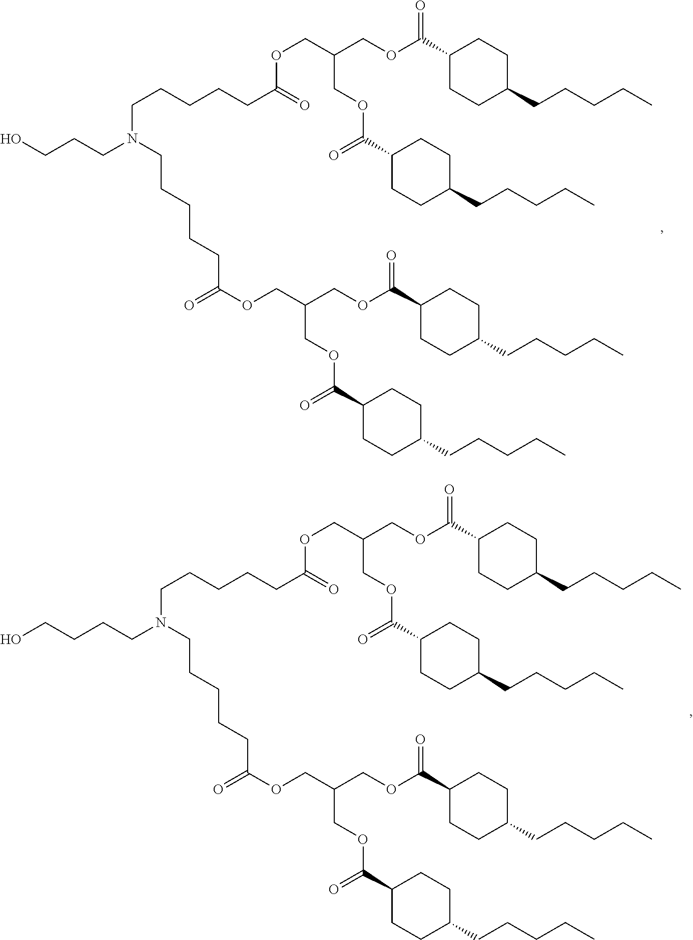

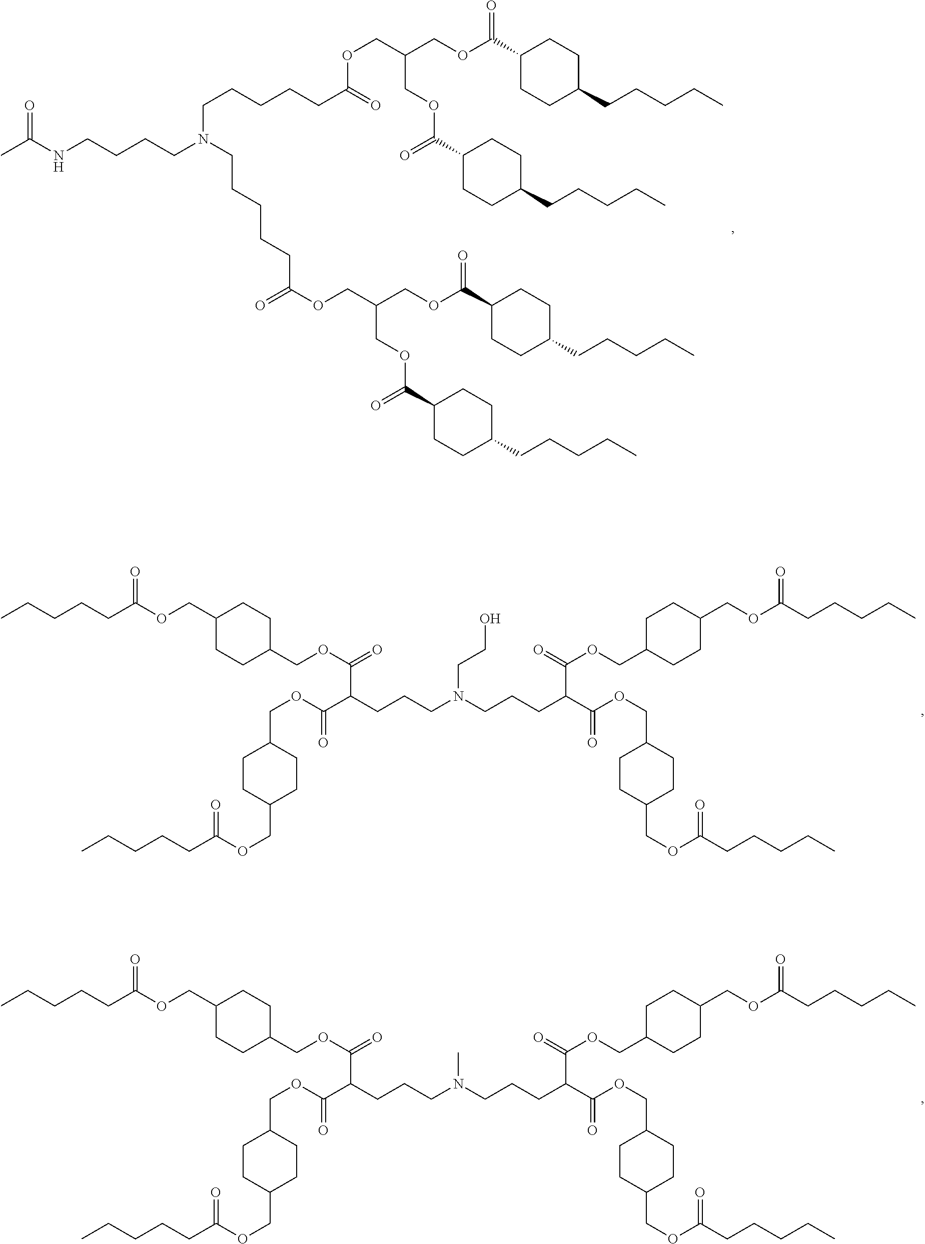

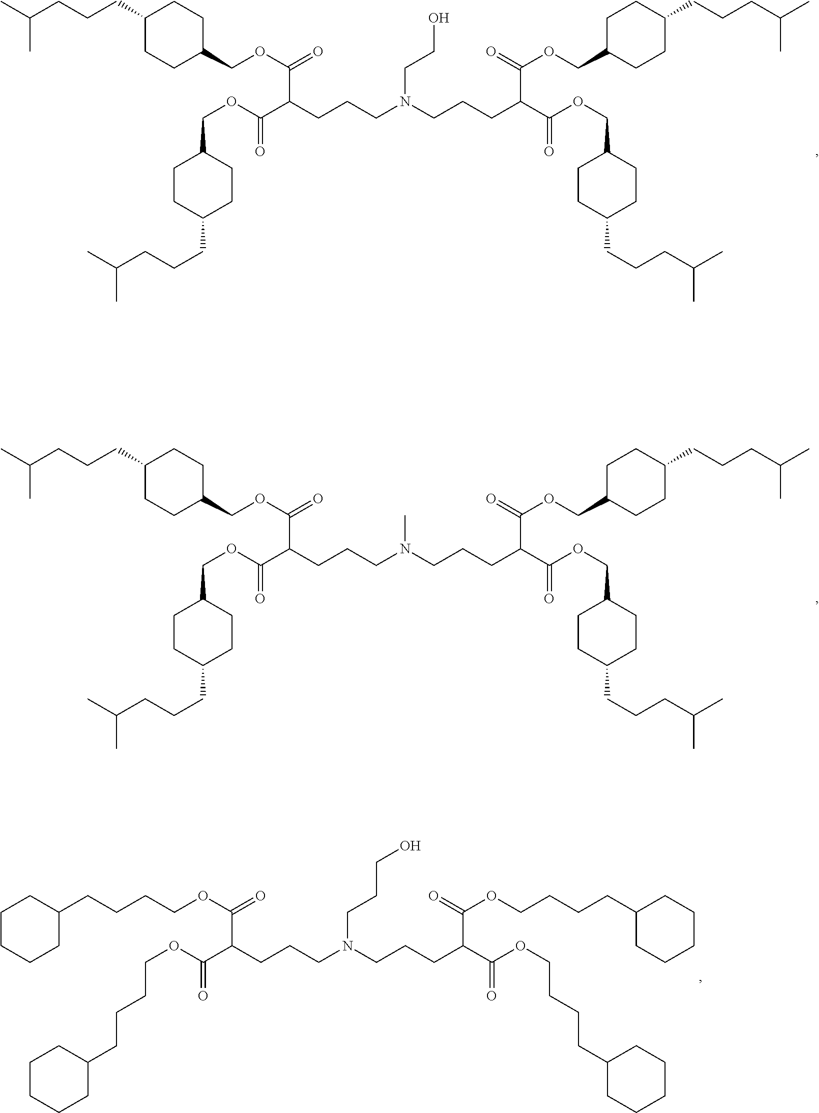

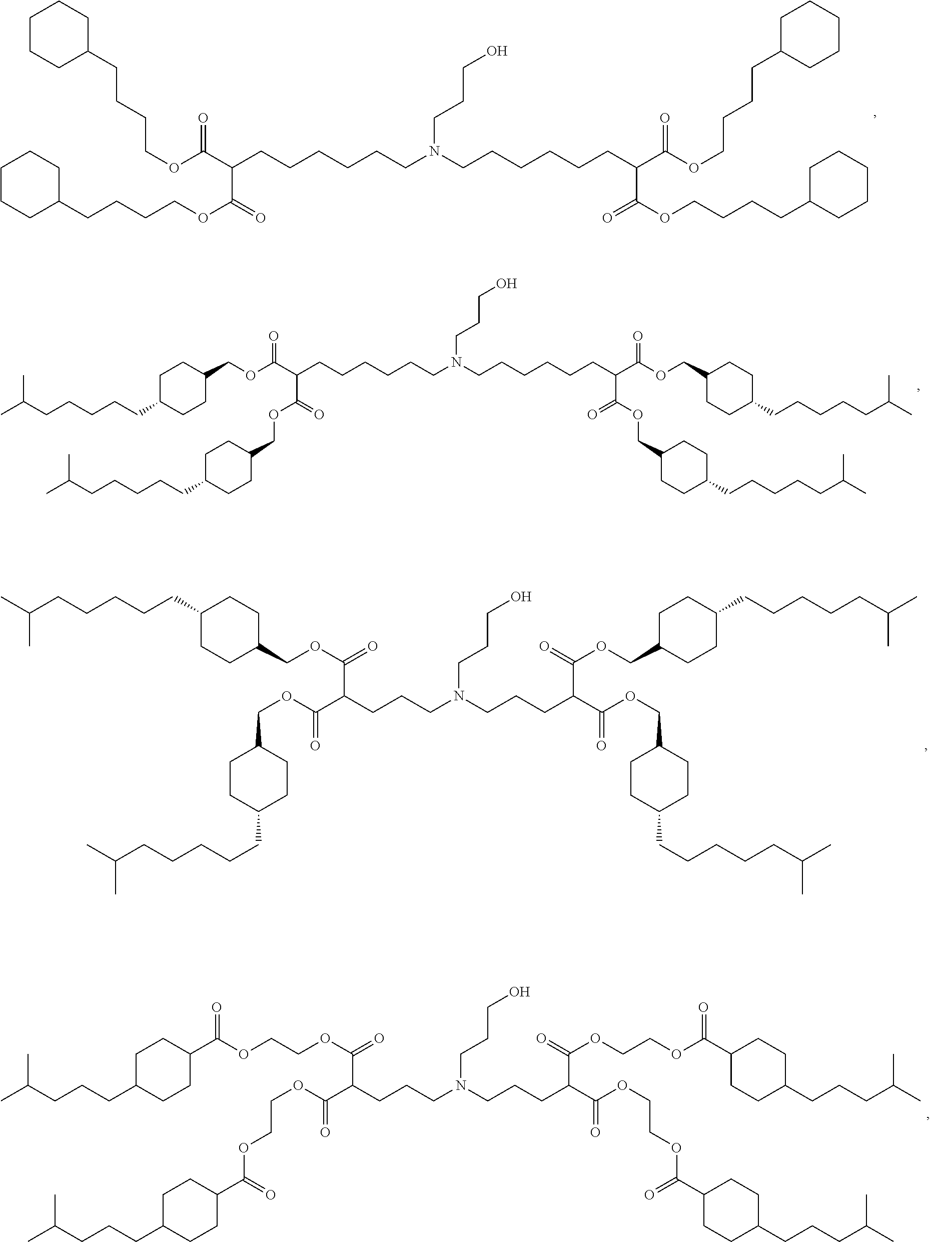

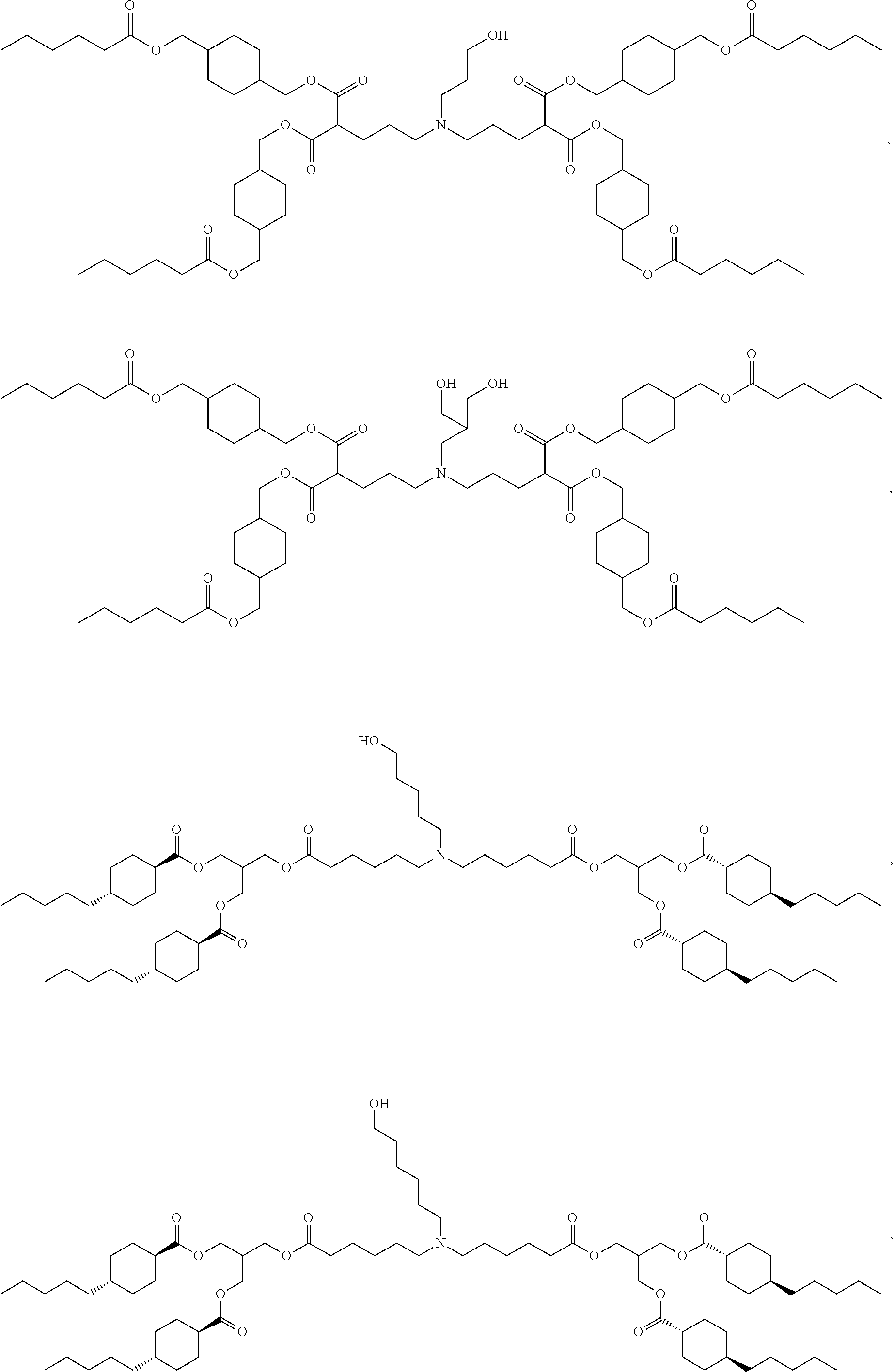

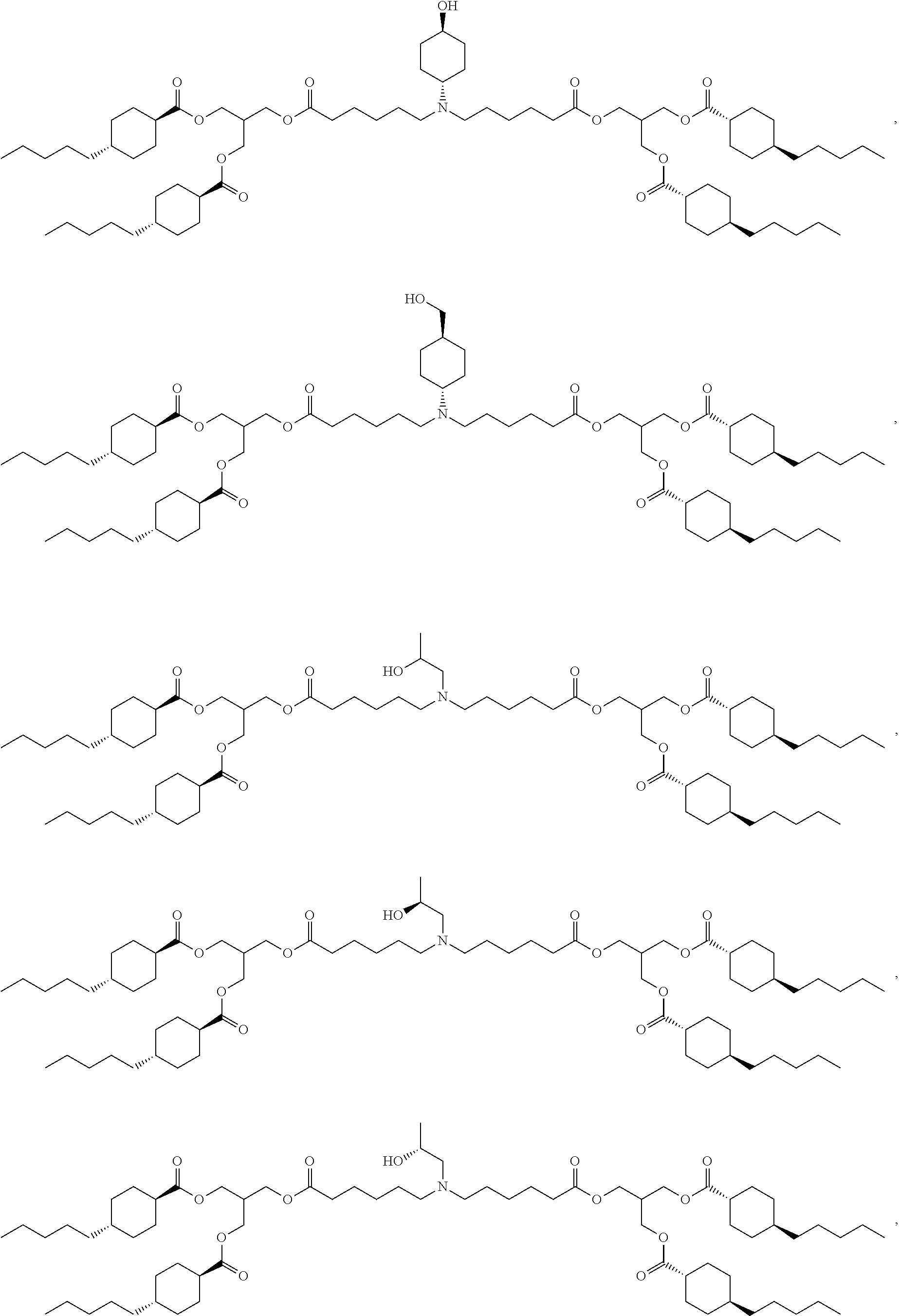

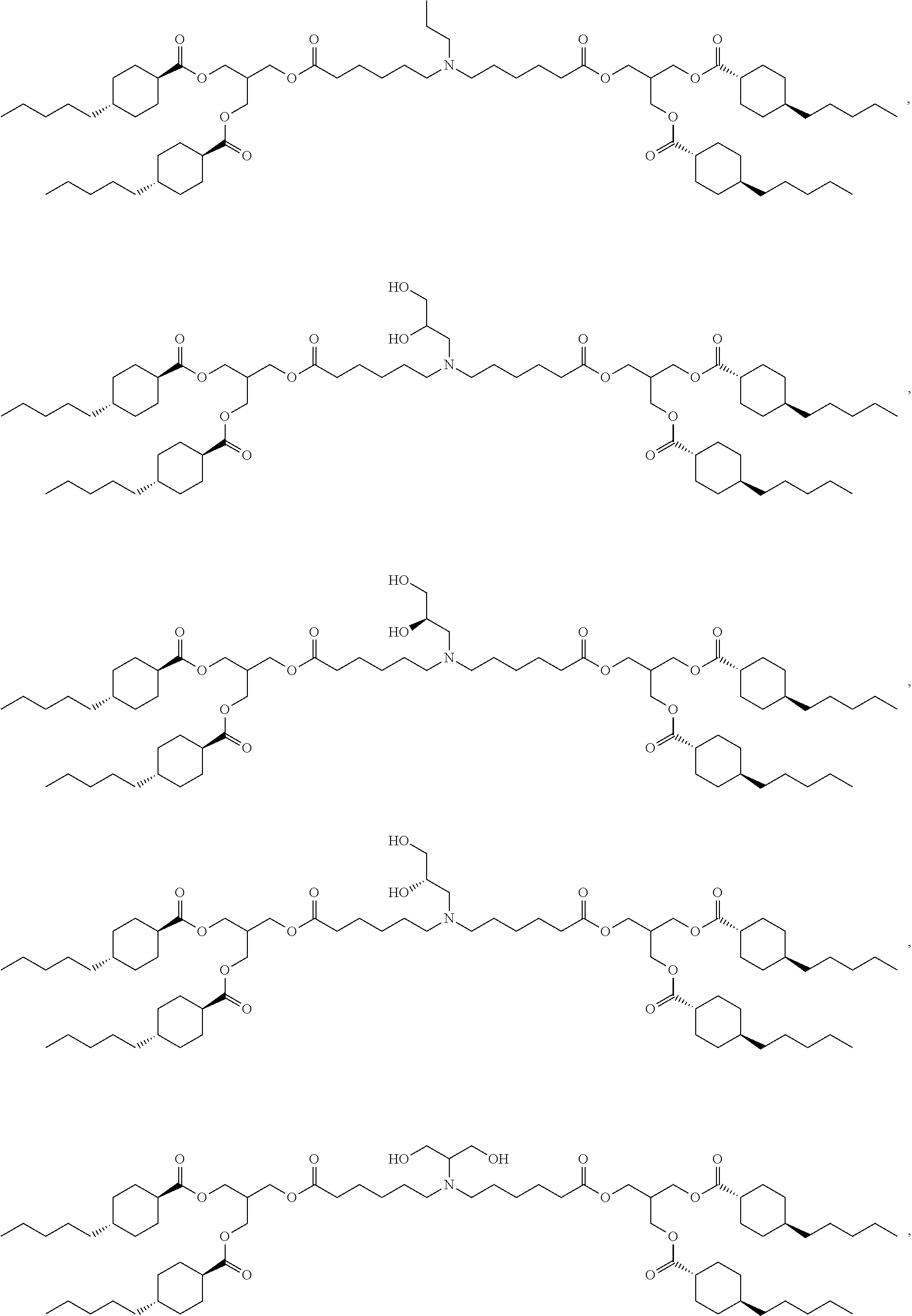

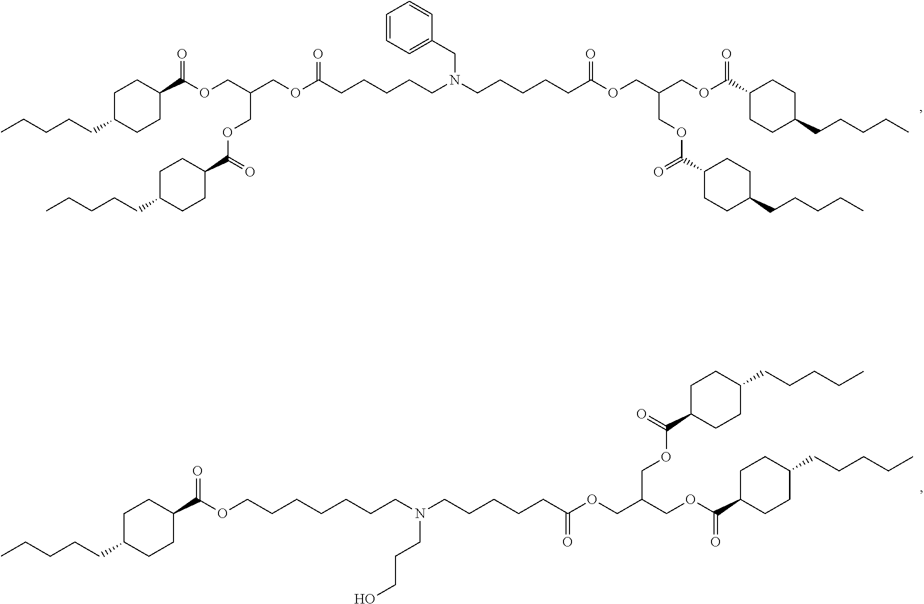

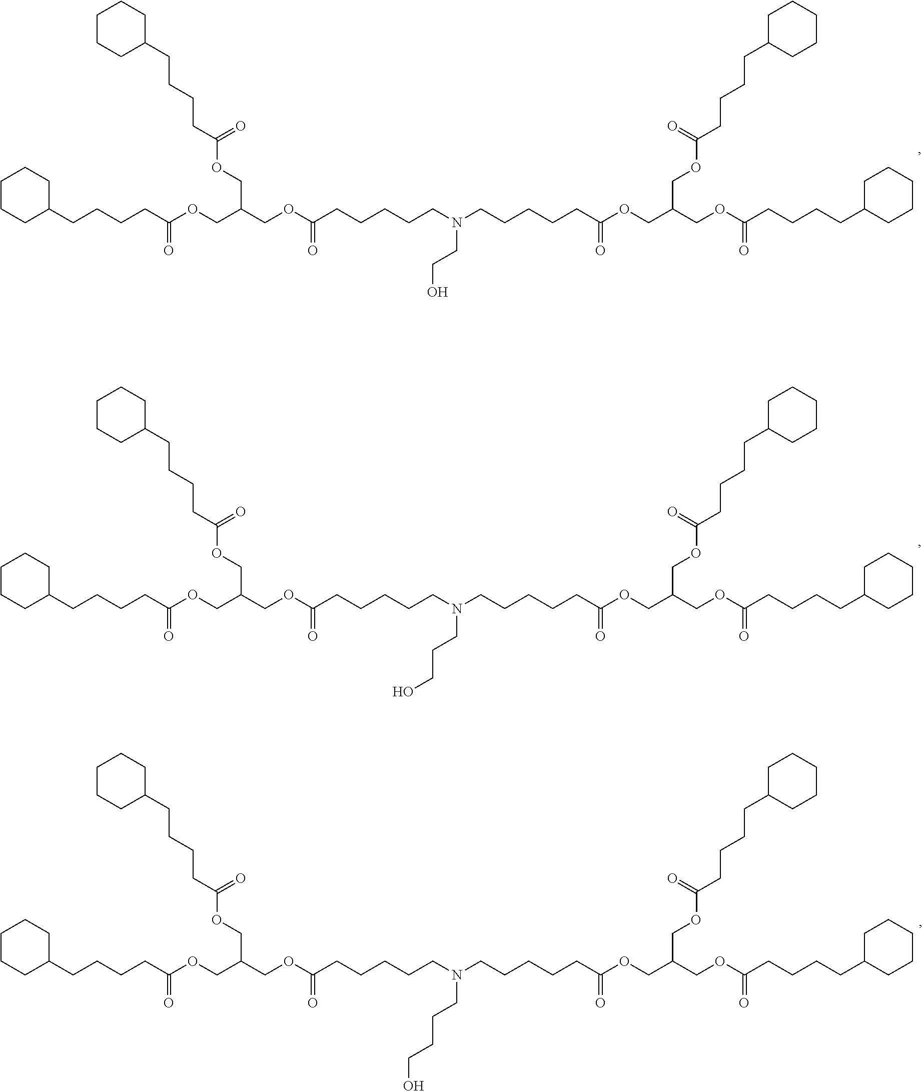 [SMILES: O=C(CCCCCN(CCO)CCCCCC(=O)OCC(COC(=O)CCCCC1CCCCC1)COC(=O)CCCCC1CCCCC1)OCC(COC(=O)CCCCC1CCCCC1)COC(=O)CCCCC1CCCCC1 ; O=C(CCCCCN(CCCO)CCCCCC(=O)OCC(COC(=O)CCCCC1CCCCC1)COC(=O)CCCCC1CCCCC1)OCC(COC(=O)CCCCC1CCCCC1)COC(=O)CCCCC1CCCCC1 ; O=C(CCCCCN(CCCCO)CCCCCC(=O)OCC(COC(=O)CCCCC1CCCCC1)COC(=O)CCCCC1CCCCC1)OCC(COC(=O)CCCCC1CCCCC1)COC(=O)CCCCC1CCCCC1]

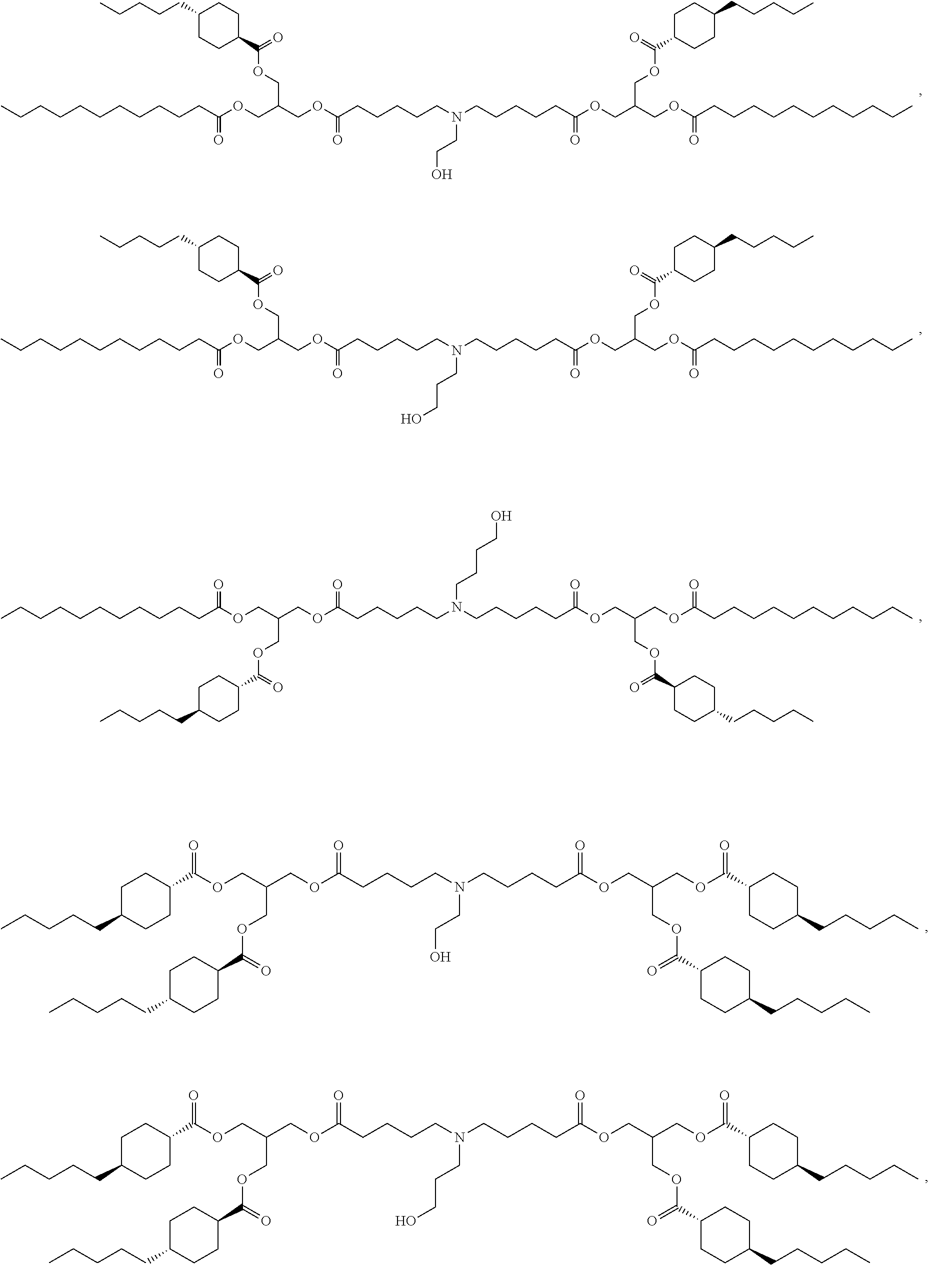

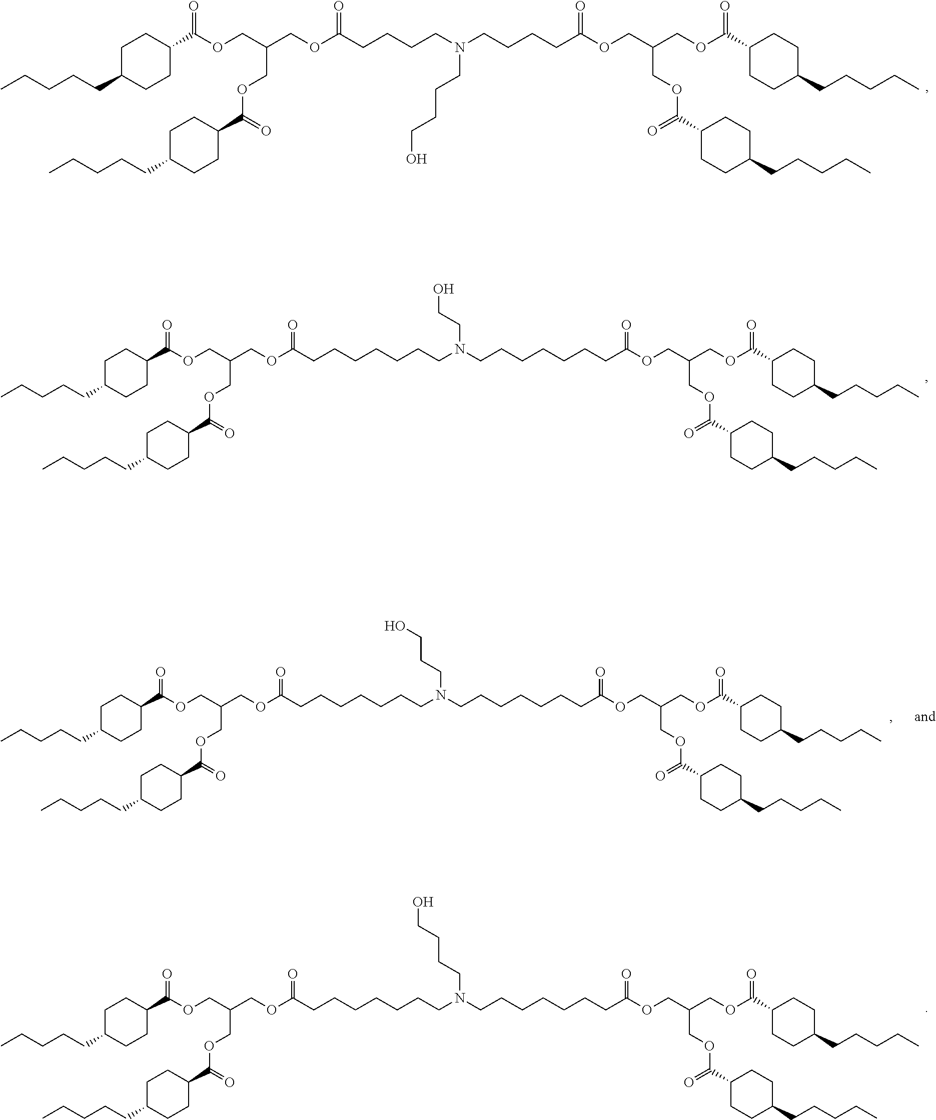

68. A composition comprising at least one lipid nanoparticle comprising at least one compound of Formula (I) of claim 1.

69.-74. (canceled)

75. A composition comprising at least one lipid nanoparticle comprising at least one compound of Formula (II) of claim 22.

76.-102. (canceled)

103. A pharmaceutical composition, comprising a composition of claim 68 and at least one pharmaceutically-acceptable excipient or diluent.

104. A method of delivering at least one nucleic acid to at least one cell comprising contacting the at least one cell with a composition of claim 68.

105. A method of genetically modifying at least one cell comprising contacting the at least one cell with a composition of claim 68.

106.-108. (canceled)

109. A method of treating at least one disease or disorder in a subject in need thereof comprising administering to the subject at least one therapeutically effective amount of the composition of claim 68.

110.-112. (canceled)

113. A pharmaceutical composition, comprising a composition of claim 75 and at least one pharmaceutically-acceptable excipient or diluent.

114. A method of delivering at least one nucleic acid to at least one cell comprising contacting the at least one cell with a composition of claim 75.

115. A method of genetically modifying at least one cell comprising contacting the at least one cell with a composition of claim 75.

116. A method of treating at least one disease or disorder in a subject in need thereof comprising administering to the subject at least one therapeutically effective amount of the composition of claim 75.
